SEQUENCE LISTING (124 sequences)

SEQ ID NO: 1 - protein, 12 aa, Artificial Sequence
  Synthetic Construct
EVKALAEKVLKK

SEQ ID NO: 2 - protein, 18 aa, Artificial Sequence
  Synthetic Construct
SAVALSAVALSKPGHVNA

SEQ ID NO: 3 - protein, 17 aa, Artificial Sequence
  Synthetic Construct
AVALSAVALSKPGHVNA

SEQ ID NO: 4 - protein, 16 aa, Artificial Sequence
  Synthetic Construct
VALSAVALSKPGHVNA

SEQ ID NO: 5 - protein, 19 aa, Artificial Sequence
  Synthetic Construct
FSAVALSAVALSKPGHVNA

SEQ ID NO: 6 - protein, 18 aa, Artificial Sequence
  Synthetic Construct
VAGVTLASASTLDKDIKD

SEQ ID NO: 7 - protein, 19 aa, Artificial Sequence
  Synthetic Construct
LKDVLSSYLSTSSSSSTSK

SEQ ID NO: 8 - protein, 15 aa, Artificial Sequence
  Synthetic Construct
ALSAVALSKPGHVNA

SEQ ID NO: 9 - protein, 17 aa, Artificial Sequence
  Synthetic Construct
AQNGNTNKIEVDNIVYK

SEQ ID NO: 10 - protein, 18 aa, Artificial Sequence
  Synthetic Construct
VAGVTLASASTLDKDVKE

SEQ ID NO: 11 - protein, 20 aa, Artificial Sequence
  Synthetic Construct
VIVVVAAIGGGLNNKGKSSS

SEQ ID NO: 12 - protein, 13 aa, Artificial Sequence
  Synthetic Construct
DEVKALAEKVLKK

SEQ ID NO: 13 - protein, 15 aa, Artificial Sequence
  Synthetic Construct
GNDTPADSAVKARIV

SEQ ID NO: 14 - protein, 11 aa, Artificial Sequence
  Synthetic Construct
HDVIQNALNAK

SEQ ID NO: 15 - protein, 15 aa, Artificial Sequence
  Synthetic Construct
LSSYLSTSSSSSTSK

SEQ ID NO: 16 - protein, 13 aa, Artificial Sequence
  Synthetic Construct
FSQATNAYFIKGA

SEQ ID NO: 17 - protein, 14 aa, Artificial Sequence
  Synthetic Construct
AADKSQVKVGVLQL

SEQ ID NO: 18 - protein, 14 aa, Artificial Sequence
  Synthetic Construct
AESSDTNLVNAKAA

SEQ ID NO: 19 - protein, 18 aa, Artificial Sequence
  Synthetic Construct
ATLAGVGVSGFAATTVHA

SEQ ID NO: 20 - protein, 14 aa, Artificial Sequence
  Synthetic Construct
ALDVDGIIAQLKDA

SEQ ID NO: 21 - protein, 14 aa, Artificial Sequence
  Synthetic Construct
VQAAQAGDTKPIEV

SEQ ID NO: 22 - protein, 20 aa, Artificial Sequence
  Synthetic Construct
VNAAQNGNTNKIEVDNIVYK

SEQ ID NO: 23 - protein, 17 aa, Artificial Sequence
  Synthetic Construct
VNAAQNGNTNKIEVDNI

SEQ ID NO: 24 - protein, 18 aa, Artificial Sequence
  Synthetic Construct
SINRDDYNKAVSDGQDKL

SEQ ID NO: 25 - protein, 17 aa, Artificial Sequence
  Synthetic Construct
QSQFAQEQSEAAKATQA

SEQ ID NO: 26 - protein, 16 aa, Artificial Sequence
  Synthetic Construct
AFDNTDTSLDSTFKSA

SEQ ID NO: 27 - protein, 16 aa, Artificial Sequence
  Synthetic Construct
AIAAITDTMKKEGLAE

SEQ ID NO: 28 - protein, 19 aa, Artificial Sequence
  Synthetic Construct
DANKIKEQLEEVGATVTLK

SEQ ID NO: 29 - protein, 14 aa, Artificial Sequence
  Synthetic Construct
DTSGKAGTTKISNV

SEQ ID NO: 30 - protein, 17 aa, Artificial Sequence
  Synthetic Construct
EVASKTNDIAGDGTTTA

SEQ ID NO: 31 - protein, 14 aa, Artificial Sequence
  Synthetic Construct
GLALITAVPQVVRA

SEQ ID NO: 32 - protein, 16 aa, Artificial Sequence
  Synthetic Construct
VTDTSGKAGTTKISNV

SEQ ID NO: 33 - protein, 16 aa, Artificial Sequence
  Synthetic Construct
NKVGPKEYIPELNKSL

SEQ ID NO: 34 - DNA, 21 bp, Artificial Sequence
  Synthetic Construct
caatctgaatgaacagttgtc

SEQ ID NO: 35 - DNA, 20 bp, Artificial Sequence
  Synthetic Construct
tatcttgaccaaacttgacg

SEQ ID NO: 36 - protein, 8 aa, Artificial Sequence
  Synthetic Construct
NPSRQERR

SEQ ID NO: 37 - protein, 8 aa, Artificial Sequence
  Synthetic Construct
NPRRQERR

SEQ ID NO: 38 - protein, 8 aa, Artificial Sequence
  Synthetic Construct
NPSRRERR

SEQ ID NO: 39 - protein, 8 aa, Artificial Sequence
  Synthetic Construct
NPRRRERR

SEQ ID NO: 40 - protein, 5 aa, Artificial Sequence
  Synthetic Construct
PDENK

SEQ ID NO: 41 - protein, 5 aa, Artificial Sequence
  Synthetic Construct
KDENK

SEQ ID NO: 42 - protein, 5 aa, Artificial Sequence
  Synthetic Construct
PDEKK

SEQ ID NO: 43 - protein, 5 aa, Artificial Sequence
  Synthetic Construct
KDEKK

SEQ ID NO: 44 - protein, 6 aa, Artificial Sequence
  Synthetic Construct
VHTAPK

SEQ ID NO: 45 - protein, 6 aa, Artificial Sequence
  Synthetic Construct
KHTAPK

SEQ ID NO: 46 - protein, 6 aa, Artificial Sequence
  Synthetic Construct
VKTAPK

SEQ ID NO: 47 - protein, 6 aa, Artificial Sequence
  Synthetic Construct
VHTKPK

SEQ ID NO: 48 - protein, 6 aa, Artificial Sequence
  Synthetic Construct
KKTKPK

SEQ ID NO: 49 - DNA, 22 bp, Artificial Sequence
  Synthetic Construct
gcgatcagagcgacatctataa

SEQ ID NO: 50 - DNA, 19 bp, Artificial Sequence
  Synthetic Construct
tggttagcggaggcaatac

SEQ ID NO: 51 - DNA, 20 bp, Artificial Sequence
  Synthetic Construct
tcggtggtcgtctcttctat

SEQ ID NO: 52 - DNA, 22 bp, Artificial Sequence
  Synthetic Construct
cgtcacgtctgtaccataactc

SEQ ID NO: 53 - DNA, 22 bp, Artificial Sequence
  Synthetic Construct
tgatatggcggatggtctttac

SEQ ID NO: 54 - DNA, 20 bp, Artificial Sequence
  Synthetic Construct
actgcgcacgagtttctatc

SEQ ID NO: 55 - DNA, 21 bp, Artificial Sequence
  Synthetic Construct
ctgccatacgtacaggtgaaa

SEQ ID NO: 56 - DNA, 18 bp, Artificial Sequence
  Synthetic Construct
acggaaagcggcaaagta

SEQ ID NO: 57 - DNA, 20 bp, Artificial Sequence
  Synthetic Construct
aagtgtacgcccgtttcttc

SEQ ID NO: 58 - DNA, 20 bp, Artificial Sequence
  Synthetic Construct
gttacccggttgagcattca

SEQ ID NO: 59 - DNA, 18 bp, Artificial Sequence
  Synthetic Construct
tcagcgtgccggttattt

SEQ ID NO: 60 - DNA, 20 bp, Artificial Sequence
  Synthetic Construct
cgaggcgataaaggcgataa

SEQ ID NO: 61 - DNA, 21 bp, Artificial Sequence
  Synthetic Construct
ctacaccggtggtctgaaata

SEQ ID NO: 62 - DNA, 21 bp, Artificial Sequence
  Synthetic Construct
cctacgcgagttgcgttatag

SEQ ID NO: 63 - DNA, 20 bp, Artificial Sequence
  Synthetic Construct
acacgaccaacgaagaagtc

SEQ ID NO: 64 - DNA, 22 bp, Artificial Sequence
  Synthetic Construct
ccgtaactacggtgtggtttat

SEQ ID NO: 65 - DNA, 21 bp, Artificial Sequence
  Synthetic Construct
cggtaccgttgaagtgaaaga

SEQ ID NO: 66 - DNA, 22 bp, Artificial Sequence
  Synthetic Construct
acttcgtcccatttcaggttag

SEQ ID NO: 67 - protein, 8 aa, Artificial Sequence
  Synthetic Construct
APSRQERR

SEQ ID NO: 68 - protein, 8 aa, Artificial Sequence
  Synthetic Construct
NASRQERR

SEQ ID NO: 69 - protein, 8 aa, Artificial Sequence
  Synthetic Construct
NPARQERR

SEQ ID NO: 70 - protein, 8 aa, Artificial Sequence
  Synthetic Construct
NPSAQERR

SEQ ID NO: 71 - protein, 8 aa, Artificial Sequence
  Synthetic Construct
NPSRAERR

SEQ ID NO: 72 - protein, 8 aa, Artificial Sequence
  Synthetic Construct
NPSRQARR

SEQ ID NO: 73 - protein, 8 aa, Artificial Sequence
  Synthetic Construct
NPSRQEAR

SEQ ID NO: 74 - protein, 8 aa, Artificial Sequence
  Synthetic Construct
NPSRQERA

SEQ ID NO: 75 - protein, 5 aa, Artificial Sequence
  Synthetic Construct
ADENK

SEQ ID NO: 76 - protein, 5 aa, Artificial Sequence
  Synthetic Construct
PAENK

SEQ ID NO: 77 - protein, 5 aa, Artificial Sequence
  Synthetic Construct
PDANK

SEQ ID NO: 78 - protein, 5 aa, Artificial Sequence
  Synthetic Construct
PDEAK

SEQ ID NO: 79 - protein, 5 aa, Artificial Sequence
  Synthetic Construct
PDENA

SEQ ID NO: 80 - protein, 6 aa, Artificial Sequence
  Synthetic Construct
AHTAPK

SEQ ID NO: 81 - protein, 6 aa, Artificial Sequence
  Synthetic Construct
VATAPK

SEQ ID NO: 82 - protein, 6 aa, Artificial Sequence
  Synthetic Construct
VHAAPK

SEQ ID NO: 83 - protein, 6 aa, Artificial Sequence
  Synthetic Construct
VHTAAK

SEQ ID NO: 84 - protein, 6 aa, Artificial Sequence
  Synthetic Construct
VHTAPA

SEQ ID NO: 85 - protein, 8 aa, Artificial Sequence
  Synthetic Construct
NPSRQERR

SEQ ID NO: 86 - protein, 4 aa, Artificial Sequence
  Synthetic Construct
PDEN

SEQ ID NO: 87 - protein, 6 aa, Artificial Sequence
  Synthetic Construct
VHTAPK

SEQ ID NO: 88 - protein, 7 aa, Artificial Sequence
  Synthetic Construct
MLNERVK

SEQ ID NO: 89 - protein, 8 aa, Artificial Sequence
  Synthetic Construct
NPSRQERR

SEQ ID NO: 90 - protein, 8 aa, Artificial Sequence
  Synthetic Construct
NPRRQERR

SEQ ID NO: 91 - protein, 8 aa, Artificial Sequence
  Synthetic Construct
NPSRRERR

SEQ ID NO: 92 - protein, 8 aa, Artificial Sequence
  Synthetic Construct
NPRRRERR

SEQ ID NO: 93 - protein, 8 aa, Artificial Sequence
  Synthetic Construct
NPSRQERR

SEQ ID NO: 94 - protein, 8 aa, Artificial Sequence
  Synthetic Construct
NPRRQERR

SEQ ID NO: 95 - protein, 8 aa, Artificial Sequence
  Synthetic Construct
NPSRRERR

SEQ ID NO: 96 - protein, 8 aa, Artificial Sequence
  Synthetic Construct
NPRRQRRR

SEQ ID NO: 97 - protein, 5 aa, Artificial Sequence
  Synthetic Construct
PDENK

SEQ ID NO: 98 - protein, 5 aa, Artificial Sequence
  Synthetic Construct
KDENK

SEQ ID NO: 99 - protein, 5 aa, Artificial Sequence
  Synthetic Construct
PDEKK

SEQ ID NO: 100 - protein, 5 aa, Artificial Sequence
  Synthetic Construct
KDEKK

SEQ ID NO: 101 - protein, 5 aa, Artificial Sequence
  Synthetic Construct
PDENK

SEQ ID NO: 102 - protein, 5 aa, Artificial Sequence
  Synthetic Construct
KDENK

SEQ ID NO: 103 - protein, 5 aa, Artificial Sequence
  Synthetic Construct
PDEKK

SEQ ID NO: 104 - protein, 5 aa, Artificial Sequence
  Synthetic Construct
KDEKK

SEQ ID NO: 105 - protein, 8 aa, Artificial Sequence
  Synthetic Construct
APSRQERR

SEQ ID NO: 106 - protein, 8 aa, Artificial Sequence
  Synthetic Construct
NASRQERR

SEQ ID NO: 107 - protein, 8 aa, Artificial Sequence
  Synthetic Construct
NPARQERR

SEQ ID NO: 108 - protein, 8 aa, Artificial Sequence
  Synthetic Construct
NPSAQERR

SEQ ID NO: 109 - protein, 8 aa, Artificial Sequence
  Synthetic Construct
NPSRAERR

SEQ ID NO: 110 - protein, 8 aa, Artificial Sequence
  Synthetic Construct
NPSRQARR

SEQ ID NO: 111 - protein, 8 aa, Artificial Sequence
  Synthetic Construct
NPSRQEAR

SEQ ID NO: 112 - protein, 8 aa, Artificial Sequence
  Synthetic Construct
NPSRQERA

SEQ ID NO: 113 - protein, 5 aa, Artificial Sequence
  Synthetic Construct
ADENK

SEQ ID NO: 114 - protein, 5 aa, Artificial Sequence
  Synthetic Construct
PAENK

SEQ ID NO: 115 - protein, 5 aa, Artificial Sequence
  Synthetic Construct
PDANK

SEQ ID NO: 116 - protein, 5 aa, Artificial Sequence
  Synthetic Construct
PDEAK

SEQ ID NO: 117 - protein, 5 aa, Artificial Sequence
  Synthetic Construct
PDENA

SEQ ID NO: 118 - protein, 7 aa, Artificial Sequence
  Synthetic Construct
YTRGLPM

SEQ ID NO: 119 - protein, 6 aa, Artificial Sequence
  Synthetic Construct
GKLSNK

SEQ ID NO: 120 - protein, 7 aa, Artificial Sequence
  Synthetic Construct
LSQKSVK

SEQ ID NO: 121 - protein, 11 aa, Artificial Sequence
  Synthetic Construct
  misc_feature (6)..(6): Xaa can be any naturally occurring amino acid
  misc_feature (9)..(9): Xaa can be any naturally occurring amino acid
NVGVLXPPXLV

SEQ ID NO: 122 - protein, 10 aa, Artificial Sequence
  Synthetic Construct
SGADTTFLTK

SEQ ID NO: 123 - protein, 9 aa, Artificial Sequence
  Synthetic Construct
LVGKKVQTE

SEQ ID NO: 124 - protein, 9 aa, Artificial Sequence
  Synthetic Construct
GTLIGQDYK

(19) United States
(12) Patent Application Publication
(10) Pub. No.: US 2024/0181000 A1
(43) Pub. Date: Jun. 6, 2024

(54) LGG-DERIVED PEPTIDES AND METHODS OF USE THEREOF

(71) Applicant: Ohio State Innovation Foundation, Columbus, OH (US)

(72) Inventors: Gireesh Rajashekara, Columbus, OH (US); Dipak Kathayat, Columbus, OH (US); Gary Closs, Jr., Columbus, OH (US); Dhanashree Fnu, Columbus, OH (US)

(21) Appl. No.: 18/285,034

(22) PCT Filed: Mar. 31, 2022
(86) PCT No.: PCT/US2022/022847

§ 371 Date: Sep. 29, 2023

Related U.S. Application Data
(60) Provisional application No. 63/226,611, filed on Jul. 28, 2021.
(60) Provisional application No. 63/175,763, filed on Apr. 16, 2021.
(60) Provisional application No. 63/168,713, filed on Mar. 31, 2021.

(51) Int. Cl. A61K 38/10; A61K 38/08; A61K 45/06; A61P 31/04

(52) U.S. Cl. CPC A61K 38/10; A61K 38/08; A61K 45/06; A61P 31/04

(57) ABSTRACT

Avian pathogenic bacteria, such as avian pathogenic E. coli (APEC) and Salmonella, cause severe infections in poultry products such as chickens and turkeys leading to food contamination and disease in humans. Previously, antibiotics were used to control bacterial infections and growth, contributing to more multi-drug resistant bacterial strains. The increased restrictions on the use of antibiotics in food-producing animals necessitate the development of new antibiotic alternative therapies. Here, the efficacy of novel antibacterial probiotics and peptides were tested against APEC and Salmonella infections in chickens. The alternative treatment methods disclosed herein are needed to combat these foodborne pathogens in avian animals and to help decrease the use of conventional antibiotics in poultry.

### CROSS REFERENCE TO RELATED APPLICATIONS

This application claims the benefit of U.S. Provisional Patent Application Ser. No. 63/175,763 filed Apr. 16, 2021, U.S. Provisional Patent Application Ser. No. 63/168,713 filed Mar. 31, 2021, and U.S. Provisional Application Ser. No. 63/226,611 filed Jul. 28, 2021, which are expressly incorporated herein by reference.

### STATEMENT REGARDING FEDERALLY SPONSORED RESEARCH

This invention was made with government support under grant/contract nos. 20156800423131 and 20173864026916 awarded by the U.S. Department of Agriculture/National Institute of Food and Agriculture. The government has certain rights in the invention.

### FIELD

The present disclosure relates to the field of antimicrobial and antibacterial treatments against E. coli and Salmonella in avian species.

### BACKGROUND

E. coli is a gram-negative bacterium commonly found in the lower intestine of most organisms. Some E. coli strains are harmless; however, others are pathogenic causing serious illness in humans and other animals. Pathogenic E. coli causes infections in the gastrointestinal, skin, respiratory, and urinary systems leading to neonatal meningitis, inflammation, septicemia, mastitis, pericarditis, etc. In particular, avian pathogenic E. coli (APEC) is a group of E. coli strains that cause a variety of respiratory and skin infection in chickens, turkeys, and other avian species. APEC is the most common bacterial pathogen in chickens, costing the poultry industry millions of dollars in economic losses worldwide. APEC remains abundant in chicken farms with the disease rapidly progressing in chickens within 24-48 hours and can only be cured through the use of antimicrobial drugs. However, effective treatment to combat APEC is limited, thus impacting human health worldwide.

Similarly, Salmonella is a pathogenic gram-negative bacterium causing infections in livestock and humans. Salmonella infection primarily presents with gastrointestinal and inflammatory symptoms in humans and animals. Salmonella enterica is a Salmonella species that has a variety of serovars including Typhimurium (ST) and Enteritidis (SE). Both serovars exist as common pathogens to humans and animals. Salmonella infections arise from consumption of infected or improperly cooked livestock products, such as poultry products (chicken, turkey, etc.). The poultry industry is also economically impacted worldwide by Salmonella outbreaks imposing great risks in human populations. Treatment of Salmonella in avian populations also requires antimicrobial therapy; however, treatments remain limited to prevent spread and infections to humans.

APEC, ST, and SE are foodborne pathogens responsible for severe economic loss to the poultry industry worldwide. The rapid spread of these pathogens also contributes to disease in humans. In addition, the increased use of antibiotics in the poultry industry has led to the emergence and spread of antibiotic-resistant strains of APEC, ST, and SE. Therefore, there is a need to address these problems mentioned above and other shortcomings associated with treating and preventing avian bacterial infections.

### SUMMARY

In one aspect, disclosed herein is a composition comprising: an antimicrobial peptide comprising an amino acid sequence at least 80% identical to SEQ ID NO:21 or SEQ ID NO:32, or a functional variant thereof; and a pharmaceutically acceptable carrier.

In some embodiments, the antimicrobial peptide comprises an amino acid sequence at least 90% identical to SEQ ID NO:21 or SEQ ID NO:32. In some embodiments, the antimicrobial peptide comprises SEQ ID NO: 21. In some embodiments, the antimicrobial peptide comprises SEQ ID NO: 32

In some embodiments, the antimicrobial peptide is 10-20 amino acids in length.

In one aspect, disclosed herein is a method of treating or preventing a bacterial infection in a subject comprising administering to the subject an effective amount of an antimicrobial peptide comprising an amino acid sequence at least 80% identical to SEQ ID NO:21, SEQ ID NO:32, SEQ ID NO:85, SEQ ID NO:86, or SEQ ID NO:87, or a functional variant thereof.

In some embodiments, the antimicrobial peptide comprises an amino acid sequence at least 90% identical to SEQ ID NO:21, SEQ ID NO:32, SEQ ID NO:85, SEQ ID NO:86, or SEQ ID NO:87. In some embodiments, the antimicrobial peptide comprises an amino acid sequence to SEQ ID NO: 21. In some embodiments, the antimicrobial peptide comprises an amino acid sequence to SEQ ID NO: 32. In some embodiments, the antimicrobial peptide comprises an amino acid sequence to SEQ ID NO: 85. In some embodiments, the antimicrobial peptide comprises an amino acid sequence to SEQ ID NO: 86. In some embodiments, the antimicrobial peptide comprises an amino acid sequence to SEQ ID NO: 87.

In some embodiments, the antimicrobial peptide is administered alone or in combination with an additional antimicrobial peptide. In some embodiments, the antimicrobial peptide is administered in combination with an antibiotic therapy. In some embodiments, the antibiotic therapy is selected from a tetracycline, a sulfonamide, an aminoglycoside, a quinolone, or a β-lactam.

In some embodiments, the bacterial infection is caused by a bacterial overgrowth.

In some embodiments, the antimicrobial peptide targets a bacterial membrane. In some embodiments, the bacterial membrane is an outer membrane. In some embodiments, the bacterial membrane comprises a MlaA-OmpC/F protein system.

In some embodiments, the bacterial infection is caused by an Escherichia coli (E. coli) bacterium. In some embodiments, the E. coli is an avian pathogenic E. coli (APEC). In some embodiments, the bacterial infection is caused by a Salmonella bacterium.

In some embodiments, the subject is a chicken.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIGS. 1A-1B show that viability of APEC decreases when incubated with L. rhamnosus GG and B. lactis Bb12. FIG. 1A shows the viability of APEC in co-culture assay when incubated together with different probiotics. APEC O78 culture grown alone in co-culture media was used as a control. FIG. 1B shows the viability of APEC in trans-well migration assay. L. rhamnosus GG and B. lactis Bb12 cultures were aliquoted into the tube containing filter, whereas APEC O78 culture was aliquoted into the microcentrifuge tube below the filter. APEC O78 culture grown with APEC 078 culture above the filter was used as a control, LGG: Lacticaseibacillus rhamnosus GG, Bb12: Bifidobacterium lactis Bb12, LA: Lactobacillus acidophilus, Lbrev: Levilactobacillus brevis, *P<0.05, ***P<0.001, two-way ANOVA Bonferroni posttest.

FIG. 2 shows scanning electron microscopy (SEM) images showing morphology of untreated APEC or APEC treated with CF Ss (cell-free supernatants) of L. rhamnosus GG and B. lactis Bb12. APEC was treated with CFSs prepared from 24 h grown culture of L. rhamnosus GG and B. lactis Bb12 for 2 h at 37° C. with shaking at 200 rpm. Bars: 1 μM.

FIGS. 3A-3B show that L. rhamnosus GG and B. lactis Bb12 predominantly produces lactic acid. FIG. 3A shows the concentrations of organic acids in CFSs of different probiotics when cultured alone. FIG. 3B shows the concentrations of organic acids in CFSs of different probiotics when co-cultured with APEC. MRS media was used as a control in monoculture study and APEC 078 culture grown alone in co-culture media was used as a control in co-culture study, LGG: Lacticaseibacillus rhamnosus GG, Bb12: Bifidobacterium lactis Bb12, LA: Lactobacillus acidophilus, Lbrev: Levilactobacillus brevis.

FIG. 4 shows the percent of original inocula of APEC O78 adhered and invaded in HT-29 cells when pretreated with cell-free supernatants (CFSs) of L. rhamnosus GG and B. lactis Bb12. LGG: Lacticaseibacillus rhamnosus GG, Bb12: Bifidobacterium lactis Bb12, DMEM: Dulbecco's Modified Eagle Medium, **P<0.01, ***P<0.001, two-way ANOVA Bonferroni posttest.

FIGS. 5A-5B show the efficacy of L. rhamnosus GG and B. lactis Bb12 and their combination tested in chickens administered orally for 14 days. FIG. 5A shows that APEC load in cecum of chickens treated with L. rhamnosus GG, B. lactis Bb12 or L. rhamnosus GG and B. lactis Bb12 combination compared to PC (APEC infected but not probiotic treated; positive control) group. FIG. B shows that body weight gain of chickens treated with L. rhamnosus GG, B. lactis Bb12 or L. rhamnosus GG and B. lactis Bb12 combination compared to PC and NC (non-APEC infected and non-probiotic treated; negative control) groups, LGG: Lacticaseibacillus rhamnosus GG, Bb12: Bifidobacterium lactis Bb12, *P<0.05, **P<0.01, Man Whitney U test.

FIGS. 6A-6D show that abundance of Enterobacteriaceae is decreased in the cecum of chickens. FIG. 6A shows the alpha-diversity (Shannon index). FIG. 6B shows the beta-diversity (Bray-Curtis dissimilarity index) of cecal microbial community of chickens treated with L. rhamnosus GG compared to PC (APEC infected but not probiotic treated; positive control) and NC (non-APEC infected and non-probiotic treated; negative control) groups. FIG. 6C shows the relative abundance of cecal microbiota at the phylum level in chickens treated with L. rhamnosus GG compared to PC and NC groups, LGG: Lacticaseibacillus rhamnosus GG. FIG. 6D shows the relative abundance of cecal microbiota at the family level in chickens treated with L. rhamnosus GG compared to PC and NC groups, LGG: Lacticaseibacillus rhamnosus GG, *P<0.05, Man Whitney U test.

FIG. 7 shows that growth (%) of APEC when treated with different peptides at 12 mM concentrations. Peptides were added to the APEC suspension in a 96-well plate, and plate was incubated in TECAN Sunrise™ absorbance microplate reader at 37° C. with OD₆₀₀ measurement set at every 30 min for 12 hr. The sequences in FIG. 7 are FSAVALSAVALSKPGHVNA (SEQ ID NO: 5), AESSDTNLVNAKAA (SEQ ID NO: 18), VQAAQAGDTKPIEV (SEQ ID NO: 21), AFDNTDTSLDSTFKSA (SEQ ID NO: 26), and VTDTSGKAGTTKISNV (SEQ ID NO: 32).

FIGS. 8A-8B show the growth and viability of APEC in co-culture (MRS+LB). FIG. 8A shows the growth of APEC in co-culture (MRS+LB) media as compared to LB media. FIG. 8B shows the viability of APEC at 24 hour in co-culture media (MRS+LB) adjusted to different pH as compared to 0 h. APEC O78 culture grown in media with normal pH (6.8) was used as a growth control. Two independent experiments with replicates (n=2) were conducted.

FIGS. 9A-9D show the standard curves of organic acids generated through LC-MS/MS. Standard curves of (A) lactic acid, (B) acetic acid, (C) propionic acid, and (D) butyric acid generated through LC-MS/MS. FIG. 9A shows the standard curve of lactic acid. FIG. 9B shows the standard curve of acetic acid. FIG. 9C shows the standard curve of propionic acid. FIG. 9D shows the standard curve of butyric acid.

FIGS. 10A-10B show the quantification of L. rhamnosus GG expression. FIG. 10A shows the standard curve for L. rhamnosus GG quantitation. Curve was generated through qPCR Ct values obtained in 10-fold serial dilutions of L. rhamnosus GG DNA extracted from OD₆₀₀ 1.0 LGG (˜10⁹ CFU/mL) culture. FIG. 10B shows the quantification of L. rhamnosus GG in cecum of chickens at day 15.

FIG. 11 shows the LC-MS showing the peak intensities and retention times of peptides that showed growth inhibitory activity against APEC. The sequences in FIG. 11 are VQAAQAGDTKPIEV (SEQ ID NO: 21), AFDNTDTSLDSTFKSA (SEQ ID NO: 26), and VTDTSGKAGTTKISNV (SEQ ID NO: 32).

FIG. 12 shows the fragmentation peaks of peptides that showed growth inhibitory activity against APEC. The fragmentation peaks were used to determine the sequence of peptides. The sequences in FIG. 12 are VQAAQAGDTKPIEV (SEQ ID NO: 21), AFDNTDTSLDSTFKSA (SEQ ID NO: 26), and VTDTSGKAGTTKISNV (SEQ ID NO: 32).

FIGS. 13A-13C show the growth pattern of APEC O78 with and without peptide at 3-21 mM concentration. FIG. 13A shows the minimal inhibitory concentrations of the PN3 peptide to be 12 mM. FIG. 13B shows the minimal inhibitory concentrations of the PN5 peptide to be 15 mM. Various concentrations of peptides were added to the APEC suspension (5×10⁵ CFU/mL) in a 96-well plate. The plate was incubated in a TECAN Sunrise™ absorbance microplate reader at 37° C., measuring the OD 600 set at every 30 mins for 12 hrs. After 12 hrs. incubation the bacteria were serially diluted and plated to count CFU/mL and calculated the inhibitory percentage using (Control-treated)/treated*100. FIG. 13C shows that the bacterial inhibition percentage is increased as the peptide concentration increased.

FIGS. 14A-14B show the growth pattern of APEC O78, treated with a combination of peptides at 3-21 mM concentration with respect to no peptide control. FIG. 14A shows that the minimal inhibitory concentrations of the peptides in combination (PN3 & PN5) was 15 mM. The two peptides were added together to the APEC suspension (5×10⁵ CFU/mL) in a 96-well plate. The plate was incubated at 37° C. in a TECAN Sunrise™ absorbance microplate reader, measuring the OD 600 set at every 30 mins for 12 hrs. After incubation, the bacteria were serially diluted and plated to measure CFU/mL and calculate the inhibitory percentage using (Control-treated)/treated*100. FIG. 14B shows that bacterial inhibition percentage is increased as the peptide concentration increased.

FIGS. 15A-15B show the growth inhibition percentage of various APEC serotypes and strains. FIG. 15A shows the growth of inhibition percentage after treating with 12 mM PN3 peptide. FIG. 15B shows the growth of inhibition percentage after treating with 15 mM PN5 peptide. APEC serotypes and strains (5×10⁵ CFU/mL) were treated with peptides and grown at 37° C. for 12 hrs, followed by plating on LB agar and CFU/mL was calculated. Bacterial inhibition percentage was calculated by comparing with no peptide control.

FIGS. 16A-16B show the effect of the peptide on the growth of various probiotic strains after treating at their minimal inhibitory concentrations of 12 and 15 mM of PN3 and PN5, respectively. FIG. 16A shows the effect of 12 mM of PN3 peptide. FIG. 16B the effect of 15 mM of PN5 peptide. The bacteria (5×105 CFU/mL) were treated with peptides and grown at 37° C. for 24 hrs in an anaerobic condition except for E. coli Nissel 1917 and E. coli G58-1, which has grown in aerobic conditions for 12 hrs. After incubation, serially dilute the culture and counted the CFU/mL Bacterial inhibition percentage was calculated by comparing with no peptide treated control. Both the peptides inhibited the growth of BB12, E. coli Nissel 1917 and E. coli G58-1 altogether and showed various percentage of inhibition on the growth of E. fecalis, S. bovis, L. brevis (65-77% viable cells). LGG remained unaffected by PN3 and slightly affected by PN5. Both the peptides promoted the growth of the B. longum, B. adolescentis, and B. theoiotamicron

FIGS. 17A-17C show that the PN3 and PN5 peptide remain stable after being subjected to proteolytic degradation and heat treatment. FIG. 17A shows the peptides were incubated with proteinase K (20 mg/mL) at 37 C for 2 hrs, followed by inactivation at 100° C. for 10 min. FIG. 17B shows heat stability for PN3 peptide. FIG. 17C shows heat stability for PN5 peptide. For the heat stability assay, the peptides were subjected to temperatures 80, 100 and 121° C. for 60, 30 and 20 mins, respectively. Then, the cells (5×105 CFU/mL) were treated with proteinase K and heat-subjected peptides at their MIC and grown at 37° C. with measuring the OD 600 set at every 30 mins 12 hrs. Bacterial inhibition percentage was calculated by comparing with no peptide treated control.

FIGS. 18A-18B show the growth of APEC O78 at MIC of the peptides after growing the bacteria for 13 passages with a sublethal concentration (0.75×MIC) of peptides. FIG. 18A shows APEC O78 growth at MIC of PN3. FIG. 18B shows APEC O78 growth at MIC of PN5. The bacteria (5×105 CFU/mL) grown for 13 passages were treated with peptides at their MIC and grown at 37° C. The OD 600 was measured at every 30 mins for 12 hrs. Sublethal concentrations exposure did not cause bacterial resistance against the peptides.

FIG. 19 shows that growth pattern of STEC strains with or without peptide at minimal inhibitory concentrations of the peptides PN3 and PN5. O157:H7 and 026 (5*10≡CFU/mL) in a 96-well plate were treated with peptides and the plate was incubated in a TECAN Sunrise™ absorbance microplate reader at 37° C. with measuring the OD 600 set at every 30 mins for 12 hrs. After 12 hrs. incubation.

FIGS. 20A-20D show the growth and persistence of Salmonella enterica serovar Typhimurium (ST) mixed with other co-cultures. FIG. 20A shows ST growth and persistence in mixed media and co-cultured with Lactobacillus acidophilus (LA). FIG. 20B shows ST growth and persistence in mixed media and co-cultured with Lacticaseibacillus rhamnosus GG (LGG). FIG. 20C shows ST growth and persistence in mixed media and co-cultured with Bifidobacterium lactis (Bb12). FIG. 20D shows ST growth and persistence in mixed media and co-cultured with Levilactobacillus brevis (Lbrev). MRS-LB mixed media (black bar) alone vs. co-cultured (checkered bar). Each probiotic (100 ml of 108 CFU/mL) was individually co-cultured with ST (100 ml of ˜5×10⁷ CFU/mL) and incubated anaerobically for 24 h at 37° C.

FIGS. 21A-21D show the growth and persistence of Salmonella enterica serovar Enteritidis (SE) mixed with other co-cultures. FIG. 21A shows SE growth and persistence in mixed media and co-cultured with Lactobacillus acidophilus (LA). FIG. 21B shows SE growth and persistence in mixed media and co-cultured with Lacticaseibacillus rhamnosus GG (LGG). FIG. 21C shows SE growth and persistence in mixed media and co-cultured with Bifidobacterium lactis (Bb12). FIG. 21D shows SE growth and persistence in mixed media and co-cultured with Levilactobacillus brevis (Lbrev). MRS-LB mixed media (black bar) alone vs. co-cultured (checkered bar). Each probiotic (100 ml of 10⁸ CFU/mL) was individually co-cultured with SE (100 ml of ˜5×10⁷ CFU/mL) and incubated anaerobically for 24 h at 37° C.

FIGS. 22A-22B show that Salmonella is inhibited by probiotic secreted products. FIG. 22A shows ST inhibition by probiotics secreted products in a trans-well assay. FIG. 22B shows SE inhibition by probiotics secreted products in a trans-well assay. Probiotics were placed above a 0.22 μm filter with corresponding Salmonella.

FIGS. 23A-23B show that probiotic CFSs minimizes Salmonella invasion. FIG. 23A shows the effect of probiotic CFS on ST invasion in HT-29 cells. FIG. 23B shows the effect of probiotic CFS on SE invasion in HT-29 cells. Polarized HT-29 cells were infected with Salmonella enterica subsp. enterica serotype Typhimurium (A) and Salmonella enterica subsp. enterica serotype Enteritidis (B) and treated for 4 h with 12.5% and 25% CFS to determine the effect probiotics had on the invasion of Salmonella.

FIGS. 24A-24B show LC-MS/MS profiling of organic acids in probiotic CFS. FIG. 24A shows LC-MS/MS profiling of organic acids in probiotics cultured alone. FIG. 24B shows LC-MS/MS profiling of organic acids in probiotics in co-culture with ST.

FIGS. 25A-25D show the impact of the probiotic treatments against ST colonization in chicken. FIG. 25A shows the log CFU of ST colonization in bird cecum. FIG. 25B shows the percent of chickens positive for ST in spleen. FIG. 25C shows the percent of chickens positive for ST in Liver. FIG. 25D shows the body weight 10 days post infection. N=10/group; horizontal line: mean.

FIGS. 26A-26D show the efficacy of LGG in drinking water against ST colonization in chickens. FIG. 26A shows the log CFU of ST colonization in chicken cecum. FIG. 26B shows the percent of chickens positive for ST in Spleen. FIG. 26C shows the percent of chickens positive for ST in Liver. FIG. 26D shows the body weight 8 days post infection, N=15/group. Horizontal line: mean.

FIG. 27 shows the growth inhibitory activity of LGG/Bb12 derived novel peptides (12 mM) when incubated with ST (105 CFU/mL). Inhibition of PN-3 at 14 mM against ST and SE. Inhibition of PN-5 at 16 mM against ST and SE. Kinetic OD₆₀₀ measurements were taken for 12 h using Tecan microplate reader. Growth inhibitory activity of LGG/Bb12 derived novel peptides when incubated with ST. Initial screening of peptides at 12 mM with 10⁵ CFU/mL of ST. Inhibition of PN-3 at 14 mM against ST and SE (105 CFU/mL). Inhibition of PN-5 at 16 mM against ST and SE. Kinetic OD₆₀₀ measurements were taken for 12 h using Tecan microplate reader.

FIGS. 28A-28C show the LC-MS peaks and retention times of derived peptides that showed growth inhibitory activity against ST. FIG. 28A shows the LC-MS peaks and retention times for PN-2: AESSDTNLVNAKAA (SEQ ID NO:18). FIG. 28B shows the LC-MS peaks and retention times for PN-3: VQAAQAGDTKPIEV (SEQ ID NO:21). FIG. 28C shows the LC-MS peaks and retention times for PN-5: VTDTSGKAGTTKISNV (SEQ ID NO:32).

FIGS. 29A-29C show the derived peptides fragmentation peaks used for sequence/nomenclature. FIG. 29A shows PN-2 peptide fragmentation peaks. FIG. 29B shows PN-3 peptide fragmentation peaks. FIG. 29C shows PN-5 peptide fragmentation peaks.

FIGS. 30A-30C show the growth pattern of ST with and without peptide at 9-21 mM concentration. FIG. 30A shows the minimal inhibitory concentrations (MIC) for PN-3. FIG. 30B shows the minimal inhibitory concentrations (MIC) for PN-5. FIG. 30C shows that 21 mM is the minimal bactericidal concentration for PN-3 and PN-5/Various concentrations of peptides were added to the bacterial suspension (5*105 CFU/mL) in a 96-well plate. The plate was incubated in a TECAN Sunrise™ absorbance microplate reader at 37° C., measuring the OD 600 set at every 30 mins for 12 hrs., followed by bacterial enrichment to determine the minimal bactericidal concentration log CFU/mL.

FIG. 31 shows the heatmap of inhibition of various serovars of Salmonella after treating with minimal bactericidal concentrations of peptides PN3 and PN5. Salmonella serovars (5×105 CFU/mL) were treated with peptides and grown at 37° C. for 12 hrs. In addition, bacterial inhibition percentage was calculated by comparing with no peptide control. At MBC, PN3 was able to kill all the eight serotypes of Salmonella with no viable colony. At the same time, PN5 killed all serotypes except Saintpaul. Red: Bactericidal, Green: Non bactericidal.

FIGS. 32A-32B show the growth inhibition percentage of various serovars of Salmonella after treating with minimal inhibitory concentrations of peptides. FIG. 32A shows the growth inhibition percentage of various serovars of Salmonella after treating with minimal inhibitory concentrations of PN-3. FIG. 32B shows the growth inhibition percentage of various serovars of Salmonella after treating with minimal inhibitory concentrations of PN-5. Salmonella serovars (5×105 CFU/mL) were treated with peptides and grown at 37° C. for 12 hrs. In addition, bacterial inhibition percentage was calculated by comparing with no peptide control. Both the peptides inhibited the growth over 95% for all the serovars except S. Muenchen (˜70% inhibition by PN3).

FIG. 33 shows the zone of inhibition induced by LGG and Bb12 against APEC in agar-well diffusion assay.

FIGS. 34A-34B show the viability of avian pathogenic E. coli (APEC) and Salmonella enterica serovar Typhimurium (ST). FIG. 34A shows the viability of APEC when incubated with LGG and Bb12 for 24 hours in co-culture. FIG. 34B shows the viability of APEC when incubated with LGG and Bb12 for 24 hours in co-culture.

FIG. 35 shows the supernatants of LGG and Bb12 inhibited APEC growth in trans-well assay.

FIGS. 36A-36B show the profiling of organics acids in LGG and Bb12 supernatants. FIG. 36A shows the organic profiling in supernatants revealing lactic acid as a major organic acid secreted by LGG and Bb12 when incubated with APEC. FIG. 36B shows the organic profiling in supernatants revealing lactic acid as a major organic acid secreted by LGG and Bb12 when incubated with ST.

FIGS. 37A-37B show the experimental design for testing efficacy. FIG. 37A shows the experimental design for testing the efficacy of LGG and Bb12 against APEC in chickens. FIG. 37B shows the experimental design for testing the efficacy of LGG and Bb12 against ST in chickens.

FIGS. 38A-38C show the effect of LGG on chickens infected with APEC and ST. FIG. 38A shows the effect of LGG on the APEC load in chicken cecum. FIG. 38B shows the effect of LGG on the ST load in chicken cecum. FIG. 38C shows the effect of LGG on the ST load in chicken spleen.

FIG. 39 shows that LGG increased the body weight gain of chickens.

FIGS. 40A-40D show the impact of LGG on cecal microbiota of chickens. FIG. 40A shows alpha diversity, FIG. 40B shows beta diversity, FIG. 40C shows relative abundance at the phylum level, and FIG. 40D shows relative abundance level at the genus level.

FIGS. 41A-41D show the inhibition of APEC growth by peptides at different concentrations.

FIG. 41A shows inhibition of APEC growth by P1, FIG. 41B shows inhibition of APEC growth by P2, FIG. 41C shows inhibition of APEC growth by P3, and FIG. 41D shows inhibition of APEC growth by P4.

FIGS. 42A-42D show the activity of peptides against different APEC serotypes/strains. FIG. 42A shows activity of P1, FIG. 42B shows activity of P2, FIG. 42C shows activity of P3, and FIG. 42D shows activity of P1, P2, and P3 on commensal/beneficial microbes.

FIG. 43A-43I shows the activity of peptides against Salmonella infection. FIG. 43A-D shows the activity of peptides against ST. FIG. 43E-H shows the activity of peptides against SE.

FIGS. 44A-44B show experimental design for testing efficacy. FIG. 44A shows the experimental design for testing the efficacy of peptides against APEC in chickens. FIG. 44B shows the experimental design for testing the efficacy of peptides against ST in chickens.

FIGS. 45A-45F show the effect of 50 mg/kg of peptides on APEC load in the internal organs of chickens. FIG. 45A shows the effect of peptides in chicken cecum. FIG. 45B shows the effect of peptides in chicken lungs. FIG. 45C shows the effect of peptides in chicken kidneys. FIG. 45D shows the effect of peptides in chicken heart. FIG. 45E shows the effect of peptides in chicken liver. FIG. 45F shows the effect of peptides on chicken body weight.

FIGS. 46A-46C show the effect of 100 mg/kg peptides on APEC load in internal organs. FIG. 46A shows the effect of peptides in chicken cecum. FIG. 46B shows the effect of peptides on chicken body weight. FIG. 46C shows the effect of peptides (100 mg/kg) lung, kidney, heart, and liver of chickens.

FIGS. 47A-47B show the effect of peptides (P-1 and P-2) on Salmonella load. FIG. 47A shows the effect of P-1 and P-2 on Salmonella load in chicken cecum. FIG. 47B shows the effect of P-1 and P-2 on Salmonella load in chicken liver.

FIG. 48 shows the cytological profiling of APEC treated with peptides using confocal microscopy. Peptides affect APEC membrane as evident by loss of red-stained membrane after the peptide treatment.

FIG. 49 shows the cytological profiling of APEC treated with peptides using transmission electron microscopy.

FIG. 50 shows the cytological profiling of ST treated with peptides using confocal microscopy. Peptides affect ST membrane as evident by loss of red-stained membrane after the peptide treatment.

FIG. 51 shows the cytological profiling of ST treated with peptides using transmission electron microscopy.

FIGS. 52A-52B show the efficacy of peptides in a wax moth (Galleria mellonella) larva model. FIG. 52A shows the survival curve of larvae either untreated or treated with peptides. FIG. 52B shows the APEC load in larvae either untreated or treated with peptides. PC, infected and vehicle (sterile water containing DMSO)-treated larvae; KAN, infected and kanamycin (50 mg/kg body weight)-treated larvae.

FIG. 53 shows the effect of peptide treatment on expression of genes essential for maintaining outer membrane integrity in APEC.

FIGS. 54A-54B show the effect of peptides treatment on bacterial membrane proteins. FIG. 54A shows the expression levels of OmpC proteins in APEC. FIG. 54B shows the expression levels of MlaA proteins in APEC.

FIGS. 55A-55C show the Alanine scanning of peptides identified AAs crucial for peptides activity. FIG. 55A shows alanine scanning results of P1, FIG. 55B shows alanine scanning results of P2, FIG. 55C shows alanine scanning results of P3.

FIGS. 56A-56D show the APEC load in ceca of chickens (at 7 dpi [days post infection]) treated with peptides. FIG. 56A shows the APEC load after treatment with 50 mg/kg body weight of peptides. FIG. 56B shows the APEC load after treatment with 100 mg/kg body weight of peptides.

FIG. 56C shows the effect on chicken body weight after treatment with 50 mg/kg of peptides. FIG. 56D shows the effect on chicken body weight after treatment with 100 mg/kg of peptides. PC, infected but not treated chickens; NC, noninfected and nontreated chickens.

FIGS. 57A-57D show the APEC load of chickens (at 7 dpi [days post infection]) treated with either peptides P-1 or P-2. FIG. 57A shows the APEC load in cecum after treatment with P-1 peptide. FIG. 57B shows the effect on chicken body weight after treatment with P-1 peptides. FIG. 58C shows the APEC load in cecum after treatment with P-2 peptide. FIG. 58D shows the effect on chicken body weight after treatment with P-2 peptides. Peptides P1 and P2 were administered in various doses such as 50, 100, and 200 mg/L in drinking water. *, P<0.05; ***, P<0.001, Tukey's test; PC, infected but not treated chickens; NC, noninfected and nontreated chickens. Both the P1 and P2 at the dose of 50 mg/L reduced APEC colonization by 1.089 (p<0.05) and 1.5 (p<0.01) log CFU/gm, respectively and no significant effect was observed on the bodyweight.

FIGS. 58A-58B show the APEC load after treatment 8 days dpi with LGG and 50 mg/mL of peptides in drinking water. FIG. 58A shows the APEC load in chicken ceca. FIG. 58B shows the effect on chicken body weight. *, P<0.05; **, P<0.01, Tukey's test; PC, infected but not treated chickens; NC, noninfected and nontreated chickens, LGG, LGG treated not infected. LGG, P1, LGG-P1, P2 and LGG-P2 reduced APEC colonization in ceca by 1.23, 1.57, 1.67, 1.02, and 1.48 Log CFU/gm, respectively and no significant effect was observed on the bodyweight.

FIGS. 59A-59C show the impact of 100 mg/kg peptides on cecal microbiota of chickens infected with APEC. FIG. 59A shows effect of peptides on commensal/beneficial microbes, FIG. 59B shows alpha diversity, and FIG. 59C shows beta diversity.

FIGS. 60A-60B show the impact of 100 mg/kg peptides on cecal microbiota of chickens infected with APEC. FIG. 60A shows relative abundance at the phylum level, and FIG. 60B shows relative abundance at the genus level.

FIGS. 61A-61C show the impact of 50 mg/kg of peptides on cecal microbiota of chickens infected with Salmonella. FIG. 61A shows the effect of peptides on commensal and probiotic bacteria. FIG. 61B shows effect of peptides on alpha diversity of cecal microbiota . . . . FIG. 61C shows effect of peptides on beta diversity of cecal microbiota.

FIGS. 62A-62B show impact of 50 mg/kg of peptides on cecal microbiota of chickens infected with Salmonella. FIG. 62A shows relative abundance at the phylum level. FIG. 62B show relative abundance at the family level.

FIGS. 63A-63B show the inhibition percentage (%) of APEC growth by peptides at different concentrations. FIG. 63A shows the inhibition percentage of APEC growth by 6 mM peptides. FIG. 63B shows the inhibition percentage of APEC growth by 12 mM peptides. Peptides were added to the wells of the 96-well plate containing APEC suspension (5×105 CFU/mL) and incubated at 37° C. in TECAN Sunrise™ absorbance microplate reader with kinetic absorbance measurement set at every 30 mins for 12 h. The inhibition (%) was calculated using the formula: (OD₆₀₀ DMSO treated well-OD₆₀₀ peptide treated well)/OD₆₀₀ DMSO treated well×100%.

FIGS. 64A-64B show the Shannon's diversity index measuring the microbial richness in cecum of chickens treated with various doses of peptides. FIG. 64A shows the Shannon's diversity at 50 mg/kg of peptides. FIG. 64B shows the Shannon's diversity at 100 mg/kg of peptides. NC: non-infected and non-treated chickens, PC: infected but not treated chickens.

FIGS. 65A-65B show the Principal Coordinates Analysis (PCoA) plot comparing the microbial communities (weighted unifrac beta-diversity) in cecum of chickens treated with peptides. FIG. 65A shows the PCoA after 50 mg/kg of peptides. FIG. 65B shows the PCoA after 100 mg/kg of peptides. NC: non-infected and non-treated chickens, PC: infected but not treated chickens.

FIG. 66 shows the schematic diagram for the experimental design to test the efficacy of peptides in commercial broiler chickens. Peptides were administered through orally twice a day from day 1 to day 7 either at 50 mg/kg or 100 mg/kg dose. On day 2, chickens were infected orally with Rif^{γ} APEC O78 (1-2×10⁹ CFU/chicken). At day 9, chickens were euthanized, necropsied and cecum and internal organs (lung, liver, heart, and kidney) were processed for quantification of APEC load. The body weight of chickens was measured at day 9.

FIG. 67 shows the percent Inhibition of S. typhimurium (ST) growth by peptides at 12 mM. The inhibition (%) was calculated using the formula (Percent inhibition=(OD₆₀₀ DMSO treated well-OD₆₀₀ peptide treated well)/OD₆₀₀ DMSO treated well×100%). Two independent experiments were conducted and average plotted.

FIGS. 68A-68B show the dose response analysis showing percent inhibition of S. typhimurium (ST) and S. enteritidis (SE). FIG. 68A shows the ST growth when treated with peptides at 6 mM, 12 mM, 15 mM, and 18 mM. FIG. 68B shows the SE growth when treated with peptides at 15 mM and 18 mM. Plates were incubated in TECAN Sunrise™ absorbance microplate reader over 12 h at 37° C. Percent inhibition=(OD₆₀₀ DMSO treated well-OD₆₀₀ peptide treated well)/OD₆₀₀ DMSO treated well×100%. Two independent experiments were conducted.

FIGS. 69A-69D show the Effect of peptides against ST in chickens treated with 50 mg/kg per body weight twice daily for 7 days. FIG. 69A shows the effect of treatment on the reduction of ST in cecum. FIG. 69B shows the effect of peptide treatment on percent of birds positive for ST in liver with enriched with TTB. FIG. 69C shows the effect of peptide treatment on percent of birds positive for ST in spleen with enriched with TTB. FIG. 69D shows the effect of peptide treatment on bird weight at day 10. PC=ST challenged but untreated positive control.

FIG. 70 shows the alpha diversity measuring microbial richness and evenness in cecum of chickens treated with peptides. Shannon's diversity index. NC=untreated, not challenged negative control. PC=challenged but untreated positive control.

FIGS. 71A-71C show the analysis of cecal microbial community. FIG. 71A shows the full analysis of cecal microbiota community within chickens. FIG. 71B shows the Bray-Curtis distance plot analyzing beta diversity comparing cecum microbial communities by group. Microbial relative abundance in chicken cecum at the phylum level. FIG. 71C shows the Bray-Curtis distance plot analyzing beta diversity comparing cecum microbial communities by group. Microbial relative abundance in chicken cecum at the family level. NC=untreated, not challenged negative control. PC=challenged but untreated positive control.

FIGS. 72A-72B show the relative importance of amino acid residues using alanine screening.

FIG. 72A shows the relative importance of amino acids in peptide P-1. FIG. 72B shows the relative importance of amino acids in peptide P-2. Relative Importance was calculated using formula: (percent growth in analogue-percent growth in original peptide)/(percent growth in DMSO-treated control-percent growth in original peptide)×100.

FIG. 73 shows the Leica TCS SP6 confocal microscopy images of untreated, and peptide treated S. typhimurium (ST). ST was treated with 5× peptide MIC, incubated for 3 h, and stained for 45 minutes with red FM4-64 membrane stain, green nucleus SYTO-9 stain, Salmonella membrane was clearly visible in PC sample (white arrows), whereas no or minimally (blue arrows) visible membrane was observed in peptide treated samples. Superimposed images (FM4-64 plus SYTO-9) showed nuclear material of Salmonella enclosed by membrane in PC (white arrows), whereas no membrane was visible (orange arrows) covering the nuclear material in peptide-treated cultures.

FIG. 74 shows the Hitachi H-7500 Transmission electron microscopy images of untreated, and peptide treated S. typhimurium (ST). ST was treated with 10× peptide MIC and incubated for 3 h. Images displayed at 8× and 20× magnification. Clearly demarcated membrane encircling the dense cytoplasmic contents was observed in untreated S. typhimurium (white arrows), whereas the membrane was either sloughed/shed (blue arrows), or flaccid (orange arrows), in ST treated with peptides.

FIG. 75 shows the S. typhimurium load in ceca of chickens (at 7 dpi [days post infection]) treated with peptides P1 and P2 in various doses such as 50, 100, and 200 mg/L in drinking water. *, P<0.05; Dunnett's test; PC, infected but not treated chickens; NC, noninfected and nontreated chickens. Both the P1 and P2 at 200 mg/L and 100 mg/L concentrations reduced S. typhimurium colonization by 0.78 (p<0.05) and 0.69 (p<0.05) log CFU/gm, respectively.

FIG. 76 shows the S. typhimurium load in ceca of chicken (at eight dpi [days post infection]) treated with LGG (10\hat{ }8 CFU/mL) and peptides P1 and P2 with 200 and 100 mg/L concentration in drinking water, respectively. *, P<0.05; Dunnett's test; PC, infected but not treated chickens; NC, noninfected and nontreated chickens. Chickens treated with LGG, LGG-P1, and LGG-P2 reduced S. typhimurium colonization in ceca by 1.88, 0.53, and 1.86 Log CFU/gm, respectively.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, “composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprises” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

“Amino acid” is used herein to refer to a chemical compound with the general formula: NH₂—CRH COOH, where R, the side chain, is H or an organic group. Where R is organic, R can vary and is either polar or nonpolar (i.e., hydrophobic). The following abbreviations are used throughout the application: A=Ala=Alanine, T=Thr=Threonine, V=Val=Valine, C=Cys=Cysteine, L=Leu=Leucine, Y=Tyr=Tyrosine, I=Ile=Isoleucine, N=Asn=Asparagine, P=Pro=Proline, Q=Gln=Glutamine, F=Phe=Phenylalanine, D=Asp=Aspartic Acid, W=Trp=Tryptophan, E=Glu=Glutamic Acid, M=Met=Methionine, K=Lys=Lysine, G=Gly=Glycine, R=Arg=Arginine, S=Ser=Serine, H=His=Histidine. Unless otherwise indicated, the term “amino acid” as used herein also includes amino acid derivatives that nonetheless retain the general formula.

As used herein, “polypeptide” or “peptide” refers to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to. For example, “reduces bacterial growth” means reducing the rate of growth of a bacterium relative to a standard or a control.

As used herein, the terms “treating” or “treatment” of a subject includes the administration of a drug to a subject with the purpose of curing, healing, alleviating, relieving, altering, remedying, ameliorating, improving, stabilizing, or affecting a disease or disorder, or a symptom of a disease or disorder. The terms “treating”, and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

As used herein, “culture” or “cell culture” is the process by which cells are grown under controlled conditions, generally outside their natural environment. After the cells of interest have been isolated from living tissue, they can subsequently be maintained under carefully controlled conditions. These conditions vary for each cell type, but generally consist of a suitable vessel with a substrate or medium that supplies the essential nutrients (amino acids, carbohydrates, vitamins, minerals), growth factors, hormones, and gases (CO₂, O₂), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture. “Cell culture” also refers to the culturing of cells derived from multicellular eukaryotes, especially animal cells, in contrast with other types of culture that also grow cells, such as plant tissue culture, fungal culture, and microbiological culture (of microbes).

The term “administering” or “administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “antimicrobial” refers to an agent that kills microorganisms or stops or inhibits their growth. The term “antibacterial” refers to an agent that is proven to kill bacteria or stops or inhibits bacterial growth.

The term “antibiotic” refers to a type of antimicrobial substance active against bacteria. These are a type of antimicrobial agent for fighting bacterial infections, and antibiotic medications are widely used in the treatment and prevention of such infections. They may either kill or inhibit the growth of bacteria.

As used herein, the term “probiotic” refers to live microorganisms promoted with claims that they provide health benefits when consumed, generally by improving or restoring the gut flora.

The term “microbiota” refers to the range of microorganisms that may be commensal, symbiotic, or pathogenic found in and on all multicellular organisms, including plants and animals. These include bacteria, archea, protists, fungi, and viruses and have been found to be crucial for immunologic, hormonal, and metabolic homeostasis of the host.

“Antibiotic resistance” as used herein refers to when microbes evolve mechanisms that protect them from the effects of antimicrobials. This specifically refers to bacteria that become resistant to antibiotics.

As used herein, “colonization” refers to the biological process by which species spread to new areas. In reference to microorganisms, “colonization” means the formation of communities of microorganisms on surfaces.

The term “biofilm” refers to any syntrophic microorganisms in which cells stick to each other and often also to a surface. The adherent cells become embedded within a slimy extracellular matrix that is composed of extracellular polymeric substances. The cells within the biofilm produce the extracellular polymeric substances components, which are typically a polymeric combination of polysaccharides, proteins, lipids, and DNA. Biofilms may form on living or non-living surfaces and can be prevalent in natural, industrial, and hospital settings.

“Serotype” or “serovar” as used herein refers to a distinct variation within a species of bacteria or virus or among immune cells of different individuals. These microorganisms, viruses, or cells are classified together based on their surface antigens, allowing the epidemiologic classification of organisms to the subspecies level.

The term “pathogen” refers to any organism that can produce disease. A pathogen may also be referred to as an infectious agent, or simply a germ.

As used herein, the term “avian” refers to anything related to or derived from birds, such as chickens, turkeys, quail, or ducks.

The term s “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

As used herein, a “pharmaceutically acceptable carrier” refers to a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

### Compositions and Methods of Use

In one aspect, disclosed herein is a composition comprising: an antimicrobial peptide comprising an amino acid sequence at least 80% identical to SEQ ID NO: 18, SEQ ID NO: 21, or SEQ ID NO: 32, or a functional variant thereof, and a pharmaceutically acceptable carrier. In some embodiments, the amino acid sequence comprises at least 90% identity to SEQ ID NO: 18, SEQ ID NO:21, or SEQ ID NO:32. These antimicrobial peptides can have an amino acid sequence with 80%, 81%, 82%, 83%, 84%, 85%, 86%, 87%, 88%, 89%, 90%, 91%, 92%, 93%, 94%, 95%, 96%, 97%, 98%, 99%, and 100% identity to SEQ ID NO: 18, SEQ ID NO: 21, or SEQ ID NO: 32. In some embodiments, the amino acid sequence comprises SEQ ID NO: 5. In some embodiments, the amino acid sequence comprises SEQ ID NO: 18. In some embodiments, the amino acid sequence comprises SEQ ID NO: 21. In some embodiments, the amino acid sequence comprises SEQ ID NO: 26. In some embodiments, the amino acid sequence comprises SEQ ID NO: 32. In some embodiments, the amino acid sequence is 10-20 amino acids in length. Viewed in terms of the number of amino acids that can vary, disclosed herein is a sequence comprising 1, 2, 3, 4, 5, 6, 7, 8, 9, 10, or more amino acids that can vary compared to SEQ ID NO: 5, SEQ ID NO: 18, SEQ ID NO: 21, SEQ ID NO: 26, or SEQ ID NO: 32. One of skill in the art will understand that the sequence can vary and still retain function of SEQ ID NO: 5, SEQ ID NO: 18, SEQ ID NO: 21, SEQ ID NO: 26, or SEQ ID NO: 32 to act as a antimicrobial peptide. Therefore, contemplated herein are sequences which vary from SEQ ID NO: 5, SEQ ID NO: 18, SEQ ID NO: 21, SEQ ID NO: 26, or SEQ ID NO: 32, but which retain their ability to act as antibacterial peptides.

In one aspect, disclosed herein is a composition comprising: an antimicrobial peptide consisting of an amino acid sequence at least 80% identical to SEQ ID NO: 18, SEQ ID NO: 21, or SEQ ID NO: 32, or a functional variant thereof, and a pharmaceutically acceptable carrier. In some embodiments, the amino acid sequence consists of an amino acid sequence at least 90% identity to SEQ ID NO: 18, SEQ ID NO:21, or SEQ ID NO:32. These antimicrobial peptides can have an amino acid sequence with 80%, 81%, 82%, 83%, 84%, 85%, 86%, 87%, 88%, 89%, 90%, 91%, 92%, 93%, 94%, 95%, 96%, 97%, 98%, 99%, and 100% identity to SEQ ID NO: 18, SEQ ID NO: 21, or SEQ ID NO: 32. In some embodiments, the amino acid sequence consisting of SEQ ID NO: 5. In some embodiments, the amino acid sequence consisting of SEQ ID NO: 18. In some embodiments, the amino acid sequence consisting of SEQ ID NO: 21. In some embodiments, the amino acid sequence consisting of SEQ ID NO: 26. In some embodiments, the amino acid sequence consisting of SEQ ID NO: 32. In some embodiments, the amino acid sequence is 10-20 amino acids in length. Viewed in terms of the number of amino acids that can vary, disclosed herein is a sequence consisting of 1, 2, 3, 4, 5, 6, 7, 8, 9, 10, or more amino acids that can vary compared to SEQ ID NO: 5, SEQ ID NO: 18, SEQ ID NO: 21, SEQ ID NO: 26, or SEQ ID NO: 32.

In one aspect, disclosed herein is a composition comprising: an antimicrobial peptide comprising or consisting of an amino acid sequence at least 80% identical to a sequence listed in Table 5, or a functional variant thereof, and a pharmaceutically acceptable carrier. In one aspect, disclosed herein is a composition comprising: an antimicrobial peptide comprising or consisting of an amino acid sequence at least 80% identical to a sequence listed in Table 15, or a functional variant thereof; and a pharmaceutically acceptable carrier. In one aspect, disclosed herein is a composition comprising: an antimicrobial peptide comprising or consisting of an amino acid sequence at least 80% identical to a sequence listed in Table 17, or a functional variant thereof, and a pharmaceutically acceptable carrier. In one aspect, disclosed herein is a composition comprising: an antimicrobial peptide comprising or consisting of an amino acid sequence at least 80% identical to a sequence listed in Table 22, or a functional variant thereof; and a pharmaceutically acceptable carrier. In one aspect, disclosed herein is a composition comprising: an antimicrobial peptide comprising or consisting of an amino acid sequence at least 80% identical to a sequence listed in Table 25, or a functional variant thereof; and a pharmaceutically acceptable carrier. In one aspect, disclosed herein is a composition comprising: an antimicrobial peptide comprising or consisting of an amino acid sequence at least 80% identical to a sequence listed in Table 26, or a functional variant thereof; and a pharmaceutically acceptable carrier. In one aspect, disclosed herein is a composition comprising: an antimicrobial peptide comprising or consisting of an amino acid sequence at least 80% identical to a sequence listed in Table 28, or a functional variant thereof, and a pharmaceutically acceptable carrier.

In one aspect, disclosed herein is a composition comprising: an antimicrobial peptide comprising or consisting of an amino acid sequence listed in Table 5, or a functional variant thereof, and a pharmaceutically acceptable carrier. In one aspect, disclosed herein is a composition comprising: an antimicrobial peptide comprising or consisting of an amino acid sequence listed in Table 15, or a functional variant thereof; and a pharmaceutically acceptable carrier. In one aspect, disclosed herein is a composition comprising: an antimicrobial peptide comprising or consisting of an amino acid sequence listed in Table 17, or a functional variant thereof, and a pharmaceutically acceptable carrier. In one aspect, disclosed herein is a composition comprising: an antimicrobial peptide comprising or consisting of an amino acid sequence listed in Table 22, or a functional variant thereof; and a pharmaceutically acceptable carrier. In one aspect, disclosed herein is a composition comprising: an antimicrobial peptide comprising or consisting of an amino acid sequence listed in Table 25, or a functional variant thereof; and a pharmaceutically acceptable carrier. In one aspect, disclosed herein is a composition comprising: an antimicrobial peptide comprising or consisting of an amino acid sequence listed in Table 26, or a functional variant thereof, and a pharmaceutically acceptable carrier. In one aspect, disclosed herein is a composition comprising: an antimicrobial peptide comprising or consisting of an amino acid sequence listed in Table 28, or a functional variant thereof; and a pharmaceutically acceptable carrier.

In one aspect, disclosed herein is a method of treating or preventing a bacterial infection in a subject comprising administering to the subject an effective amount of an antimicrobial peptide comprising an amino acid sequence at least 80% identical to SEQ ID NO: 18, SEQ ID NO:21, SEQ ID NO:32, SEQ ID NO:85, SEQ ID NO:86, or SEQ ID NO:87, or a functional variant thereof. In some embodiments, the amino acid sequence comprises at least 90% identity to SEQ ID NO: 18, SEQ ID NO:21, SEQ ID NO:32, SEQ ID NO:85, SEQ ID NO:86, or SEQ ID NO:87, or a functional variant thereof. These antimicrobial peptides can have an amino acid sequence with 80%, 81%, 82%, 83%, 84%, 85%, 86%, 87%, 88%, 89%, 90%, 91%, 92%, 93%, 94%, 95%, 96%, 97%, 98%, 99%, and 100% identity to SEQ ID NO: 18, SEQ ID NO: 21, or SEQ ID NO: 32. In some embodiments, the antimicrobial peptide comprises an amino acid sequence to SEQ ID NO: 5. In some embodiments, the antimicrobial peptide comprises an amino acid sequence to SEQ ID NO: 18.

In some embodiments, the antimicrobial peptide comprises an amino acid sequence to SEQ ID NO: 21. In some embodiments, the antimicrobial peptide comprises an amino acid sequence to SEQ ID NO: 26. In some embodiments, the antimicrobial peptide comprises an amino acid sequence to SEQ ID NO: 32. In some embodiments, the antimicrobial peptide comprises an amino acid sequence to SEQ ID NO: 85. In some embodiments, the antimicrobial peptide comprises an amino acid sequence to SEQ ID NO: 86. In some embodiments, the antimicrobial peptide comprises an amino acid sequence to SEQ ID NO: 87. In some embodiments, the antimicrobial peptide comprises an amino acid sequence to SEQ ID NO: 88. In some embodiments, the antimicrobial peptide comprises an amino acid sequence to SEQ ID NO: 118. In some embodiments, the antimicrobial peptide comprises an amino acid sequence to SEQ ID NO: 119.

In some embodiments, the method of treating and preventing a bacterial infection in a subject comprises administering whole live Lacticaseibacillus rhamnosus GG (LGG) against APEC, ST, SE, or other E. coli and Salmonella serovars. Lactic acid bacteria are recognized as a food-safety probiotic with anti-infection, immunoregulatory, anti-oxidative, and intestinal microecology regulation mechanisms. LGG is a lactic acid bacterium and an important probiotic that is fixed in the gastrointestinal (GI) tract system. Since LGG is known to promote GI tract health, the delivery of the whole live LGG as a treatment prevents colonization of APEC, ST, and SE bacteria in chickens.

In other embodiments, the probiotics LGG and/or Bifidobacterium lactis (Bb12) and their derived peptides AESSDTNLVNAKAA [PN2], VQAAQAGDTKPIEV [PN3], AFDNTDTSLDSTFKSA [PN4], and VTDTSGKAGTTKISNV [PN5] which comprise SEQ ID NO:18, SEQ ID NO: 21, SEQ ID NO: 26, and SEQ ID NO: 32, respectively for use in subjects such as chickens, wherein peptides are used alone or in combination, or whole LGG is used. In some embodiments, the probiotic LGG and its derived peptides P1: NPSRQERR, P2: PDENK, P3: VHTAPK, and P4: MLNERVK, which comprise SEQ ID NO: 85, SEQ ID NO: 86, SEQ ID NO: 87 and SEQ ID NO: 88, respectively for use in subjects such as chickens, wherein peptides are used alone or in combination, or whole LGG is used. In some embodiments, the antimicrobial peptide is administered alone or in combination with an additional antimicrobial peptide. In some embodiments, the antimicrobial peptide is administered in combination with an antibiotic therapy. In some embodiments, the antibiotic therapy is selected from a tetracycline, a sulfonamide, an aminoglycoside, a quinolone, or a β-lactam. The antimicrobial peptides disclosed herein can be administered alone. The antimicrobial peptides disclosed herein can also be administered as a combination therapy with other antimicrobial peptides or antibiotics to improve efficacy against infection. Known antibiotic therapies have conferred antibiotic-resistance against some pathogens, however delivery of antimicrobial peptides with these antibiotics promises retention of therapeutic effectiveness against foodborne pathogens. In some embodiments, the antimicrobial peptides are water-soluble compositions. The hydrophilic nature of these antimicrobial peptides allows for preparation in an aqueous solvent. A water-based preparation of the antimicrobial peptides described herein allows for stable delivery of the therapeutic composition to a subject with little toxic effects.

In some embodiments, treating or preventing bacterial infection, or bacterial overgrowth, in a subject is achieved by administering to the subject Lactobacillus rhamnosus GG (LGG) or Bifidobacterium lactis (Bb12) and their derived peptides which produces peptides AESSDTNLVNAKAA [PN2], VQAAQAGDTKPIEV [PN3], AFDNTDTSLDSTFKSA [PN4], and VTDTSGKAGTTKISNV [PN5] which comprise SEQ ID NO:18, SEQ ID NO: 21, SEQ ID NO: 26, and SEQ ID NO: 32, respectively. In other embodiments, treating or preventing bacterial infection, or bacterial overgrowth, in a subject is achieved by administering one or more of the following peptides to the subject in need thereof, wherein the peptides are produced by Lactobacillus rhamnosus GG (LGG) and/or Bifidobacterium lactis (Bb12) and their derived peptides and comprise AESSDTNLVNAKAA [PN2], VQAAQAGDTKPIEV [PN3], AFDNTDTSLDSTFKSA [PN4], and VTDTSGKAGTTKISNV [PN5], which comprise SEQ ID NO:18, SEQ ID NO: 21, SEQ ID NO: 26, and SEQ ID NO: 32, respectively. In some embodiments, treating or preventing bacterial infection, or bacterial overgrowth, in a subject is achieved by administering to the subject the probiotic LGG which produces peptides P1: NPSRQERR, P2: PDENK, P3: VHTAPK, and P4: MLNERVK, which comprise SEQ ID NO: 85, SEQ ID NO: 86, SEQ ID NO: 87 and SEQ ID NO: 88, respectively. In some embodiments, treating or preventing bacterial infection, or bacterial overgrowth, in a subject is achieved by administering one or more of the following peptides to the subject in need thereof, wherein the peptides are produced by the probiotic LGG and comprise P1: NPSRQERR, P2: PDENK, P3: VHTAPK, and P4: MLNERVK, which comprise SEQ ID NO: 85, SEQ ID NO: 86, SEQ ID NO: 87 and SEQ ID NO: 88, respectively. In some embodiments, the bacterial infection is caused by a bacterial overgrowth. Bacterial overgrowth occurs when bacterial colonies overpopulate a given area or tissue that would normally not exist in large numbers. The overgrowth of bacteria often leads to bacterial infections that can impact multiple organ systems. Antibiotics are the traditional therapeutic agents delivered to combat bacterial infections. However, to date many bacteria have developed antibiotic-resistant strains leading to the necessity of new treatment compositions.

In some embodiments, the antimicrobial peptide targets a bacterial membrane. In some embodiments, the bacterial membrane is an outer membrane. Antimicrobial peptides are small peptides that interact with bacterial membranes. Specifically, these peptides penetrate bacterial membranes causing rupture and bacterial death. Because these peptides target bacterial structures rather than biological mechanisms, such as DNA replication and protein translation, they reduce the occurrence of bacterial drug resistance. In some embodiments, the bacterial membrane comprises a MlaA-OmpC/F protein system. The Mla-OmpC/F bacterial protein system is an essential component of the outer membrane of gram-negative bacteria. The MlaA-OmpC/F system as druggable target in APEC for drug development against APEC and related pathogens such as human ExPECs and other pathogenic E. coli including antibiotic-resistant strains. The OmpC/F component maintains outer membrane lipid asymmetry to prevent exposure to toxins, and thus is vital for bacterial viability. The composition disclosed herein can target the MlaA-OmpC/F proteins on the bacterial outer membrane to increase bacterial death with minimal resistance.

E. coli, Salmonella, and other related serotypes are common bacterial pathogens impacting human and animal populations. With the rise of antibiotic-resistance in poultry farms, pathogens can easily spread leading to subsequent bacterial infections causing economic devastation to avian and human health worldwide. The composition and methods disclosed herein can be used for treating and preventing E. coli and Salmonella pathogenicity in avian populations and inhibit further spread into human populations.

In some embodiments, the bacterial infection is caused by an Escherichia coli (E. coli) bacterium. In some embodiments, the E. coli is an extra-intestinal pathogenic E. coli (ExPEC). In some embodiments, the E. coli is an avian extra-intestinal pathogenic E. coli (APEC). In some embodiments, the E. coli is a human extra-intestinal pathogenic E. coli. In some embodiments, the human extra-intestinal pathogenic E. coli is a uropathogenic E. coli (UPEC) or a neonatal meningitis E. coli (NMEC). In some embodiments, the E. coli is an enterotoxigenic E. coli. In some embodiments, the bacterial infection is caused by a Salmonella bacterium. Salmonella. In some embodiments, the Salmonella bacterium is a Salmonella enterica serovar typhimurium (ST) bacterium or a Salmonella enterica serovar Enteritidis (SE) bacterium. In some embodiments, the Salmonella bacterium is a Salmonella enterica serovar Anatum, a Salmonella enterica serovar Albany, a Salmonella enterica serovar Brenderup, a Salmonella enterica serovar Javiana, a Salmonella enterica serovar Heidelberg, a Salmonella enterica serovar Muenchen, a Salmonella enterica serovar Newport, or a Salmonella enterica serovar Saintpaul.

In some embodiments, the subject is a bird. In some embodiments, the subject is a chicken. In some embodiments, the subject is a turkey. In some embodiments, the subject is a duck. In some embodiments, the subject is a quail. In some embodiments, the subject can be any poultry animal, including broilers, layers, breeders, and turkeys. In some embodiments, the subject is a companion bird, including parrots, parakeets, cockatiels, cockatoos, and macaws. In some embodiments, the subject is a human. In some embodiments, the subject can be any livestock animal, including cattle, sheep, pigs, and horses. In some embodiments, the subject can be any domesticated animal, including dogs and cats.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. In Vitro and In Vivo Evaluation of Lacticaseibacillus rhamnosus GG and Bifidobacterium lactis Bb12 Against Avian Pathogenic Escherichia coli and Identification of Novel Probiotic-Derived Bioactive Peptides

Avian pathogenic E. coli (APEC), an extra-intestinal pathogenic E. coli (ExPEC), is one of the most common bacterial pathogens of poultry. APEC continues to pose a formidable challenge to the poultry industry worldwide despite improvements in the poultry production systems over the years. APEC infects all species of poultry, including broilers, layers, breeders, and turkeys of all ages (9.52 to 36.73% prevalence), and in all types of production systems. In the United States (US), it is estimated that at least 30% of commercial flocks are affected by APEC at any point of time. APEC causes a wide range of localized and systemic infections in poultry, including yolk sac infection, omphalitis, respiratory tract infection, swollen head syndrome, septicemia, polyserositis, coligranuloma, enteritis, cellulitis and salpingitis; collectively referred as colibacillosis. Colibacillosis results in high morbidity and mortality (up to 20%) and decreased meat (2% decline in body weight) and egg production (loss up to 15%). More severely, in young chickens, APEC is associated with up to 53.5% mortality. Further, APEC is also responsible for 36-43% of carcass condemnations at slaughter. Altogether, APEC infections result in multi-million dollars annual losses to all facets of the poultry industry and remain as a serious impediment to the sustainable poultry production worldwide.

APEC has been also reported as a potential food-borne zoonotic pathogen, which can be transmitted to humans through consumption of contaminated poultry products. In particular, APEC has genetic similarities with human ExPECs [uropathogenic E. coli (UPEC) and neonatal meningitis E. coli (NMEC)], possesses virulence genes characteristics of UPEC/NMEC, and cause urinary tract infection and meningitis in rodent models as similar to UPEC and NMEC. Colicin-V (ColV) plasmids specific to APEC have been also detected in human clinical E. coli isolates suggesting evidence of potential foodborne transmission of APEC from poultry to humans even though concrete evidence is still lacking. In addition, APEC is also considered as a source of antibiotic resistance genes (ARGs) to human pathogens, which can make the human infections difficult to treat. Thus, APEC is a threat to both poultry and human health.

Antibiotics are commonly used to control APEC infections in poultry. However, APEC resistance to multiple antibiotics, including tetracyclines, sulfonamides, aminoglycosides, quinolones, and β-lactams, has been reported worldwide. Up to 92% of APEC isolated in the US, Europe and Australia were resistant to three or more antibiotics, particularly against tetracyclines, aminoglycosides and sulfonamides. Further, many countries (particularly US and European Union) have recommended the limited use of antibiotics in food-producing animals, including poultry, with a goal of reducing the selection pressure and subsequent emergence and transmission of antibiotic-resistant bacteria to humans. However, limiting on-farm use of antibiotics could significantly increase morbidity and mortality, thereby compromising production efficiency, food safety and security. Therefore, there is a critical need for developing new effective alternatives to antibiotics which can enhance the poultry health and production, mitigate antibiotic-resistance problem, promote antibiotic stewardship, and safeguard the human health.

Probiotics are defined as live microorganisms which when administered in adequate amounts confer health benefits to the host. Probiotics exhibit antibacterial activities, promote the growth, maintain the healthy gut, and strengthen the immune system; therefore, can serve as alternatives to antibiotics to control the bacterial infections as well as to enhance the production. Probiotics exert antibacterial effects through different mechanisms of action, such as i) enhancement of epithelial barrier functions, ii) competitive exclusion of pathogenic microorganisms, iii) production of antimicrobial substances and iv) modulation of the host immune system. Lacticaseibacillus rhamnosus, Lactobacillus acidophilus, Lactobacillus johnsonii, Limosilactobacillus reuteri, Lactiplantibacillus plantarum subsp. plantrarum, Limosilactobacillus fermentum, Lactobacillus helveticus, Lactobacillus gasseri, Bifpdobacterium bifidum, B. lactis, B. infantis, and B. breve are the most commonly used probiotics in humans and food animals. These probiotics have shown antimicrobial activities against various bacterial and viral pathogens, including Helicobacter, Salmonella, Listeria, Shigella, E. coli, Vibrio, Campylobacter, and rotavirus. Other probiotics have also shown proven benefits to the poultry health, particularly against Salmonella and Campylobacter. L. plantarum subsp. plantarum, L. reuteri, Ligilactobacillus salivarius, Lacticaseibacillus casei and E. coli Nissle 1917 reduced the Salmonella colonization. L. salivarius, L. johnsonii and L. casei reduced the colonization of Campylobacter. L. plantarum subsp. plantarum reduced the APEC colonization. However, there is overall lack of studies specifically demonstrating the activity of probiotic species against APEC. Further, information is also lacking on the bioactive substances secreted/released by these probiotics as well as their interactions with commensal microbes and/or pathogens in the gut which limits the understanding of the probiotic's mechanism(s) of action and reproducible use in industrial settings.

The objective of this study is to identify the probiotic species effective against APEC infection in poultry. Here, probiotic bacteria, L. rhamnosus GG was identified as effective in reducing APEC colonization in chickens. Also, it was identified that novel peptides derived from L. rhamnosus GG are inhibitory to APEC growth. Further, the interactions of L. rhamnosus GG with commensal microbes and APEC in the gut microbiome of chickens was investigated. Results show that L. rhamnosus GG can be developed as a preventative measure against APEC infections in chickens.

Materials and Methods Bacterial strains and culture conditions: The commensal and probiotic bacteria used in this study along with their culture conditions and media requirements for growth are listed in Table. 1. BD GasPak™ EZ container system (Becton, Dickinson and Company, NJ, USA) or MiniMacs anaerobic workstation (Microbiology International, MD, USA) was used to grow commensal and probiotic bacteria requiring the anaerobic conditions. APEC serotype O78, primarily used in this study, was kindly provided by Dr. Johnson (University of Minnesota, Saint Paul, MN, USA) and was isolated from lung of a Turkey clinically diagnosed with colibacillosis. Other APEC serotypes O1, O2, O8, O15, O18, O35, O109, and O115 were kindly provided by Drs. Nolan and Logue (University of Georgia, Athens, GA, USA). Luria-Bertani (LB) broth (BD Difco™) was used for the routine propagation of APEC serotypes. APEC serotypes stored at −80° C. in glycerol were grown overnight in LB broth at 37° C. with shaking at 200 rpm.

Agar-well diffusion assay: To determine the inhibitory activity of commensal and probiotic bacteria against APEC, agar-well diffusion assay was conducted as described previously. Briefly, LB agar plate was spread with 100 μL of APEC O78 (107 CFU/mL) and 100 μL of fully grown stationary phase whole cultures (adjusted to OD₆₀₀: 1) of commensal and probiotic bacteria were aliquoted into the wells bored in the agar plate. The plate was incubated at 37° C., and zone of inhibition was measured at 12 h and 24 h post-incubation. The inhibitory activity of L. rhamnosus GG and B. lactis Bb12 was also tested with different culture volumes (200 μL, 150 μL and 50 μL) and against other APEC serotypes as described above. Assay was also conducted with cell-free supernatants (CF Ss) of L. rhamnosus GG and B. lactis Bb12 and supernatant-free L. rhamnosus GG and B. lactis Bb12 itself. CFSs were prepared by centrifugation of whole cultures at 10,000×g for 10 min at 4° C. followed by filtration through 0.22 μm filter. The supernatant-free cultures were washed once and resuspended in PBS to check the activity of L. rhamnosus GG and B. lactis Bb12 itself. Two independent experiments were conducted.

Co-culture assay: To determine the anti-APEC activity of L. rhamnosus GG and B. lactis Bb12 in liquid media, co-culture assay was conducted as previously described. Briefly, 10⁷ CFU/mL of L. rhamnosus GG or B. lactis Bb12 and APEC O78 were incubated together in 5 mL of co-culture media (contains 100% MRS and 100% LB; pH 6.75 at 0 h) at 37° C. under anaerobic conditions with shaking at 50 rpm followed by the quantification of viable APEC O78 every 12 h until 24 h. Lactobacillus acidophilus and Levilactobacillus brevis were used for the comparison of anti-APEC activity as these two Lactobacillus species are commonly used probiotics in animal and human studies and several commercial probiotics currently being used in poultry industry contain these Lactobacillus species in their formulations. Two independent experiments were conducted.

Trans-well migration assay: To determine if the anti-APEC activity of L. rhamnosus GG and B. lactis Bb12 is due to bacterial cells itself or due to bacteria secreted/released products, trans-well migration assay was conducted. Assay was conducted using 0.22 μm Ultrafree-MC microcentrifuge tubes with removable filters (Millipore Sigma, MA, USA). Briefly, 16-18 h grown L. rhamnosus GG and B. lactis Bb12 cultures were aliquoted into the tube containing filter, whereas APEC O78 culture (107 CFU/mL) was aliquoted into the microcentrifuge tube below the filter. The filter tube was removed before aliquoting 700 μL of APEC culture into the microcentrifuge tube, then filter tube was inserted back and 700 μL of L. rhamnosus GG/B. lactis Bb12 culture was added above the filter in the filter tube. Sufficient volume (700 μL) was added to the microcentrifuge tube to allow contact with the tube containing the filter. The tubes were incubated at 37° C. under anaerobic conditions with shaking at 50 rpm. The viability of APEC O78 was quantified at 12 h and 24 h post-incubation. Two independent experiments were conducted.

Effect of pH on anti-APEC activity: It is reported in studies that Lactobacillus and Bifidobacterium strains exhibit antimicrobial activity by lowering the pH of the media. To observe the pH change, pH of the L. rhamnosus GG and B. lactis Bb12 cultures grown in co-culture media (MRS+LB) were measured every 12 h until 48 h in a separate experiment. To determine the effect of pH on L. rhamnosus GG and B. lactis Bb12 inhibitory activity against APEC O78, co-culture media (MRS+LB) was adjusted to different pH (4.0, 4.5, 5.0, 5.5 and 6.0) using 3 M HCL and tested for anti-APEC activity as described above. Additionally, pH-tolerance of APEC O78 was determined by growing APEC in LB media adjusted to different pH (4.0, 4.5, 5.0, 5.5 and 6.0) for 24 h. Two independent experiments were conducted.

Characterization of nature of antibacterial product(s): To understand the nature of secreted/released product(s), CF Ss of 24 h grown L. rhamnosus GG and B. lactis Bb12 cultures were subjected to heat and proteolytic enzyme treatments as described previously. The CFSs were subjected for heat (121° C.; autoclave) or proteinase K (1 mg/mL, 37° C. for 3 h) treatment and tested for inhibitory activity against APEC O78 in an agar-well gel diffusion assay as described above. Further, L. rhamnosus GG and B. lactis Bb12 CFSs were fractionated using a Amicon® Ultra centrifugal filter (Millipore Sigma) with mol. wt. cut-off (MWCO) of 3 kDa. The filtrates containing products less than 3 kDa were tested for inhibitory activity and compared with the inhibitory activity of unfractionated CFSs of L. rhamnosus GG and B. lactis Bb12 cultures. Two independent experiments were conducted.

Profiling of organic acids production: To quantify the organic acids in the CFSs of L. rhamnosus GG and B. lactis Bb12 cultures, LC-MS/MS coupled with isotope-labeled chemical derivatization method was used as described previously. For the preparation of CFSs, L. rhamnosus GG and B. lactis Bb12 were grown overnight, adjusted to OD₆₀₀ 1.0 (˜10⁹ CFU/mL) and sub-cultured (500 μL) in fresh media (14.5 mL) for 24 h at 37° C. under anaerobic conditions. The quantity of organic acids in the CFSs of L. rhamnosus GG/B. lactis Bb12 and APEC O78 co-cultures was also determined as above. Lactobacillus acidophilus and Levilactobacillus brevis were used to compare the organic acids profiles. The LC-MS/MS Poroshell 120 SB C18 column containing solvent A; H₂O+0.1% formic acid and solvent B; acetonitrile (MeCN)+0.1% formic acid was used for the LC-MS/MS analysis. Standard solutions of acetic, propionic, butyric, and lactic acids (Sigma Aldrich, MO, USA) were used to generate the calibration curve and quantify the concentration of organic acids in the CFSs. Sodium ¹³C-lactic acid was used as an internal standard.

Identification of bioactive peptides: To identify the bioactive peptides present in L. rhamnosus GG and B. lactis Bb12 CFSs, LC-MS/MS was used as described previously. To prepare the CFSs, L. rhamnosus GG and B. lactis Bb12 were grown anaerobically for 24 h, centrifuged (1000 rpm, 10 min, 25° C.) and washed with sterile water. The L. rhamnosus GG and B. lactis Bb12 pellets were resuspended in sterile water containing 2% glucose and incubated for 24 h under anaerobic conditions. The L. rhamnosus GG and B. lactis Bb12 cultures were then centrifuged (1000 rpm, 10 min, 4° C.) and CFSs were separated by filtering through 0.2 μm filter. Lactobacillus acidophilus and Levilactobacillys brevis were used to compare the peptides profiles. To prepare the samples for LC-MS/MS run, CFSs (1.8 mL) were passed three times through HyperSep™ Hypercarb™ SPE cartridge (50 mg; ThermoFisher Scientific, MA, USA). The cartridge was washed twice with water (150 μL) to remove salts and peptides were eluted (20 μL) using 50% MeCN and 0.1% trifluoroacetic acid (TFA). The elutes (0.5 μL) were injected into LC-MS/MS EasySpray C18-Fusion column set at HCD (higher energy collision dissociation) and CID (ion-trap-based collision-induced dissociation) collision energy settings. The solvent A; H₂O+0.1% formic acid and solvent B; MeCN+0.1% formic acid were used. The data generated were analyzed using ProteomeDiscoverer 2.2 software (ThermoFisher Scientific) using UniProt Lactobacillus or Bifidobacterium database with settings of no modifications and non-specific cleavage.

The five common highly abundant peptides in CFSs of both L. rhamnosus GG and B. lactis Bb12 (FSAVALSAVALSKPGHVNA (SEQ ID NO:5), AESSDTNLVNAKAA (SEQ ID NO:18), VQAAQAGDTKPIEV (SEQ ID NO:21), AFDNTDTSLDSTFKSA (SEQ ID NO:26) and VTDTSGKAGTTKISNV (SEQ ID NO:32)) were synthesized (GenScript, NJ, USA) and tested for anti-APEC activity by conducting kinetic time-inhibition assay as described previously. Briefly, peptides dissolved in dimethyl sulfoxide (DMSO) at 200 mM concentrations were added (12 mM; final concentration of 6% DMSO) to APEC suspension (105 CFU/mL; LB media) in a 96-well plate. The plate was then incubated in TECAN Sunrise™ absorbance microplate reader (NC, USA) at 37° C. with OD₆₀₀ measurement set at every 30 mins for 12 h. Untreated APEC (0% DMSO) and APEC treated with 6% DMSO were included as controls. DMSO at 6% in our earlier study showed no significant effect on APEC growth, only when used at 8% concentration significant effect on APEC growth was observed. Three independent experiments were conducted.

Cell culture studies: The anti-APEC activity of L. rhamnosus GG and B. lactis Bb12 CF Ss was studied in cell culture model using polarized HT-29 (Human Colorectal Adenocarcinoma Cell Line; ATCC HTB-38) cells, maintained in complete Dulbecco's modified Eagle's medium (DMEM, Gibco, MA, USA) supplemented with 10% fetal bovine serum (FBS, Gibco), 2 mM L-glutamine, 5 mM galactose, 1% penicillin-streptomycin (PS) solution and 0.1 mM non-essential amino acids (NEAA). To evaluate the effect of CFSs of L. rhamnosus GG and B. lactis Bb12 on APEC O78 adhesion to HT-29 cells, 10% CFSs from the 24 h grown L. rhamnosus GG and B. lactis Bb12 cultures were added to the wells containing HT-29 monolayers, which was incubated for 3 has described previously. The CFSs diluted to 10% were used as they were non-toxic to HT-29 cells as well as non-inhibitory to APEC growth at this concentration (data not shown). Prior to treatment with CFSs, the polarized HT-29 cells were washed and incubated for 2 h in DMEM containing no antibiotics and FBS. After treatment, the HT-29 cells were washed with DPBS, infected with APEC 078 (MOI 100), and incubated for 3 h. For infection, the logarithmic phase grown APEC O78 was pelleted, washed, and resuspended in DMEM at OD₆₀₀ 0.05 (5×10⁷ CFU/mL). The infected HT-29 cells were washed three times and the adherent APEC O78 was enumerated after lysis with 0.5% Triton X-100 followed by serial dilution (10-fold) and plating on LB agar plate.

To determine the effect of CFSs on APEC O78 invasion, the HT-29 cells were pre-treated with CFSs and infected with APEC O78 as described above. Following 3 h incubation with APEC 078, the HT-29 cells were washed three times and treated with DMEM containing 150 μg/mL gentamicin for 1 h. The HT-29 cells were washed twice with DPBS, lysed and invaded APEC O78 was quantified as described above. Two independent experiments were conducted with three replicates in each experiment. The effect of pre-treatment (3 h) of L. rhamnosus GG and B. lactis Bb12 cells itself after separation of culture supernatant by centrifugation and washing as above was also determined. The washed L. rhamnosus GG and B. lactis Bb12 pellets were resuspended in DMEM at OD₆₀₀ 1.0 (˜10⁹ CFU/mL) prior to adding into the wells containing HT-29 monolayers and procedure as above was followed.

Scanning electron microscopy (SEM): To determine the modes of action of L. rhamnosus GG and B. lactis Bb12, APEC O78 was treated with CFSs of L. rhamnosus GG and B. lactis Bb12 and imaged using Hitachi S-4700 scanning electron microscope as described previously. Briefly, APEC O78 culture adjusted at OD₆₀₀ 1.0 (1×10⁹ CFU/mL) was treated with CFSs prepared from 24 h grown L. rhamnosus GG and B. lactis Bb12 for 2 h at 37° C. with shaking at 200 rpm. Following treatment, culture was processed for SEM as described previously.

Efficacy of L. rhamnosus GG and B. lactis Bb12 in chickens: Animal study was approved by The Ohio State University Institutional Animal Care and Use Committee (IACUC, protocol #2010 A00000149). Chickens were euthanized using C02 following American Veterinary Medical Association (AVMA) guidelines. Standard animal husbandry practices were followed throughout the experiment. Feed and water were provided ad libitum.

The efficacy of L. rhamnosus GG and B. lactis Bb12 and their combination (1:1) was tested in one-day-old specific pathogen free (SPF) Leghorn chickens (n=10/group). From day 1, L. rhamnosus GG and B. lactis Bb12 were administered orally (200 μL in PBS; 108 CFU/chicken), once a day, until day 14. On day 7, chickens were infected orally with rifampicin resistant (Rif) APEC O78 (7.5×10⁷ CFU/chicken) as described previously. Before the infection, random cloacal swabs were collected from each group (n=2/group) to confirm the absence of APEC. Chickens infected with APEC but not treated with probiotic (positive control, PC) and not infected with APEC and not treated with probiotic (negative control, NC) were included as controls. On day 15, chickens were euthanized, and tissues (cecum, liver, and heart) were aseptically collected for APEC quantification. The tissues were homogenized in PBS and the suspensions were ten-fold serially diluted and plated on MacConkey agar plates containing 50 μg/mL rifampicin. Body weight of chickens was measured at day 1 and 15.

The L. rhamnosus GG-specific quantitative polymerase chain reaction (qPCR) was performed to assess the presence of L. rhamnosus GG in the cecum of L. rhamnosus GG-treated chickens as previously described. The primers (SEQ ID NO: 34-35) (Table 7) were obtained from Integrated DNA Technologies (IDT). The qPCR (two-step) was performed using Maxima SYBR Green/ROX qPCR master mix (ThermoFisher Scientific) following the manufacturer's instructions in a RealPlex² Mastercycler® (Eppendorf, CT, USA) with single cycle of 95° C. for 10 min and 40 cycles of amplification with 95° C. for 15 secs denaturing and 60° C. for 1 min annealing temperatures. PureLink™ Microbiome DNA Purification Kit (Thermofisher Scientific) was used to extract the microbial DNA from the cecal contents (approximately 0.2 g) of the chickens. RNase A treatment (2-3 μL of 100 mg/mL solution per sample; Qiagen, MD, USA) was performed to remove the RNA. DNA quantity and quality were measured using NanoDrop 2000c Spectrophotometer (ThermoFisher Scientific). The standard L. rhamnosus GG qPCR curve was used to enumerate the L. rhamnosus GG which was generated by making 10-fold serial dilutions of L. rhamnosus GG DNA extracted (MasterPure™ DNA Purification Kit; Epicentre, WI, USA) from OD₆₀₀ 1.0 L. rhamnosus GG (˜10⁹ CFU/mL) culture. The qPCR was also performed for microbial DNA extracted from cecal contents of NC chickens to confirm the specificity of L. rhamnosus GG primers.

Cecal microbiome analysis: To investigate the impact of L. rhamnosus GG treatment on the cecal microbiome of chickens, 16S rRNA based microbiome study was conducted as previously described. DNA was extracted from 0.2 g of cecal contents using PureLink™ Microbiome DNA Purification Kit (ThermoFisher Scientific) and treated with RNase A (2-3 μL of 100 mg/mL solution per sample; Qiagen). DNA quantity and quality were measured using NanoDrop 2000c Spectrophotometer (ThermoFisher Scientific). The extracted DNA samples were subjected to 16S rRNA V4-V5 sequencing at the molecular and cellular imaging center (MCIC) (mcic.osu.edu/genomics/illumina-sequencing). Amplicon libraries were prepared using IFU KAPA HiFi HotStart ReadyMixPCR Kit (Roche, NJ, USA) and PCR clean-up was performed using Agencourt AMPure XP beads (BECKMAN COULTER Life Sciences, CA, USA). Nextera XT DNA Library Preparation Kit (Illumina, CA, USA) was used to generate Illumina library and sequencing was performed using Illumina MiSeq platform generating paired end 300-bp reads.

For the microbiota analysis, QMIIE (Quantitative Insights Into Microbial Ecology) 2 bioinformatics platform (qiime2.org/) was used. Quality control of the raw reads was performed using FastQC 0.11.8 (Babraham Bioinformatics). Trimmomatic-0.33 was used to trim the adaptor and other illumina-specific sequences (www.usadellab.org/cms/?page=trimmomatic). The trimmed sequences (fastq.gz) were imported into the QIIME 2 as a manifest file format (PairedEndManifestPhred33V2). The feature table construction and additional filtering of the sequences was performed using DADA2. The taxonomic analysis was performed using Naive Bayes classifiers trained on Silva 132 99% OTUs (silva-132-99-nb-classifier.qza) database. The phylogenetic diversity was analyzed using align-to-tree-mafft-fasttree pipeline and alpha (Shannon's diversity index) and beta diversity (Bray-Curtis distance) were analyzed using core-metrics-phylogenetic pipeline (docs.qiime2.org/2019.7/tutorials/moving-pictures/). The statistical difference (P<0.05) in the taxonomic composition between the L. rhamnosus GG treated, PC (APEC infected but not treated with L. rhamnosus GG), and NC (non-APEC infected and non-L. rhamnosus GG treated) groups was determined using Mann-Whitney U test. The alpha and beta diversity were analyzed using Kruskal-Wallis and PERMANOVA tests (P<0.05), respectively.

Statistical analysis: The statistical significance (P<0.05) of bacterial viability reduction and inhibition of adhesion and invasion was calculated using two-way ANOVA followed by Bonferroni post-test. The statistical significance (P<0.05) of treatment on reduction of APEC load and increment in body weight was calculated using Mann-Whitney U test.

### Results

L. rhamnosus GG and B. lactis Bb12 Induced Strong Zone of Inhibition Against APEC Serotypes

Of the several whole cultures of commensal and probiotic bacteria tested (Table 1), L. rhamnosus GG and B. lactis Bb12 induced large zone of inhibition against APEC O78 at 12 h (14.5±0.5 and 13.5±0.5) and 24 h (12.5±0.5 and 11.5±0.5) post-incubation in agar-well diffusion assay (Table 2). Enterococcus faecalis, Levilactobacillus brevis, Bifidobacterium adolescentis and Bacteroides thetaiotaomicron also induced zone of inhibition at 12 h (9.5±0.5, 9.5±0.5, 12.5±0.5 and 9.5±0.5, respectively); however, no zone of inhibition was observed at 24 h. The decrease in inhibition at 24 h might be due to lack of continuous production of inhibitory substances in solid media by commensal and probiotic bacteria as stationary phase grown cultures were used in the assay. Slight but not measurable zone of inhibition was also observed with Lactobacillus acidophilus, Streptococcus bovis and Bifidobacterium longum (data not shown). No zone of inhibition was observed with E. coli Nissle 1917 and E. coli G58-1.

The zone of inhibition induced by L. rhamnosus GG and B. lactis Bb12 against APEC O78 was volume dependent. Large zone of inhibition was observed when incubated with 200 μL (18.5±0.5 and 17.5±0.5) of culture volume followed by 150 (16.5±0.5 and 15.5±0.5), 100 μL (14.5±0.5 and 13.5±0.5) and 50 μL (10.5±0.5 and 10.0±1.0). Further, L. rhamnosus GG and B. lactis Bb12 also induced similar zone of inhibition against other multiple pre-dominant APEC (O1, O2, O8, O15, O18, O35, O109 and O115) serotypes (Table 8).

No Viable APEC was Detected when APEC was Incubated with L. rhamnosus GG and B. lactis Bb12

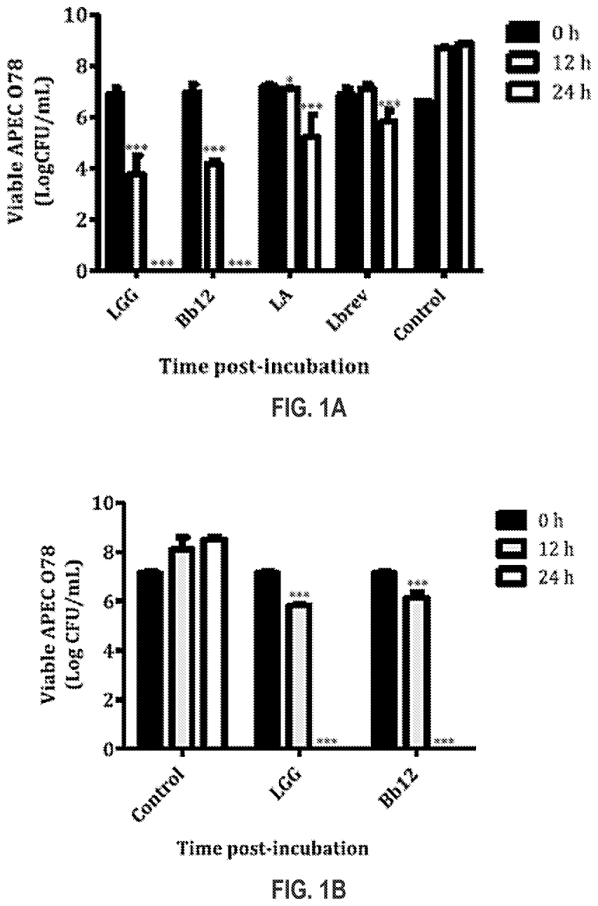

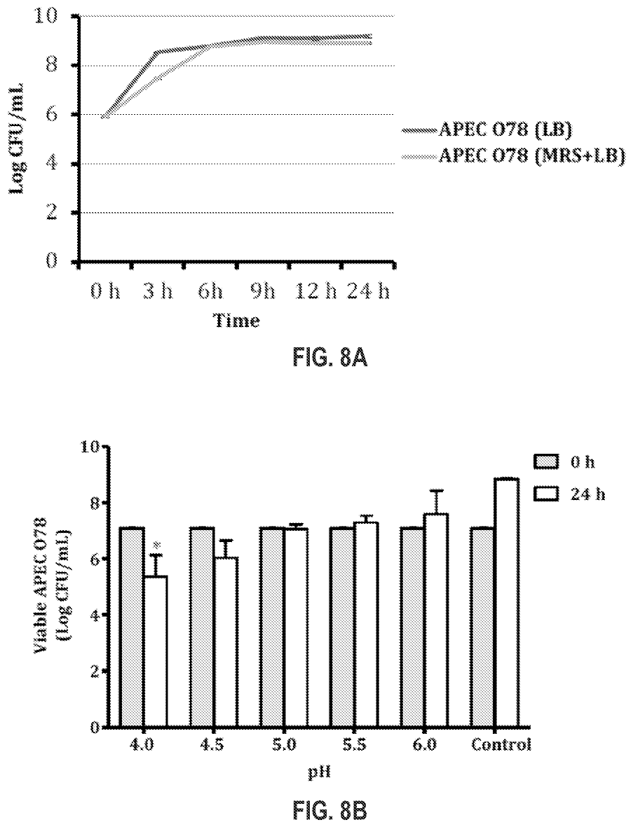

The growth of APEC was not compromised in co-culture (100% MRS+100% LB) media as compared to LB media (FIG. 8A). The significant reduction (P<0.001) in viable APEC was observed at 12 h when incubated with L. rhamnosus GG and B. lactis Bb12 in co-culture media; whereas no reduction was observed when incubated with L. acidophilus and L. brevis. At 24 h, no viable APEC was recovered when incubated with L. rhamnosus GG and B. lactis Bb12; whereas slight reduction (<2 logs) was also observed when incubated with L. acidophilus and L. brevis (FIG. 1A).

L. rhamnosus GG and B. lactis Bb12 Secreted/Released Products are Responsible for Anti-APEC Activity

As observed in co-culture assay, both L. rhamnosus GG and B. lactis Bb12 CFSs significantly (P<0.001) reduced the viable APEC population at 12 h and no viable APEC was detected at 24 h (FIG. 1B) in trans-well migration assay. Further, L. rhamnosus GG and B. lactis Bb12 CFSs also induced the zone of inhibition similar to L. rhamnosus GG and B. lactis Bb12 whole culture in agar-well diffusion assay (Table 3). However, no zone of inhibition was induced by L. rhamnosus GG and B. lactis Bb12 cells itself after CF Ss were separated and cells resuspended in PBS. Further, the heat and proteolysis treated CF Ss of L. rhamnosus GG and B. lactis Bb12 retained the anti-APEC activity similar to untreated CFSs (Table 3). Similarly, fractionated (<3 kDa) CFSs of L. rhamnosus GG and B. lactis Bb12 also exhibited the anti-APEC activity similar to unfractionated CFSs (Table 3), showing that secreted/released products are heat stable, proteolysis resistant and of low mol. wt. in size.

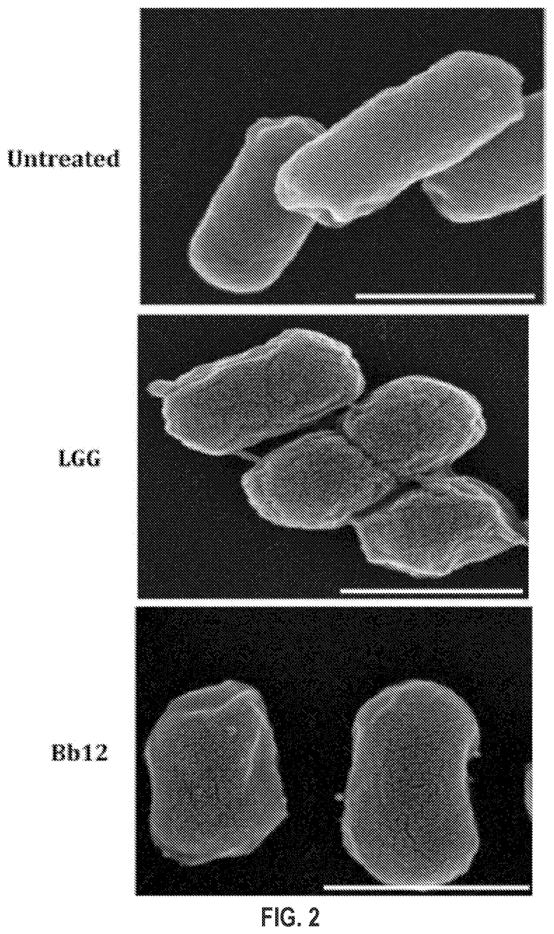

The shortened bacterial cells measuring ˜0.5-1 μM with bulbous swelling were observed after treatment with L. rhamnosus GG and B. lactis Bb12 CFSs as compared to untreated bacterial cells which measured ˜1.5-2 μM in length (FIG. 2).

Activity of L. rhamnosus GG and B. lactis Bb12 is pH-Independent

It has been previously shown that probiotic lactic-acid bacteria exert inhibitory effect against pathogenic bacteria by lowering the pH. Therefore, the change in pH when APEC was co-cultured with L. rhamnosus GG, B. lactis Bb12, L. acidophilus, and L. brevis was monitored. At 24 h, the lowest pH was observed when APEC was co-cultured with B. lactis Bb12 (4.12), followed by L. rhamnosus GG (4.37), L. acidophilus (4.66) and L. brevis (4.96) (Table 4). The viability of APEC in co-culture media adjusted to different pH (ranging 4.0 to 6.0) was also quantified in order to determine the effect of pH on L. rhamnosus GG and B. lactis Bb12 anti-APEC activity. No significant effect on the viability of APEC was observed, except at pH 4.0 (FIG. 8B); however, significant number (˜5.4 logs) of APEC was still viable even at pH 4 after 24 h compared to no viable APEC recovered when incubated with L. rhamnosus GG and B. lactis Bb12 (FIG. 8B). APEC was also pH-tolerant up to pH 4.0 when incubated for 24 h in LB media alone adjusted to different pH (ranging 4.0 to 6.0; data not shown). These studies show that pH alone is not responsible for anti-APEC activity of L. rhamnosus GG and B. lactis Bb12.

L. rhamnosus GG and B. lactis Bb12 Contain Lactic Acid and Multiple Small Peptides in their Cell-Free Supernatants

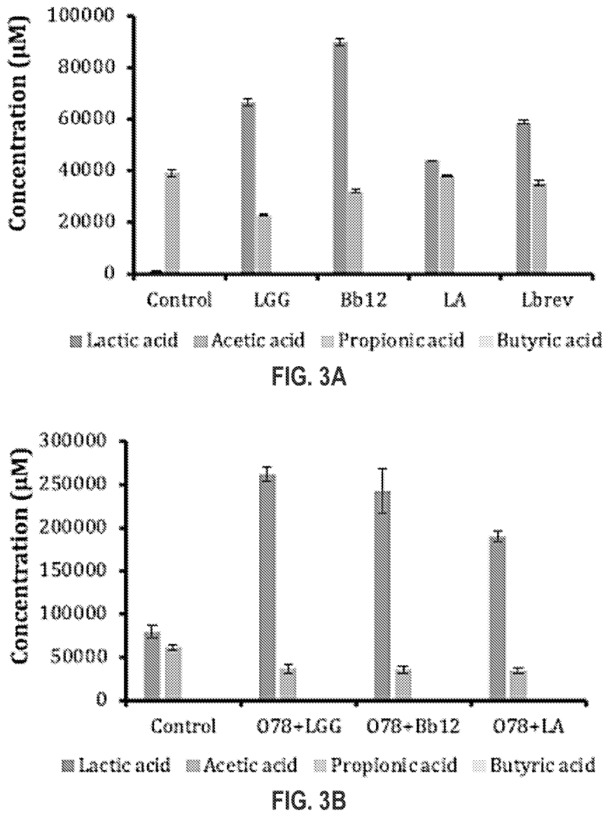

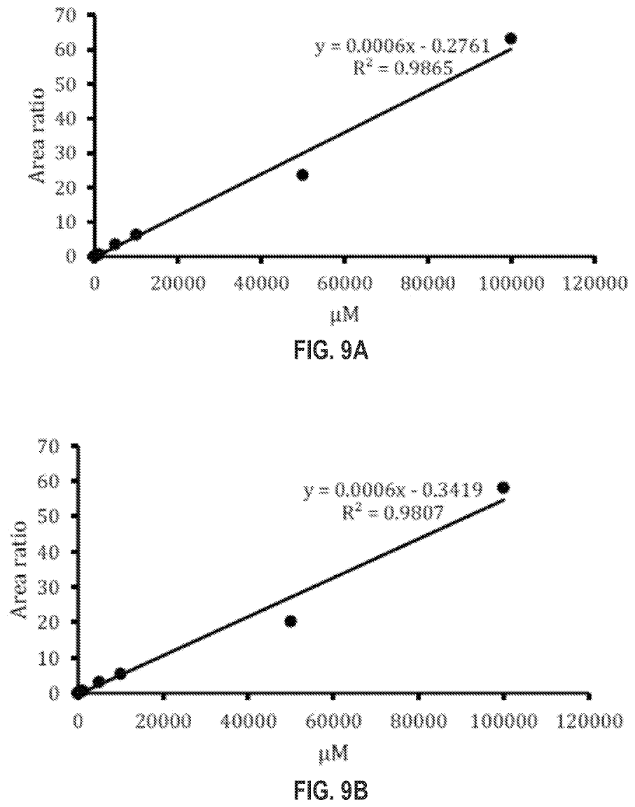

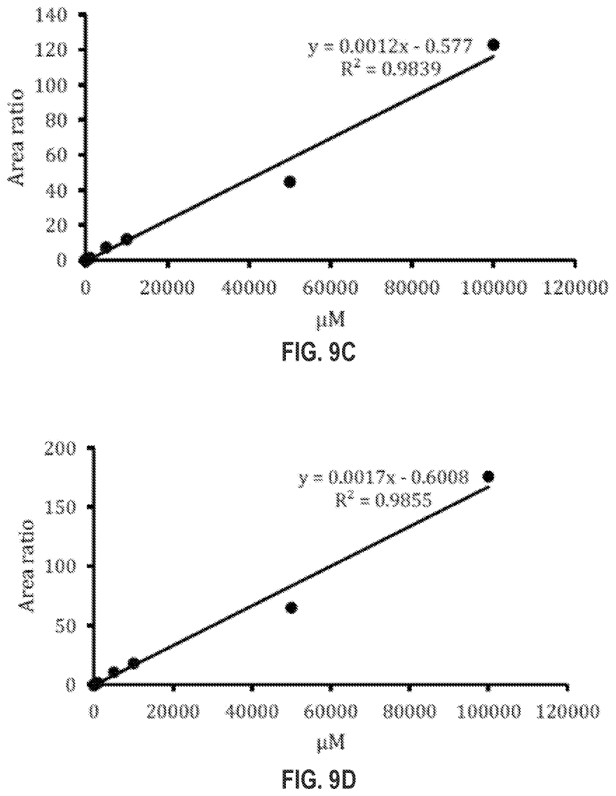

LC-MS/MS coupled with isotope-labeled chemical derivatization method was used to quantify the organic acids produced by L. rhamnosus GG, B. lactis Bb12, L. acidophilus, and L. brevis. The standard curves of lactic (y=0.0006×−0.2761, R²=0.9865), acetic (y=0.0006×−0.3419, R²=0.9807), propionic (y=0.00012×−0.577, R²=0.9839) and butyric acids (y=0.0017×−0.6008, R²=0.9855) were generated to quantitate the concentration of organic acids in CFS (FIGS. 9A, 9B, 9C, 9D). Lactic acid was predominantly present in all CFSs (FIG. 3A). The highest concentration of lactic acid was observed in CFS of B. lactis Bb12 (0.090M) followed by L. rhamnosus GG (0.067M), L. brevis (0.059M) and L. acidophilus (0.044M). Interestingly, higher concentrations of lactic acid were produced by L. rhamnosus GG (0.26M), B. lactis Bb12 (0.24M) and L. acidophilus (0.19M) when co-cultured with APEC compared to monoculture (FIG. 3B).

LC-MS/MS analysis of CFSs eluted through HyperSep™ Hypercarb™ SPE cartridge was also performed to identify the bioactive molecules secreted/released by L. rhamnosus GG, B. lactis Bb12, L. acidophilus, and L. brevis. At HCD (higher energy collision dissociation) setting, 57 peptides (Dataset 1) were identified, whereas 152 peptides were identified at CID (ion-trap-based collision-induced dissociation) setting (Dataset 2). A total of 33 peptides (SEQ ID NOS: 1-33) (Table 5) of mol. wt. less than 3 kDa were identified in common in both HCD and CID settings. Consistent with strong anti-APEC activity of L. rhamnosus GG and B. lactis Bb12 compared to L. acidophilus and L. brevis, these peptides were mostly present in CFSs of L. rhamnosus GG and B. lactis Bb12.

L. rhamnosus GG and B. lactis Bb12 Cell-Free Supernatants Reduced the Adhesion and Invasion of APEC in HT-29 Cells

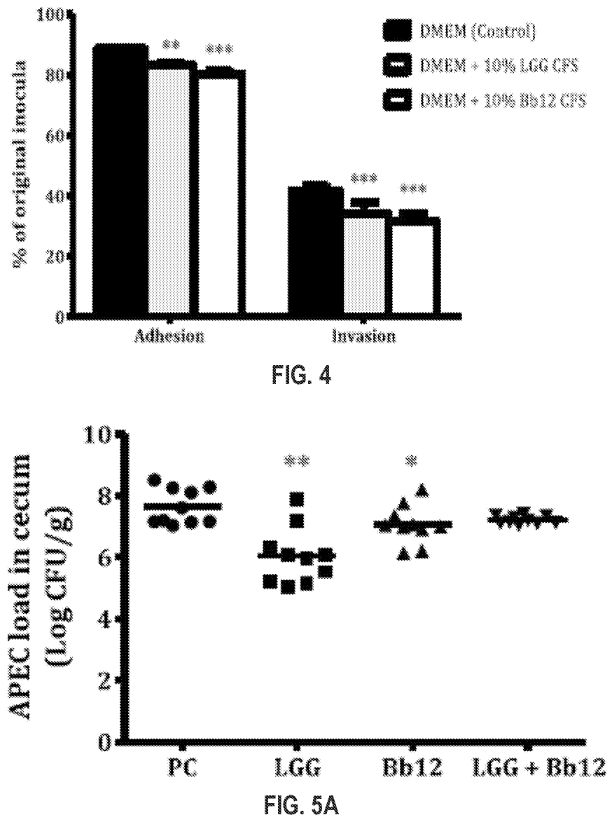

The HT-29 cells were pre-treated with 10% CFSs (concentration non-toxic to HT-29 cells and non-inhibitory to APEC growth) for 3 h to determine the effect of CFSs on adhesion and invasion of APEC. Both the CFSs significantly reduced (P<0.05) the percent of original inoculant of APEC adhered and invaded in HT-29 cells (FIG. 4). However, no effect on the adhesion and invasion was observed when HT-29 cells were pre-treated with L. rhamnosus GG and B. lactis Bb12 cells itself after CFSs were separated and cells resuspended in DMEM (data not shown).

L. rhamnosus GG Reduced the Colonization of APEC in Cecum of Chickens

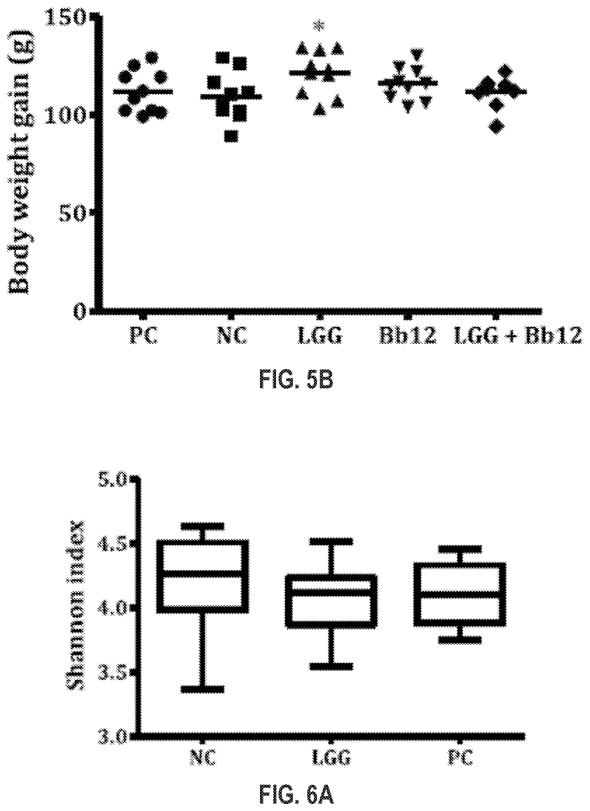

The efficacy of L. rhamnosus GG and B. lactis Bb12 and their combination (1:1) was tested in chickens by administering orally (10⁸ CFU/chicken) for 14 days. The L. rhamnosus GG treatment significantly reduced (P<0.001; 1.6 logs) the APEC load in cecum 7 days post-infection as compared to APEC infected but not probiotic treated (PC; positive control) chickens (FIG. 5A). On the other hand, only 0.6 log APEC reduction was observed in B. lactis Bb12 treated chickens. No L. rhamnosus GG treated chickens were positive for APEC in internal organs (liver and heart); whereas 10% and 20% of chickens were APEC positive in B. lactis Bb12 treated and untreated groups, respectively (data not shown). Surprisingly, the combination treatment with L. rhamnosus GG and B. lactis Bb12 only resulted in 0.4 log reduction in APEC load.

L. rhamnosus GG treatment also significantly (P<0.05; 12 g in two-weeks) increased the body weight gain of chickens as compared to not APEC infected and not probiotic treated (NC; negative control) chickens (FIG. 5B). Whereas no significant increase in body weight was observed in chickens treated with B. lactis Bb12 (6.6 g) or L. rhamnosus GG and B. lactis Bb12 combination (2.6 g).

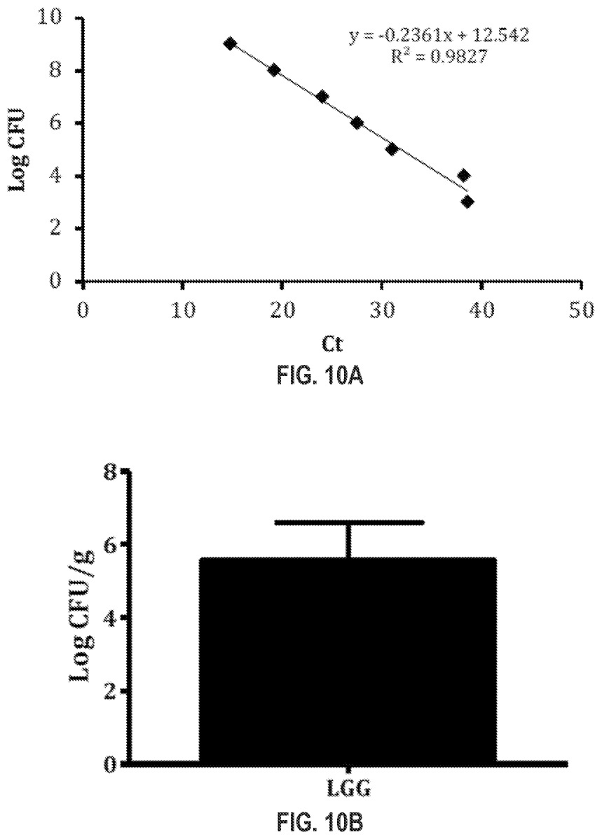

L. rhamnosus GG-specific qPCR was performed to quantitate L. rhamnosus GG in the cecum of L. rhamnosus GG-treated chickens. The standard L. rhamnosus GG qPCR curve was generated (FIG. 10A) and used to quantitate L. rhamnosus GG in cecum. At day 15, ˜6.3 logs of L. rhamnosus GG (on average) was enumerated in cecum (FIG. 10B). No amplification of L. rhamnosus GG was observed in cecal contents of NC chickens. These results demonstrate that L. rhamnosus GG can resist the low gastric pH and high intestinal bile salt concentrations of the chicken's gut.

L. rhamnosus GG Reduced the Enterobacteriaceae (Escherichia-Shigella) Abundance in Cecum of Chickens

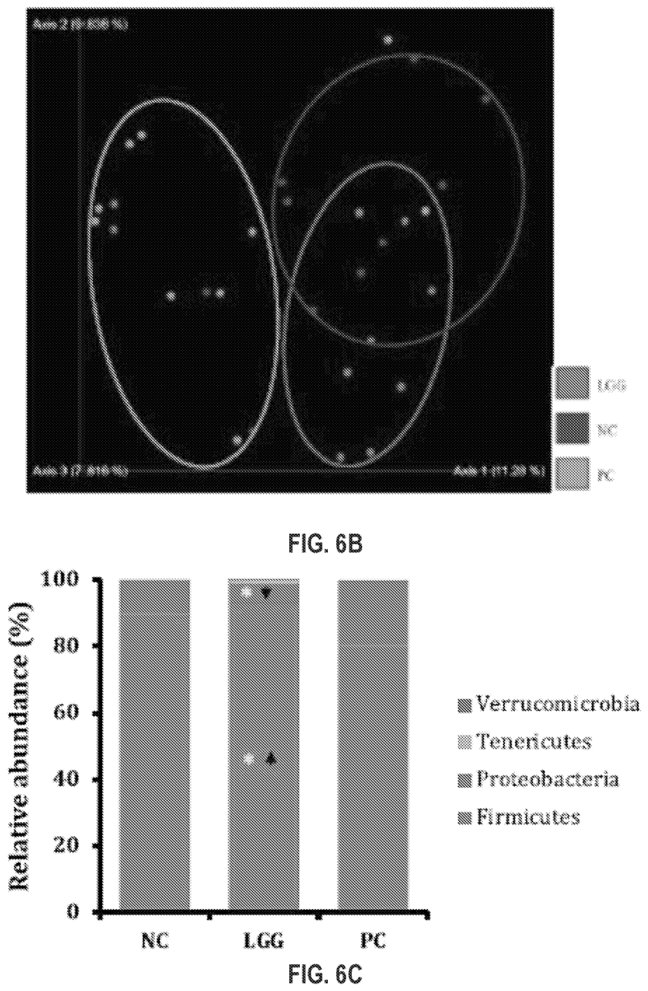

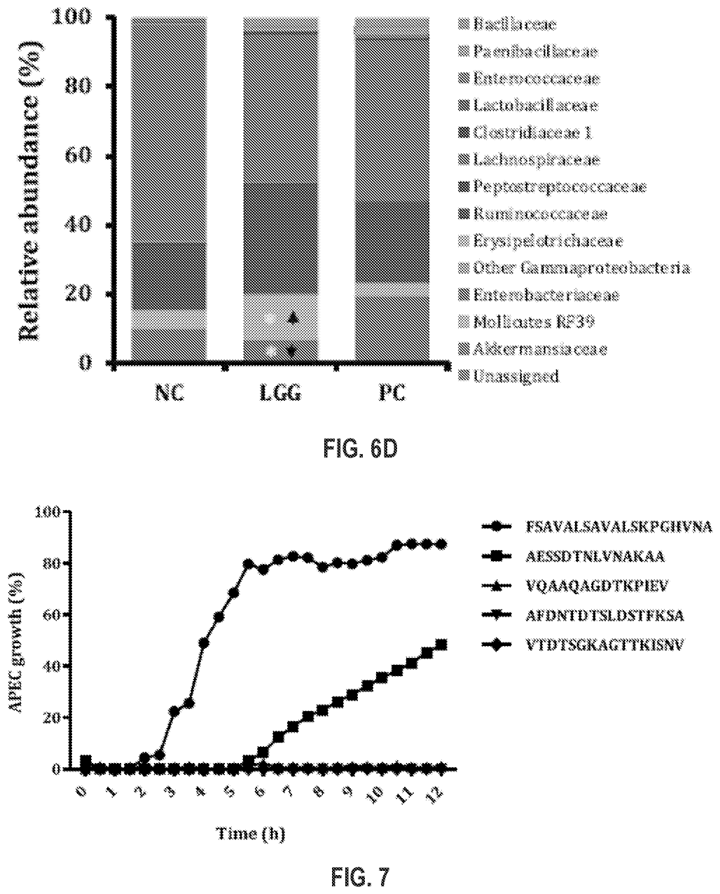

The analysis of alpha-diversity (or Shannon index) revealed no significant difference in the microbial richness between the treatment groups (FIG. 6A). However, the microbial community of APEC infected but not treated (PC) chickens was dissimilar to L. rhamnosus GG treated and non-infected and non-treated (NC) chickens when beta-diversity was analyzed using Bray-Curtis dissimilarity index (FIG. 6B). The microbial communities of L. rhamnosus GG treated and NC chickens were similar, showing that L. rhamnosus GG moderated the APEC induced alterations of microbial community in the cecum of chickens.

The L. rhamnosus GG treatment significantly (P<0.05) increased (80.22% to 92.98%) the Firmicutes abundance, whereas decreased (19.72% to 6.11%) the Proteobacteria abundance as compared to PC chickens (FIG. 6C). Specifically, in Firmicutes, the abundance of bacteria belonging to Erysipelotrichia (3.64% to 14.23%) class, or Erysipelotrichales (3.64% to 12.99%) order was increased. On the other hand, in Proteobacteria, the abundance of bacteria belonging to Gammaproteobacteria (19.72% to 6.72%) class, or Enterobacteriales (19.57% to 6.11%) order was decreased. At the family level, the abundance of Enterobacteriaceae (19.57% to 6.11%) and Enterococcaceae (1.03% to 0.09%) was significantly decreased (P<0.05), whereas the abundance of Erysipelotrichaceae was significantly (P<0.05) increased (3.64% to 12.99%) (FIG. 6D). At the genus level, the abundance of Escherichia-Shigella (16.45% to 4.20%), Enterococcus (1.03% to 0.09%), Flavonfractor (6.73% to 2.24%), and Lachnospiraceae (uncultured) (4.51% to 0%) was significantly decreased (P<0.05), whereas abundance of Erysipelatoclostridium (3.60% to 12.93%), Negativibacillus (0% to 1.54%), DTU089 (0% to 1.02%), Butyricicoccus (1.08% to 2.45%), Blautia (0% to 2.10%), and Lactobacillus (0.03% to 0.56%) was increased (Table 6).

Compared to NC chickens, the abundance of Bacillales (0% to 3.61%) order was significantly increased in L. rhamnosus GG treated chickens. At the family level, the abundance of Bacillaceae (0% to 3.61%), Clostridiaceae 1 (0% to 0.64%) and Ruminococcaceae (18.73% to 32.35%) was increased, whereas abundance of Lachnospiraceae (62.85% to 42.58%) was significantly decreased (FIG. 6D). At the genus level, the abundance of Bacillus (0% to 3.61%), DTU089 (0% to 1.02%), and Negativibacillus (0% to 1.54%) was significantly (P<0.05) increased.

The abundance of bacteria belonging to Clostridia (82.51% to 70.77%) class, or Clostridiales (82.51% to 70.77%) order was significantly (P<0.05) decreased in PC chickens as compared to NC chickens. At the family level, the abundance of Lachnospiraceae (62.85% to 46.38%) was decreased (FIG. 6D). At the genus level, the abundance of Candidatus soleaferrea (1.31% to 0.34%) and Caproiciproducens (0.77% to 0.05%) was significantly decreased, whereas abundance of Flavonfractor (2.68% to 6.73%) and Lachnospiraceae (uncultured) (0% to 4.51%) was significantly increased (Table 6).

Peptides Identified in the Cell-Free Supernatants of L. rhamnosus GG and B. lactis Bb12 are Inhibitory to APEC

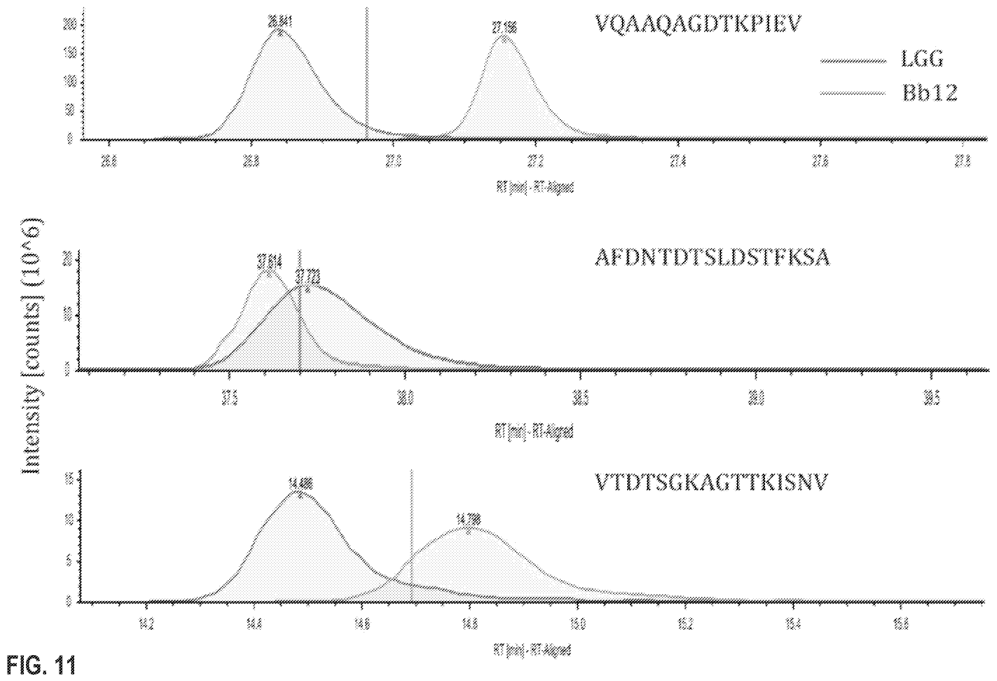

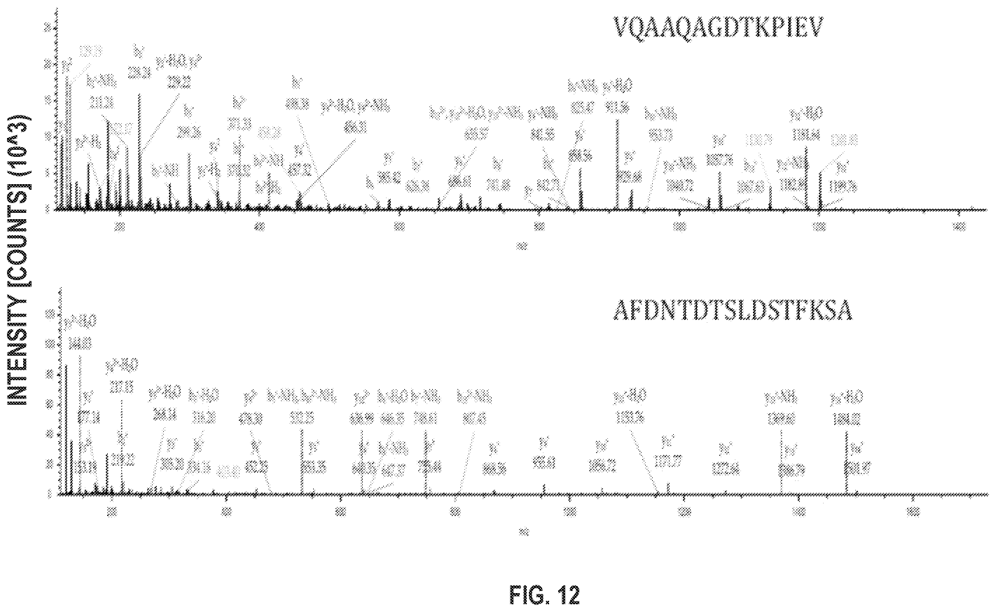

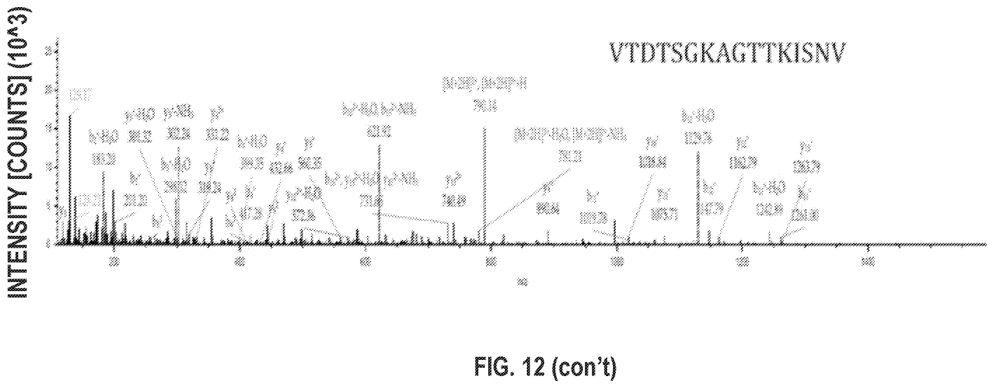

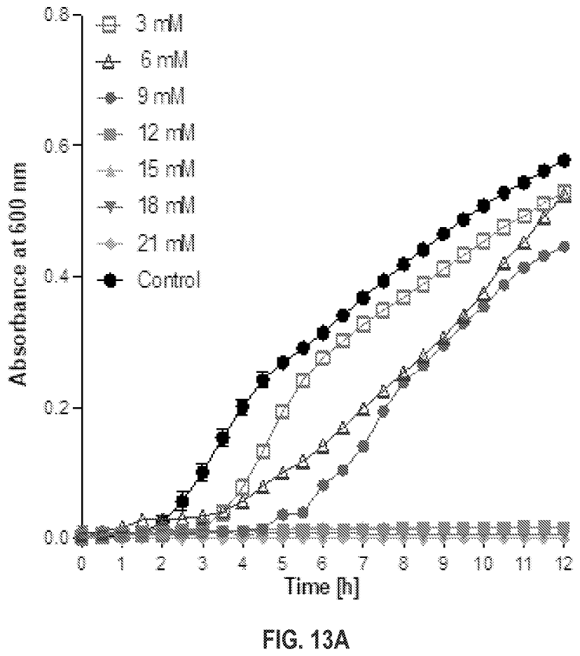

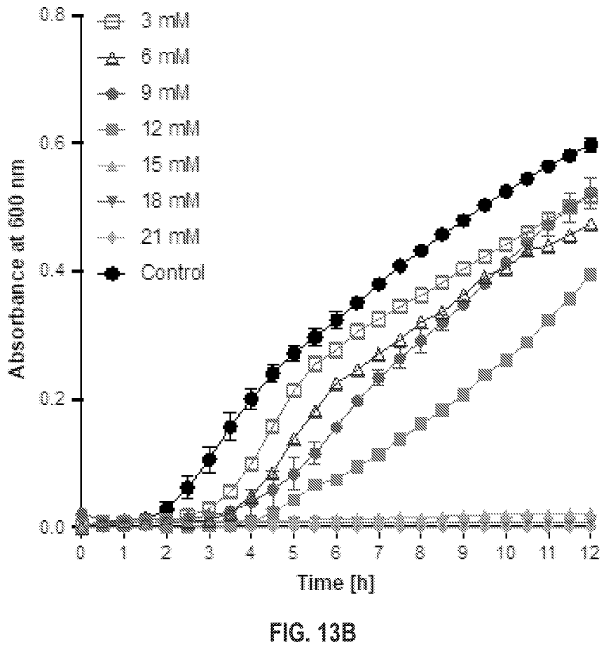

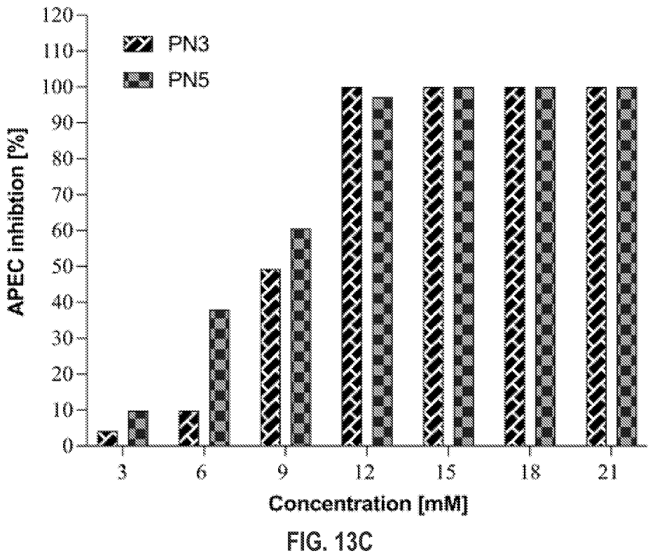

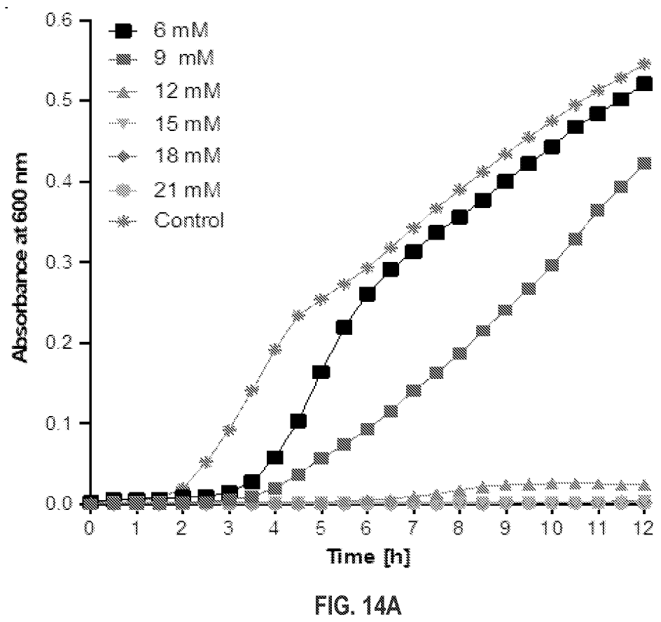

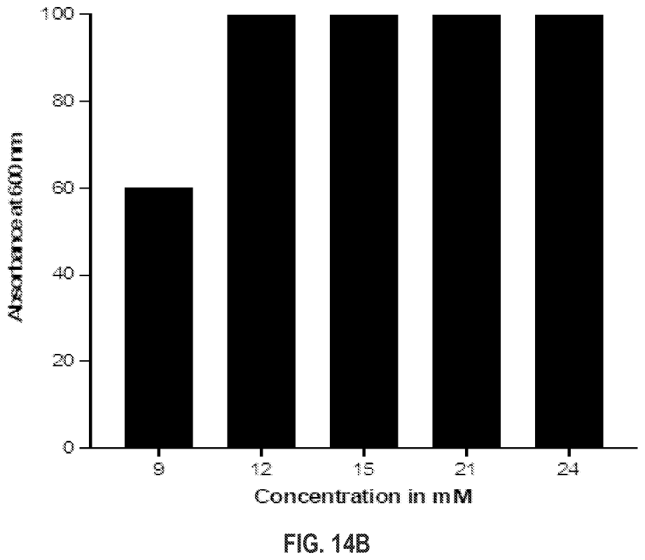

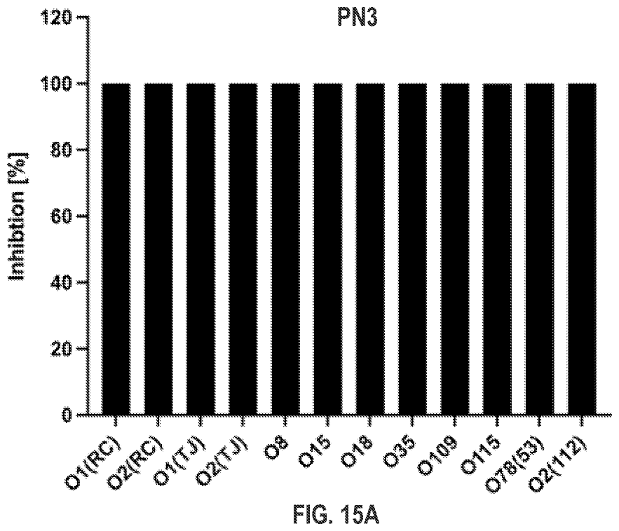

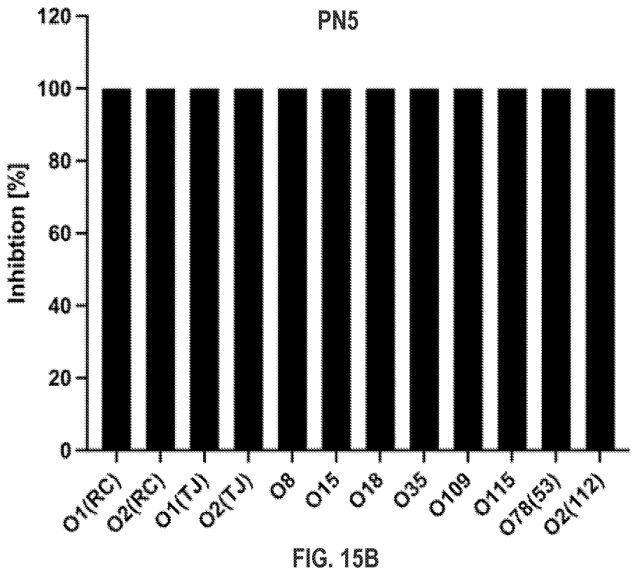

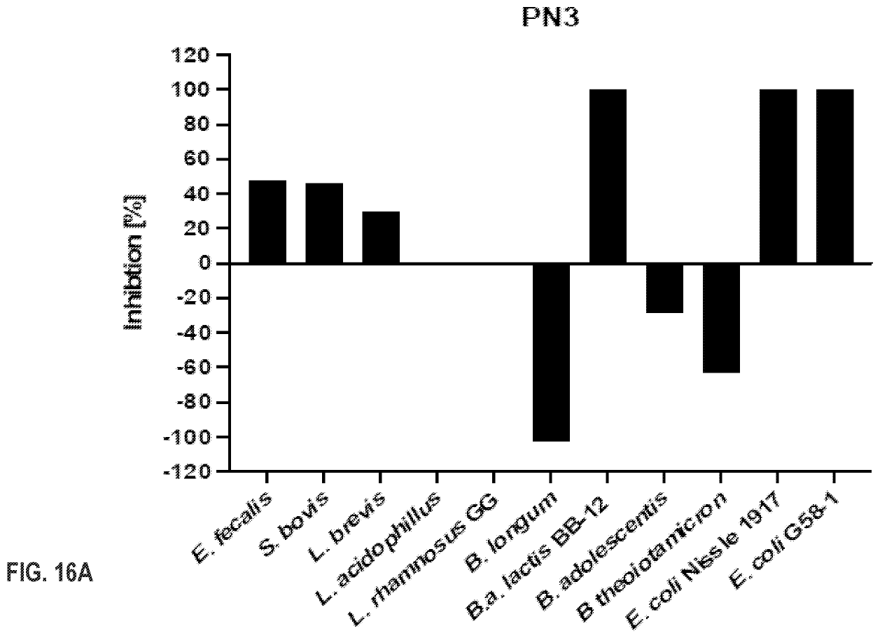

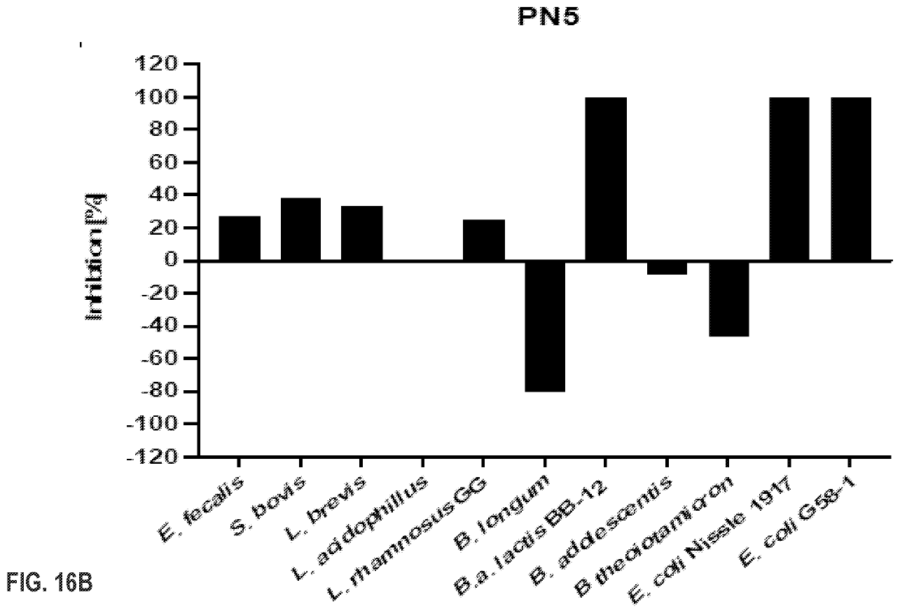

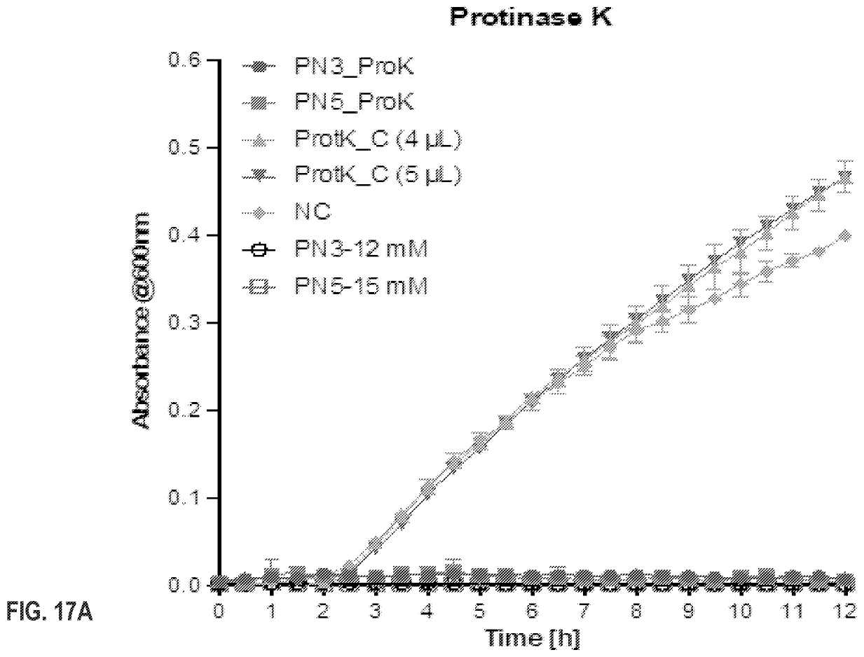

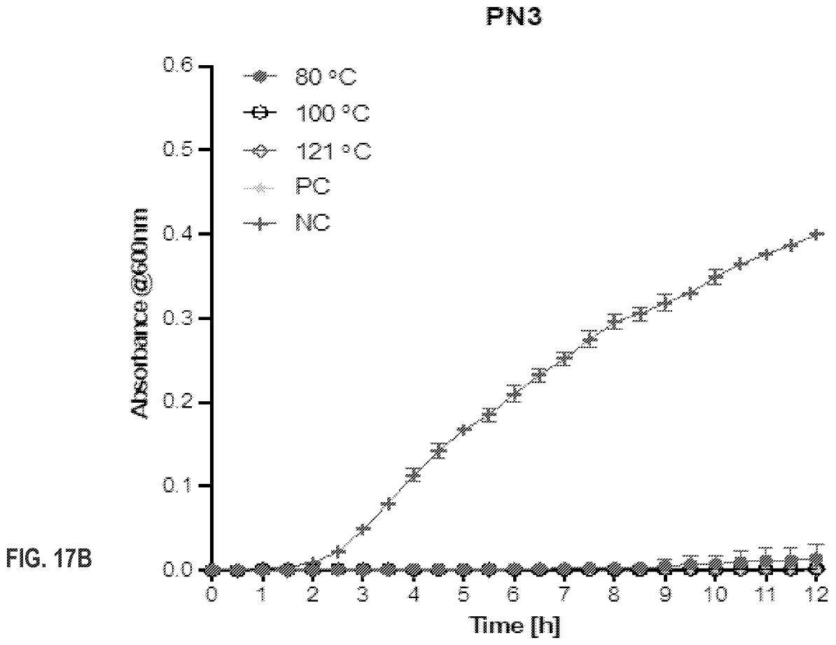

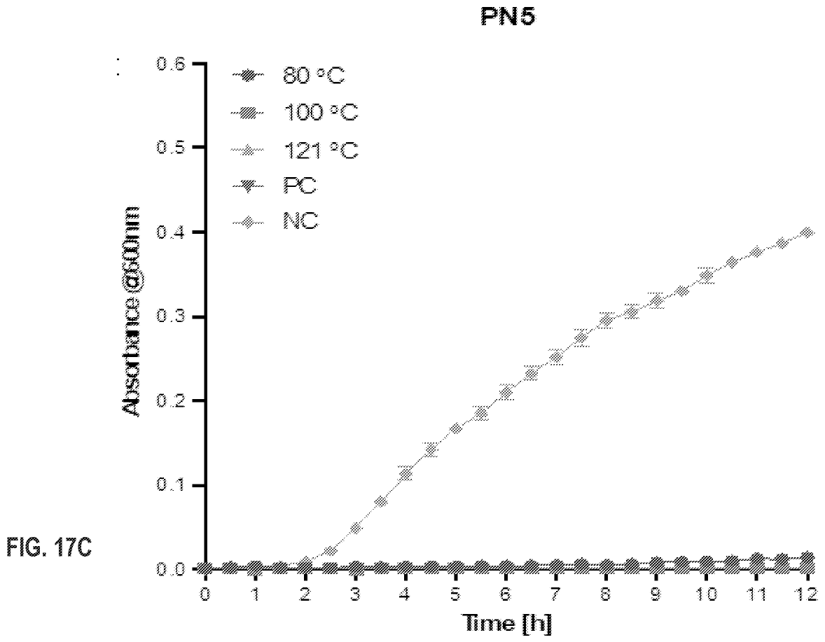

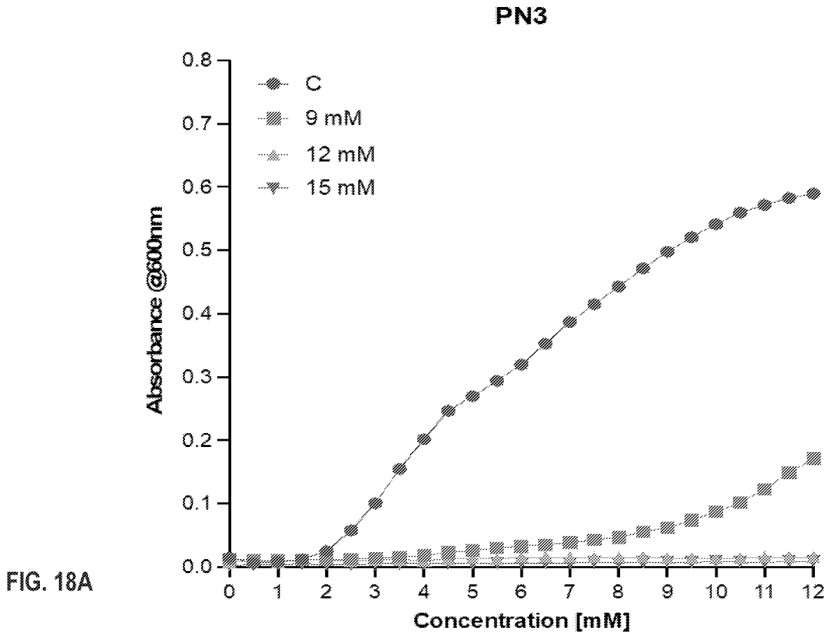

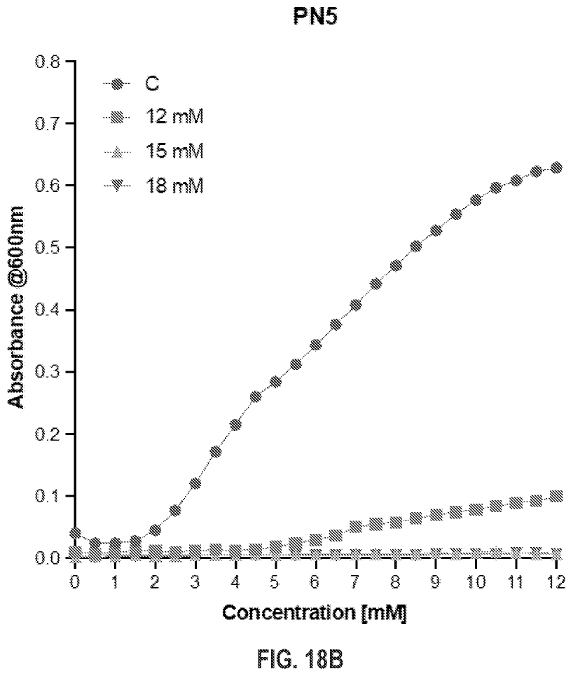

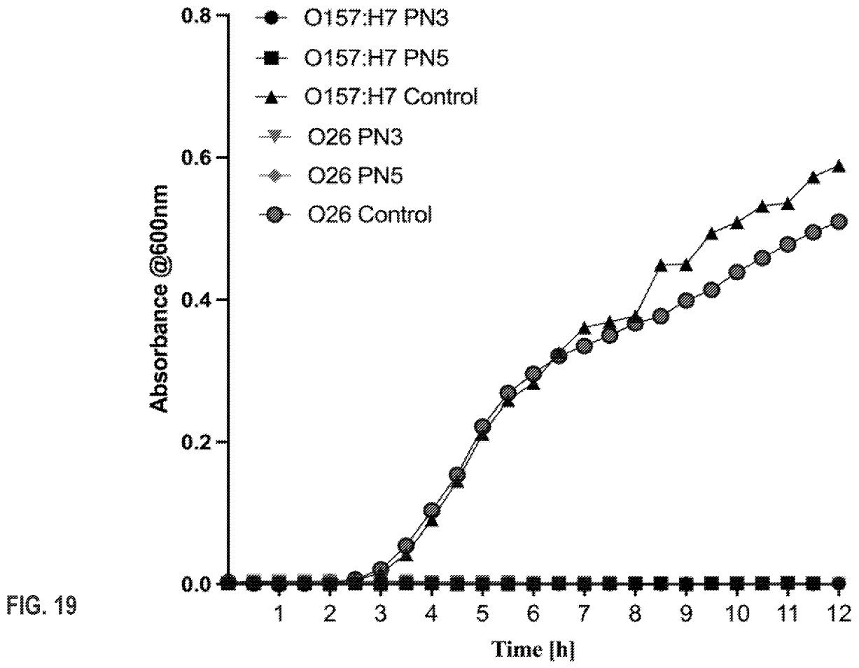

Out of 33 peptides identified by LC-MS/MS both at HCD and CID settings, five highly abundant common peptides present in both L. rhamnosus GG and B. lactis Bb12 were tested for anti-APEC activity. Three peptides (VQAAQAGDTKPIEV (SEQ ID NO:21), AFDNTDTSLDSTFKSA (SEQ ID NO:26) and VTDTSGKAGTTKISNV (SEQ ID NO:32)) were completely inhibitory to APEC (FIG. 7) at 12 mM concentration. The mass spectrometry (MS) peaks for these peptides are shown in FIGS. 11 and 12. Peptide AESSDTNLVNAKAA (SEQ ID NO:18) was slightly inhibitory to APEC, whereas FSAVALSAVALSKPGHVNA (SEQ ID NO:5) did not affect the APEC growth.

### Discussion

The efficacy of L. rhamnosus GG has been demonstrated to reduce infections caused by different bacterial pathogens in different animal hosts. L. rhamnosus GG administration reduced the S. infantis colonization in jejunum and its translocation to internal organs of piglets, S. typhimurium colonization in jejunum of piglets, and S. typhimurium-induced deaths in mouse model. Similarly, L. rhamnosus GG reduced the jejunal and ileal lesions caused by S. enterica serovar in piglets. Further, the culture supernatant of L. rhamnosus GG increased the resistance to systemic E. coli K1 infection in neonatal rats by reducing intestinal bacterial colonization, translocation, and dissemination to extra-intestinal sites. The mortality of mice was reduced when L. rhamnosus GG was administered in experimental model of septic peritonitis by preventing systemic bacteremia. L. rhamnosus GG supplementation also reduced the mortality in fish (red tilapia) challenged with Aeromonas veronii. These results demonstrate that L. rhamnosus GG is a preventative against APEC infection in chickens.

Previously, several antimicrobial peptides have been isolated and characterized from L. rhamnosus GG and other Lactobacillus sps. A 37.3 kDa postbiotic, HM0539, was identified in L. rhamnosus GG (ATCC 53103) supernatant through LC-MS/MS analysis. HM0539 showed beneficial effects against E. coli K1 infection in neonatal rats by promoting maturation of intestinal defense; however, effect on growth of E. coli K1 was not evaluated. Similar to the findings here, multiple small peptides (NPSRQERR (SEQ ID NO:85), PDENK (SEQ ID NO:86), VHTAPK (SEQ ID NO:87), MLNERVK (SEQ ID NO:88), YTRGLPM (SEQ ID NO:118), GKLSNK (SEQ ID NO:119) and LSQKSVK (SEQ ID NO:120)) of <1 kDa mol. wt. were identified in L. rhamnosus GG conditional media, they also showed growth inhibitory activity against Enteroaggregative E. coli (EAEC) 042 and APEC serotypes. Two major secreted proteins, p75 (major secreted protein 1; Msp1) and p40 (major secreted protein 2; Msp2), resembling cell wall hydrolases were identified in L. rhamnosus GG supernatant with reported functions in promoting the survival and growth of intestinal epithelial cells. In another study, a 1.3 kDa peptide was isolated from supernatant of L. gasseri SF1109 with anti-bacterial, anti-biofilm and immunomodulatory activities against Pseudomonas aeruginosa and E. coli. A 1.1 kDa peptide (NVGVLXPPXLV (SEQ ID NO:121); acidocin LCHV) was purified from supernatant of L. acidophilus n.v. Er 317/402 strain Narine that has broad spectrum of activity against Gram-positive and Gram-negative pathogens. Peptides (SGADTTFLTK (SEQ ID NO:122), LVGKKVQTE (SEQ ID NO:123) and GTLIGQDYK (SEQ ID NO:124)) isolated from supernatant of L. plantarum CECT 749 have also displayed antifungal activity against Aspergillus parasiticus and Penicillium expansum. These findings show that small peptides can be used as new therapeutics against APEC infections. From this study, three novel peptides (VQAAQAGDTKPIEV (SEQ ID NO:21), AFDNTDTSLDSTFKSA (SEQ ID NO:26) and VTDTSGKAGTTKISNV (SEQ ID NO:32)) were identified in the cell-free supernatant of L. rhamnosus GG that have anti-APEC activity (FIG. 7, Table 5). In the current study, five highly abundant peptides were tested for their bioactivity.

The abundance of bacteria belonging to phylum Proteobacteria, particularly Enterobacteriaceae family (Escherichia-Shigella), were decreased in gut microbiota of chickens treated with L. rhamnosus GG (FIG. 6, Table 6). The increase in phylum Proteobacteria which includes many opportunistic bacteria is associated with low productivity and pro-inflammatory cytokine profile in chickens. The Proteobacteria abundance was also decreased when L. rhamnosus GG was supplemented in mice having dysbiosis of colon microbiota induced by experimental sepsis. Similar to the findings here, the abundance of Akkermansia, a genus belonging to phylum Firmicutes, was increased in those mice treated with L. rhamnosus GG. The L. rhamnosus GG treatment in those mice reduced the sepsis-induced mortality by modulating the microbiota dysbiosis, by decreasing the Enterobacteriaceae and Enterococcaceae abundance, Firmicutes abundance was also increased in pre-weaning piglets treated with L. rhamnosus GG. L. rhamnosus GG treatment in those piglets was proven beneficial for intestinal health as it enhanced the biological, physical, and immunological barriers of intestinal mucosa. Contrary to Proteobacteria, the increase in phylum Firmicutes is associated with high productivity and anti-inflammatory cytokine profile in chickens. The abundance of bacteria belonging to genus Escherichia was also decreased in gut microbiota of children's who consumed L. rhamnosus GG indicating the ability of L. rhamnosus GG to prevent bacterial infections. The increased abundance of bacteria belonging to Erysipelotrichaceae family was observed in L. rhamnosus GG treated chickens, which is reported to be associated with improved growth and feed conversion in chickens. These results demonstrate that L. rhamnosus GG can modulate the gut microbiota composition in different hosts to resist bacterial infections. Interestingly, Flavonfractor abundance was also increased in S. typhimurium infected chickens, similar to what was observed in APEC infected chickens in this study. This shows Flavonfractor to be a potential gut microbial marker to monitor enteric infections in chickens.

The adhesion and invasion of APEC to HT-29 cells was reduced when pre-treated with sub-inhibitory concentration of L. rhamnosus GG supernatant (FIG. 4). It is possible that pre-treatment of L. rhamnosus GG supernatant enhanced the integrity of HT-29 colorectal epithelial cells, thus improving the epithelial barrier function and decreasing the adhesion and invasion of APEC. Similar to what was observed in this study, pre-treatment of L. rhamnosus GG supernatant reduced the adhesion, invasion, and translocation of E. coli K1 to human colorectal epithelial (Caco-2) monolayer cells. The pre-treatment of L. rhamnosus GG supernatant also inhibited the adherence of S. aureus to primary human keratinocytes and adhesion and invasion to human osteoblast (HOB) cells. However, in contrast to this finding, pre-treatment of L. rhamnosus GG cells itself decreased the intracellular invasion of S. infantis in porcine jejunal epithelial (IPEC-J2) cells and adhesion, invasion, and transcytosis of E. coli K1 in Caco-2 cells. Interestingly, the simultaneous addition (no-pre-treatment) of L. rhamnosus GG also reduced the adhesion, invasion, and translocation of C. jejuni to chicken (B10X1) and pig (CLAB) small intestinal epithelial cell lines. These findings demonstrate that L. rhamnosus GG itself or its cell-free supernatant can exhibit anti-bacterial effects to competitively exclude different pathogens at infection sites; thereby, preventing the diseases.

As reported in other studies, L. rhamnosus GG effect against APEC can be multi-factorial that includes production of lactic acid, secretion/release of small peptides and others. The shortened cells with bulbous swelling were observed in SEM after APEC was treated with L. rhamnosus GG supernatant (FIG. 2). Similar morphology was observed when E. coli was treated antimicrobial peptides, gramicidin S and α-helical peptidyl-glycylleucine-carboxyamide (PGLa), showing the damage of the bacterial cell envelope upon treatment with L. rhamnosus GG CFS.

In summary, this study evaluated different probiotic and commensal bacteria and identified L. rhamnosus GG as a preventative measure against APEC infection in chickens. Multiple small novel bioactive peptides that can be developed as non-antibiotic therapeutics against APEC in the future. Also, L. rhamnosus GG interactions with APEC and commensal microbes in the gut microbiota of chickens were uncovered which can facilitate the understanding of mechanism behind L. rhamnosus GG anti-bacterial effects.

### Example 2. Lacticaseibacillus rhamnosus GG (LGG) Reduces Salmonella Colonization in Chickens

Salmonella is a leading bacterial cause of foodborne illness in the US and worldwide. Salmonella enterica is a major pathogen in humans and animals and consists of more than 2000 serotypes. The serotypes are dispersed in nature and commonly inhabit the intestinal tract of mammals, birds, and other animals. In poultry, the pathogenicity of Salmonella varies based on strain and serotype, host, breed, and age. Commonly, birds show no clinical signs of the disease when challenged with non-typhoidal strains of Salmonella, but the bacteria can invade and be present in the cecum, liver, and spleen. Likelihood of clinical disease and mortality is heightened when birds are infected within the first 24 hours of life. Hindering Salmonella from entering the food chain is a high priority. Transmission of Salmonella to humans most frequently occurs through the ingestion of food contaminated by animal feces or cross contamination from poultry products. According to the Centers for Disease Control and Prevention (CDC), poultry and poultry associated products are the most common source for human Salmonella infection. Additionally, a study evaluated Salmonella infection by food source and identified poultry and eggs as causing 19% and 15% of cases in the US, respectively. In humans, salmonellosis is associated with gastroenteritis, enteric fever, or no symptoms (asymptomatic). The increasing consumption of poultry products and presence of Salmonella in carcasses further contributes to the public health risk of Salmonella. Salmonella enterica serovar typhimurium (ST) and Salmonella enterica serovar Enteritidis (SE) (paratyphoid/non-typhoid serovars) remain among the more prevalent serovars isolated from human infection; accounting for 16% and 20% of US human Salmonellosis from 2004-2016, respectively. They are two of the five serotypes responsible for half of antimicrobial resistant Salmonella infections in the US. Globally, nontyphoid Salmonella gastroenteritis remains a concern with over 93 million cases and 155,000 deaths each year. Controlling Salmonella is difficult because of its high tolerance to environmental stress, widespread distribution, adaptability, and most pertinently-its increasing antibiotic resistance.

Previously, antibiotics have been used to prevent and treat bacterial infections, promote growth, and improve production of food animals. The use of antibiotics in production animals has been credited to increasing contamination of poultry and food products with antibiotic-resistant Salmonella. These findings, coupled with consumer concerns over the spread of antibiotic-resistant pathogens causing antibiotic resistant infections in humans, have shifted the culture and laws concerning the use of antibiotics on farms. Therefore, the Food and Drug Administration (FDA) banned the use of fluoroquinolones in poultry production because of their link to antibiotic resistant bacteria in humans. Additionally, they banned the use of antibiotics for growth promotion (70 FR 44105; FDA GFI #209; FDA, 2013). Although there is a need to diminish antibiotic-resistant bacteria, the reduction in antimicrobials on the farm may lead to an increase in foodborne pathogens on meat and other animal byproducts if other alternative treatments are not incorporated. Currently, there is no consistent data showing that live commercial vaccines for poultry offer cross protection against multiple serovars. Management alone cannot eliminate these pathogens, so it is necessary to implement innovative and effective ways to mitigate the emergence of antibiotic-resistant bacteria and foodborne pathogens. Targeting the control of the foodborne pathogens in the pre-harvest stage can additionally improve the animal welfare.

Probiotics are live microorganisms that can offer health benefits to the host when given in adequate amounts. Particularly, probiotics can produce antimicrobial compounds (organic acids and bacteriocins), occupy adhesion sites, or inhibit the growth or presence of entero-pathogens through competitive exclusion. Lactic acid bacteria (LAB) (Lactobacillus and Bifidobacterium) are the major beneficial microbes in foods. Strains of these genera have been proven to have antibacterial activity against Salmonella. Importantly, some LABs are able to survive gastric passage and transiently colonize the mammalian intestinal mucosa. Bifidobacteria and Lactobacillus are already components of the human gut microbiota and have associations with positive host health. Species belonging to these two genera have a history of being safe to use in human applications. Lactobacillus can be found in infant food, cultured milks, and pharmaceuticals. However, the antimicrobial activity of a probiotic is strain dependent; so, investigations are needed to prove the effectiveness of a particular probiotic strain.

In this study, the aim was to characterize the efficacy of probiotics (Lacticaseibacillus rhamnosus GG (LGG), Lactobacillus acidophilus (LA), Levilactobacillys brevis (Lbrev), and Bifidobacterium animalis subsp. lactis (Bb12)) in inhibiting ST and SE. Additionally, here the anti-Salmonella characteristics of the probiotic was defined, and novel bioactive peptides were isolated that were also efficient in inhibiting Salmonella.

### Results

### LGG. Bb12, and LA Possessed Anti-Salmonella Efficacy

Probiotic bacteria were used in an agar well diffusion assay to screen which ones would possess anti-Salmonella activity. Whole cultures of LGG and Bb12 were able to inhibit (13±0.5 mm and 14.5±0.25 mm inhibition zone, respectively) ST in an agar well diffusion assay at 24 h substantially greater than Escherichia coli Nissle 1917 (EcN) (0.0±0.0 mm) and Lbrev (0.0±0.0 mm) (Table 9). There was a noticeable zone of inhibition for LGG (15.5±0.5 mm) and Bb12 (16.0±0.5 mm) at 12 hours. LA showed inhibition against ST comparable to LGG and Bb12 at 12 hours but the zone of inhibition decreased to 11.5 mm by 24 hours, still noticeably larger than EcN and Lbrev. Lbrev showed a minimal inhibition at 12 h (10 mm) and no inhibition at 24 hours (Table 9). EcN showed no inhibition throughout the duration of the study and was omitted from further analyses.

At 24 h, heat treatment had no effect on the antibacterial activity of LA while LGG and Bb12 shown a slight reduction of inhibition when heated, compared to their respective unheated whole cultures (LGG: 13±0.5 mm-10±0.5 mm; BB12: 14.5±0.25 mm-11±0.25 mm) (Table 9). LA, LGG, and Bb12 cultures showed no change in inhibition when cooled to 4° C. (Table 9).

### LA, LGG, and Bb12 Significantly Inhibited the Growth and Persistence of ST & SE in Co-Culture Assays.

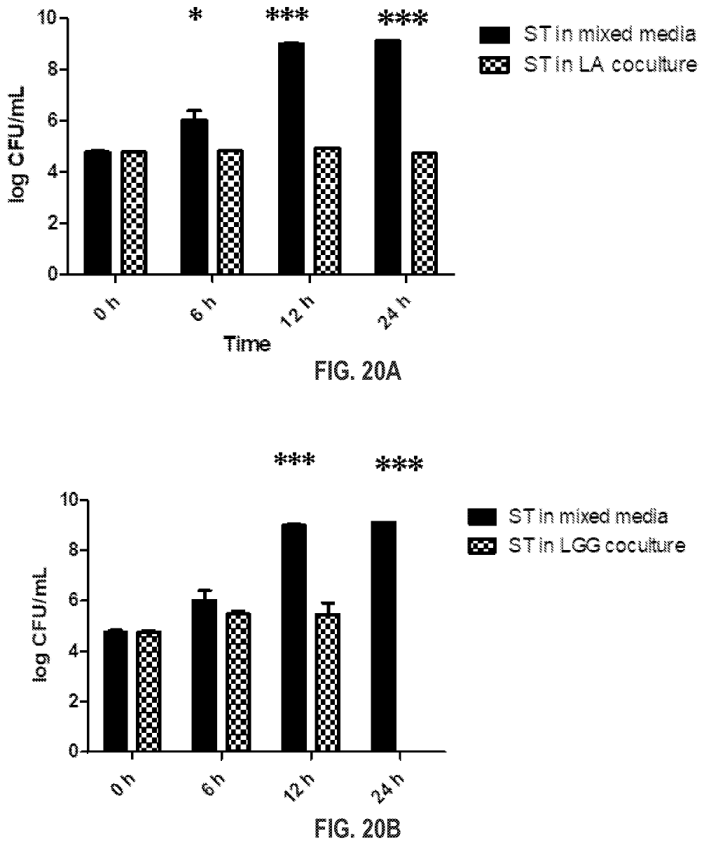

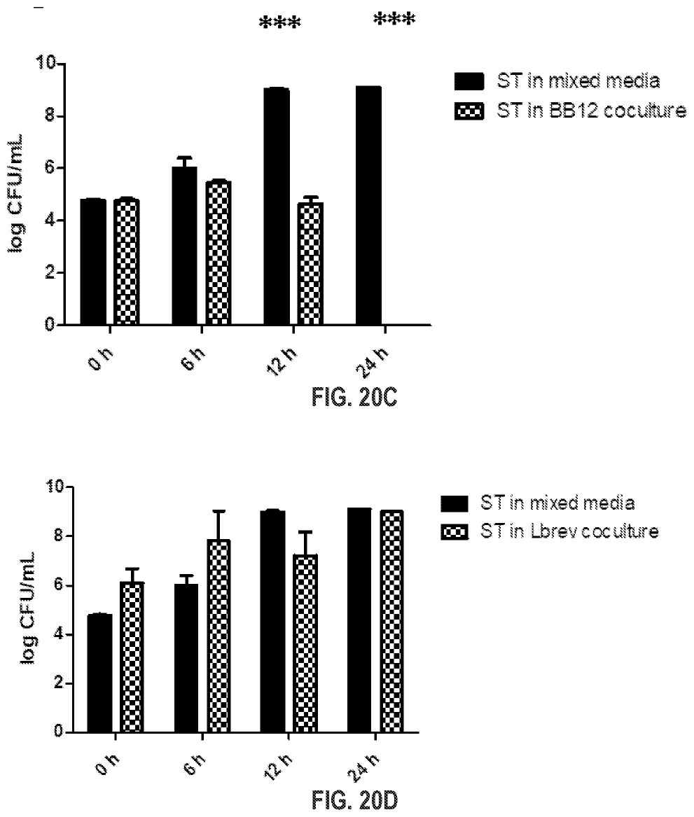

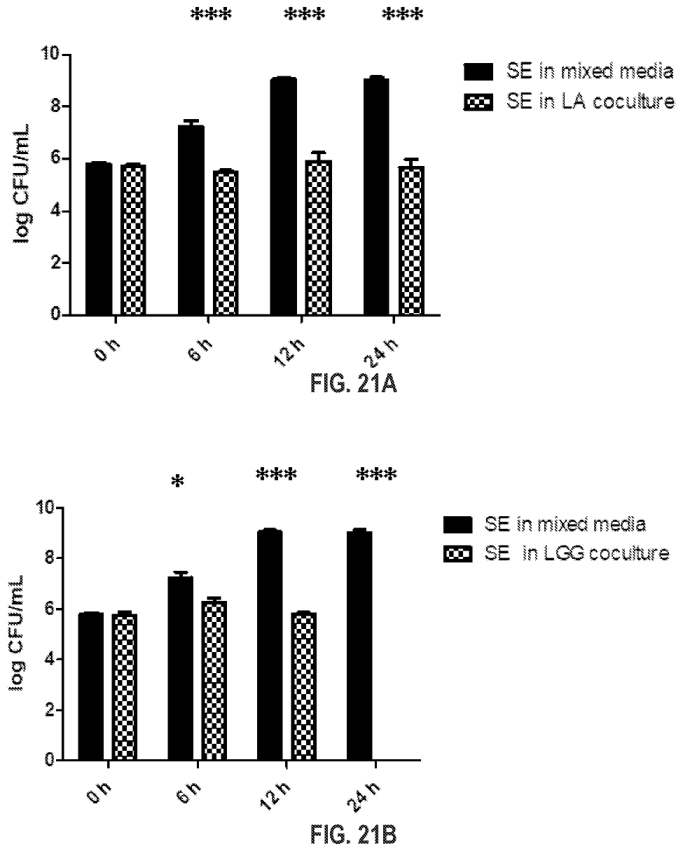

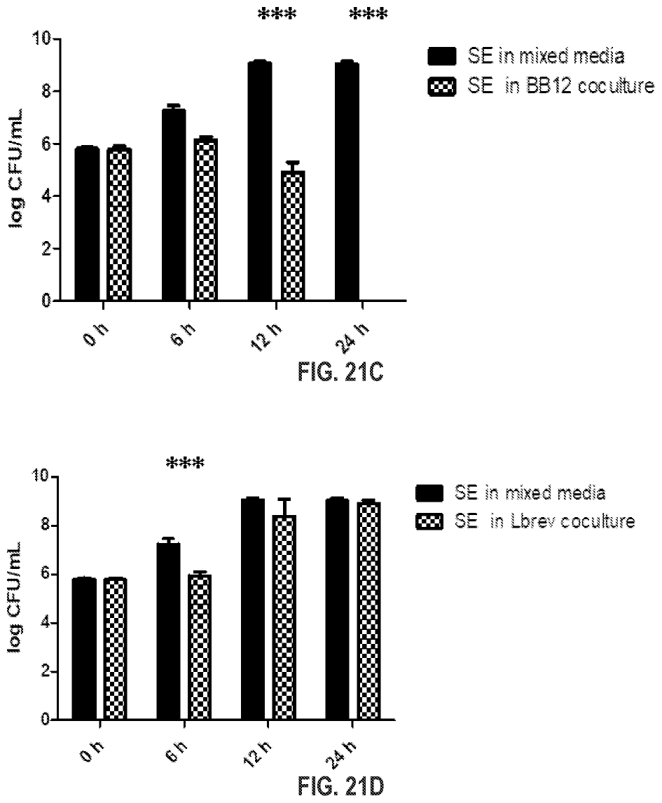

Probiotics were simultaneously co-cultured with a Salmonella strain to investigate their ability to inhibit the growth of either ST or SE. In both the ST-LA and SE-LA co-cultures, LA began to significantly inhibit the growth of Salmonella as early as 6 h (FIGS. 20A and 21A). The co-culture of LA with ST significantly reduced ST by 1 log at 6 h (P<0.05), while by 12 h the reduction of ST increased to 4 logs and remained at 4 logs throughout the duration of the study, P<0.001 (FIG. 20A). Culturing LA with SE in MRS-LB media significantly reduced SE at 6 h (1.7 log; P<0.001), by 12 h the reduction of SE increased (3.15 logs; P<0.001) and remained significantly lower (˜3 logs; P<0.001) than the ST alone control at 24 h (FIG. 21A). LGG and Bb12 significantly inhibited the growth of SE at 6 h by ˜ 1 log; P<0.05 (FIG. 21B and FIG. 21C) in their respective co-cultures. Although LGG and Bb12 showed no significant inhibition of ST at 6 h, by 12 h LGG significantly inhibited ST by 3.5 logs (P<0.001) and SE by 3.25 logs (P<0.001) (FIG. 20B and FIG. 21B). Similarly, at 12 h, Bb12 inhibited ST and SE by 4 logs (P<0.001) (FIG. 20C and FIG. 21C). Notably, both LGG and Bb12 fully cleared ST and SE at 24 h (P<0.001) (FIGS. 20 and 21). After 24 hours, Lbrev did not significantly reduce or clear ST or SE (FIG. 20D and FIG. 21D).

The pH of each co-culture became more acidic over time, showing that pH to have a role in ST/SE inhibition (Table 10). Although the pH of the Lbrev co-cultures reached an acidic environment (ST-Lbrev 4.79±0.10; SE-Lbrev 4.88±0.07) (Table 10) they did not inhibit ST/SE at 24 h. This is in direct contradiction to the co-cultures of LA, LGG, and Bb12, where pH reduction and bacteria reduction was noticed; implying pH isn't the only factor involved in inhibition.

### Secreted Products of LGG and Bb12 Possessed Inhibitory Characteristics in a Transwell Assay

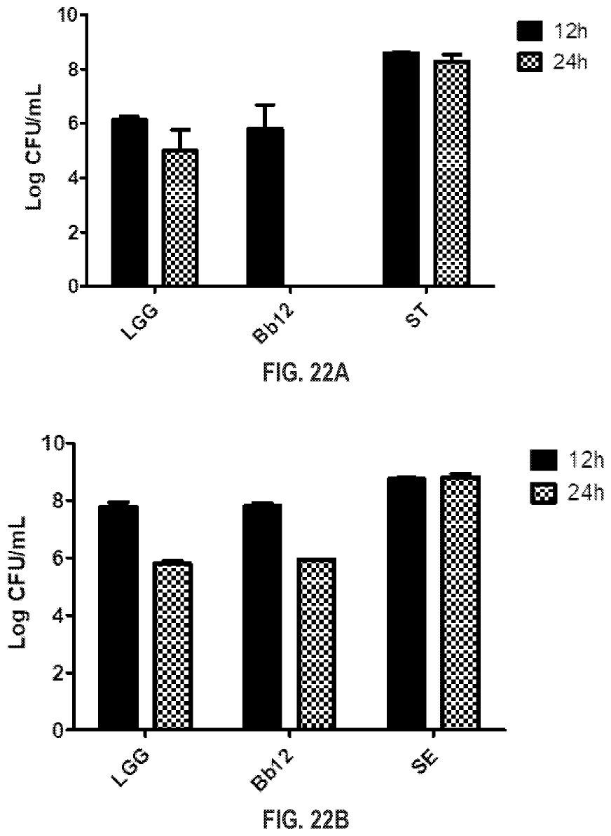

The secreted products of LGG and BB12 were tested in a transwell assay to determine if the antagonistic effects are due to the bacteria secreted/released products. The secreted products of LGG, and Bb12 yielded a 2.45, and 2.79 log numerical reduction of ST at 12 h compared to the ST control (FIG. 22A) in a transwell assay. At 24 h, the secreted products of LGG increased that difference to ˜3.3 logs. The secreted products of Bb12 completely inhibited the presence of all viable ST at 24 hours (FIG. 22A). Although, no secreted products of any probiotics caused a complete inhibition of SE there was still a noticeable difference. At 24 hours both LGG and Bb12 caused a ˜3 log reduction of SE (FIG. 22B). These results showed that the secreted products of the probiotics have anti-Salmonella properties.

Lactic Acid Predominates Other Organic Acids of L. rhamnosus GG and B. lactis Bb12 and Contained Multiple Small Peptides in their Cell-Free Supernatants

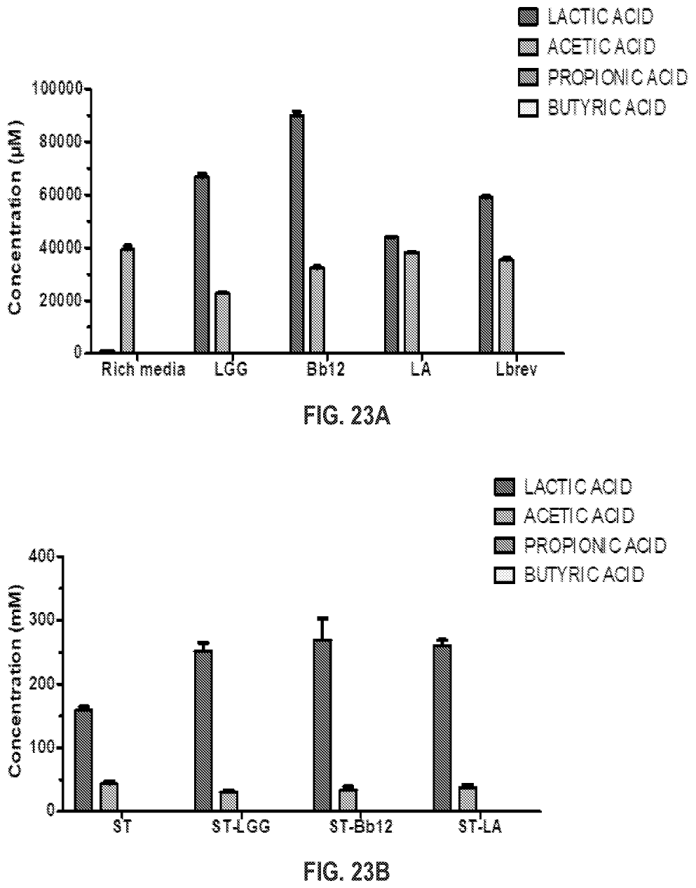

The organic acids in CFS of L. rhamnosus GG, B. lactis Bb12, L. acidophilus, and L. brevis probiotics were quantified to investigate the contributing factors of Salmonella inhibition. Lbrev and LA were included in the analyses to determine if they had a different composition than the effective probiotics LGG and BB12. LC-MS/MS and isotope-labeled chemical derivatization method was used to identify and quantify the organic acids produced by the probiotics. To quantitate the concentration, standard curves were generated for each organic acids (lactic acid: y=0.0006x−0.2761, R²=0.9865, acetic acid: y=0.0006x−0.3419, R²=0.9807, propionic acid: y=0.00012x−0.577, R²=0.9839, and butyric acid: y=0.0017x−0.6008, R²=0.9855). Lactic acid was the major organic acid in the CFS of LA (0.044M), LGG (0.067M), Bb12 (0.090M), and Lbrev (0.059M) (FIG. 23A). When the probiotics (LA, LGG, and BB12) were co-cultured with ST, lactic acid still predominated (ST-LGG 251.24 mM, STLA 269.03 mM, ST-Bb12 158.85 mM). Acetic acid was the next most abundant organic acid (ST-Bb12 33.36 mM, ST-LA 37.39 mM, ST-LGG 30.24 mM) (FIG. 23B).

Further, CFSs were eluted using HyperSep™ Hypercarb™ SPE cartridge and analyzed by LC-MS/MS to identify the bioactive molecules in CFS of LA, LGG, Bb12, and Lbrev. A total of 152 peptides and 57 peptides were identified using CID (ion-trap-based collision-induced dissociation) and HCD (higher energy collision dissociation) settings, respectively (data not shown). There were 33 peptides common between the two settings with molecular weight less than 3 KDa (Table 15)) and were mostly found in of LGG and Bb12 consistent with their strong anti-Salmonella activity.

### Heat and Protease Resistant Secreted Products of LGG and BB12 Inhibit Salmonella

The secreted products were further analyzed in an agar well diffusion assay to inhibit Salmonella. The CFS of LGG and Bb12 both had a zone of inhibition against ST of 12.5±0.0 mm at 12 hours. At 24 hours, LGG yielded 9±1.0 mm and Bb12 yielded 9±0.5 (Table 11). There was no noticeable difference in the zone of inhibition at 24 hours when the CFS was heated to 121° C. or cooled to 4° C., proving the temperature tolerance of the secreted products. The CFS of LGG and Bb12 was passed through a 3 kDa Amicon Ultra centrifugal filter, and the fractionate product still inhibited ST in an agar well diffusion assay (Table 11). When treated with proteinase K, the secreted products of LGG and Bb12 retained anti-Salmonella (ST inhibition: LGG 12±0.0 mm, Bb12 11±0.0 mm; SE inhibition: LGG 12±0.5 mm, Bb12 11.5±0.1 mm) (Table 11 and Table 12) abilities at 24 hours, showing that secreted/released products are heat stable, proteolysis resistant and of low mol. Wt. in size.

### Cell Free Supernatant of Probiotics Prevent Invasion of Salmonella in Polarized HT-29 Cells

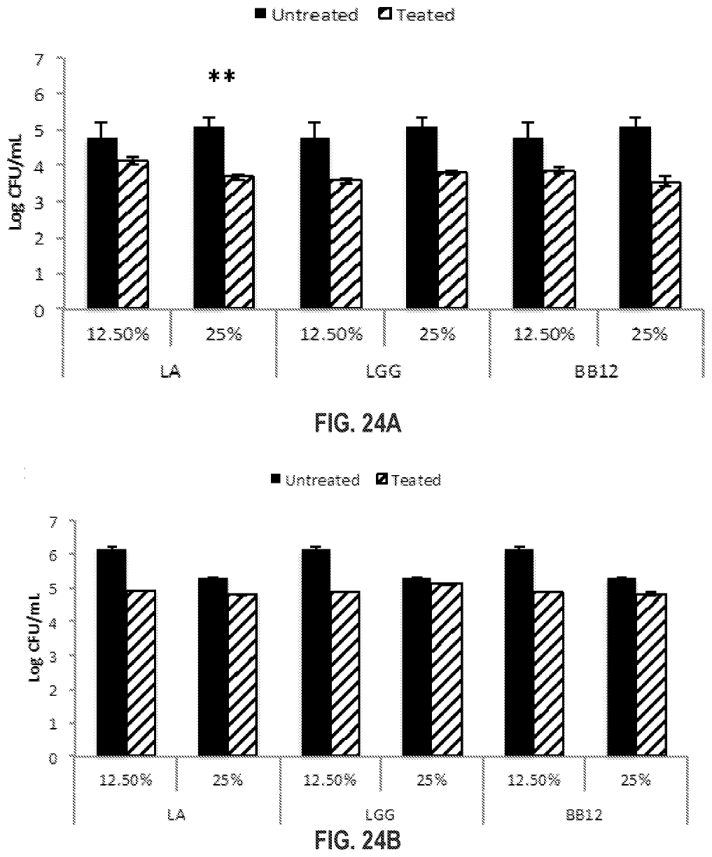

To further analyze the efficacy of the cell free supernatant (CFS) of probiotics, CFS of LA, LGG, and BB12 were analyzed in a cell culture model. LA was included because of previous inhibition with whole culture. Polarized HT-29 cells infected with Salmonella were treated with 12.5% and 25% CFS of LGG, or Bb12. The 12.5% and 25% CFS of the probiotics were not toxic to the cells. In the SE infected cells, 12.5% of the CFS yielded the larger reduction (FIG. 24B), while the 25% of CFS was most effective for ST infection (FIG. 24A). There was a 1.27, and 1.28 log reduction of SE from 12.5% supernatants of LGG, and Bb12, respectively (P<0.05) (FIG. 24B). There was a 1.29, and 1.5 log reduction of ST from 25% supernatants of LGG, and Bb12, respectively (P<0.05) (FIG. 24A).

### LGG Reduced Colonization of ST in Chicken Cecum and Conferred Protection Against Other Serotypes In Vitro

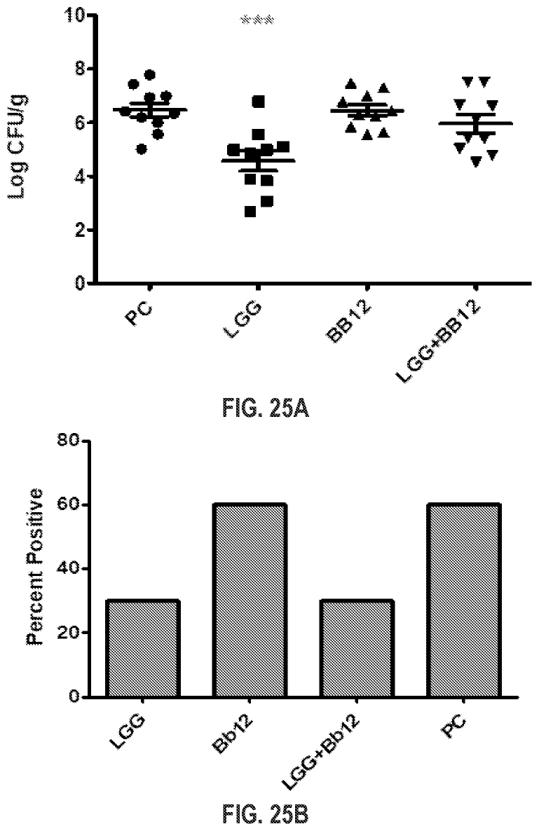

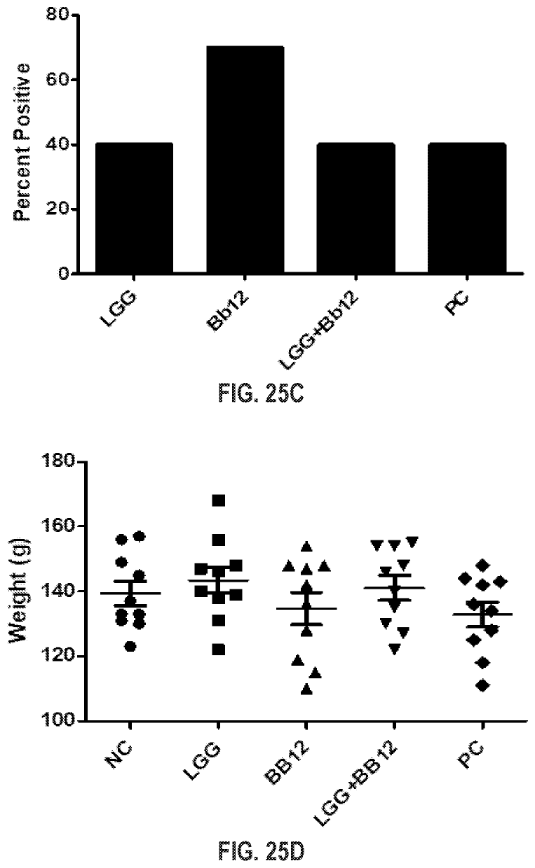

The efficacy of LGG, Bb12, and LGG+Bb12 (1:1 combination) was evaluated in one-day-old chickens using SPF layers. The probiotics were administered orally for 16 days (108 CFU/chicken daily). LGG reduced Salmonella in cecum by ˜1.9 logs (P<0.001) (FIG. 25A) ten days post infection. The LGG treated group showed a 30% reduction in number of birds positive for ST in spleen compared to the ST untreated control (FIG. 25B). Neither the Bb12 nor the LGG+Bb12 groups were able to reduce the amount of ST in the cecum. However, the LGG+BB12 treatment reduced the percent of birds positive for ST in spleen by 30% (FIG. 25B). None of the treatment groups reduced ST in the liver (FIG. 25C). Probiotic treatment did not alter necropsy weights of birds (FIG. 25D).

LGG Efficacy Remained when Supplemented in Drinking Water and Reduced ST in Cecum

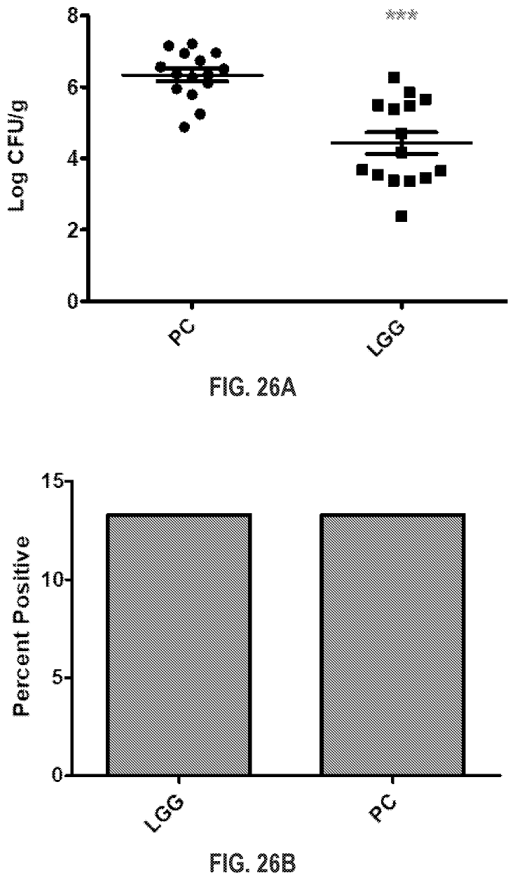

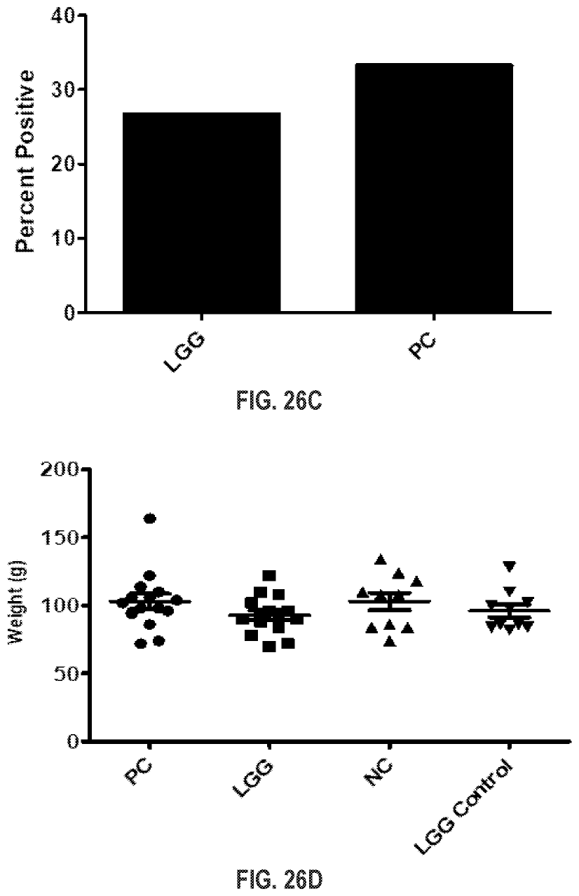

LGG was supplemented continuously in the drinking water of one-day-old SPF layers for 13 days to further investigate the delivery of probiotics to chickens. Similar to the first chicken trial, LGG reduced ST by ˜1.91 logs in the cecum compared to untreated PC (P<0.001) (FIG. 26A). The colonization of ST in the visceral organs (spleen and liver) was inconsistent (FIGS. 26B and 26C). Particularly, both LGG and PC birds were 13.3% positive for ST in the spleen (FIG. 26B). LGG treated birds were 26.7% positive and PC control birds were 33.3% positive for ST in the liver (FIG. 26C). Consistent with the previous trial, treatment had no effect on the weight of birds at the close of the study (FIG. 26D).

### LGG Inhibited Prevalence of Food Safety Relevant Serotypes

LGG efficacy against ST, led to testing its efficacy against other serotypes (S. Anatum, S. Albany, S. Brenderup, S. Javiana, S. Heidelberg, S. Muenchen, S. Newport, and S. Saintpaul) in an agar well diffusion assay. LGG inhibited the growth of all other Salmonella serotypes after 24 hours (Table 13). The largest inhibition was observed in S. Muenchen (17±0.50 mm).

### Novel Peptides Identified in Probiotics Inhibit ST

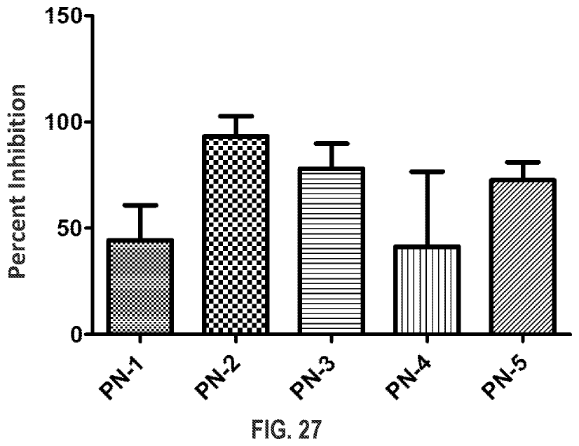

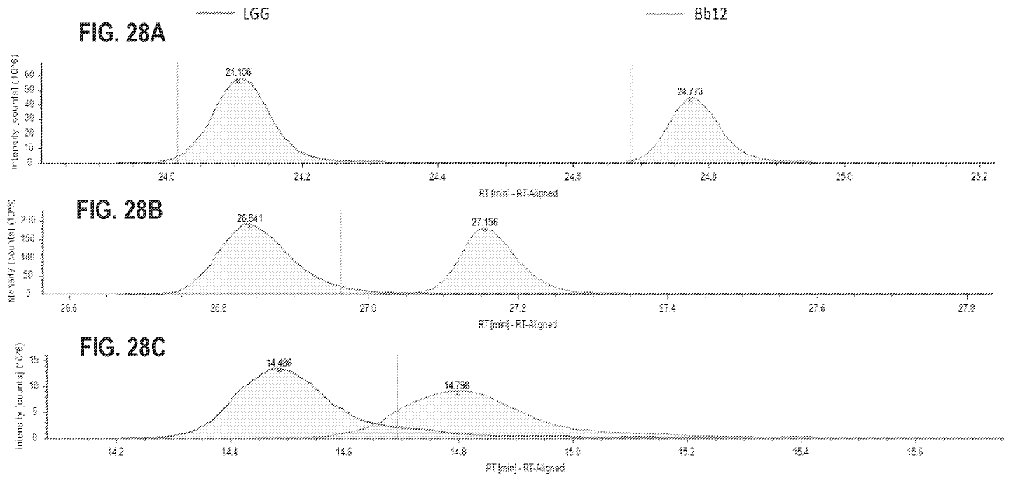

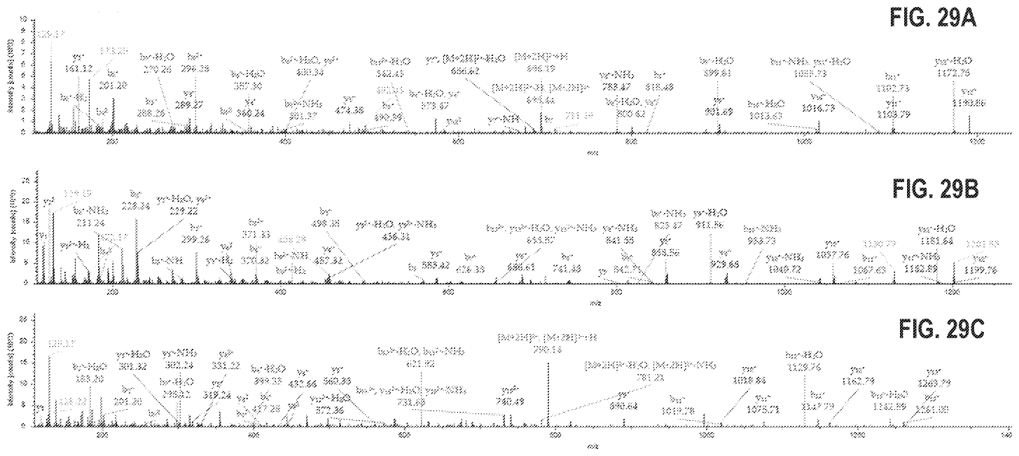

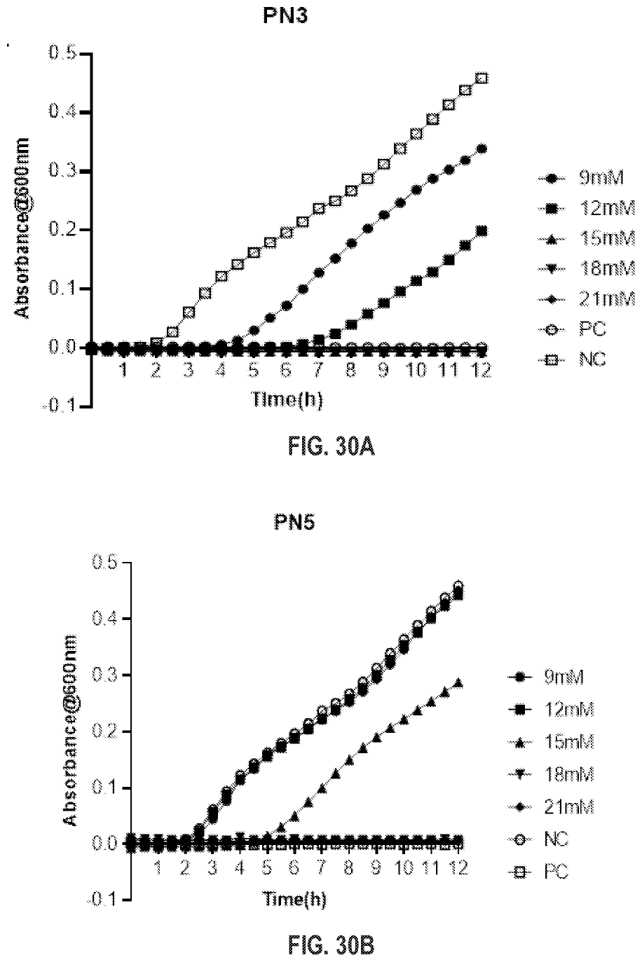

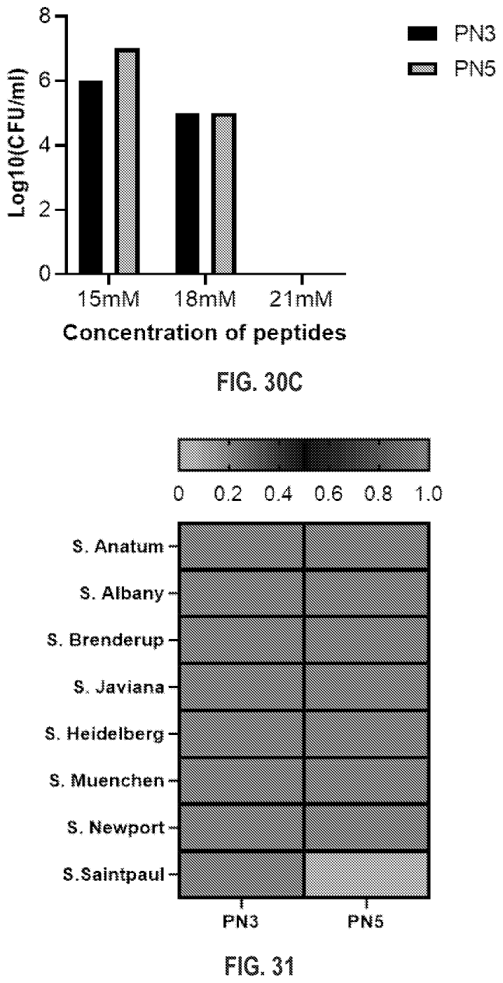

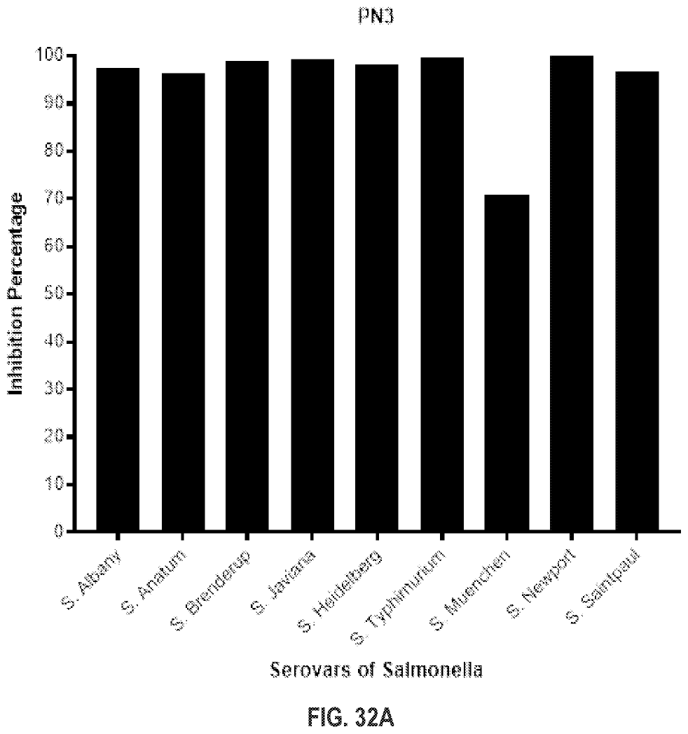

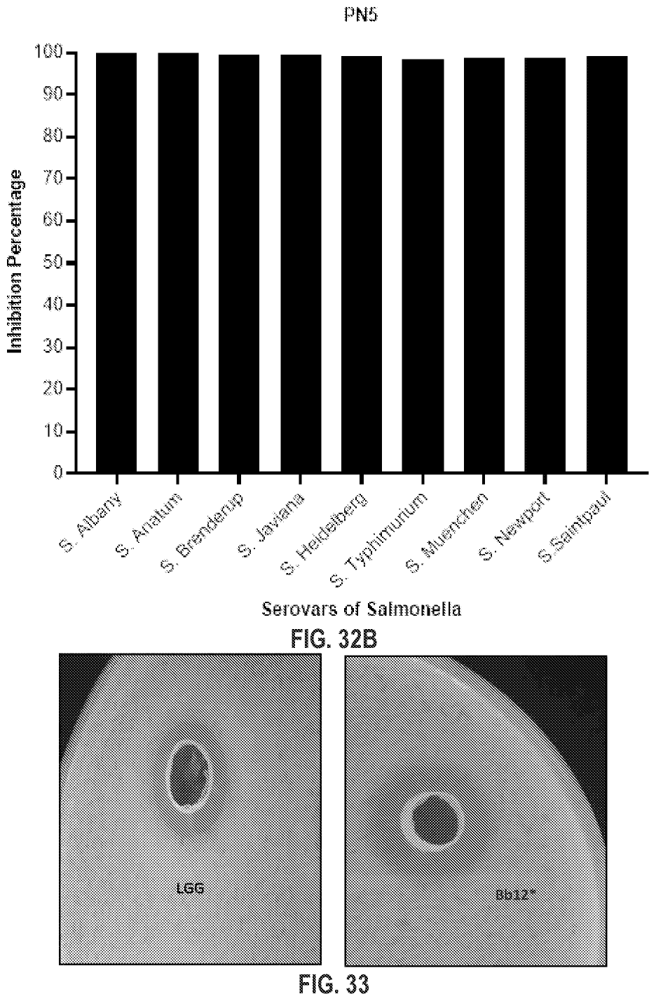

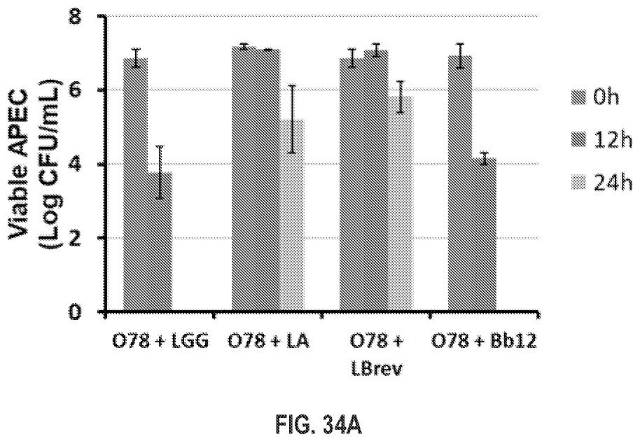

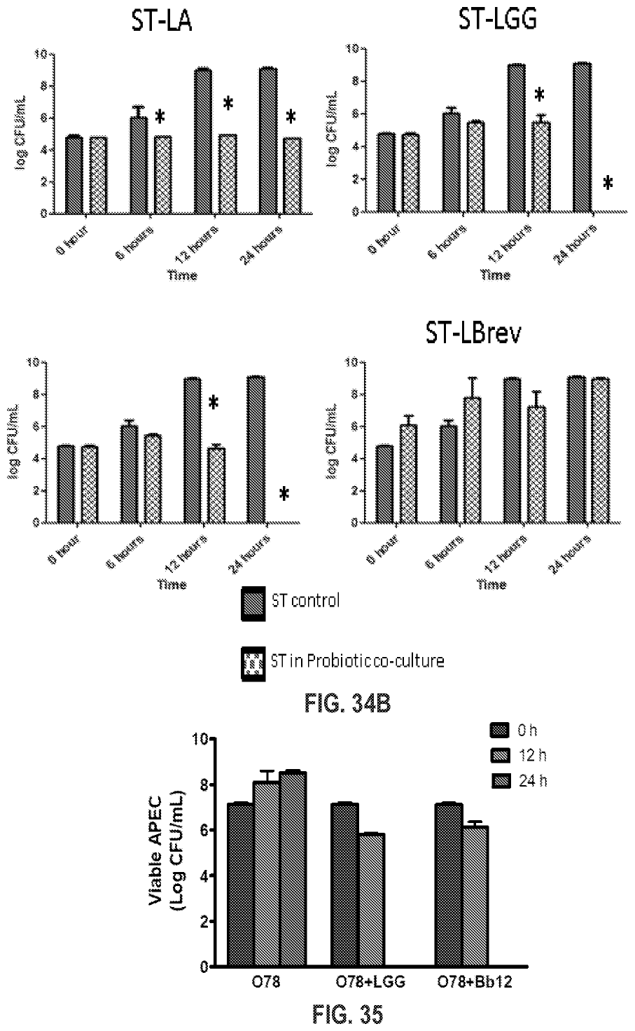

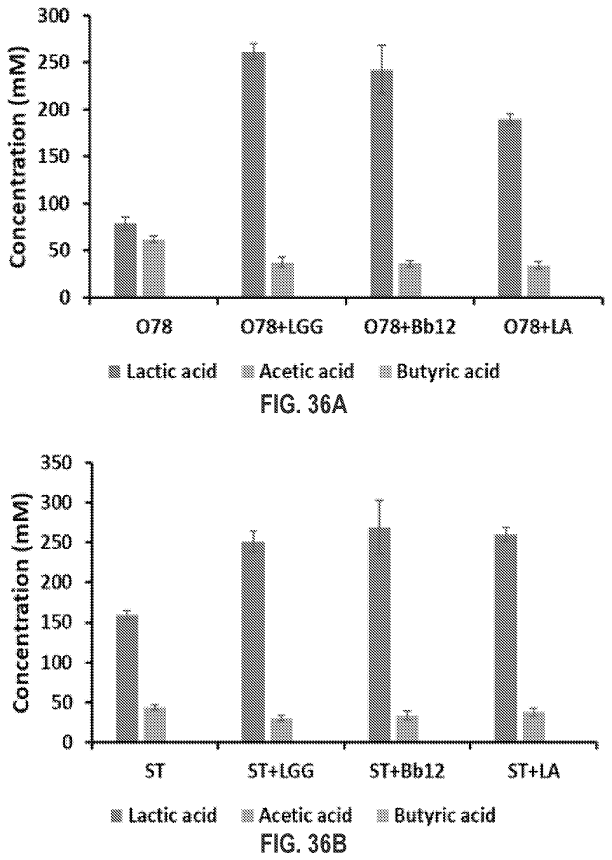

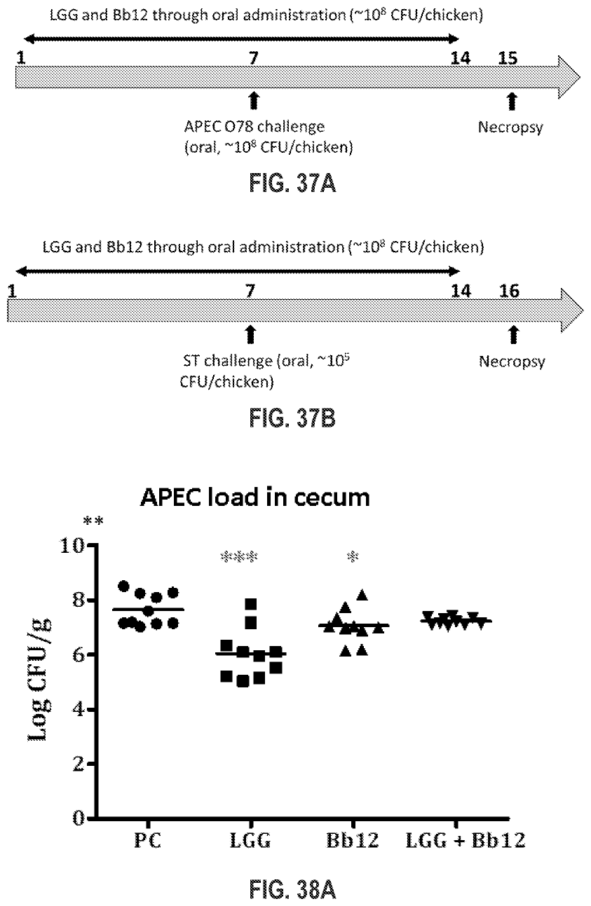

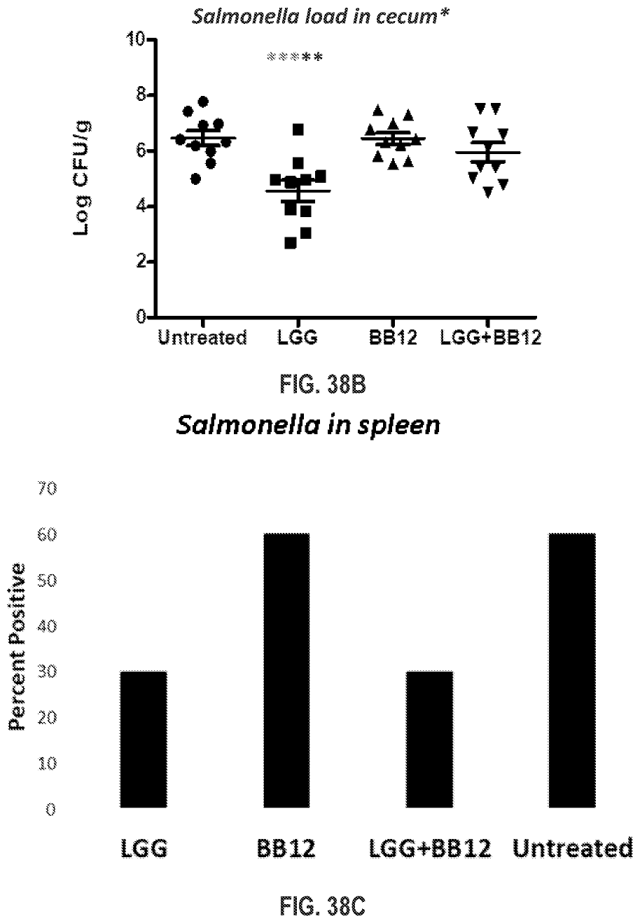

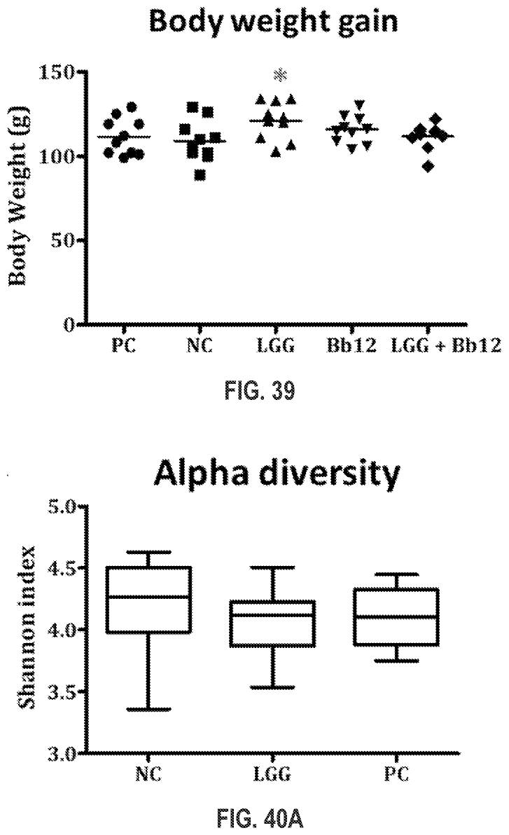

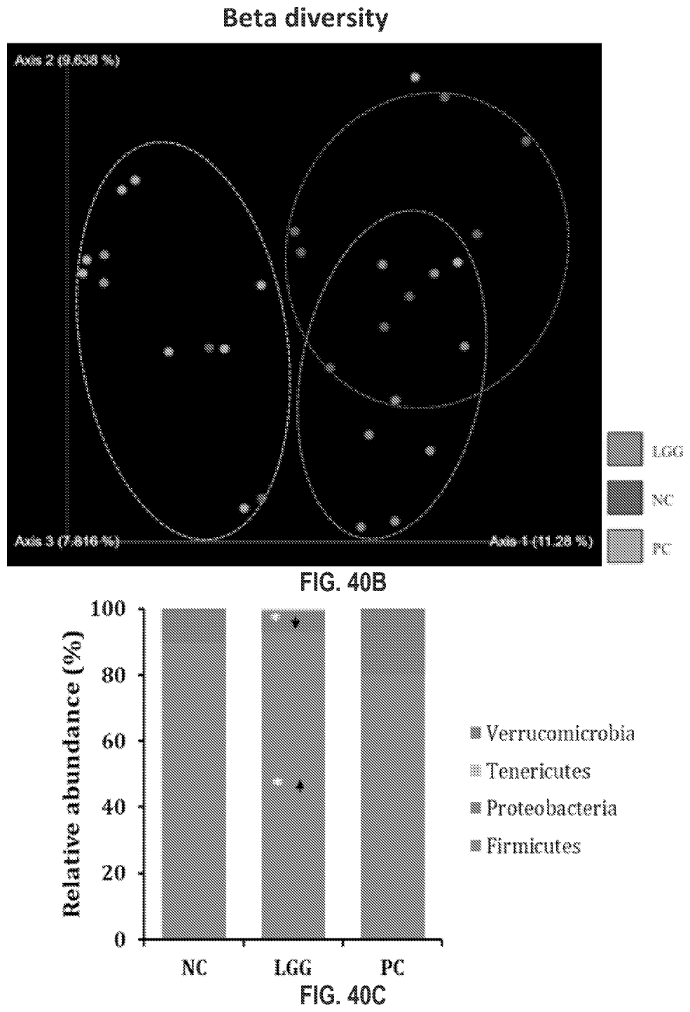

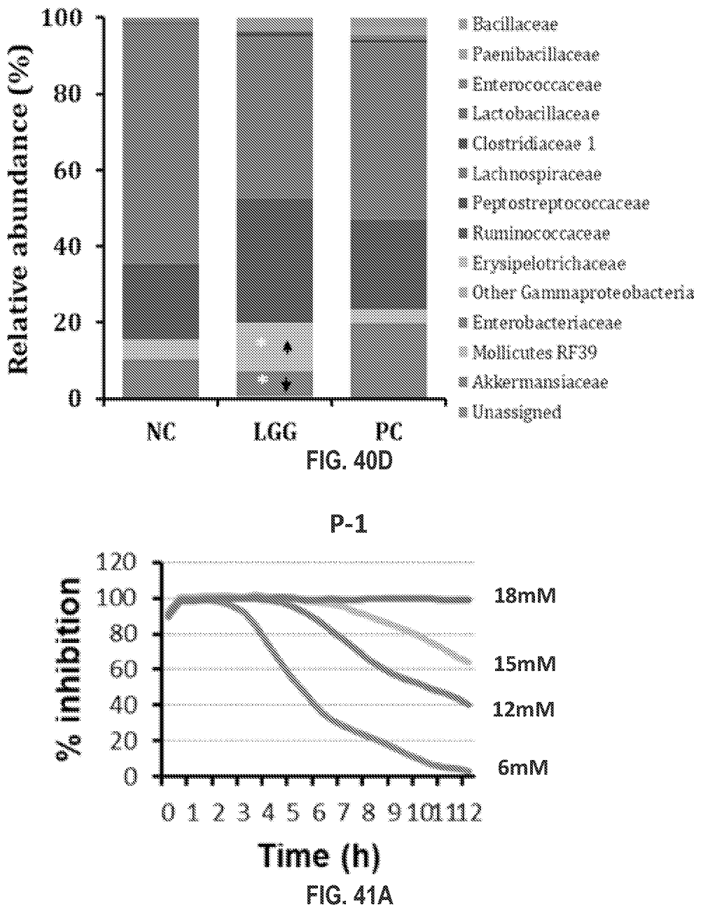

Five peptides were selected for synthesis from the 33 peptides (Table 15) based on their charge, hydrophobicity, and abundance in LGG and Bb12, consistent with strong anti-Salmonella characteristics. Abundance in LGG and Bb12 was used because of their most efficacy against Salmonella in the previous experiments. The selected 5 peptides were (PN-1: FSAVAL SAVAL SKPGHVNA (SEQ ID NO: 5), PN-2: AESSDTNLVNAKAA (SEQ ID NO: 18), PN-3: VQAAQAGDTKPIEV (SEQ ID NO:21), PN-4: AFDNTDTSLDSTFKSA (SEQ ID NO:26) and PN-5: VTDTSGKAGTTKISNV (SEQ ID NO:32)) (Table 15). They were tested at 12 mM against ST for 12 hours, PN-2, completely inhibited ST at 12 mM (FIG. 27). Molecular weight and Retention times of PN-2 (A), PN-3 (B), and PN-5 (C) that showed highest inhibition are shown in FIGS. 28 and 29.

### Discussion

Despite the implementation of Salmonella control programs, foodborne salmonellosis transmission remains a global agricultural and human health concern. Increasing antibiotic resistance, more stringent laws, and the increasing global trade and consumption of poultry and poultry products necessitate the need for novel ways of controlling Salmonella infection in the pre-harvest stage. In this study the ability of probiotics and their derived products to inhibit S. typhimurium and S. enteritidis was evaluated. In vitro LGG and Bb12 were able to inhibit the growth of Salmonella in an agar well diffusion assay (Table 1). This was consistent with other studies that showed the ability of probiotics to inhibit Salmonella in agar well diffusion assays. These studies showed that L. rhamnosus inhibited the growth of multiple Gram-negative bacteria including S. typhimurium, E. coli, and Shigella sonnei, Staphylococcus aureus, and Clostridium difficile. Similar to the results seen here (Table 9 and 11), Lactobacillus paracasei subp. paracasei M5-L and L. rhamnosus J10-1 showed heat stability when they were still able to inhibit Shigella, Salmonella, and E. coli in agar well assay. This shows that a probiotic or peptide can be applied commercially without decreasing the efficacy during feed processing.

Additionally, the antagonistic abilities of the probiotics were confirmed in a co-culture assay where LGG and Bb12 fully cleared ST and SE after 24 hours (FIGS. 20 and 21). A study from Belgium supports these findings, showing that when LGG was added to their co-culture, the growth of Salmonella was greatly inhibited. There have been reports in literature suggesting the anti-bacterial properties of probiotics are multifactorial. Acidic pH and organic acids have been shown as the reason for this effect. Although this study shows that pH may play a role, this data shows that pH alone is not responsible for the inhibition of Salmonella. In the co-culture study, Lbrev co-cultures with ST and SE reached an acidic environment at 24 h (pH4.79, pH4.88 respectively, Table 2) but was unsuccessful at inhibiting Salmonella (8.99 logs of ST present and 8.92 logs of SE, FIGS. 20D and 21D) compared to LGG and BB12 (0 log of ST or SE at 24 hours, FIGS. 20B & 20C, FIGS. 21B& 21C). The antagonistic properties are pH dependent because when the pH of LGG and Bb12 CF S are neutralized they were unable to inhibit ST and SE in the agar well diffusion assay (data not shown). Organic acids may play a role in inhibition; in this study lactic and acetic acid are the predominant organic acids present in LGG (FIGS. 23A and 23B). This data was similar to De Keersmaecker et al. who also saw high levels of lactic acid in LGG's supernatant. Results from one study suggested that lactic acid might only be efficient at reducing Salmonella in the nutrient rich media (MRS). it was observed that when using PBS, the anti-Salmonella effect of lactic acid was absent. Here, the derived peptides retain efficacy outside of nutrient rich media. Importantly, the anti-Salmonella characteristics in this study were not sensitive to proteinase K. Similar proteinase K results were observed in another study but it was still concluded that organic acids, and not bacteriocins, were the reason for the inhibition. Based on the data here, it was concluded that organic acids and small peptides both play a role in inhibition. This is supported by LC-MS/MS analysis, which identified peptides present in the probiotics CFS that possessed anti-Salmonella activity.

A total of 33 novel peptides were identified from LC-MS/MS analysis of the probiotics CFS and 5 were selected to test antimicrobial efficacy. These five were chosen based on their charge, hydrophobicity, and abundance in LGG and Bb12. Three of these 5 peptides (PN-2, PN-3, PN-5) when tested at 12 mM (PN-2: AESSDTNLVNAKAA (SEQ ID NO:18), PN-3: VQAAQAGDTKPIEV (SEQ ID NO:21), PN-5: VTDTSGKAGTTKISNV (SEQ ID NO:32)) showed inhibition of 70% or greater against ST after 12 hours. PN-2 was completely inhibited at 12 mM (FIG. 27). The use of antimicrobial peptides to inhibit pathogenic bacteria can lead to less expensive and effective ways to control pathogens in food animals. Moreover, antimicrobial peptides can serve as a great alternative to conventional antibiotics because of the broad-spectrum activity and reduced risk of resistance. The small MW allows passage through the outer membrane and other defense mechanisms of Gram-negative bacteria.

LGG significantly reduced the colonization of ST in the cecum by ˜1.9 logs at 10 days post infection. This was consistent with other studies examining the role of Lactic Acid Bacteria (LAB) against poultry. Previous studies have shown that probiotics are effective in inhibiting the replication of enteropathogens in in vivo experiments. For example, a group of researchers from Massey University pre-fed mice L. rhamnosus strain HN001 for 7 days and then challenged them with S. typhimuriurm. Quantitative counts of Salmonella from spleen and liver indicated that probiotic fed mice had significantly lower pathogen burdens than the non-treated mice, approximately a 2-log reduction in both liver and spleen. Phagocytic responses and peritoneal leucocytes were significantly higher among the probiotic fed mice. In this experiment a highly pathogenic stain of Salmonella (S. typhimurium ATC strain 1772) was used, and the probiotic was still able to increase host survival (10% mortality post challenge) in comparison to the non-treated mice (93% post challenge mortality). This was consistent with a previous study showing that feeding mice Bifidobacterium lactis (strain HN019) increased their survival after Salmonella infection. In poultry, a recent study showed a combination of three LAB probiotics as a batch culture (1:1:1 probiotic mix) significantly reduced S. typhimurium cecal colonization by 1 log in turkey poults. Although a 2-log reduction was observed here, combining the most effective probiotics did not increase efficacy (FIG. 25A). A recent study showed that the mean percent value of cecum positive for S. typhimurium was lower in birds consistently fed a Bacillus based probiotic. It also showed that probiotic supplementation partly restored the hen's gut microbiota that was affected by ST infection. It observed inconsistent percentage of bird's positive for Salmonella in internal tissues, even amongst their challenged controls. This observation was confirmed in the inconsistent colonization of internal issues observed here. Even with enrichment, the positive control birds did not yield 100 percent colonization in liver or spleen (FIGS. 25 and 26). Lastly, a study examining the effect of organic acids and probiotics on broilers infected with Salmonella reported that the broilers treated with organic acids alone in the water did not successfully inhibit SE 5 days post challenge. Additionally, it was not able to consistently lower SE in crops of organic acid treated birds in short time intervals; however, reduced Salmonella in the crop in probiotic treated group and the organic acid+probiotic group was observed. In this study, probiotics supplementation in water, yielded an almost two log reduction a week after challenge, showing that the anti-Salmonella properties of probiotics are multifactorial involving organic acids and small peptides in acidic environments.

### Conclusion

LGG is effective at inhibiting Salmonella in vitro and successfully reduced the bacterial colonization in chicken's cecum. The ability of the probiotics to inhibit Salmonella when supplemented in water has implication for application under farm settings. The stepwise methodology allowed identification of anti-Salmonella properties of LGG and conclude that it is heat stable and not proteinase sensitive. The ability of the derived peptides to inhibit the growth of Salmonella without the probiotic culture led to the finding that both the acidic pH and small peptides present in the cell-free supernatant of LGG are responsible for the anti-Salmonella effect.

### Materials and Methods

Bacterial strains and growth conditions: Salmonella typhimurium LT2 (ST) (John Gunn, OSU, Columbus) and Salmonella Enteritidis (SE) (laboratory collection) were used to study the antagonistic ability of probiotics on Salmonella. ST and SE were grown in Luria Bertani (LB) broth at 37° C., for 18-24 h, with shaking at 180 rpm. Nalidixic acid resistant ST and SE were generated through spontaneous mutation by plating ST and SE on LB agar containing 100 μg/mL of nalidixic acid. L. acidophilus NCFM (LA; David Francis, SDSU), L. rhamnosus GG (LGG; ATCC 53703), L. brevis (Lbrev; David Francis, SDSU), Escherichia coli Nissle 1917 (EcN; Dr. Ulrich Sonnenborn, Ardeypharm GmbH, Herdecke, Germany) and B. animalis subsp. Lactis (Bb12; Christian Hansen, Ltd, Horsholm, Denmark) were cultured using MRS (de Man, Rogosa and Sharpe) media under anaerobic condition; MRS was supplemented with 0.05% cysteine hydrochloride for Bb12. Anaerobic conditions for the probiotic strains were achieved using the GasPak™ EZ Anaerobe Container System Sachets (BD diagnostics, NJ, USA). LA, LGG, Lbrevis and Bb12 were grown at 37° C. for 18-24 h under stationary condition.

Agar well diffusion assay: The agar well diffusion method, as described previously, was adapted, and used to determine the anti-Salmonella ability of commensal bacteria. One hundred microliters of each overnight grown probiotic bacteria (LA, LGG, Bb12, L. brev, and EcN) was placed in the wells (aseptically punctured) of ST or SE (˜5×10⁷ CFU/mL) inoculated agar plates. The average zones of inhibition (mm) were measured at 12 and 24 h after plates were incubated in aerobic conditions at 37° C. The whole cultures of probiotic bacteria that showed inhibition were then heated to 121° C. (autoclaved) and cooled to 4° C. for 30 minutes to determine their temperature tolerance. Experiment was replicated. Media only controls were used to ensure the MRS media did not cause the inhibition.

Efficacy of probiotics in co-culture assay: Each probiotic (LA, LGG, Bb12, and L. brev) (100 μl of 10⁸ CFU/mL) was individually co-cultured with (100 μl of ˜5×10⁷ CFU/mL) ST or SE in 7 ml of co-culture (MRS-LB, 1:1 ratio) media and incubated at 37° C. anaerobically for 24 has described previously. For enumeration, the co-cultures were plated on LB plates containing nalidixic acid at 0, 6, 12, and 24 hours. The experiment was repeated to ensure accuracy. The pH of each co-culture was measured at each time point. Multiple controls were included in this assay, including ST or SE inoculated MRS-LB controls, ST or SE grown in LB alone, and media controls for MRS, LB, and MRS-LB.

Trans-well assay: The effective probiotics in the previous experiments were used to test the antimicrobial activity of their secreted products. A 750 μl of 16-18 h grown LGG, and Bb12 cultures were placed above the filter in a 0.22 μm Ultrafree-MC micro-centrifuge filter tube (Millipore Sigma). Simultaneously, 750 μl of ST or SE culture (˜5×10⁷ CFU/mL) were placed below the filter and incubated at 37° C. under anaerobic conditions with shaking at 50 rpm. Volume used (750 μL) was sufficient to allow contact with the tube containing the filter. A positive control with 750 μl of corresponding Salmonella was placed above the filter and used for growth comparison and denoted as ST or SE on the respective graphs. ST and SE were appropriately enumerated on LB plates containing nalidixic acid at 12 h and 24 h post-incubation. Two replicates of this study were conducted.

Effect of probiotic Cell free supernatant (CFS) on the invasion of Salmonella in polarized HT-29 cells: Polarized human colorectal adenocarcinoma (HT-29; ATCC HTB-38) cells were incubated and maintained at 37° C. in a humidified atmosphere with 5% CO₂ in complete Dulbecco's modified Eagle's medium (DMEM, Gibco) supplemented with 10% fetal bovine serum (FBS, Gibco), 5 mM galactose, 2 mM L-glutamine, 1% penicillin-streptomycin (PS), and 0.1 mM non-essential amino acids (NEAA). ST and SE were adjusted to mid-logarithmic phase, pelleted, washed in DPBS, and re-suspended in DMEM. The HT-29 cells in 96-well cell culture plate, protocol as described in, were then infected with ST or SE (˜5×10⁷ CFU/mL) for 1 h. To allow invasion, infected cells were washed thrice and treated with DMEM containing 150 μg/mL gentamicin for 1 h. The HT-29 cells were then washed thrice with DPBS and then treated for 4 h with 12.5% and 25% (lowest concentration not inhibitory to Salmonella) of the probiotic CFS filtered through a 3 KDa Amicon filter. Untreated Salmonella infected cells were used as controls. After washing thrice, cells were lysed with 0.1% of Triton X-100 and Salmonella was enumerated on LB plates supplemented with nalidixic acid (as described earlier) to evaluate the probiotics' ability to inhibit ST/SE's invasion of the cells.

Quantification of organic acids in probiotics: As described previously, liquid chromatography—with tandem mass spectrometry (LC-MS/MS) was used with an isotope-labeled chemical derivatization method to quantify the organic acids present in LA, LGG, Bb12, and Lbrev. LC-MS/MS analyses were performed at The Mass Spectrometry and Proteomics Facility, The Ohio State University. Briefly, the probiotics were grown overnight, adjusted to OD₆₀₀ 1.0, and subcultured in new media for 24 h at 37° C. under anaerobic conditions. LC-MS/MS analysis was conducted using LC-MS/MS Poroshell 120 SB C18 column with solvent A; H₂O±0.1% formic acid and solvent B; MeCN+0.1% formic acid. Lactic acid, acetic acid, propionic acid, and butyric acid (Sigma Aldrich) were used as standard solutions and sodium ¹³C-lactic acid was used as the internal standard. This protocol was repeated with CFS of probiotics cultured with ST.

Effect of temperature and proteinase K on cell free supernatants of probiotics: The CFS were prepared through centrifugation at 10,000×g for 10 min at 4° C. and then filtered through a 0.22 μm filter. CFS of LGG and Bb12 were heated to 121° C. (autoclaved), cooled to 4° C. (refrigerated), and treated with proteinase K (1 mg/mL, 37° C. for 3 h) as described, to confirm and further characterize the antimicrobial properties of the probiotics' secreted products. The treated CFS were then placed in the agar well diffusion method as described above with the necessary controls. A 3 kDa Amicon Ultra centrifugal filter was used to fractionate LGG and Bb12 CFS product(s) of mol. wt. <3 kDa. The filtrate was used to confirm inhibition in agar well diffusion. Proteinase K treatment and subsequent inhibition assay was repeated for SE to confirm role of small peptides.

Effect of LGG and BB12 against ST infected chickens: The animal study was approved by The Ohio State University Animal Care and Use Program and performed following the Institutional Animal Care and Use Committee (IUACUC) protocol #2010 A00000149. One-day-old specific pathogen free (SPF) layer chickens were obtained from a Salmonella free flock at The Ohio State University and were provided with feed and water ad libitum. Chickens (n=10/group) were treated with 200 μL in PBS (10⁸ CFU/chicken) of LGG, Bb12, or LGG+Bb12 (1:1 combination) daily using oral gavage from day 1 until day 16 of age. On day 7 of age, chickens were orally infected with approximately 10⁴ nalidixic acid resistant ST. On day 17 of age, chickens were euthanized, weights were recorded, and the cecum, liver, and spleen were aseptically collected to determine the probiotics' ability to reduce ST infection. The cecum was suspended in PBS, homogenized, ten-fold serially diluted, and plated on XLT4 agar plates supplemented with 50 μg/ml nalidixic acid and incubated for 18 h at 37° C. The liver and spleen were suspended in PBS and the undiluted homogenized suspension was plated directly and incubated for 18 h at 37° C. Additionally, one milliliter of the undiluted homogenized liver and spleen was enriched in 9 ml of tetrathionate broth for 18 h at 37° C. After incubation they were plated on XLT4 plates supplemented with nalidixic acid to determine the birds positive for ST in each tissue. The positive control group (PC) was infected with Salmonella but not treated with probiotics and denoted as untreated on corresponding figures. The negative control group (NC) was not infected with Salmonella and not treated with probiotics.

Effect of LGG administered in water against ST infected chickens: One-day-old specific pathogen free (SPF) layer chickens (n=15/group) were obtained from a Salmonella free flock at The Ohio State University and were provided with feed and water ad libitum. LGG (10⁸ CFU/mL) was administered from 1 until 13-days of age continuously in drinking water, changed daily. The volume of drinking water needed was calculated and adjusted based on the standard requirements of chickens. On day 15, chickens were euthanized, and the cecum, liver, and spleen were aseptically collected to determine if probiotics administered in drinking water could retain anti-Salmonella activity. The tissues were processed using the aforementioned procedure. The PC was infected with Salmonella but not treated with probiotics and denoted as untreated on corresponding figures. The NC was not infected with Salmonella and not treated with probiotics.

Effect of LGG on multiple Salmonella serotypes: Multiple Salmonella serotypes (laboratory collection) that are commonly isolated from humans, animals, or produce were used in an agar well assay to test the efficacy of LGG against other Salmonella serotypes. Measurements were taken at 24 hours. The serotypes tested included S. anatum, S. albany, S. brenderup, S. javiana, S. heidelberg, S. muenchen, S. newport, and S. saintpaul.

Identification of bioactive probiotic derived peptides: LGG and Bb12 supernatants were used to identify derived peptides using LC-MS/MS as described previously. The bacteria were grown at 37° C. for 24 h under anaerobic conditions, centrifuged (1000 rpm, 10 min, 25° C.) and washed with sterile water. The pellets were then re-suspended in sterile water containing 2% glucose and incubated for 24 h. The probiotic cultures were then centrifuged (1000 rpm, 10 min, 4° C.) and the supernatants were separated and filtered using 0.2 μm filter. The supernatants (volume: 1.8 mL) were passed through HyperSep™ Hypercarb™ SPE cartridge (50 mg; ThermoFisher Scientific) and repeated three times. The cartridge was then washed twice with 150 μL of water to remove salts and then eluted twice (20 μL) using 50% acetonitrile (MeCN) and 0.1% trifluoroacetic acid (TFA). The eluted solutions (0.5 μL) were then injected in LC-MS/MS EasySpray C18-Fusion column set at different collision energy (HCD: higher energy collision dissociation and CID: ion-trap-based collision-induced dissociation) settings. The LC-MS/MS analysis was conducted at the Mass Spectrometry and Proteomics Facility, The Ohio State University. The two solvents used were solvent A (H₂O±0.1% formic acid) and solvent B (Acetonitrile (MeCN)+0.1% formic acid). The data was then analyzed using Proteome Discoverer 2.2 software (Thermo Fisher Scientific) using UniProt Lactobacillus or Bifidobacterium database with settings of no modifications and non-specific cleavage.

Five peptides in the CFS of both LGG and Bb12 (FSAVALSAVALSKPGHVNA (SEQ ID NO:5), AESSDTNLVNAKAA (SEQ ID NO:18), VQAAQAGDTKPIEV (SEQ ID NO:21), AFDNTDTSLDSTFKSA (SEQ ID NO:26) and VTDTSGKAGTTKISNV (SEQ ID NO:32)) were selected for synthesis based on their charge, hydrophobicity, and abundance in LGG and Bb12. Peptides were synthesized (GenScript) and tested for their ability to inhibit Salmonella in an inhibition assay. Briefly, peptides dissolved in dimethyl sulfoxide (DMSO) were added at 12 mM to Salmonella (105 CFU/mL) inoculated LB wells in a 96-well plate that was then incubated at 37° C. with OD₆₀₀ measurement set every 30 mins for 12 h in a TECAN Sunrise™ absorbance microplate reader. PN-3 and PN-5 were subsequently tested at 14 mM and 16 mM against ST and SE. Sterile media (NC) and DMSO treated, Salmonella infected wells (PC) were used as controls. OD values in the PC wells were used to calculate the growth inhibitory activity of the peptides—(OD₆₀₀ PC-OD₆₀₀ peptide treated well)/OD₆₀₀ PC×100%.

Statistical Analyses: Data generated from agar well diffusion assays were presented as means±standard deviations. Student two-tailed t-test analyses (P<0.05) were conducted for co-culture and cell culture invasion assays. ANOVA with Tukey Test (Trial 1) and Mann Whitney test (Trial 2) were analyzed for chicken studies using GraphPad PRISM Version 5. Cell culture Student two-tailed t-test analysis was done with Microsoft Excel 2010. Throughout the study *P<0.05, **P<0.01, ***P<0.001.

### Example 3. Peptides Affecting Outer Membrane Lipid Asymmetry (MlaA-OmpC/F) System Reduce Avian Pathogenic Escherichia coli (APEC) Colonization in Chickens

Avian pathogenic E. coli (APEC), an extra-intestinal pathogenic E. coli (ExPEC), causes colibacillosis in chickens and is reportedly associated with urinary tract infections and meningitis in humans. Development of resistance is a major limitation of current ExPEC antibiotic therapy. New antibacterials that can circumvent resistance problem such as antimicrobial peptides (AMPs) are critically needed. Here, the efficacy of Lactobacillus rhamnosus GG (LGG) derived peptides against APEC were evaluated and their antibacterial targets uncovered. Three peptides (NPSRQERR (SEQ ID NO:85): P1; PDENK (SEQ ID NO:86): P2, and VHTAPK (SEQ ID NO:87): P3) displayed inhibitory activity against APEC. These peptides were effective against APEC in biofilm and chicken macrophage HD11 cells. Treatment with these peptides reduced the cecum colonization (0.5 to 1.3 logs) of APEC in chickens. Microbiota analysis revealed two peptides (P1 and P2) decreased Enterobacteriaceae abundance with minimal impact on overall cecal microbiota of chickens. Bacterial cytological profiling showed peptides disrupt APEC membrane either by causing membrane shedding, rupturing or flaccidity. Further, gene expression analysis revealed that peptides downregulated the expression of ompC (>13.0 folds), ompF (>11.3 folds) and mlaA (>4.9 folds) genes responsible for maintenance of outer membrane (OM) lipid asymmetry. Consistently, immunoblot analysis also showed decreased levels of OmpC and MlaA proteins in APEC treated with peptides. Alanine scanning studies revealed residues crucial (P1: N, E, R and P; P2: D and E; P3: T, P, and K) for their activity. Overall, the study identified peptides with a new antibacterial target that can be developed to control APEC infections in chickens, thereby curtailing poultry originated human ExPEC infections.

APEC is a subgroup of ExPEC and considered as a foodborne zoonotic pathogen transmitted through consumption of contaminated poultry products. APEC shares genetic similarities with human ExPECs, including uropathogenic E. coli (UPEC) and neonatal meningitis E. coli (NMEC). The study identified LGG-derived peptides (P1: NPSRQERR (SEQ ID NO:85), P2: PDENK (SEQ ID NO:86), and P3: VHTAPK (SEQ ID NO:87)) effective in reducing APEC infection in chickens. Antimicrobial peptides (AMPs) can be used for antibacterial development because of their low propensity for resistance development and ability to kill resistant bacteria. Mechanistic studies showed peptides disrupt APEC membrane by affecting MlaA-OmpC/F system responsible for maintenance of OM lipid asymmetry, a new druggable target to overcome resistance problem in Gram-negative bacteria. Altogether, these peptides provide a valuable approach for development of novel anti-ExPEC therapies, including APEC, human ExPECs and other related Gram-negative pathogens. Further, effective control of APEC infections in chickens can curb poultry originated ExPEC infections in humans.

### Introduction

Avian pathogenic E. coli (APEC), an extra-intestinal pathogenic E. coli (ExPEC), is a severe and recalcitrant bacterial pathogen of poultry worldwide. Despite improvements in the poultry production systems over the years, APEC continues to remain as a serious problem to the poultry industry worldwide. APEC causes multiple extra-intestinal infections (yolk sac infection, omphalitis, respiratory tract infection, swollen head syndrome, septicemia, polyserositis, coligranuloma, enteritis, cellulitis and salpingitis) in poultry, collectively referred as avian colibacillosis. Colibacillosis results in significant morbidity and mortality (up to 20%) and decreased meat (2% decline in live weight) and eggs production (up to 15%)(3). Further, in young chickens, APEC can be associated with up to 53.5% mortality and can result in up to 36-43% carcasses condemnation at slaughter. Thus, colibacillosis results in multi-million dollars in annual losses to the poultry industry and remains as a serious impediment to the sustainable poultry production worldwide. Recently, APEC has been also reported as a foodborne human uropathogen which can be transmitted to humans through consumption of contaminated poultry products. Colicin-V (ColV) plasmids from poultry-associated APEC have been detected in E. coli isolates isolated from human patients with urinary tract infections, suggesting foodborne transmission of APEC from poultry to humans. Further, APEC is also considered as a source of antibiotic resistance genes (ARGs) to human pathogens which can make the human infections difficult to treat; thus, APEC is a threat to both animal and human health.

At present, antibiotics are commonly used to control APEC infections in poultry. However, APEC isolates are becoming more resistant to antibiotics, showing that the control of APEC infections will be challenging in the future. To date, APEC resistance to multiple antibiotics, including but not limited to tetracyclines, sulfonamides, aminoglycosides, quinolones, and 3-lactams, has been reported worldwide. Moreover, the control of APEC infections is complicated by increased restrictions in antibiotic usage worldwide (particularly US and European countries) in food-producing animals, including poultry, to reduce the emergence and transmission of antibiotic-resistant bacteria to humans. However, limiting on-farm use of antibiotics could significantly increase disease incidence leading to morbidity and mortality of food animals and thereby compromise production efficiency, food security and food safety. Therefore, there is an urgent need for developing new antibacterials that can circumvent resistance problem such as antimicrobial peptides (AMPs) which will consequently promote the sustainable poultry production as well as benefit the public health.

Antimicrobial peptides (AMPs) are regarded as a new category of therapeutic agents as well as alternatives to conventional antibiotics. A major strength of AMPs is their ability to kill antibiotic-resistant bacteria. AMPs are relatively small (10-50 amino acid residues), easy to synthesize, and have fast and selective antimicrobial action with low propensity for the development of resistance. The development of bacterial resistance to AMPs is less likely due to AMPs' mechanism of action involving multiple low-affinity targets rather than one defined, high-affinity target, which is a characteristic of conventional antibiotics. The multiple low affinity targets make it more difficult for bacteria to defend against AMPs by a single resistance mechanism. Further, AMPs, unlike antibiotics, do not elicit bacterial stress pathways such as SOS and rpoS responsible for inducing bacterial mutations and resistance. AMPs therefore can be used to exploit weaknesses in antibiotic-resistance mechanisms; thus, considered as the Achilles' heel of antibiotic resistance. Most AMPs exhibit a direct and rapid antimicrobial activity by disrupting the integrity of the bacterial membrane and/or by translocating into the cytoplasm of bacteria to act on intracellular targets. The differences between bacterial and mammalian membranes enable selective action of AMPs to bacterial membranes. Beside antimicrobial activity, AMPs also exhibit immunomodulatory activities, including suppression of pro-inflammatory responses, anti-endotoxin activity, stimulation of chemotaxis, and differentiation of immune cells, thereby contributing to the bacterial clearance by the host.

To date, few AMPs derived from soil bacteria such as colistin, gramicidin, vancomycin, and daptomycin have been successfully used as antibacterials to treat antibiotic-resistant bacteria. Nisin, a polycyclic antibacterial peptide derived from probiotic Lactococcus lactis, has been used as food preservative and sanitizer. Similarly, multiple AMPs (pexiganan, omiganan, Lytixar [LTX-10⁹], hlF1-11, Novexatin [NP-213], CZEN-002, LL-37, PXL01, Iseganan [IB-367], and PAC-113) derived from natural (human, bovine, porcine, and frog) and synthetic sources are in clinical development with indications against different bacterial pathogens. The synthetic AMPs ZY4, SAAP-148, arenicin-3, AMPR-11, and CSP-4 are effective against multi-drug resistant (MDR) bacterial infections. Further, AMPs (A3, P5, colicin E1, cecropin AD, cecropin A-D-Asn, cipB-LFC-LFA, sublancin, cLF36) have shown efficacy in decreasing E. coli and Clostridial burden in the gut as well as enhance the performance and immune status in pigs and chickens. Moreover, the efficacy of short or small AMPs (8-12 residues) have been also shown against Gram-negative sepsis, Staphylococcal skin infections, and bone infections.

In this study, the efficacy of probiotic Lactobacillus rhamnosus GG (LGG) derived small peptides (NPSRQERR (SEQ ID NO:85): P1, PDENK (SEQ ID NO:86): P2, and VHTAPK (SEQ ID NO:87): P3) was measured against APEC in vitro and in cultured chicken macrophage HD 11 cells, wax moth (Galleria mellonella) larva, and chickens. These peptides were identified through mass spectrometry (LC/MS) analysis of LGG culture supernatant. Antibacterial targets of peptides were uncovered using bacterial cytological profiling, gene expression and immunoblot approaches and the structure-activity relationship was probed by alanine scanning mutagenesis method. The peptides effective in reducing APEC colonization in chickens were identified and their likely mechanism of action which involves maintenance of outer membrane (OM) lipid asymmetry (MlaA-OmpC/F) system in APEC was uncovered.

### Materials and Methods

Bacterial strains, culture conditions, and media: APEC serotypes (O78, O1, O2, O8, O15, O18, O35, O109, and O115) provided by Drs. Tim Johnson (UMN, Saint Paul, MN), Lisa K. Nolan (UGA, Athens, GA), Catherine M. Logue (UGA, Athens, GA) and Roy Curtiss, III (UF, Gainesville, FL) were used in this study. Luria-Bertani (LB; BD Difco™) broth was used for routine propagation of APEC serotypes. Briefly, APEC serotypes stored at −80° C. in glycerol were inoculated in LB broth and grown overnight at 37° C. with shaking at 180 rpm. LB agar plates were used for quantification of APEC, unless otherwise indicated. (Table 19)

Peptide synthesis: All peptides (NPSRQERR (SEQ ID NO:85): P1, PDENK (SEQ ID NO:86): P2, VHTAPK (SEQ ID NO:87): P3, MLNERVK (SEQ ID NO:88): P4, YTRGLPM (SEQ ID NO: 118): P5, GKLSNK (SEQ ID NO:119): P6) used in this study were synthesized (>95% purity) through GenScript (NJ, USA). These peptides were selected because LGG showed activity against APEC. Peptides were dissolved in 100% dimethyl sulfoxide (DMSO) at concentration 300 mM and stored at −80° C. until further use.

Anti-APEC activity determination: The anti-APEC activity of peptides was determined in LB broth following CLSI guidelines. Briefly, overnight grown APEC O78 was adjusted to 5×10⁵ CFU/mL concentration in fresh LB media. One-hundred microliters of APEC suspension was aliquoted into the wells of the 96-well plate. Peptides were added to the wells at 6 mM and 12 mM concentrations and incubated at 37° C. in TECAN Sunrise™ absorbance microplate reader with kinetic absorbance measurement set at every 30 mins for 12 h. Sterile media and DMSO (solvent) were used as controls. The growth inhibitory activity of the peptides against APEC was calculated using the formula: (OD₆₀₀ DMSO treated well−OD₆₀₀ peptide treated well)/OD₆₀₀ DMSO treated well×100%.

The minimum inhibitory concentration (MIC) was determined for selected peptides (P1, P2, P3, and P4) using similar assay as above. Peptides were added to the wells of the 96-well plate at 6 mM, 9 mM, 12 mM, 15 mM, and 18 mM concentrations. The peptide concentration at which there is no visible APEC growth (no OD increase) was determined as MIC. Two independent experiments were conducted.

Anti-APEC spectrum determination: Peptides (P1, P2, and P3) were tested against multiple APEC serotypes/strains to determine their spectrum of activity. Peptides were added at their MICs and the growth inhibitory activity of the peptides was determined as described above.

Effect against beneficial microbes: Peptides (P1, P2, and P3) were tested against different commensal and probiotic bacteria to determine their specificity of activity as described previously. Peptides were added at their MICs to 100 μL of known concentration of bacterial cultures and cultures were incubated under required conditions. Following incubation, OD₆₀₀ of cultures was measured to determine the effect of peptides on bacterial growth.

MBEC™-HTP assay: To test the efficacy of the peptides (P1, P2, and P3) against biofilm protected APEC, Innovotech's MBEC Assay® was conducted as described previously(27). Briefly, 150 μL of 0.05 OD₆₀₀ (5×10⁷ CFU/mL) adjusted APEC suspension in LB media was aliquoted into the wells of MBEC™ device containing polystyrene pegs and incubated for 36 h on a rocker platform at 37° C. to allow the biofilm formation. After biofilm formation, the pegs were washed to remove loosely adherent planktonic bacteria and transferred to a new 96-well plate (challenge plate) containing peptides at MICs in 200 μL diluted (25%) LB media. The diluted LB media mimics the minimal media which stimulates biofilm formation and maintenance. The plate was incubated for 18 h at 37° C. with rotation at 150 rpm. Sterile media and DMSO (solvent) were used as controls. Following incubation, the MICs of peptides in challenge plate were recorded. The peptides exposed pegs were transferred to new 96-well plate containing PBS and sonicated for 30 mins to disrupt the biofilm. The sonicated suspensions were then ten-fold serially diluted and plated on LB agar plates to enumerate the biofilm protected bacteria and the minimum biofilm eradication concentration (MBEC) of peptides were determined. Three independent experiments were conducted.

Effect of peptides on intracellular survival of APEC in HD11 cells: To determine the efficacy of peptides (P1, P2, and P3) against intracellular APEC, gentamicin protection assay was conducted in HD 11 (chicken macrophage) cells as described previously. The cells were cultured in Iscove's Modified Dulbecco's Media (IMDM) supplemented with 10% fetal bovine serum (FBS), 2 mM L-glutamine and 2% penicillin-streptomycin (P/S) solution and maintained at 37° C. incubator with 5% C02. Cells (approximately 10⁴ cells/well) were seeded in a 96-well cell culture plate in 100 μL culture media and incubated for 36 h prior to using for the assay. Following incubation, the cells were replenished with 100 μL of fresh cell culture media with no FBS and antibiotic and incubated until infection (2 h). For the infection, the overnight grown APEC O78 was subcultured to mid-logarithmic phase, centrifuged, washed twice with PBS, adjusted to 1×10⁷ CFU/mL. One-hundred microliters of the APEC suspension (MOI: 100) was added to each well of the 96-well plate and incubated for 1 h to allow the invasion. The cells were washed twice and treated with gentamicin (150 μg/mL) to kill the extracellular APEC. The cells were treated with peptides at 12 mM, 15 mM, and 18 mM concentrations. Infected but not treated, infected and DMSO-treated, and non-infected and not treated cells were used as controls. Three independent experiments were conducted.

Bacterial cytological profiling (BCP): To identify the mode of action (MOA) of peptides (P1, P2, and P3), BCP approach was used as described previously. Briefly, 100 μL of 0.5 OD₆₀₀ (5×10⁸ CFU/mL) adjusted APEC O78 cultures were treated with 5×MIC of peptides and incubated at 37° C. for 3 h with shaking at 180 rpm. Following incubation, cultures were centrifuged, and bacteria was resuspended in 100 μL sterile water. FM4-64 (1 μg/mL; Invitrogen™ Molecular Probes™) and Syto-9 (5 μM; Invitrogen™ Molecular Probes™) stains were added to the bacterial cultures and incubated at 4° C. for 45 mins with shaking at 150 rpm. The stained bacteria were centrifuged, pelleted, and resuspended in 10 μL sterile water. The bacterial suspension (3 μL) was transferred onto glass slides containing thin layer of 1.2% agar and 20% LB media for confocal microscopy. Microscopy was performed using Leica TCS SP6 confocal scanning microscope (FM4-64-excitation 515 nm and emission 640 nm: Syto-9-excitation 485 nm and emission 498 nm) at Molecular and Cellular Imaging Center (MCIC), The Ohio State University (mcic.osu.edu/microscopy). Untreated bacterial culture was used as control.

Transmission electron microscopy: To visualize the effect of peptides (P1, P2, and P3) on APEC membrane, transmission electron microscopy (TEM) was performed as described previously. The peptide treatment was performed as described above. Briefly, 500 μL of 1.0 OD₆₀₀ (1×10⁹ CFU/mL) adjusted APEC O78 cultures were treated with 10×MIC of peptides and incubated at 37° C. for 3 h with shaking at 180 rpm. After incubation, cells were fixed overnight at room temperature with 3% glutaraldehyde & 2% paraformaldehyde), washed, and postfixed with 1% osmium tetroxide (OsO₄). The cells were then dehydrated with graded ethanol series and embedding was performed using Embed 812 Kit (Electron Microscopy Sciences, PA). Ultrathin (70 nm) sections were prepared on formvar-carbon coated grids and stained with uranyl acetate and lead citrate. Microscopy was performed using Hitachi H-7500 microscope at MCIC. Untreated bacterial culture was used as control.

Efficacy and toxicity of peptides in wax moth (Galleria mellonella) larvae model: To measure the efficacy and toxicity of peptides (P1, P2, and P3) in vivo, wax moth larvae model was used as described previously. Wax moth larvae were obtained from Vanderhorst Wholesale, Inc. (Saint Marys, OH) and were kept at 37° C. overnight before use. Healthy larvae (creamy color with no blackening) were selected and kept in sterile petri dishes before inoculation. For toxicity evaluation, larvae (n=10/group) were injected with 25.5 mM of peptides (dissolved in sterile water containing DMSO) through last left pro-leg using PB600-1 repeating dispenser (Hamilton) attached to insulin syringe (ReliOn; 31 gauze, 8 mm needle length). The injected larvae were incubated for 72 h at 37° C., and mortality was recorded every 12 h.

For efficacy evaluation, larvae (n=15/group) were pretreated (as above) and infected with rifampicin resistant (Rif) APEC O78 (2.3×10⁴ CFU/larva) through last right pro-leg within 30 mins of treatment. The larvae were incubated at 37° C. for 72 h and mortality was recorded as above. After 72 h post-infection, all dead and live larvae were surface sterilized (70% ethanol followed by sterile water) and macerated in PBS. The macerated larval suspensions were ten-fold serially diluted and plated on MacConkey agar plates containing 50 μg/mL rifampicin. Non-injected larvae and larvae injected with vehicle (sterile water containing DMSO) were included as controls in both the experiments. Two independent experiments were conducted. The statistical significance (P<0.05) of treatments on survival of larvae and APEC load inside larvae was calculated using log-rank test and one-way ANOVA followed by Tukey's post-hoc test, respectively.

Structure-activity relationship (SAR) study: To identify the crucial amino acid residues required for the peptide activity, alanine scanning libraries of peptides (P1, P2, and P3) (www.genscript.com/alanine_scanning.html) were synthesized (Table 22) and tested for anti-APEC activity as described above. The anti-APEC activity of peptide analogues (SEQ ID NO: 67-84) was then compared with the original peptide and the relative importance (% growth inhibition by peptide analogue/% growth inhibition by original peptide×100%) of each AA residue of the peptide was determined.

Arginine and lysine substituted peptide analogues: The non-essential amino acid residues identified in each peptide were substituted with arginine/lysine to test whether these substitutions enhance the anti-APEC activity of peptides. The MICs of arginine and lysine substituted peptide analogues (NPRRQERR (SEQ ID NO:37), NPSRRERR (SEQ ID NO:38), NPRRRERR (SEQ ID NO:39), KDENK (SEQ ID NO:41), PDEKK (SEQ ID NO:42), KDEKK (SEQ ID NO:43), KHTAPK (SEQ ID NO:45), VKTAPK (SEQ ID NO:46), VHTKPK (SEQ ID NO:47), and KKTKPK (SEQ ID NO:48)) were determined as above and compared with original peptides.

Resistance studies: To evaluate ability of APEC to develop resistance against peptides, sub-lethal and lethal resistance assay were performed as described previously. For sub-lethal resistance assay, peptides (P1, P2, and P3) were added at sub-inhibitory (0.75×MIC) concentrations to 100 μL of APEC suspension (5×10⁵ CFU/mL) in LB media in 1.5 mL microcentrifuge tubes. The tubes were incubated at 37° C. with shaking at 125 rpm for 24 h. After the first incubation, 20 μL of grown APEC culture was mixed with 80 μL of fresh LB media and peptides were added again at 0.75× MICs. This procedure was repeated for 13 times. Following 14^{th} passage, MIC of peptides was determined against APEC culture grown from 14^{th} passage as above. The tubes containing sterile LB media and DMSO-treated APEC suspension were used as controls. For lethal resistance assay, overnight grown APEC culture (10⁸ CFU) was plated on LB agar plates mixed with lethal (5×MIC) concentration of peptides (P1, P2, and P3) and incubated for 5 days at 37° C. Any colonies grown on agar plate were assessed for resistance by determining MIC as above. The agar plates containing sterile LB media and DMSO-mixed APEC culture were used as controls. Experiments were performed in duplicates.

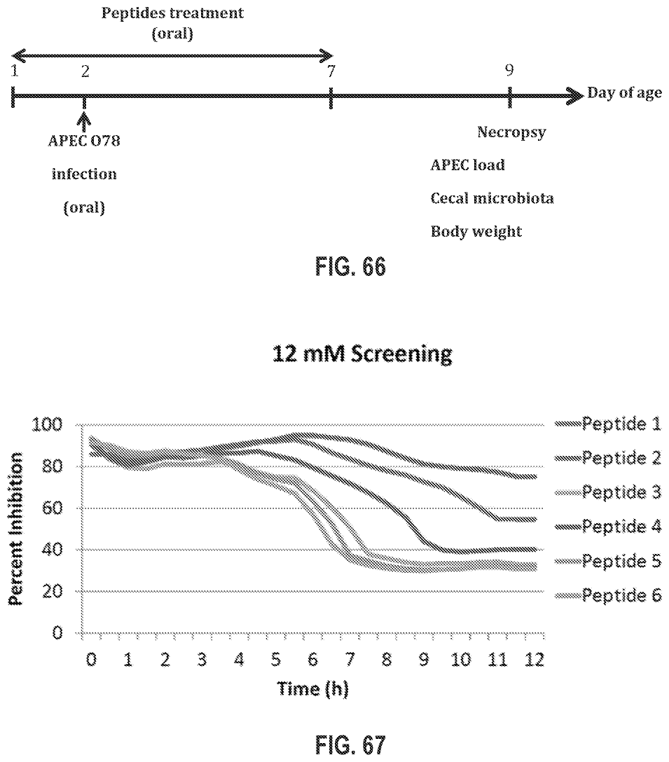

Efficacy of peptides in chickens: Animal study was approved by The Ohio State University Institutional Animal Care and Use Committee (IACUC, protocol #2010 A00000149). Chickens were euthanized using CO₂ following American Veterinary Medical Association (AVMA) guidelines. Standard animal husbandry practices were followed throughout the experiment. Feed and water were provided ad libitum. The efficacy of peptides (P1, P2, and P3) were assessed at 50 mg/kg and 100 mg/kg doses in two successive experiments using commercial broiler chickens (n=10/group) (Cobb & Ross; Case Farms Ohio Hatchery, Strasburg, OH). The schematic diagram of the experimental design is displayed in FIG. 66. Briefly, peptides dissolved in water [5 mg (50 mg/kg) or 10 mg (100 mg/kg) in 100 μL] were administered orally twice a day from day 1 (one day before APEC infection) today 7 (5 days post-infection; dpi). On day 2, chickens were infected orally with rifampicin resistant (Rif) APEC O78(27) (1-2×10⁹ CFU/chicken). At 7 dpi (day 9), chickens were euthanized, necropsied and cecum and internal organs (lung, liver, heart, and kidney) were assessed for APEC load by plating on MacConkey agar plates containing 50 μg/mL rifampicin. The body weight of chickens was measured at the day of necropsy (day 9). The positive (PC; infected but not treated) and negative (NC; non-infected and non-treated) control chickens were included in both experiments. The statistical significance (P<0.05) of treatment on reduction of APEC load and effect on body weight was calculated using one-way ANOVA followed by Tukey's post-hoc test. To test if APEC acquired resistance against peptides (P1, P2, and P3) after treatment in chickens, APEC colonies (n=25/peptide/experiment) were randomly selected from agar plates that were plated with cecum and different internal organs at the end of experiments. Bacteria were grown in LB media and MICs were determined as above against respective peptides.

Cecal microbiome analysis: To investigate the impact of peptides (P1, P2, and P3) treatment (50 mg/kg and 100 mg/kg) on cecal microbiome of chickens, 16S rRNA based metagenomic study was conducted as previously described. DNA was extracted from 0.2 g of cecal contents using PureLink™ Microbiome DNA Purification Kit (Thermofisher Scientific) and treated with RNase A (2 μL of 100 mg/mL solution per sample; Qiagen) to remove the RNA. DNA quantity and quality were measured using NanoDrop 2000c Spectrophotometer (Thermo Fisher Scientific). The extracted DNA samples were subjected to 16S rRNA V4-V5 sequencing at molecular and cellular imaging center (MCIC), Ohio Agricultural Research and Development Center (OARDC) (mcic.osu.edu/genomics/illumina-sequencing). Amplicon libraries were prepared using IFU KAPA HiFi HotStart ReadyMixPCR Kit (Roche) and PCR clean-up was performed using Agencourt AMPure XP beads (BECKMAN COULTER Life Sciences). Nextera XT DNA Library Preparation Kit (Illumina) was used to generate Illumina library and sequencing was performed using Illumina MiSeq platform generating paired end 300-bp reads.

For the metagenomic analysis, QIIME (Quantitative Insights into Microbial Ecology) 2 bioinformatics platform (qiime2.org/) was used. Quality control of the raw reads was performed using FastQC 0.11.8 (Babraham Bioinformatics). Trimmomatic-0.33 was used to trim the adaptor and other illumina-specific sequences (www.usadellab.org/cms/?page=trimmomatic). The trimmed sequences (fastq.gz) were then imported into the QIIME 2 as a manifest file format (PairedEndManifestPhred33V2). The feature table construction and additional filtering of the sequences was performed using DADA2. The taxonomic analysis was performed using Naive Bayes classifiers trained on the Silva 132 99% OTUs (silva-132-99-nb-classifier.qza) database. The phylogenetic diversity was analyzed using align-to-tree-mafft-fasttree pipeline and alpha (Shannon's diversity index) and beta diversity (Bray-Curtis distance) were analyzed using core-metrics-phylogenetic pipeline (docs.qiime2.org/2019.7/tutorials/moving-pictures/). The statistical difference (P<0.05) in the taxonomic composition between the peptides treated, PC, and NC groups was determined using Mann-Whitney U test. The alpha and beta diversity were analyzed using Kruskal-Wallis and PERMANOVA tests (P<0.05), respectively.

Gene expression analysis: In order to identify the target(s) of peptides (P1, P2, and P3), expression of genes essential for maintaining OM integrity (lptD, mlaA, bamA, lolB, pbgA, and mlaC) was quantitated as described previously. Further, expression of ompC and ompF genes was also quantitated to confirm peptides effect on MlaA-OmpC/F system. APEC O78 culture grown overnight was adjusted to OD₆₀₀ 0.1 (1×10⁸ CFU/mL) in LB media and 200 μL of APEC suspension was treated with peptides (8 replicates) for 8 h (50% lethal concentration) at 37° C. with shaking at 200 rpm as previously described(46, 47). APEC O78 culture treated with DMSO was used as control. Post-treatment, total RNA was extracted using RNeasy mini kit (Qiagen). RNA quantity and quality were measured using NanoDrop 2000c Spectrophotometer. DNA traces were removed using genomic DNA elimination mix (Qiagen). Five μg of purified RNA was used to synthesize cDNA using RT² First Strand Kit (Qiagen). The RT-qPCR was performed using Maxima SYBR Green/ROX qPCR master mix (Thermo Fisher) in a RealPlex² Mastercycler® (Eppendorf) with 55° C. annealing temperature. The primers (SEQ ID NO: 49-66) (Table 20) were designed using PrimerQuest Tool and obtained from Integrated DNA Technologies (IDT). The data were normalized to the house-keeping gene, glyceraldehyde-3-phosphate dehydrogenase (GAPDH) and relative fold change was calculated using ΔΔCt method. Two independent experiments were conducted.

Immunoblot analysis: To corroborate the findings of gene expression study, level of OmpC and MlaA proteins upon peptides (P1, P2, and P3) treatment was investigated as described previously. Peptides treated culture of APEC O78 was prepared as above and DMSO treated culture was used as control. Post-treatment, the fractions of cytoplasmic and membrane proteins was prepared using ultracentrifugation as previously described. The concentrations of fractionated membrane proteins were then normalized based on 280 nm and separated on a 12% SDS-PAGE. For immunoblot analysis, membrane proteins (25 μg) resolved on SDS-PAGE above were electrotransferred onto an Immuno-Blot® PVDF Membrane (Bio-Rad) and probed for OmpC and MlaA using anti-OmpC (1:2,000) (ThermoFisher Scientific) and anti-MlaA (1:15,000; Dr. Thomas J. Silhavy) polyclonal antibodies and goat anti-rabbit IgG HRP-conjugated secondary antibody (Sigma-Aldrich). The membrane was developed with Clarity Western ECL Substrate (Bio-Rad) and visualized in a FluorChem Q (proteinsimple) imager. The density of OmpC and MlaA proteins was quantified using ImageJ software.

Docking studies: HPEPDOCK and PEP-SiteFinder tools were used to predict the affinity and binding sites of peptides to OmpC (PDB:2J1N) and OmpF proteins (PDB:3K1B).

### Results

### Three Peptides (P1, P2, and P3) Displayed Bactericidal Activity Against APEC O78

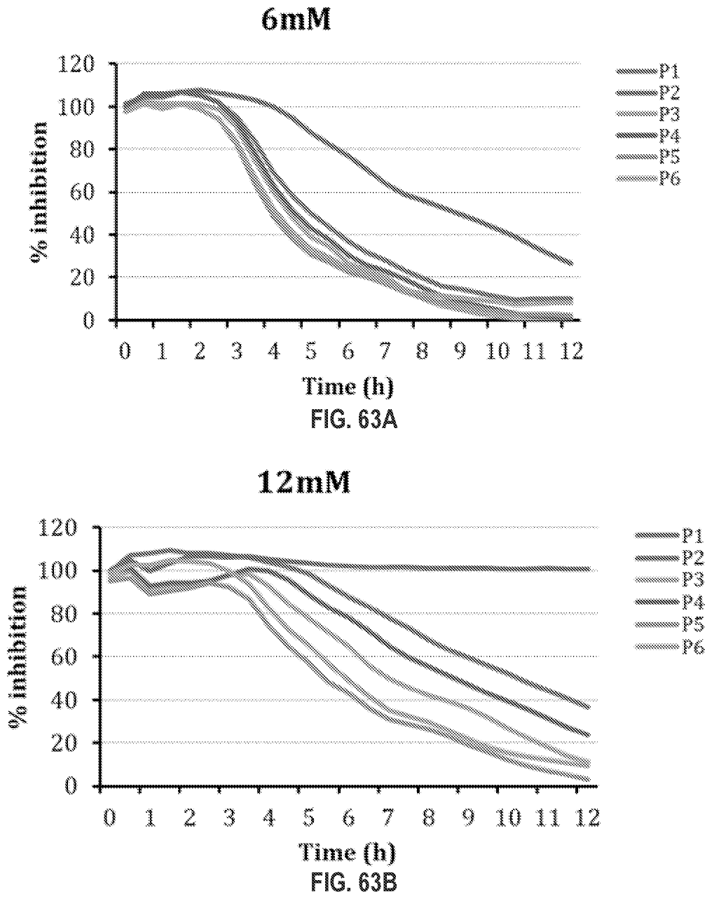

In order to assess the anti-APEC activity of peptides, peptides (NPSRQERR (SEQ ID NO:85): P1, PDENK (SEQ ID NO: 86): P2, VHTAPK (SEQ ID NO:87): P3, MLNERVK (SEQ ID NO:88): P4, YTRGLPM (SEQ ID NO:118): P5, GKLSNK (SEQ ID NO:119): P6) were added to APEC suspension at different concentrations and incubated for 12 h at 37° C. The initial testing at 6 mM and 12 mM concentrations revealed 4 peptides (P2>P1>P4>P3) inhibiting the APEC growth (>10%) (FIG. 63A). P2 inhibited 26.6% of APEC growth at 6 mM, whereas P1 inhibited 10% of APEC growth. Similarly, at 12 mM, P2 inhibited 100% of the APEC growth, whereas P1, P4 and P3 inhibited 36.8%, 24% and 11.2% of APEC growth, respectively (FIG. 63B).

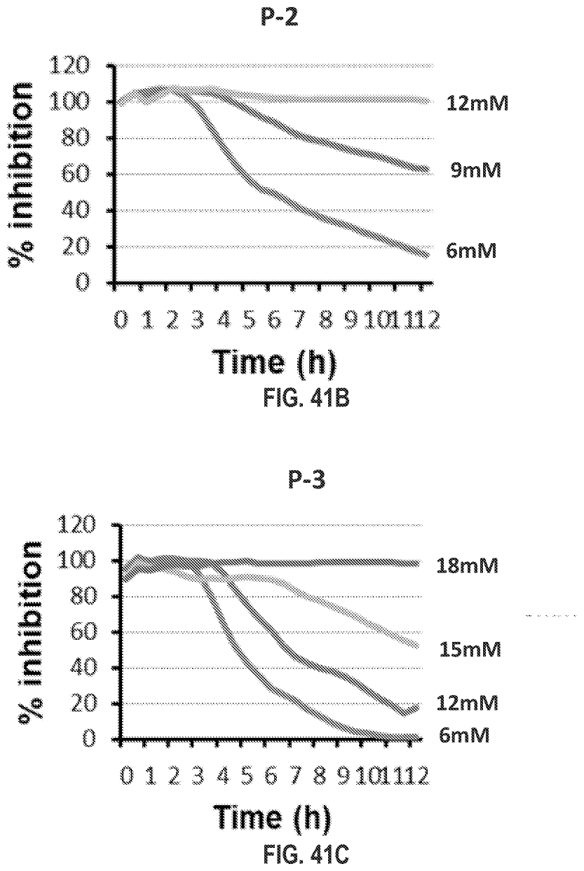

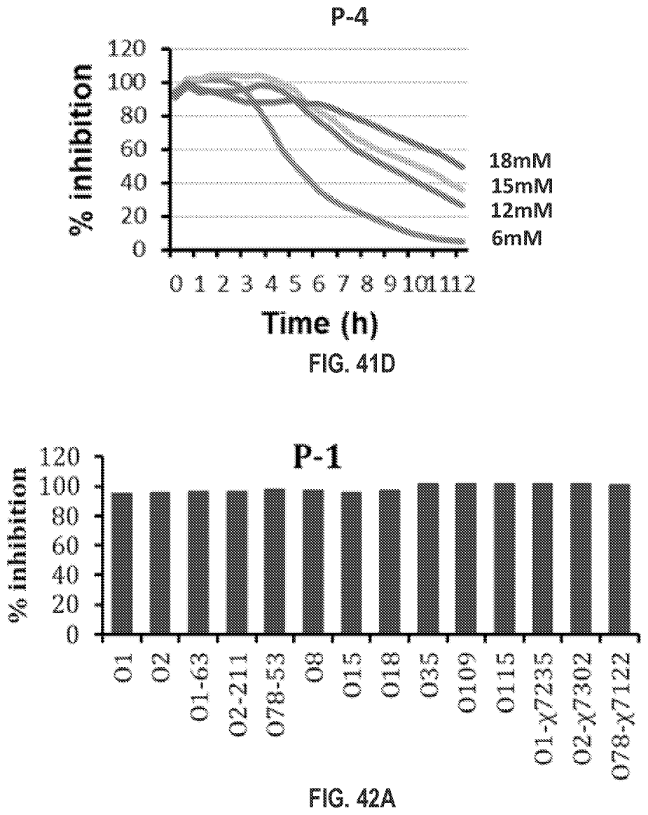

Further, to determine the MIC, dose-response study was conducted using different concentrations (6 mM, 9 mM, 12 mM, 15 mM and 18 mM) of peptides. P1, P2 and P3 displayed 100% APEC growth inhibition (i.e., MIC) at 18 mM, 12 mM and 18 mM, respectively (FIGS. 41A, 1B and 1C). However, P4 did not display 100% APEC growth inhibition at concentration up to 18 mM (FIG. 41D); therefore, only three peptides (P1, P2, and P3) were selected for further studies. The MIC₅₀ (concentration that inhibits 50% of APEC growth) of the peptides are displayed in Table 21. Additionally, the peptides treated APEC cultures at their MICs were plated on LB agar plates to determine their bacteriostatic/bactericidal activity. No viable APEC colonies were observed (data not shown) indicating bactericidal activity of the peptides.

### Peptides Displayed Activity Against Multiple Antibiotic-Resistant APEC Serotypes

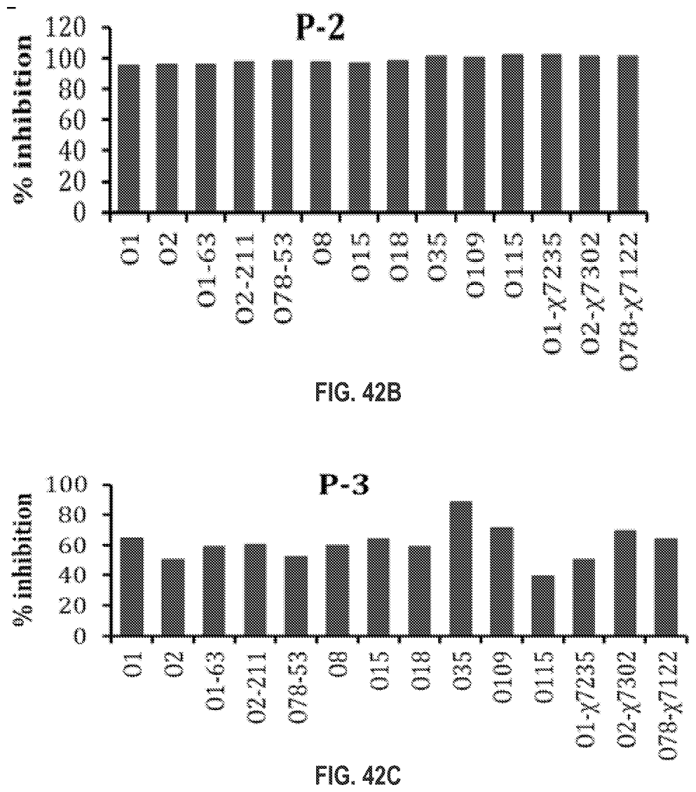

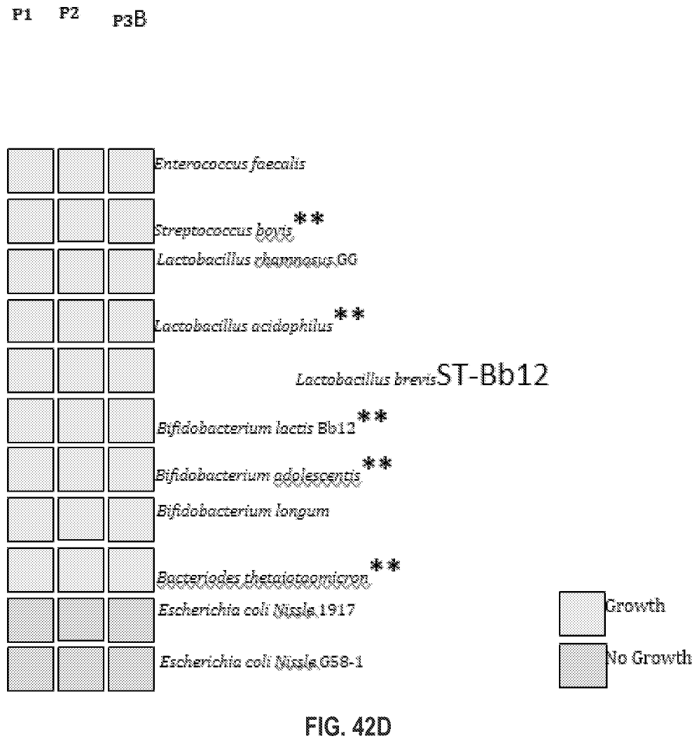

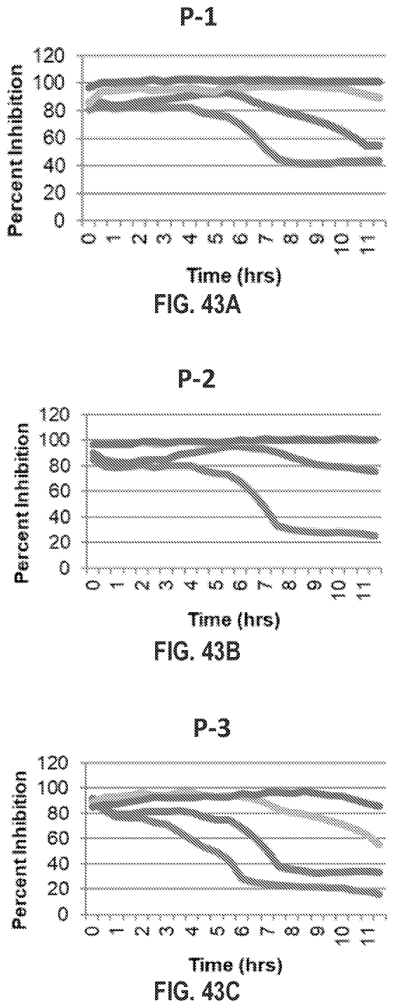

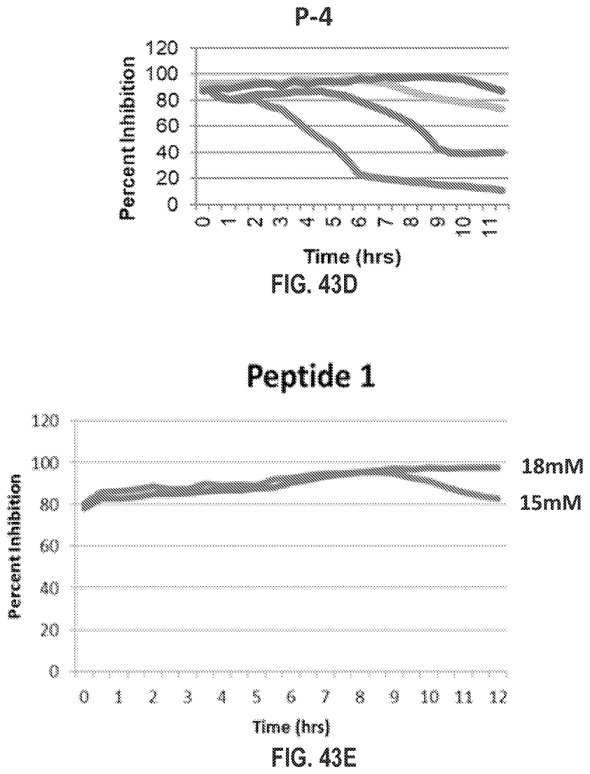

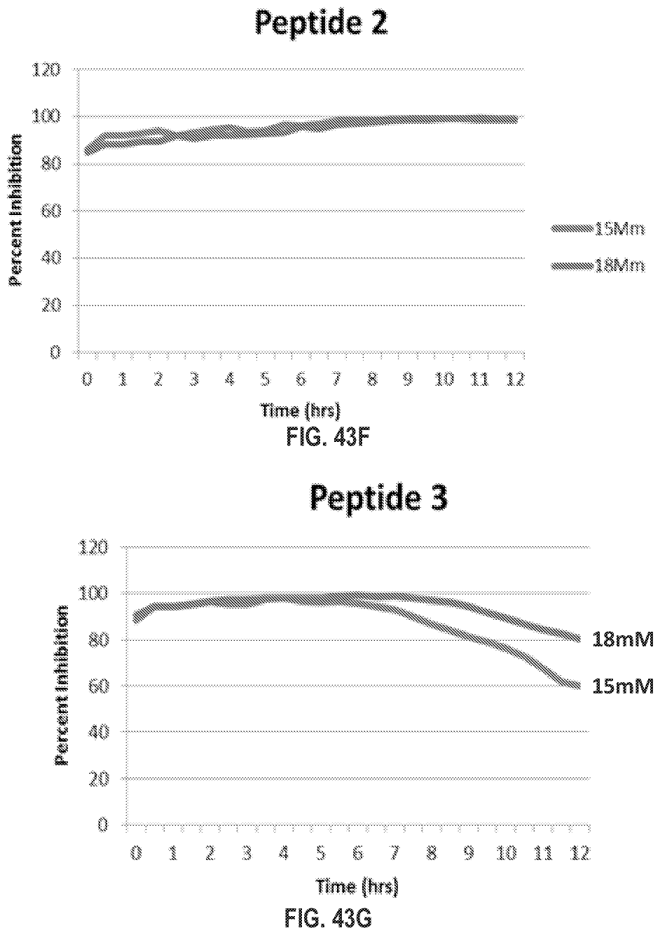

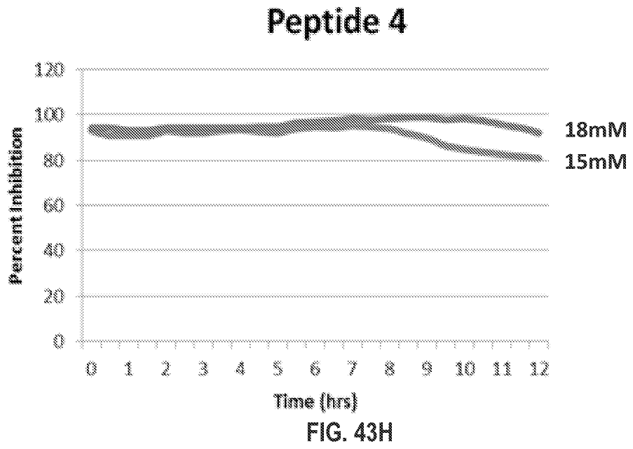

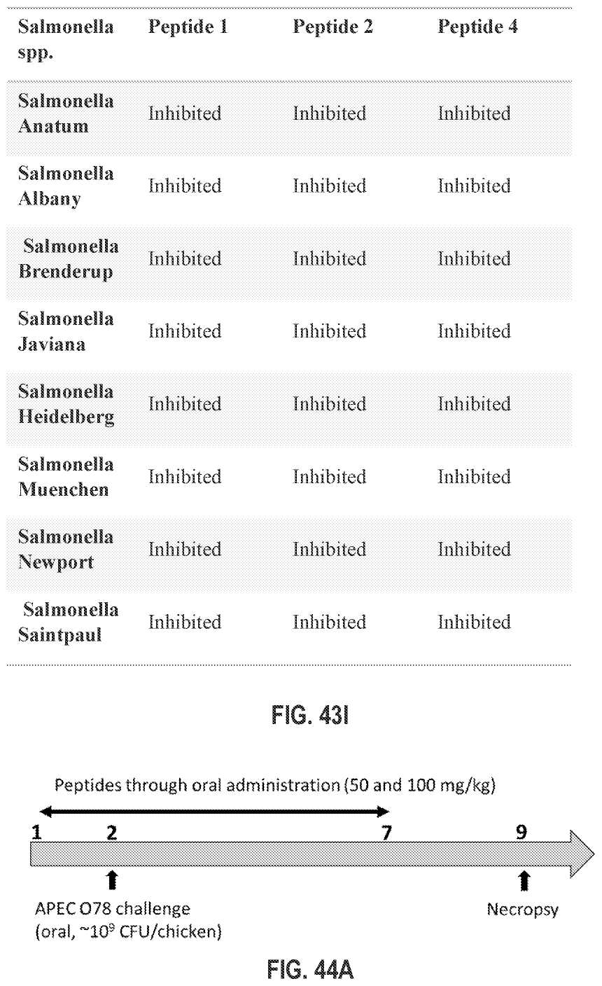

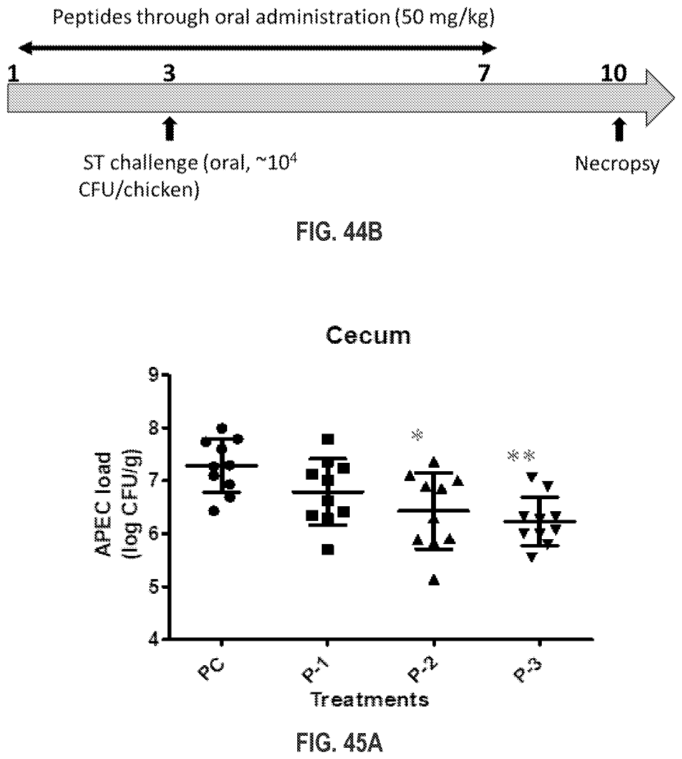

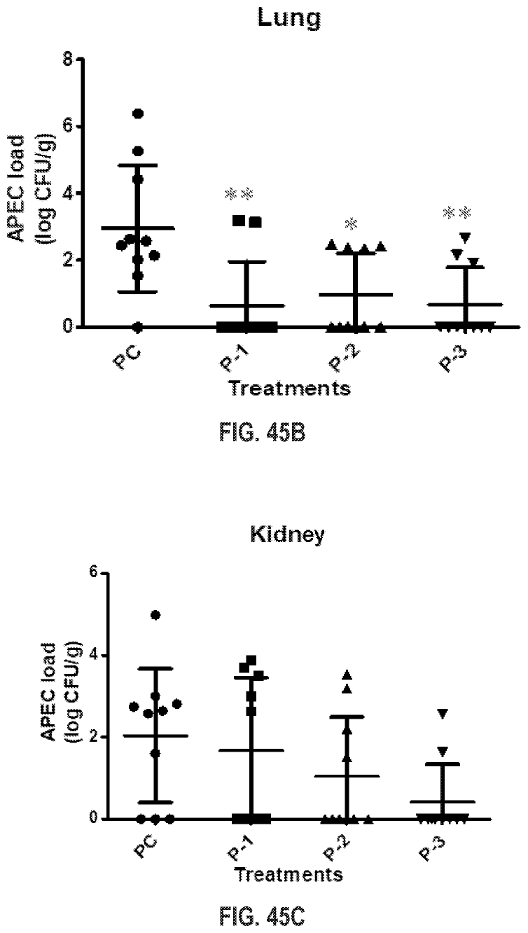

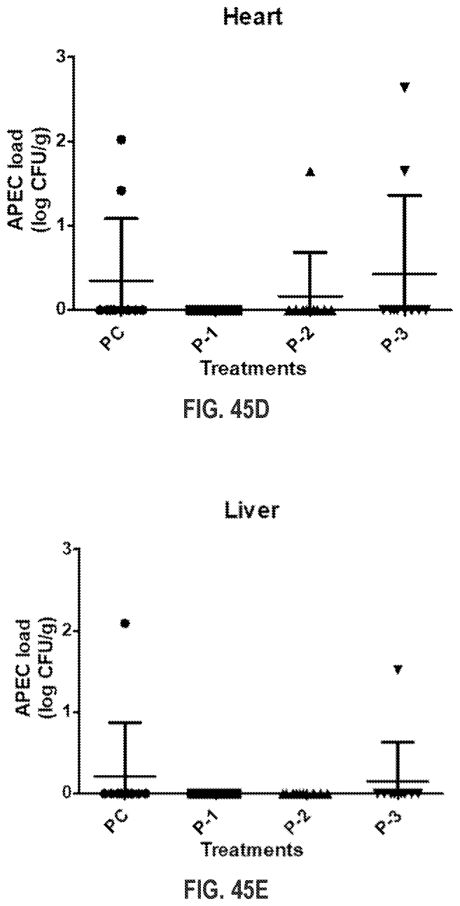

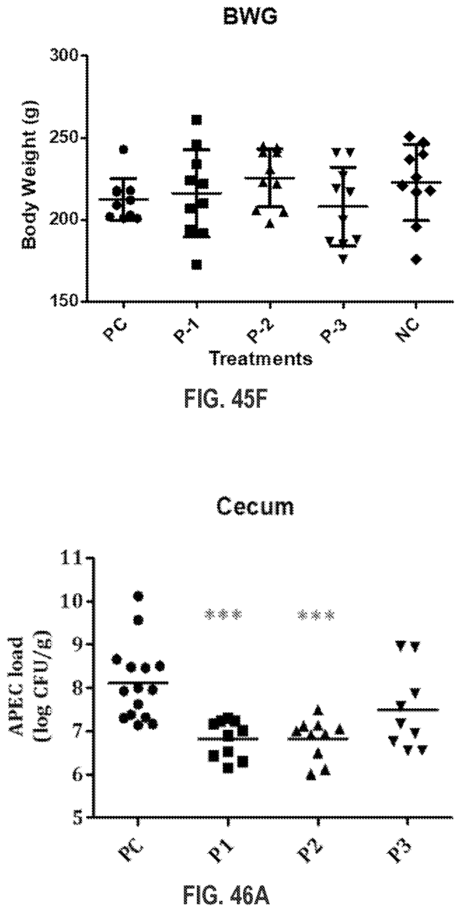

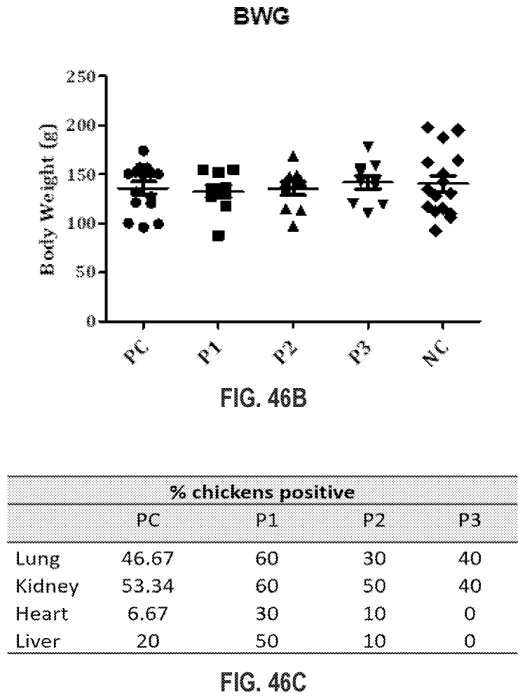

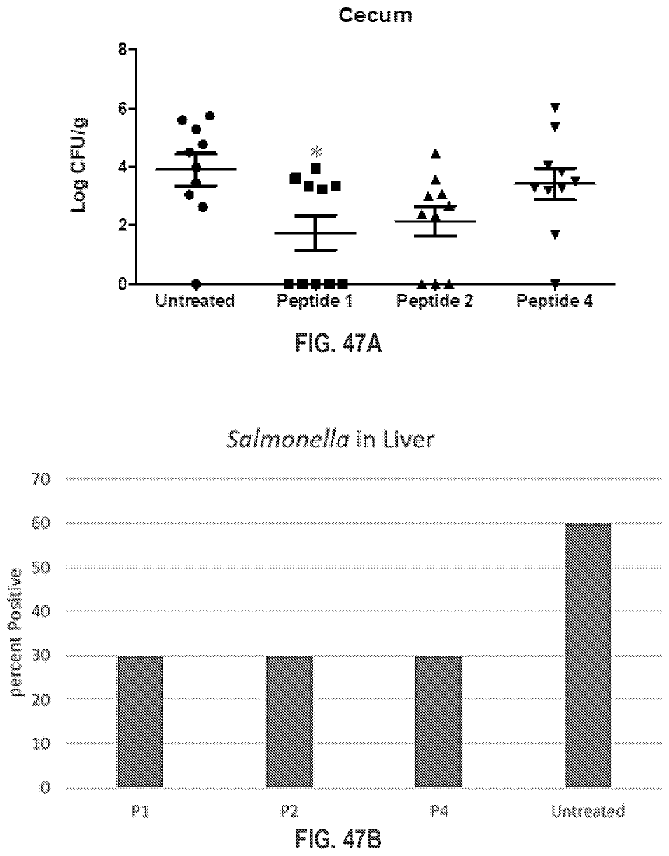

Peptides (P1, P2, and P3) were tested at their MICs against multiple APEC serotypes (O1, O2, O1-63, O2-211, O78-53, O8, O15, O18, O35, O10⁹, O115, O78-X7122, O1-X7235 and O2-X7302) to determine their spectrum of activity. These tested APEC serotypes were previously found resistant to different antibiotics, including ampicillin, tetracycline, ciprofloxacin and colistin. P1 and P2 displayed almost 100% (P1: 95.1% to 100% and P2: 95.0% to 100%) growth inhibition, whereas P3 displayed 39.4% to 88.4% growth inhibition against APEC serotypes (FIGS. 42A, 42B & 42C). These data show the broad spectral activity of peptides against multiple APEC serotypes irrespective of their resistant phenotype against antibiotics.

### Peptides Exhibited No Effect Against Gram-Positive Beneficial Bacteria

Peptides (P1, P2, and P3) were tested at their MICs against different commensal and probiotic bacteria to determine their specificity of activity. Interestingly, no effect on the growth of Gram-positive beneficial bacteria was observed (FIG. 42D). Whereas, growth of Gram-negative beneficial bacteria, particularly E. coli, was inhibited but not the B. thetaiotaomicron demonstrating activity of peptides only against E. coli and related Gram-negative bacteria.

### Peptides Eradicated the Biofilm Protected APEC in Preformed Biofilm and Cleared the Intracellular APEC in Macrophage Cells

The efficacy of peptides against biofilm protected APEC was determined using MBEC Assay®. The preformed APEC biofilm in the pegs of MBEC™ device was treated with the peptides for 18 h at MICs followed by the enumeration of APEC. All peptides (P1, P2, and P3) treatment completely eradicated the biofilm embedded APEC at their MICs (Table 15). The untreated pegs had 7.54±0.07 log CFU/mL of APEC in the biofilm.

To measure the effect of peptides on the intracellular survival of APEC, gentamicin protection assay was performed in chicken macrophage HD 11 cells infected with APEC and treated with 12 mM, 15 mM, and 18 mM concentrations of peptides. The untreated HD11 cells had 3.75±0.10 log CFU/mL of APEC (Table 15). No intracellular APEC was recovered from cells treated with P1 and P2 at 15 mM, and P3 at 18 mM. No effect on the viability of the HD 11 cells was observed with treatment up to 18 mM of peptides when determined using trypan blue exclusion test (Table 15).

### Peptides Disrupt the APEC Membrane Either by Sloughing, Rupturing, or Inducing Flaccidity

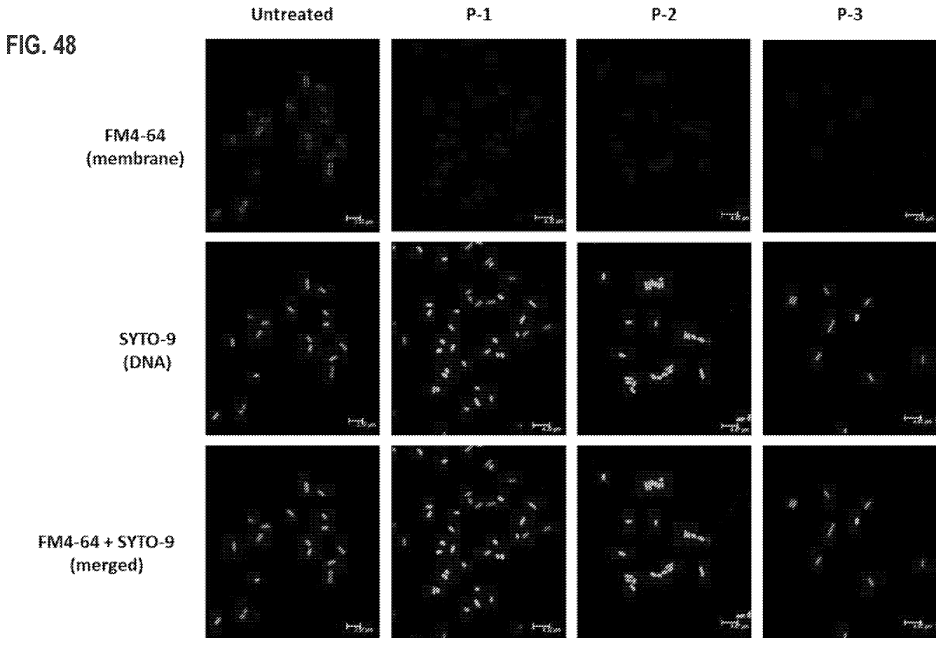

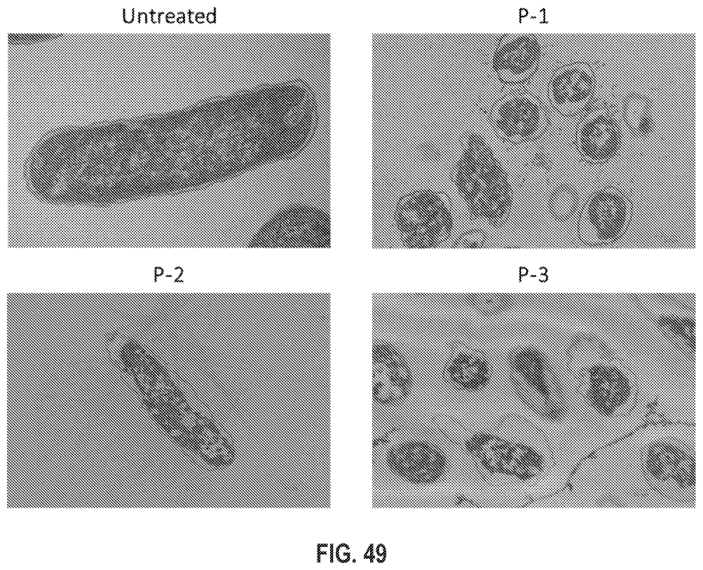

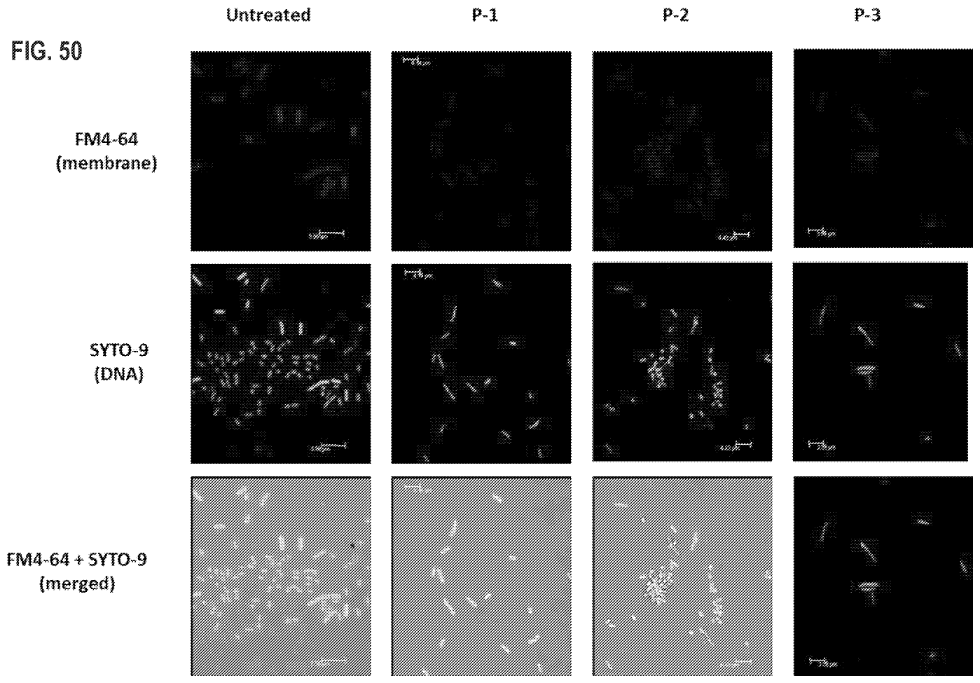

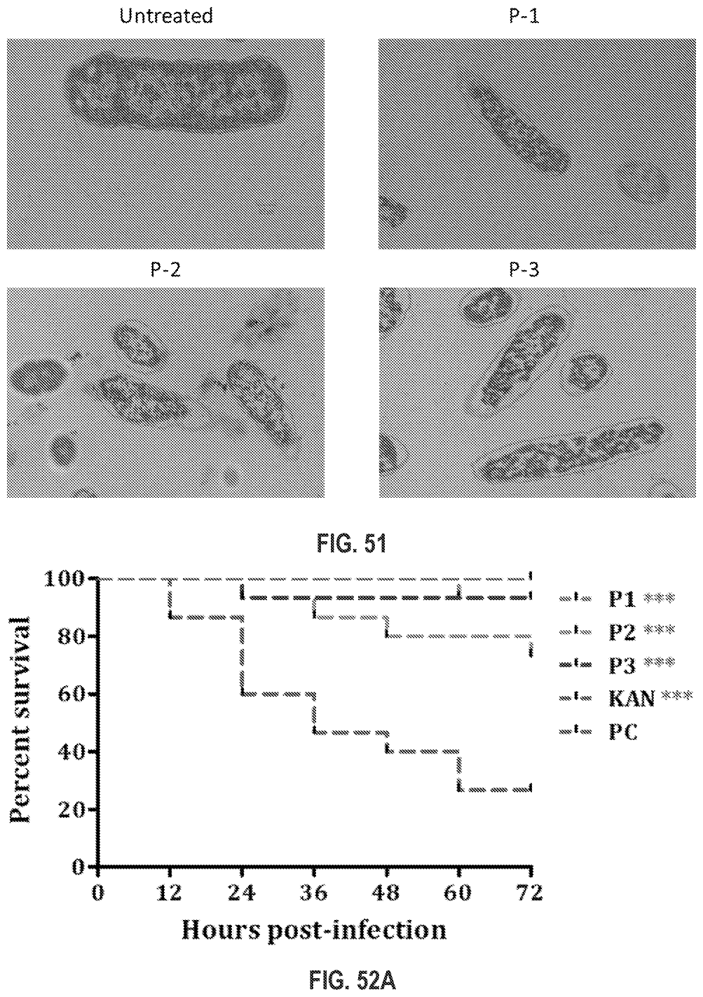

In order to identify the MOA of peptides, confocal fluorescence microscopy mediated BCP method was utilized. The membrane stain FM4-64 (red) and nuclear stain SYTO-9 (green) were used. In the untreated APEC, clearly demarcated APEC membrane encircling the chromosomes/nuclear material was visible (FIG. 48); whereas, in P1, P2 and P3 treated APEC, no or minimally visible APEC membrane was observed, showing peptides disrupt the APEC membrane. Consistence with the confocal images, in P1, P2 and P3 treated APEC, membrane either sloughed (or shed), ruptured, or flaccid was observed in TEM, showing that the peptides affect the APEC membrane (FIG. 49). In untreated APEC, clearly demarcated APEC membrane encircling the dense cytoplasmic contents was observed.

Peptides Protected the Wax Moth Larvae from APEC Infection

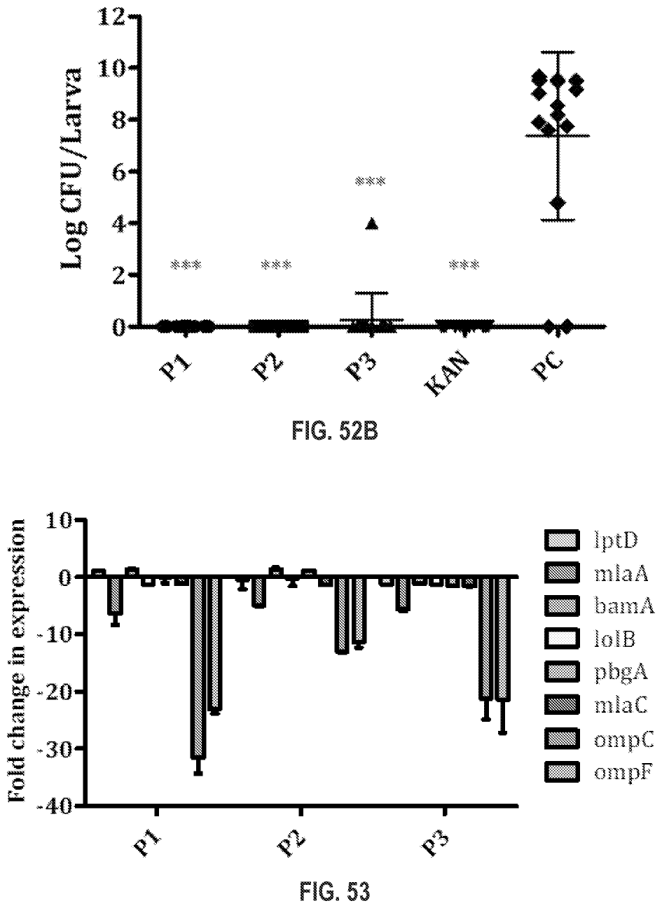

The in vivo efficacy and toxicity of peptides was measured in wax moth larvae. In order to measure the efficacy, larvae were pretreated with 25.5 mM of peptides, infected with APEC, and larval survival was assessed for 72 h and APEC load in the larvae was quantified at 72 h post-infection. A 73.34% larval mortality was observed in untreated group; whereas peptides treatment significantly (P<0.001) increased the survival of larvae (FIG. 52A) with only 6.67%, 26.67%, and 6.67% mortality in P1, P2 and P3 treated groups, respectively. Peptides also significantly (P<0.001) reduced the APEC load in the larvae (FIG. 52B). The untreated group had 7.4±3.1 log CFU/larva of APEC; whereas, no APEC was isolated in larvae treated with peptides, except for one larva in P3 treated group.

To measure the toxicity, peptides were injected into larvae (non-infected) at 25.5 mM (>MIC) concentration and larval survival was monitored for 72 h. No larval mortality was observed either in the control or the peptide treated groups in two independent experiments.

### Alanine Scanning Revealed Amino Acid Residues Critical for Anti-APEC Activity of the Peptides

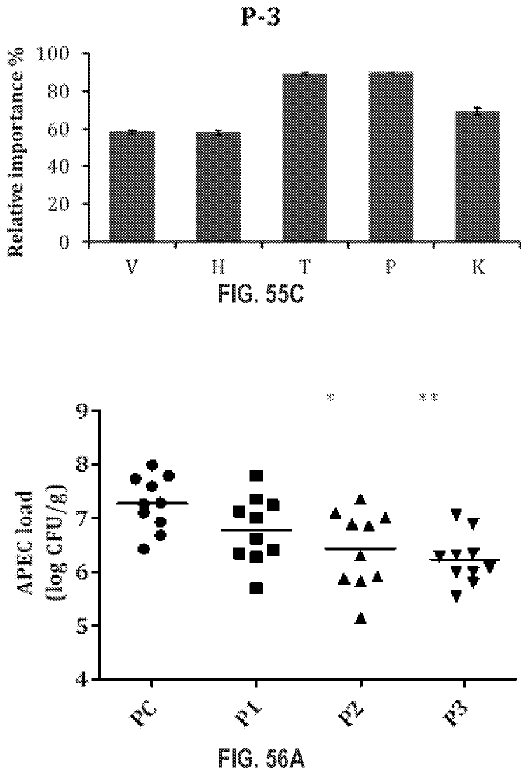

The alanine scanning libraries of peptides (Table 21) were generated by substituting alanine at each amino acid residue to identify residues important for anti-APEC activity of peptides(35). In P1 (NPSRQERR (SEQ ID NO:85)), the relative importance of N (asparagine), P (proline), glutamate (E) and R (arginine) was >50%; whereas the relative importance of S (serine) and glutamine (Q) was <45% (FIG. 55A). Interestingly, relative importance of R (arginine) varied based on the position (16%, 59.1% and 59.7% at position 4, 7 and 8, respectively) in the peptide. In P2 (PDENK (SEQ ID NO:86)), the relative importance of D (aspartate) and E (glutamate) was very high (97.1%) as compared to P (proline), N (asparagine) and K (lysine), whose relative importance was <40% (FIG. 55B). In P3 (VHTAPK (SEQ ID NO:87)), the relative importance of all AAs; V (valine), H (histidine), T (tyrosine), P (proline) and K (lysine) was >50% (FIG. 55C).

### Arginine and Lysine Substitutions Improved the Anti-APEC Activity of P1 and P2

The amino acids not critical for antibacterial effect in each peptide were either substituted with arginine (R) or lysine (K) to enhance the anti-APEC activity. Compared to native peptide P1 (NPSRQERR (SEQ ID NO:85)), the MIC of NPRRQERR (SEQ ID NO:37), NPSRRERR (SEQ ID NO:38), and NPRRRERR (SEQ ID NO:39) were decreased by 3 to 6 mM (Table 17). Similarly, the MIC of KDENK (SEQ ID NO:41) and PDEKK (SEQ ID NO:42) were also decreased by 3 mM compared to native peptide P2 (PDENK (SEQ ID NO:86)). However, the substitutions in peptide P3 (VHTAPK (SEQ ID NO:87)) increased the MIC of analogues.

Peptides Downregulated the Expression of ompC, ompF and mlaA Genes Responsible for Maintenance of OM Lipid Asymmetry

The expression of genes essential for maintaining the OM integrity, including ompC, ompF and mlaA was quantified to determine the target(s) of the peptides. Interestingly, treatment with all peptides significantly downregulated the expression of ompC (13 to 31.4 folds), ompF (11.3 to 23.0 folds), and mlaA (4.9 to 6.3 folds) genes responsible for maintenance of OM lipid asymmetry in E. coli (45, 53)(FIG. 53). No significant effect on the expression of other genes (lptD, bamA, lolB, and pbgA) was observed. Notably, the expression of mlaC gene was only slightly affected (<1.5 folds), demonstrating peptides are affecting the OM.

### Peptides Decreased the Levels of OmpC and MlaA Proteins

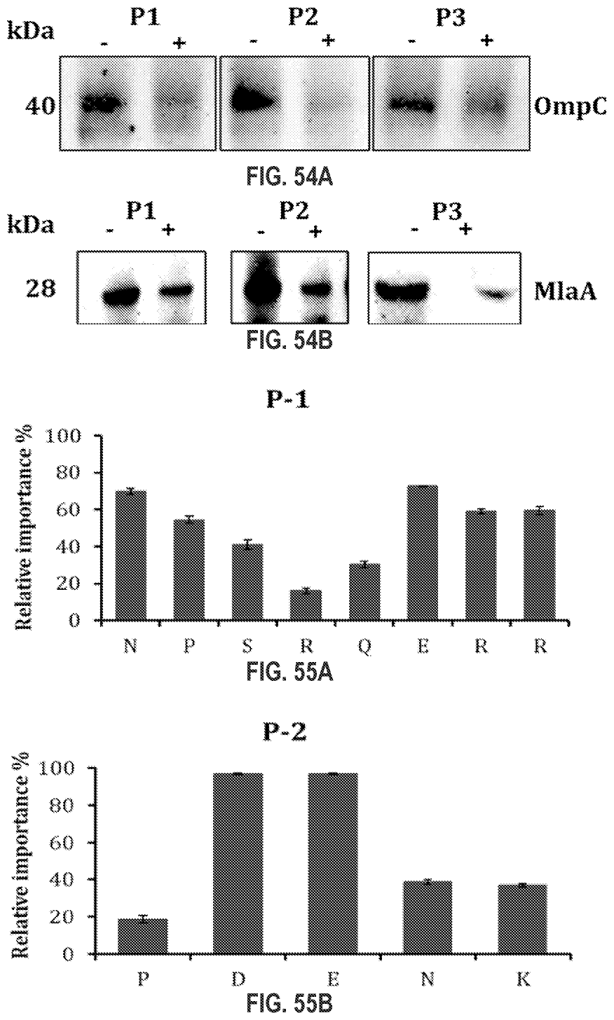

The levels of OmpC and MlaA proteins in APEC treated with peptides were assessed using anti-OmpC and anti-MlaA polyclonal antibodies. Consistence with downregulation of ompC gene, peptides treatment decreased the OmpC level in the OM of APEC compared to untreated APEC (FIG. 54A). Based on the densitometry analysis, the levels of OmpC were 4.76 to 9.27 folds (P1: 8.63 folds, P2: 9.27 folds, and P3: 4.76 folds) lower in the peptides treated APEC as compared to untreated APEC. Similarly, peptides treatment also decreased MlaA level (P1: 1.50 folds, P2: 1.40 folds, and P3: 1.98 folds) in the OM of APEC as compared to untreated APEC (FIG. 54B).

### Peptides Reduced APEC Colonization in Cecum of Chickens

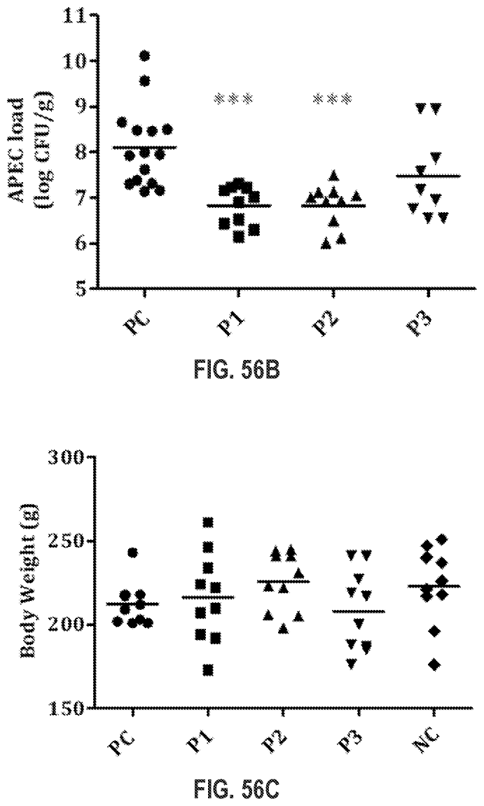

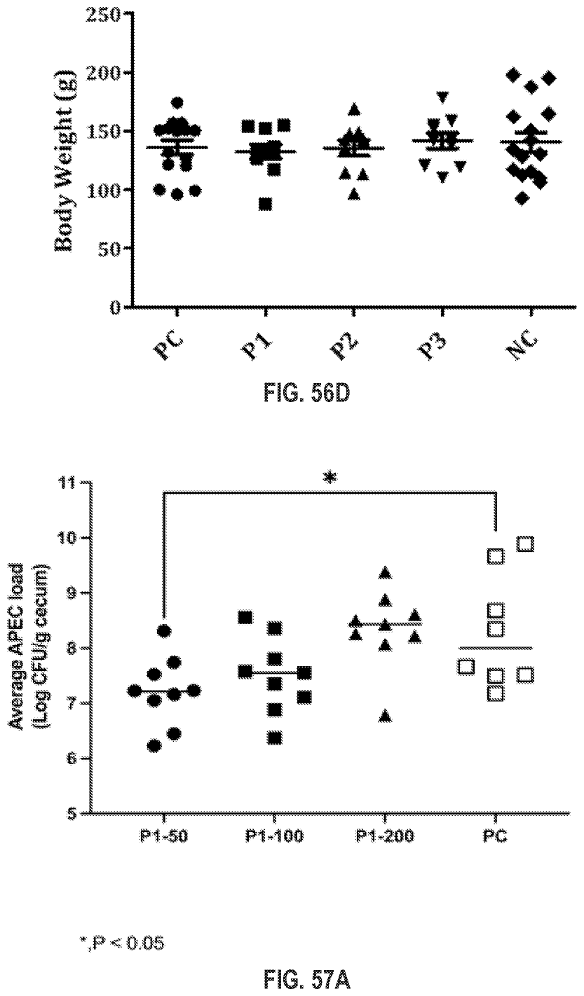

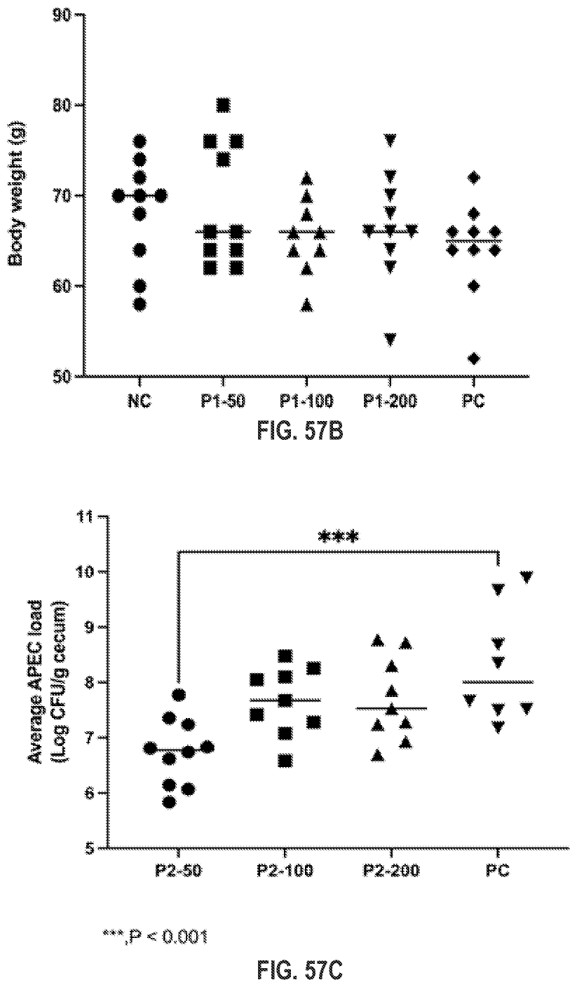

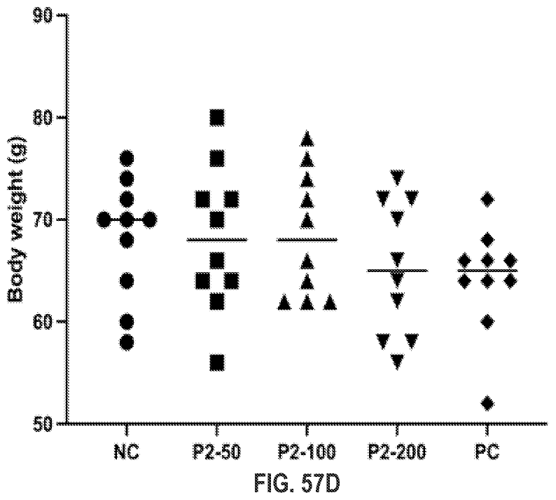

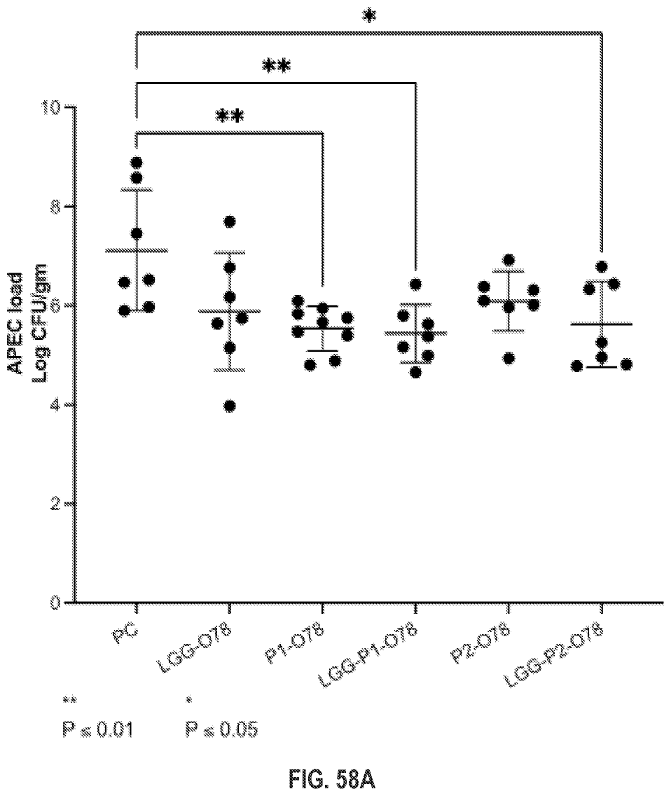

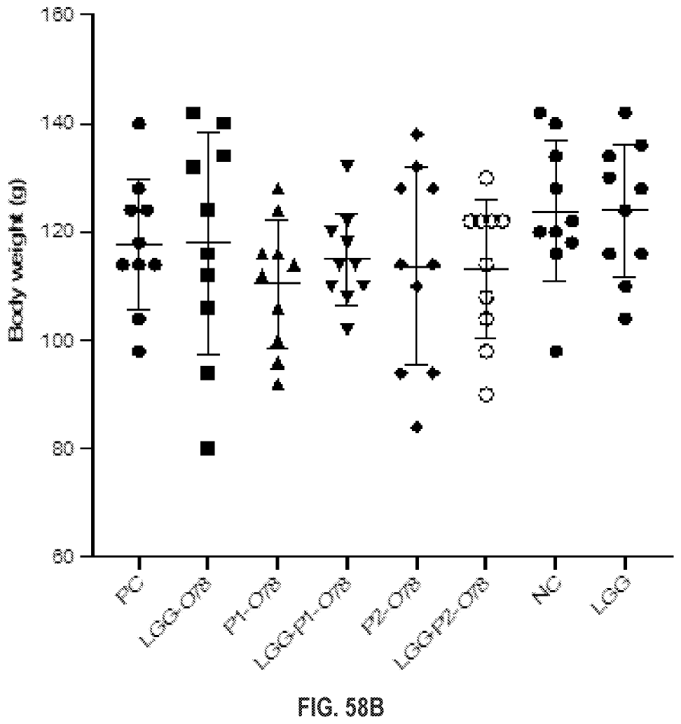

Peptides were administered at 50 mg/kg and 100 mg/kg doses in two successive experiments in order to determine their efficacy. All peptides reduced the colonization of APEC in cecum of chickens at both doses (FIG. 56). At 50 mg/kg dose, P1, P2 and P3 reduced the colonization by 0.5, 0.9 (P<0.05) and 1.1 (P<0.01) logs, respectively (FIG. 56A). At 100 mg/kg dose, P1 (1.3 logs, P<0.001) and P2 (1.3 logs, P<0.001) showed better effect in reducing APEC colonization (FIG. 56B). Surprisingly, the efficacy of VHTAPK (SEQ ID NO:87) (0.6 log) was not enhanced with the increased dose. Notably, at 50 mg/kg dose, peptides also reduced the APEC load in internal organs (lung, kidney, liver, and heart) of chickens, particularly lung (P<0.05) (Table 46C). Whereas, at 100 mg/kg dose, with exception of P1, P2 and P3 also reduced the number of chickens positive for APEC in internal organs. Further, at both the doses, no significant effect on the body weight of chickens was observed (FIGS. 56C & 56D).

P1 and P2 Decreased Enterobacteriaceae Abundance with Minimal Impact on Cecal Microbiota of Chickens

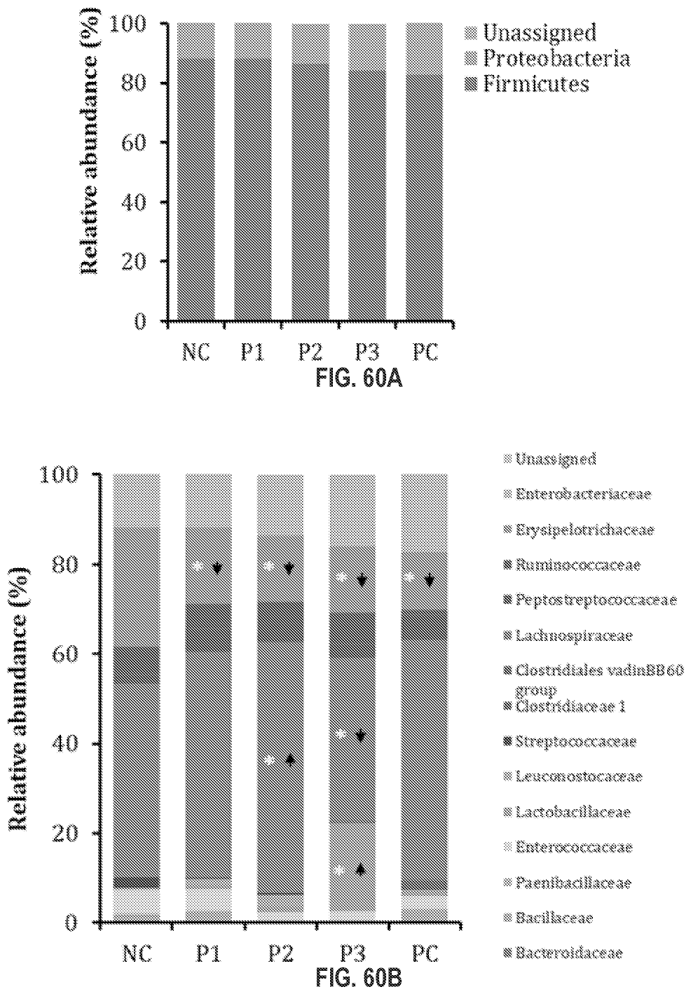

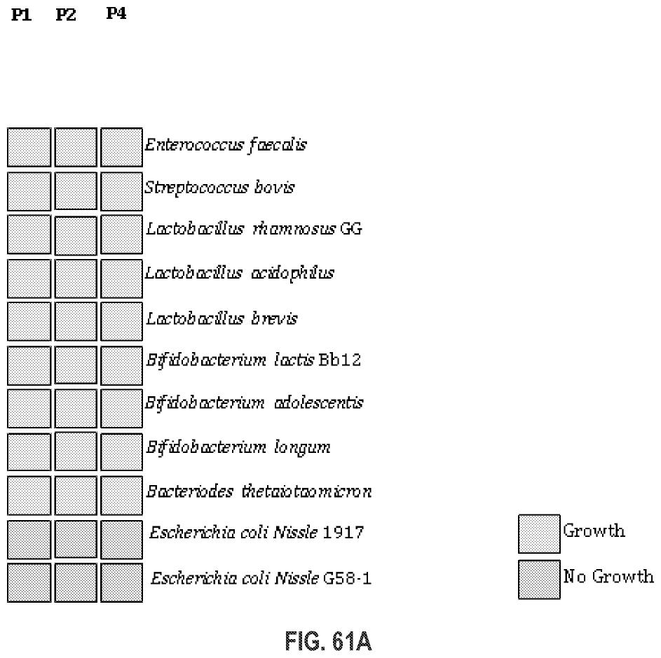

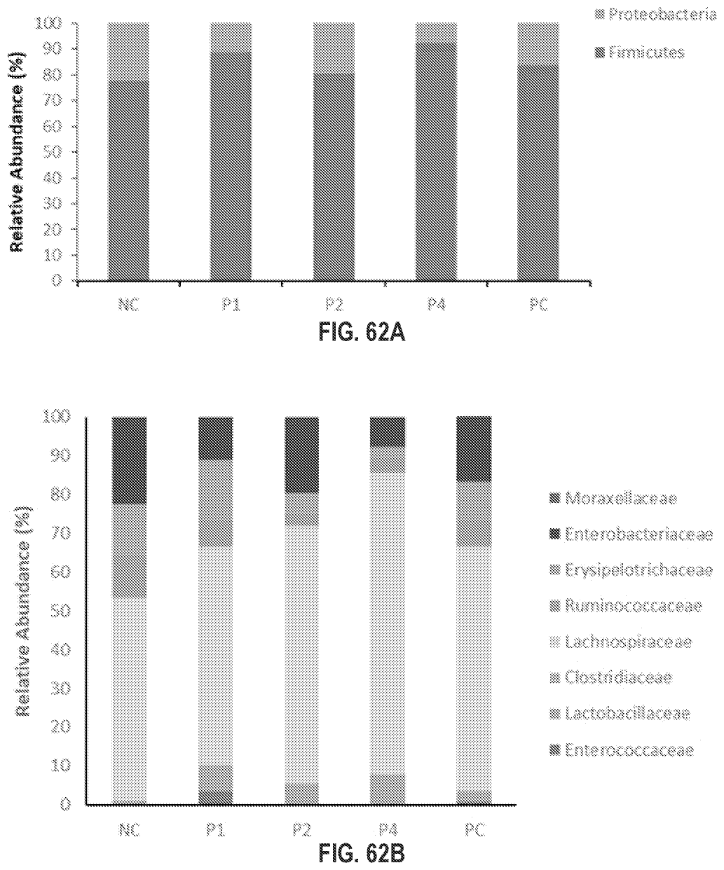

The metagenomic analysis of the cecal microbiota of chickens infected with APEC and treated with peptides was performed to investigate the effect of peptides on gut microbiota and identify the microbial markers associated with APEC infection in poultry. At the phylum level, no significant alteration was observed with the peptides treatment as compared to NC (non-infected and non-treated) chickens, except in chickens treated with P3 at 50 mg/kg dose (FIGS. 62A, 62B). P3 (50 mg/kg) decreased the Bacteroidetes abundance (56.39% to 4.78%); whereas increased the Firmicutes (39.54% to 82.47%) abundance. At the class level, all peptides (P1, P2, and P3) treatment at 100 mg/kg dose reduced Erysipelotrichia abundance (26.67% to 14.70%) as compared to NC chickens; whereas P2 increased Clostridia (51.76% to 64.92%) abundance. P3 increased Bacilli abundance (7.28% to 22.17%) and decreased Clostridia (62.63% to 46.99%) abundance as compared to PC (infected but not treated) chickens. On the other hand, peptides (P1 and P2) treatment at 50 mg/kg dose reduced Erysipelotrichia abundance (11.82% to 3.83%) as compared to PC chickens, whereas P3 increased Clostridia abundance (33.28% to 71.46%) and decreased Bacteroidia (38.09% to 4.78%) abundance as compared to NC and PC chickens. At the family level, all peptides (P1, P2, and P3) treatment at 100 mg/kg dose reduced Erysipelotrichaceae (26.67% to 14.70%) and Streptococcaceae (2.17% to 0%) abundance as compared to NC chickens, whereas P2 (51.76% to 64.92%) increased Lachnospiraceae (43.05% to 56.10%) and Lactobacillaceae (0.01% to 1.38%) abundance and decreased Clostridiaceae 1 (0.53% to 0%) abundance. P3 increased Lactobacillaceae abundance (0.74% to 19.61%) and decreased Lachnospiraceae (53.78% to 36.58%) abundance as compared to PC chickens; whereas Ruminococcaceae (1.40% to 7.35%) abundance was increased with all peptides (P1, P2, and P3) treatment and Leuconostocaceae abundance (0.31% to 0%) was decreased with P2 treatment. On the other hand, peptides (P1 and P2) treatment at 50 mg/kg dose reduced Erysipelotrichia abundance (11.82% to 3.83%) as compared to PC chickens, whereas P3 increased Lachnospiraceae abundance (30.95% to 62.09%) and decreased Bacteroidaceae (56.39% to 4.78%) abundance as compared to NC and PC chickens (FIGS. 60A, 60B). Interestingly, all peptides (P1, P2, and P3) treatment at 100 mg/kg and 50 mg/kg doses decreased Enterobacteriaceae abundance as compared to PC chickens, except P3 treated at 50 mg/kg dose (FIGS. 60A, 60B Table 18).

At the genus level, the abundance of Escherichia-Shigella was decreased in both 100 mg/kg (16.24% to 11.73%) and 50 mg/kg doses (8.14% to 4.95%) treated groups, except chickens treated with P3 at 50 mg/kg dose (Table 18). At 50 mg/kg dose, P1 and P2 decreased Erysipelatoclostridium abundance (11.82% to 3.84%) compared to PC chickens; whereas P3 decreased Bacteroides abundance (56.04% to 38.09%) compared to PC and NC chickens (Table 18). At 100 mg/kg dose, all peptides decreased abundance of [Clostridium] innocuum group (2.03% to 0.00%) compared to NC chickens (Table 18). Further, P1 decreased the abundance of Butyricicoccus (2.04% to 0.44%), Sellimonas (2.95% to 0.00%), and Lactococcus (2.18 to 0.00%). Whereas P2 decreased the abundance of Erysipelatoclostridium (24.65% to 14.71%), Flavonifractor (1.92% to 0.68%), and Sellimonas (2.95% to 0.00%) and increased the abundance of Pediococcus (0.01% to 1.38%) compared to NC chickens and increased the abundance of Weissella (0.40% to 2.43%) compared to PC chickens. P3 decreased the abundance of Erysipelatoclostridium (24.65% to 14.78%) and Butyricicoccus (2.04% to 0.19%), and increased the abundance of Lachnospiraceae (uncultured) (2.38% to 10.38%) compared to NC chickens; whereas, increased the abundance of Lactobacillus (0.00% to 19.62%) and Ruminiclostridium 9 (0.00% to 1.52%) compared to both PC and NC chickens, and increased the abundance of Clostridiales vadin BB60 group (gut metagenome) (0.00% to 0.41%) and decreased the abundance of Sellimonas (2.95% to 1.60%) compared to PC chickens. Interestingly, compared to NC chickens, PC chickens showed decreased abundance of [Clostridium] innocuum group (2.03% to 0.00%), Erysipelatoclostridium (24.65% to 12.68%), Flavonifractor (1.92% to 0.52%), Sellimonas (2.95% to 0.13%) and Lactococcus (2.18% to 0.00%).

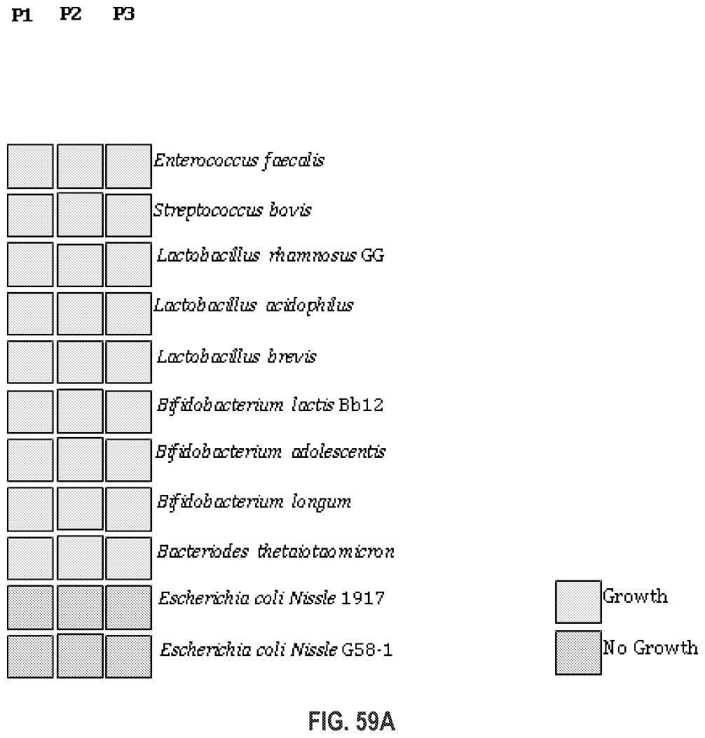

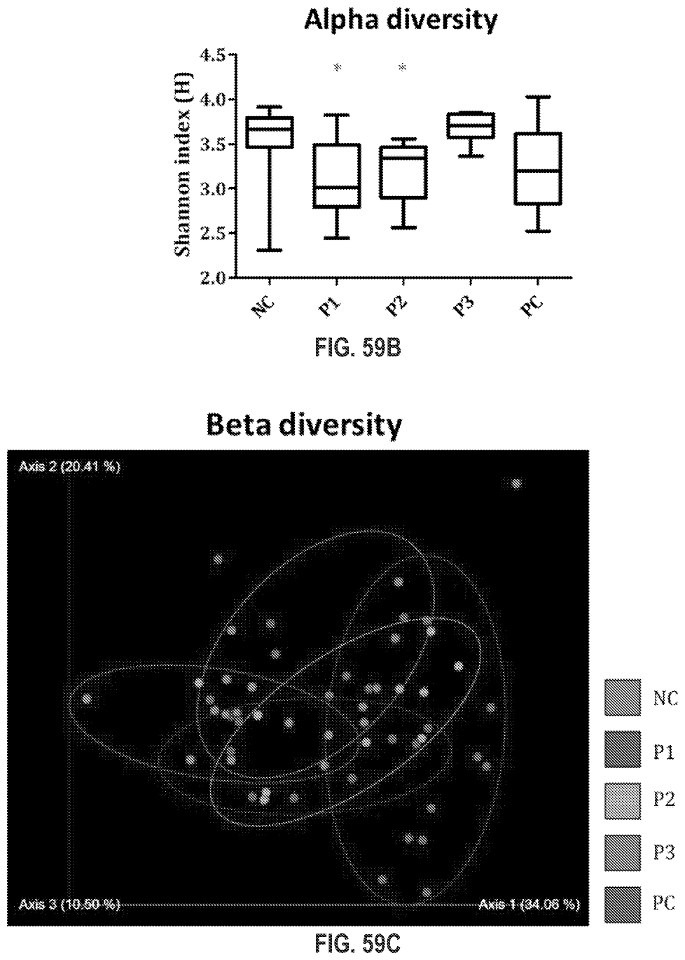

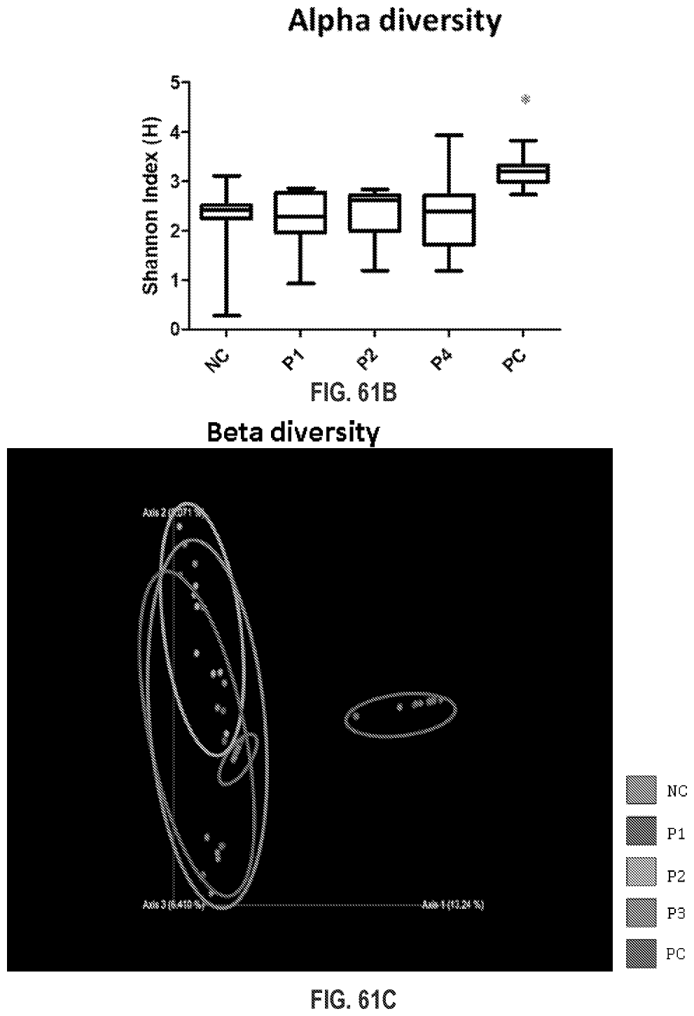

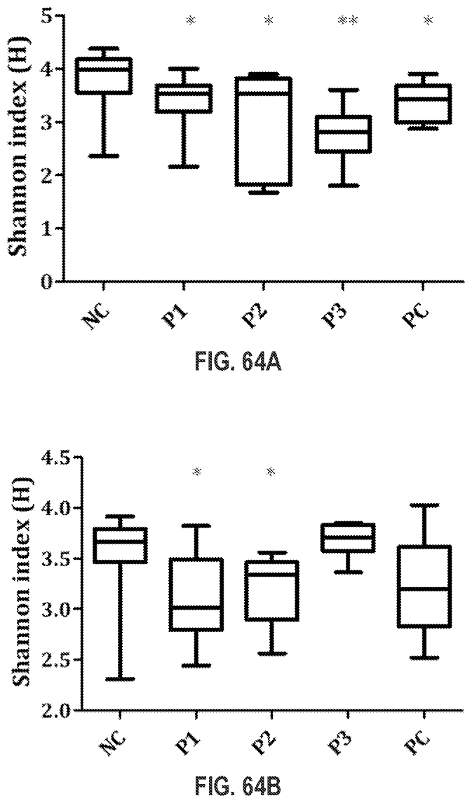

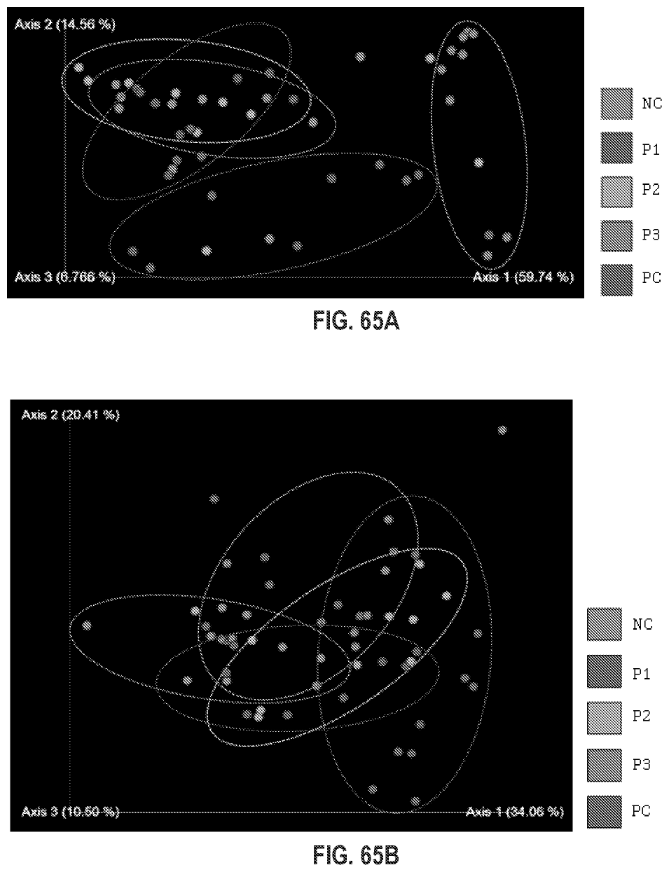

The analysis of alpha-diversity (Shannon index) revealed significant difference (P<0.05) in the microbial richness between the P1, P2 treated chickens and NC chickens, at both 100 mg/kg and 50 mg/kg doses (FIG. 64). However, significant difference (P<0.01) was only observed between P3 treated chickens and NC chickens at 50 mg/kg dose. Further, the beta-diversity (weighted unifrac) analysis showed that the microbial communities of P1 and P2 treated chickens were similar (P>0.05) to NC chickens when treated at 50 mg/kg dose; whereas microbial communities of P3 treated and PC chickens were significantly (P<0.01) different than NC chickens (FIG. 61). At 100 mg/kg dose, the microbial communities of P2, P3 treated, and PC chickens were significantly (P<0.01) different than NC chickens (FIG. 59). Overall, less impact on cecal microbiota was observed with P1 and P2 treatments as compared to P3 treatment.

### No Resistance was Observed in APEC Against Peptides In Vitro and In Vivo

The acquisition of resistance by APEC to peptides was evaluated in sub-lethal and lethal resistance assays. No change in the MICs of peptides was observed against APEC culture grown after 14 repeated passages in presence of sub-lethal concentration of peptides (0.75×MIC). Consistently, no resistant colonies were isolated when APEC culture was plated on LB agar containing lethal concentration (5×MIC) of peptides. Similarly, APEC colonies isolated from ceca and internal organs of chickens treated with peptides (from above) possessed the same MICs as above.

Peptides Bind with Higher Affinities to OmpC than OmpF

HPEPDOCK and PEP-SiteFinder tools were used to predict the affinity and binding sites of peptides with OmpC and OmpF proteins(51, 52). P1 binds with highest affinity (˜207.969 kcal/mol) to OmpC followed by P3 (˜168.294 kcal/mol) and P2 (˜135.46 kcal/mol). Further, the binding affinity of peptides to OmpF (P1: −197.146, P2: −129.838, and P3: −146.798 kcal/mol) is lower compared to OmpC. P1 binds with high propensity index (46-50) at W72, R174, Q61, and Q63 residues of OmpC C-chain and N46 residue of B-chain. P2 binds with high propensity index (30-33) at E66, Q61, Q59, and W72 residues of OmpC B-chain. P3 binds with high propensity index (34) at L20, Y22, Q33, Y35, R37, Q59, Q61, W72, R74, E10⁹, F110, G112, G113,N113, S117, Q124, and Q123 residues of OmpC B-chain and E66 residues of OmpC C-chain.

### Discussion

This study provides the use of LGG-derived small peptides (P1: NPSRQERR (SEQ ID NO:85), P2: PDENK (SEQ ID NO:86), and P3: VHTAPK (SEQ ID NO:87)) as new antibacterials against APEC infection in chickens (FIG. 56). To date, no small peptide-based (less than 10 amino acid residues) antibacterials have been evaluated specifically to treat bacterial infections in chickens. Several larger peptides (A3, P5, PABP, Cecropin A-D-Asn, Microcin J25, cLF36, cLFchimera and piscidin) have been evaluated and shown efficacy to promote growth performance, improve nutrient digestibility and gut morphology, enhance intestinal mucosal immune responses, and modulate gut microbiota in chickens. Although not targeted to any specific bacterial pathogen, it is reported that treatment with these peptides favors the proliferation of beneficial microorganisms such as Lactobacillus and Bifidobacterium and suppression of harmful microorganisms such as coliforms and Clostridium spp. Therefore, this data shows new avenues for development of small peptide-based antibacterials against bacterial infections in chickens.

Even though there are not many previous reports on treating bacterial infections in chickens using peptides, different peptides have shown efficacy against MDR bacterial infections in other animal models. A cyclic peptide ZY4 resists MDR P. aeruginosa and A. baumannii infections by decreasing susceptibility to lung infection by P. aeruginosa and suppressing dissemination of P. aeruginosa and A. baumannii to target organs in a mouse septicemia model. Similarly, arenicin-3, an amphipathic peptide, is effective in reducing bacterial (MDR E. coli and P. aeruginosa) burden in mouse models of peritonitis, pneumonia, and urinary tract infection. AMPR-11, a peptide derived from Romo1 (reactive oxygen species modulator 1), also increased the survival of mice, and decreased the bacterial load in murine model of sepsis with MDR bacteria, including methicillin-resistant S. aureus (MRSA) and carbapenem-resistant P. aeruginosa, K. pneumoniae and A. baumannii. More interestingly, SAAP-148, a synthetic peptide derived from LL-37, eradicated biofilm-associated infections with MRSA and MDR A. baumannii from wounded ex vivo human skin and murine skin in vivo. These studies show that peptides can be valuable antibacterial agents to control bacterial infections including antibiotic-resistant infections.

This study showed that that all three peptides affected the MlaA-OmpC/F system in APEC which impairs OM lipid asymmetry. The MlaA-OmpC/F system maintain OM lipid asymmetry by retrogradely transporting mislocalized phospholipid (PL) from the outer leaflet of the OM to inner leaflet or inner membrane (IM) and is regarded as a novel target for antibacterial drug development. It has been reported that MlaA interacts specifically with OmpC and OmpF and functions as a complex to maintain lipid asymmetry in the OM, which might be the reason behind peptides concurrently affecting the expression of mlaA, ompC and ompF genes in. A recent study identified peptide, arenicin-3, targeting mla operon (mlaABCDEF) in uropathogenic E. coli (UPEC; NCTC 13441, ST131) which thereby dysregulates PL transport and compromises the membrane integrity. Arenicin-3 also disrupts bacterial membrane with membrane debris surrounding the cells which is similar to what was observed in this study, thus supporting MlaA-OmpC/F system as the target of these peptides.

No resistance was observed in APEC against peptides which might be due to their MOA of targeting/disrupting bacterial OM. The antibacterial agents that target/disrupt the OM of Gram-negative bacteria can counter the resistance problem by overcoming antibiotic inactivation determinants, decreasing the development of spontaneous resistance, and impairing biofilm formation. Further, resistance is less likely to occur against OM-targeting antibacterial agents as they avoid OM barrier and action of efflux pumps that eliminate antibiotics from inside the bacterial cells, which are the most common mechanisms of antibiotic resistance. Therefore, antibacterial agents targeting OM are suitable for development as novel antibacterials in Gram-negative bacteria such as APEC.

All three peptides downregulated the expression of ompC and ompF genes as well as decreased the OmpC level in OM of APEC (FIG. 53). This showed that peptides not only inhibit the APEC growth but also reduce the APEC virulence. The role of ompC and ompF porins in APEC pathogenesis, including adhesion, invasion, colonization, and proliferation has been reported in mouse brain microvascular endothelial (BMEC) bEnd.3 cells, ducks, and mouse models. Further, the antibacterial agents that target the OM can act as antibiotic adjuvants; therefore, these peptides could also be used in combination therapy with conventional antibiotics to reduce the use of antibiotics and subsequent resistance.

Peptides (P1 and P2) treatments had minimal impact on cecal microbiota (FIG. 62), which is very significant because the conventional antibiotics cause profound changes in the intestinal microbiota, particularly diminish the abundance of beneficial commensals, and increase the abundance of potentially detrimental microorganisms, thus these peptides can be developed as safe antibacterials for use in chickens. The decreased abundance of Erysipelatoclostridium was observed in APEC infected chickens compared to non-infected chickens in this study. Bacteria belonging to Erysipelotrichaceae are reported as performance enhancers in broiler chickens; therefore, the exposure of chickens to pathogens such as APEC can reduce the productivity by altering the Erysipelotrichaceae population in gut of chickens. Further, similar to decreased Sellimonas abundance observed in APEC infected chickens in this study, the abundance of Sellimonas was also decreased in Salmonella typhimurium infected hens, showing a role for Sellimonas in resisting APEC infection. The abundance of Flavonifractor, which is reported to provide colonization resistance against S. enteritidis, was also decreased in APEC infected chickens in this study. These findings show that genera belonging to Erysipelatoclostridium, Sellimonas, and Flavonifractor could be used as gut microbial markers to monitor APEC infection in chickens

Interestingly, peptide P2 increased the abundance of lactic acid bacteria Pediococcus and Weissella which is the reason behind better anti-APEC activity compared to other peptides. Bacteria belonging to Pediococcus are regarded as good probiotics to chickens. Similarly, Weissella has been also used as probiotic in chickens. The greater reduction in APEC load in cecum of chickens was observed when chickens were treated with increased dose of P1 and P2. This is due to large variability in the gut microbiota profile of P3 treated chickens at 50 mg/kg and 100 mg/kg doses. Gut microbiota can act as an invisible organ to modulate the functions of drugs by affecting drug absorption, toxicity, metabolism, and bioavailability.

In summary, this study identified peptides (P1: NPSRQERR (SEQ ID NO:85), P2: PDENK (SEQ ID NO:86), and P3: VHTAPK (SEQ ID NO:87)) having an effect on APEC in vitro, in vivo in wax moth larva model, and in commercial broiler chickens with minimal or no toxicity. These peptides therefore are used as novel antibacterial agents against APEC as an alternative to antibiotics. Further, this study revealed a new druggable target, MlaA-OmpC/F system in APEC which can facilitate further drug development against APEC and related pathogens such as human ExPECs and other pathogenic E. coli including antibiotic-resistant strains. Moreover, effective control of APEC infections in chickens can help in curbing poultry originated ExPEC infections in humans.

### Example 4. Small Antimicrobial Peptides Reduce Salmonella in Chickens

Salmonella is the most frequently reported bacterial cause of foodborne illness in the US. S. Enteritidis and S. typhimurium are consistently among the top serovars isolated in human infection, making them serovars of public health concern. The Centers for Disease Control and Prevention estimate that about 1.35 million infections, 26,500 hospitalizations, and 420 deaths are caused by Salmonella annually in the US. The annual cost of medical treatment for Salmonella-related foodborne illnesses in the US is estimated to be $3.7 billion. Poultry products (eggs and poultry meat) have been considered the main vehicles of Salmonella infections in humans. Poultry, particularly chickens and turkeys, are frequently colonized with Salmonella without detectable symptoms (sub-clinical carriers/healthy carriers) through horizontal and vertical transmission at the primary production level. This phenomenon of Salmonella being present in visually healthy poultry is the preeminent human transmission risk factor; allowing bacteria to easily transmit from eggs and poultry to humans. Controlling Salmonella is difficult because it is environmentally persistent, easily adaptable, widely distributed, and increasing in antibiotic resistance.

Previously, antibiotics have been widely used to control Salmonella infections. However, in 2012 the FDA recommended the limited use of antibiotics in food-producing animals, and then later included the phasing out of antibiotics in the production use for prevention and growth in food animals (FDA Guidance for Industry #209, #213). Despite the need to reduce the overuse of antibiotics to control antibiotic resistant bacteria, the reduction in antimicrobials can subsequently lead to an increase in foodborne pathogens on poultry and in poultry products. Therefore, there is a dire need for developing and implementing effective alternative strategies to reduce Salmonella prevalence, minimize human infection, and simultaneously promote proper antibiotic stewardship.

Antimicrobial peptides (AMP) are alternatives to current antibiotics. The ability of AMPs to inhibit the emergence of antibiotic resistant bacteria has been deemed an advantage over antibiotics. In addition to low propensity for resistance development, the of AMPs as novel therapeutics is supported by the relatively small size and fast and selective antibacterial action. The confidence that AMPs as better at avoiding resistance is rooted in the mechanism of action. AMPs involve multiple low affinity targets, which complicates bacteria's ability to defend against a singular resistance mechanism. Conventional antibiotics use a high affinity target which allows bacteria to quickly defend and display resistance. AMPs antibacterial activity involves disrupting the bacterial cell membrane, degrading the cell walls, and/or acting on intracellular targets following translocation into the bacteria's cytoplasm. Membrane depolarization, creation of pores allowing cellular contents to leak, and alteration of the lipid bilayer could all be actions caused by AMPs leading to disruption in bacterial membrane functions. Unlike many current antibiotics that elicit bacterial stress pathways such as RpoS, AMPs do not do it and is thus better at avoiding bacterial mutations. More importantly, AMPs are not a threat to mammalian cells because of the functional difference between microbial and mammalian membranes. AMPs carry additional advantages in addition to the broad-spectrum antibacterial properties. Some AMPs display antiviral, antifungal, and anti-parasitic properties. AMPs also possess immunomodulatory properties, which can aid in protection and simultaneously enhance animal health and performance. Small or short peptides are (typically <18 residues) easier to synthesize making them a more feasible option to be adopted as therapies in comparison to other peptides. For the purpose of this study short or small peptides consist of 10 or less amino acid residues.

The objective of this study was to test the efficacy of Lactobacillus rhamnosus GG (LGG) derived small peptides (P1-NPSRQERR (SEQ ID NO:85), P2-PDENK (SEQ ID NO:86), P3-VHTAPK (SEQ ID NO:87), P4-MLNERVK (SEQ ID NO:88), P5-YTRGLPM (SEQ ID NO:118), and P6-GKLSNK (SEQ ID NO:119)) against Salmonella enterica subsp. enterica serovar Typhimurium LT2 (ST), in chickens and to subsequently test the ability to offer cross protection against Salmonella enterica subsp. enterica serovar Enteritidis (SE) and other serovars in vitro. Since Salmonella is a pathogen of food safety concern, the ability of peptides to retain inhibition qualities when heated or treated with protease was additionally tested according to poultry industry standards. Most importantly, it was sought to identify which small peptides would be best at retaining antibacterial capabilities in chickens and evaluate the role, if any, peptides have on the cecum microbial community of chickens.

### Results

### Peptides Display Anti-Salmonella Activity

Peptides (P1, P2, P3, P4, P5, P6) were added to ST at 12 mM concentration for 12 hours at 37° C. The initial screening showed the four best peptides at inhibiting ST growth was P1, P2, P3, and P4 (FIG. 67). At 12 mM, P2 inhibited ST by 75%, P1 by 55%, P4 by 40%, and P3 by 33% (FIG. 67).

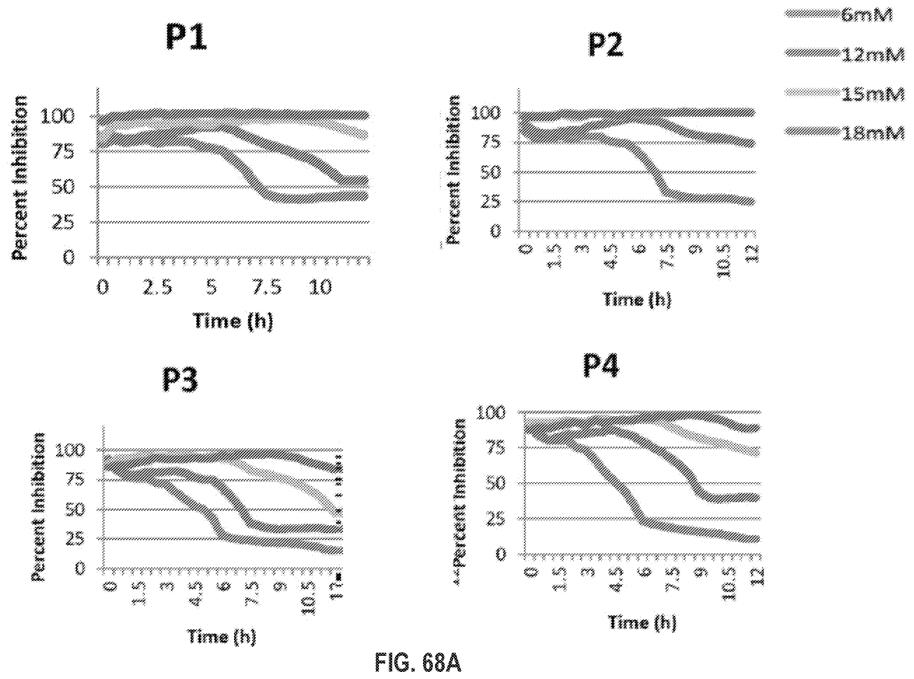

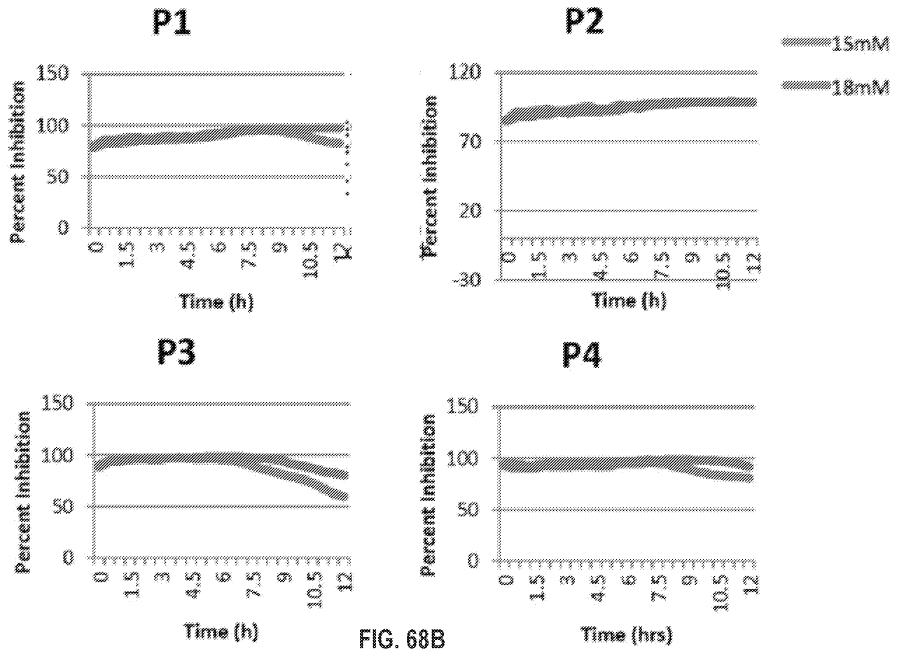

To further determine the minimum inhibitory concentration of peptides, a dose response analysis was conducted using peptides P1, P2, P3, and P4 at 6 mM, 12 mM, 15 mM, and 18 mM. P1 and P2 completely inhibited the growth of ST at 18 mM and 15 mM respectively (FIG. 68A). Neither P3 nor P4 completely inhibited ST; however, P4 resulted in ˜90 percent inhibition (FIG. 68A). Further, the peptides were also tested against SE at 15 mM and 18 mM. P1 and P2 showed 100 percent inhibition at 18 mM and 15 mM respectively (FIG. 68B). P4 resulted in 95% inhibition of SE growth at 18 mM. P3, similar to ST results, did not inhibit more than 85% of SE growth and thus was not chosen for further analyses (FIG. 68B). Based on these results, the MICs of P1, P2, and P4 were determined to be 18 mM, 15 mM, and 18 mM, respectively (FIG. 68B).

Peptides Retain their Inhibitory Characteristics Against Other Serovars

P1, P2, and P4 inhibition against public health relevant serovars ST and SE, led to testing the spectrum of activity at the previously determined MICs against other non-typhoidal Salmonella serovars that are commonly implicated in foodborne illnesses. Table 23 shows that all serotypes tested (S. Anatum, S. Albany, S. Brenderup, S. Javiana, S. Heidelberg, S. Muenchen, S. Newport, S. Saintpaul) were inhibited completely by P1, P2, and P4 at the respective MICs. This supports the idea that the peptides have broad spectral inhibitory activity against multiple Salmonella serovars from varied sources.

### Peptides are Heat and Protease Resistant and Inhibit Biofilm Protected Salmonella

P1, P2, and P4 were exposed to 86° C. for 6 minutes or treated with protease (200 μg/mL at 50° C. for 30 minutes) to determine if either one had an effect on the anti-Salmonella ability of the peptides. Neither heat nor the protease treatment had any effect on the MIC of the peptides (Table 24.)

Additionally, P1, P2, and P4 retained inhibitory characteristics against biofilm protected Salmonella in a Innovotech's MBEC Assay® (Innovotech Inc. AB, Canada). Specifically, all viable biofilm protected Salmonella (both ST and SE) was eradicated in the wells treated with peptides at their MICs (Table 25). In the MBEC assay, the untreated wells yielded 6.46±0.09 log CFU/mL biofilm protected ST (Table 25). Similarly, untreated wells yielded 6.77±0.02 log CFU of biofilm promoted SE (Table 25).

Peptides have No Effect on Gram-Positive Commensal Bacteria

P1, P2, and P4 were tested at the respective MICs against both Gram-positive and Gram-negative commensal bacteria to further understand the inhibitory characteristics. No growth inhibition was observed against any Gram-positive commensal bacteria (Enterococcus faecalis, Streptococcus bovis LGG, Lactobacillus acidophilus, Lactobacillus brevis, Bifidobacterium lactis Bb12, Bifidobacterium longum, and Bifidobacterium adolescentis) when treated with any of the peptides. Similarly, the peptides showed no inhibition on the growth of Gram-negative bacteria Bacteroides thetaiotaomicron. However, E. coli Nissle 1917 and E. coli G58-1 were inhibited, showing that P1, P2, and P4 retained inhibition characteristics against the related Gram-negative E. coli bacteria tested.

### Peptides Reduce ST Colonization in Chicken Cecum

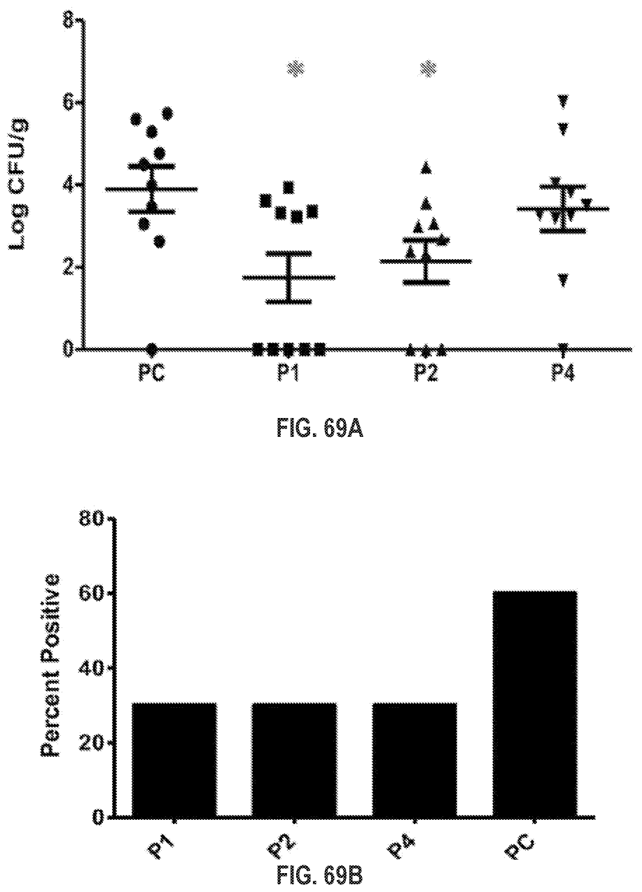

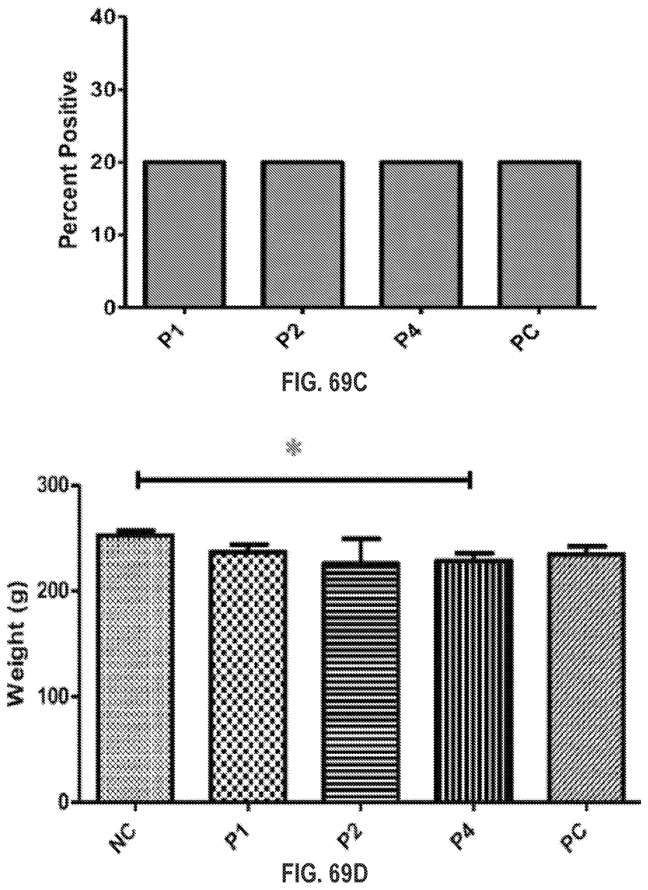

One-day-old SPF layers were treated with P1, P2, or P4 with 50 mg/kg twice daily for 7 days to test their efficacy against Salmonella infection. Since, both ST and SE showed consistent and similar results in vitro, as a proof of concept only ST was used in the chicken experiment. At day 10 (7 days post ST infection), P1-NPSRQERR (SEQ ID NO:85) and P2-PDENK (SEQ ID NO:86) significantly reduced the colonization of ST in the cecum (FIG. 69A) by 2.2 logs and 1.8 logs respectively (P<0.05; Mann Whitney test). Additionally, all peptides reduced the percent of birds positive for ST in the liver by 30 percent (FIG. 69B). There was not consistent ST colonization in the spleen, so there was no observed difference between peptide treated groups and untreated (PC) group (FIG. 69C). Furthermore, treatment with P1 or P2 did not affect the body weights of chickens (FIG. 69D).

### Peptides do not Affect Cecal Microbial Richness and Diversity

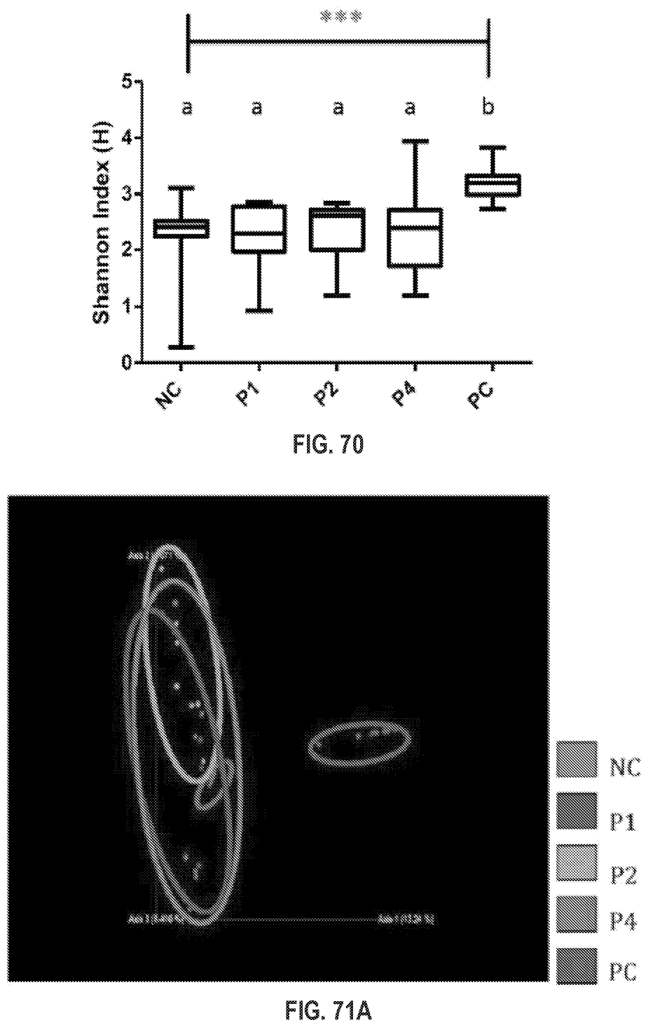

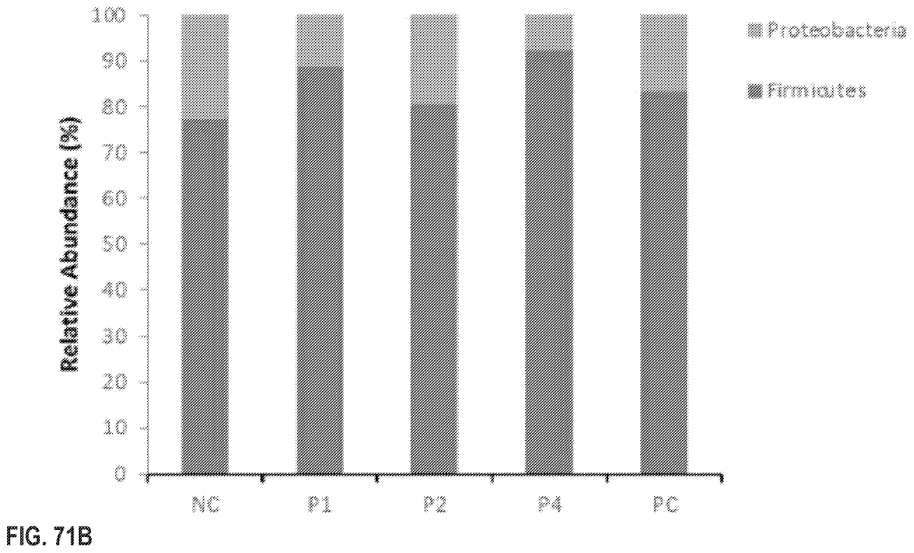

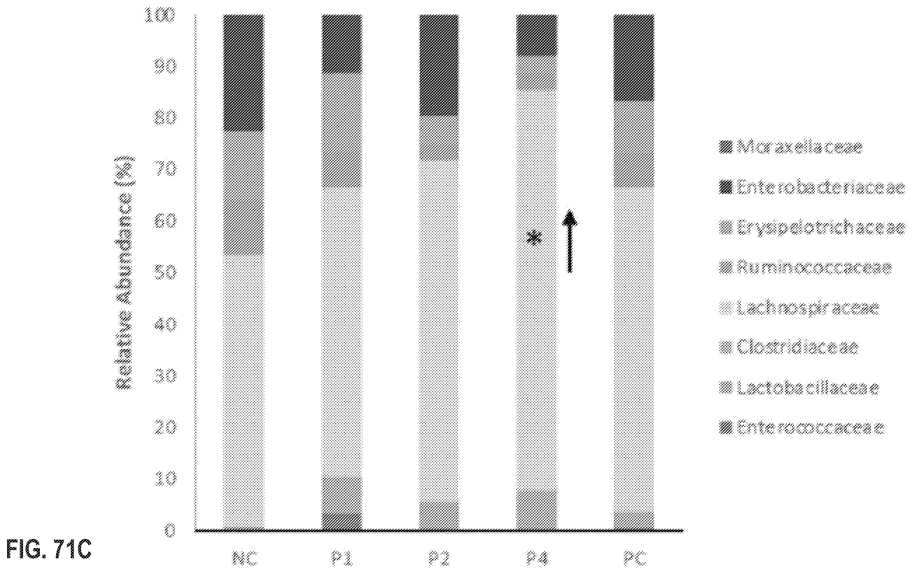

Analysis of alpha diversity was done using Shannon index to determine the difference in microbial richness and diversity. Peptide treated birds showed no significant difference in comparison to the non-treated, non-infected control (NC) (FIG. 70). However, NC and all peptide treated groups showed significant differences in microbial richness and diversity compared to the Salmonella infected positive control (PC; infected but untreated) group (P<0.001) (FIG. 70). Similarly, all peptide treated groups and NC group showed significant differences when compared to PC group in beta diversity (P<0.001) (FIG. 71A).

At the phylum level, cecal microbiota was primarily composed of Proteobacteria and Firmicutes. There were no significant differences in relevant abundance among the groups at the Phylum level (FIG. 71). Similarly, P1, P2 nor PC showed any significant differences in relative abundance at the family level. However, P4 did significantly increase the relative abundance of Lachnospiraceae from 53% to 78% in comparison to the negative control (FIG. 71C). At the genus level P4 (11.71%) and PC (4.91%) significantly increased the Lachnospiraceae (uncultured) in comparison to NC (0.37%) (Table 29). Large standard error (>4) in P1 (8.62%, P=0.057) prevented it from being significant. P1 (4.80%) and P4 (3.16%) reduced the percentage of pathogenic Escherichia-Shigella when compared to PC (11.61%).

### Glutamic Acid and Aspartate are Critical in Salmonella Inhibition

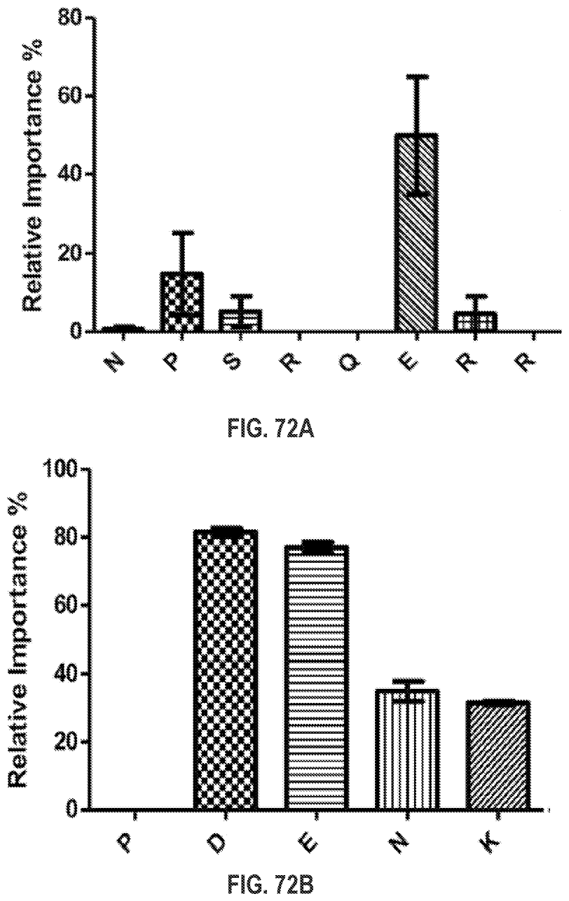

Following the success in chickens, P1 (NPSRQERR (SEQ ID NO:85)) and P2 (PDENK (SEQ ID NO:86)) were used to determine the important amino acid residues necessary for anti-Salmonella activity. The complete list of alanine substituted amino acid with its correlating sequence and peptide is listed in Table 28 (SEQ ID NO: 105-117). In P1, glutamic acid was the most essential amino acid with a mean relative importance of 50% (FIG. 72A). Proline (15%) was the next amino acid in ranking order of importance, however like the remaining residues, it displayed a relative importance of less than 20 percent. This shows that in P1 the ranking of amino acids is E>P>N═S (FIG. 72A). For P2, there was more of a distinct ranking of amino acids. Aspartate was the most essential amino acid with a relative importance of 82%, immediately following glutamic acid with 77% (FIG. 72B). Glutamic acid (E) showed high importance in both peptides, demonstrating that it may be essential in Salmonella inhibition. Asparagine was the third most important residue with 35% relative importance followed by lysine with 32% (FIG. 72B). This information demonstrates that the relevant importance of residues in P2 are Aspartate (D)>Glutamic acid (E)>Asparagine (N)>Lysine (K). Proline was the only amino acid residue in P2 to display zero relative importance demonstrating it is non-essential in anti-Salmonella activity (FIG. 72B). The alanine scanning analyses were only done with ST, both ST and SE consistently showed the same growth and inhibitory characteristics showing the same amino acids would be relevant for SE.

### Lysine Substitution Improved Anti-Salmonella Activity of P2

The arginine (R) or lysine (K) substitutions were made in each peptide to enhance the anti-Salmonella activity. Arginine showed inconsistent results against Salmonella when substituted in place of other amino acid residues (SEQ ID NO: 89-104) (Table 26). Particularly, when arginine replaced the non-essential amino acid serine, it improved the efficacy of P1 against ST, lowering MIC from 18 mM to 15 mM (Table 26). This is in contrast to the observed change in MIC when arginine replaced glutamine (from 18 mM to >18 mM). No arginine substitution improved the MIC of the P1 against SE (Table 26). However, lysine improved the efficacy of P2 against ST and SE when it was used to replace the non-essential amino acid, proline (Table 26). In both instances, the MIC improved from 15 mM to 12 mM (Table 26). Replacing asparagine with Lysine did not alter a change in MIC (Table 26).

### Microscopy Images Show Peptides Disrupt Salmonella Membrane

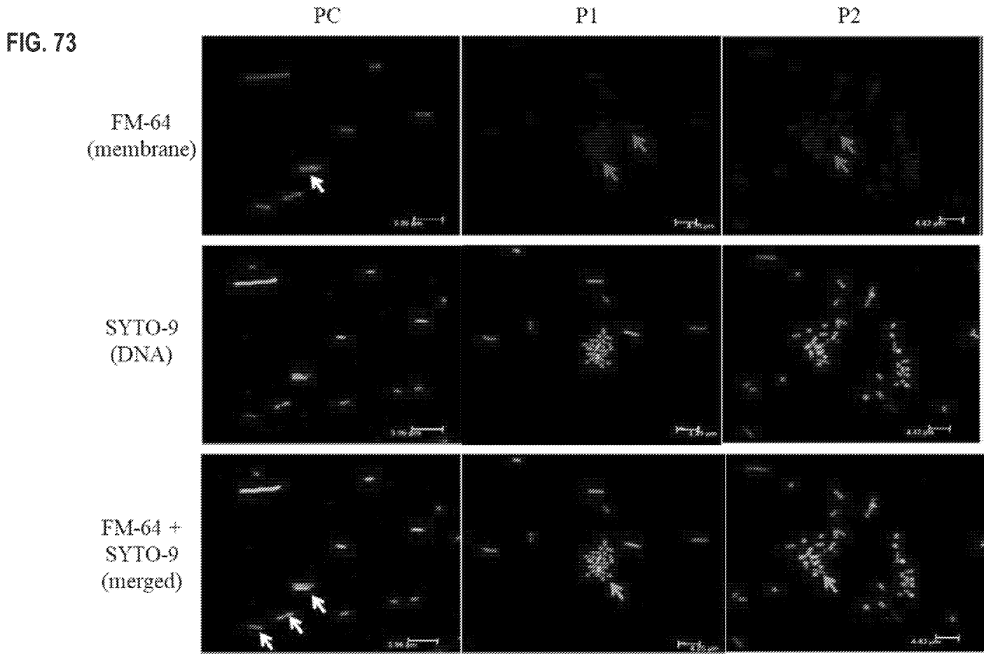

To better understand the mode of action of peptides P1 and P2, bacterial cytological profiling was done using confocal microscopy. Only P1 and P2 were included for microscopic studies because of their increased efficacy both in vitro and in vivo. The red FM4-64 membrane stain revealed that the untreated ST (PC) had a defined membrane enclosing bacterial contents (FIG. 73). There was little to no red stain present in the P1 and P2 treated bacteria. Additionally, when FM6-64+SYTO-9 (Green colored DNA stain) stains were merged, the untreated ST shows DNA content surrounded by a defined red membrane, which is absent in the peptide treated bacteria (FIG. 73).

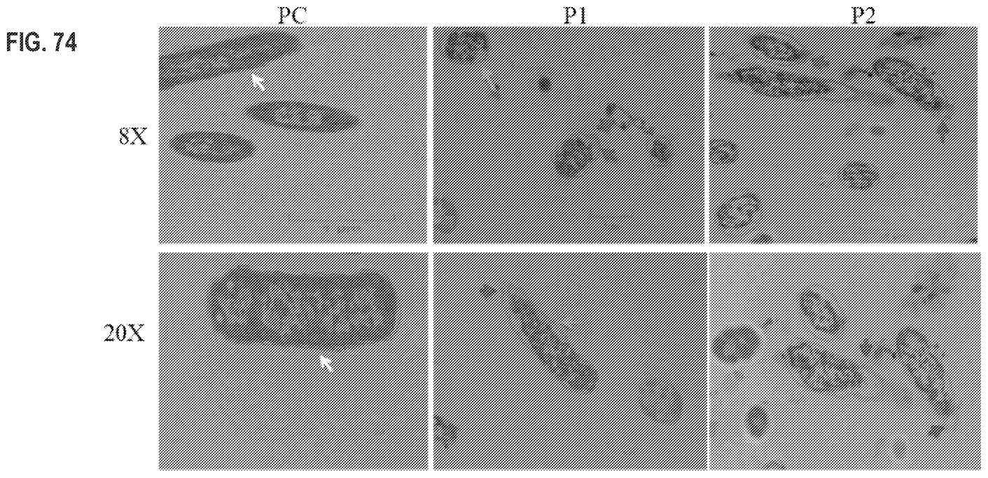

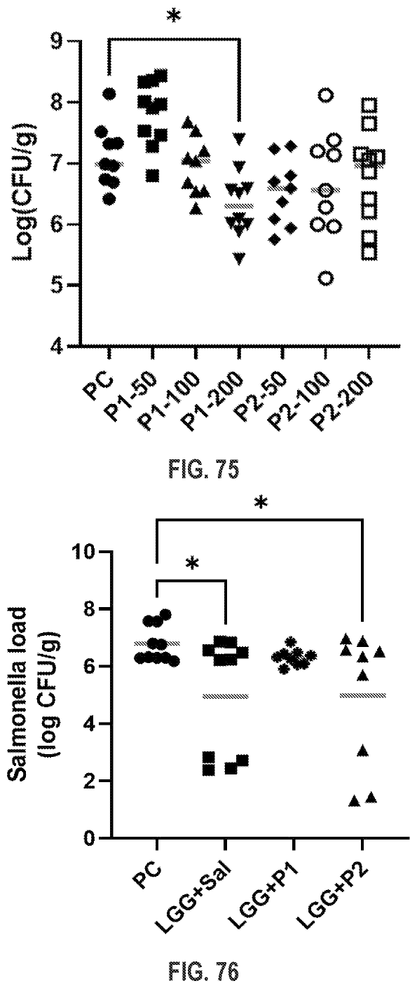

The TEM images further confirmed the results from confocal images. The P1 and P2 treated samples show a disruption of the membrane characterized by sloughed or flaccid appearance of the membrane (FIG. 74). In sharp contrast, untreated ST (PC) shows an intact bacterial cell membrane (FIG. 74).

### Discussion

Salmonella remains a significant food safety and public health issue. This can be partly attributed to the increasing antibiotic resistance observed. There has not been much progress with introducing new and clinically relevant classes of antibiotics that are effective against Gram-negative bacteria without having issues with rapid resistance development. The outer membrane of Gram-negative bacteria has added to the difficulty in discovering new and effective antimicrobials. The tight packing of lipopolysaccharides and negative charge helps Gram negative bacteria evade most hydrophobic molecules. This also demonstrates that targeting the cell membrane of Gram-negative bacteria can aid in inhibition; however, there are not many antibiotics that can successfully do it, and the ones that are approved have very narrow index and report other deleterious effects. Previously, studies have shown that probiotics and their derived peptides possess anti-Salmonella capabilities. Moreover, previous studies confirmed that the small peptides derived from LGG are major contributors to this inhibitory effect. In this study, the efficacy of LGG-derived small peptides (P1-NPSRQERR (SEQ ID NO:85), P2-PDENK (SEQ ID NO:86), P4-MLNERVK (SEQ ID NO:88)) was tested against Salmonella in chickens and whether they can offer antagonistic abilities against multiple serovars in vitro.

This study shows that small peptides (<10 amino acid residues), a phenomenon not thoroughly studied in literature, have antagonistic effect against Salmonella. There are, however, studies evaluating larger peptides and their role against Gram-negative bacteria. Particularly, peptides Alyserin (identified in secretions form frog skin) and A3APO (from synthetic peptide library) were tested against both E. coli and Salmonella and showed inhibition but did not negatively affect L. lactis. This is similar to what was observed, peptides in this study were inhibitory against Gram-negative bacteria (including commensal E. coli) but had no effect on Gram-positive beneficial bacteria. This antagonist effect was heat and protease resistant, necessary characteristics for use in commercial feed settings.

Antimicrobial peptides have also been shown to have antagonistic effects against pathogenic bacteria in murine models. In one single treatment, the antimicrobial peptide SAAP-148 peptide was effective as a topical ointment against methicillin-resistant Staphylococcus aureus (MRSA) and multiple drug resistant A. baumannii in mice and ex vivo human skin. Similarly, AA139 reduced bacteria load by multiple logs in murine models challenged with P. aeruginosa and multiple drug resistant E. coli. Although, there are not extensive studies analyzing the inhibitory effects of antimicrobial peptides against Gram-negative bacteria in chickens, some studies describe analyzing the role of peptides on broiler growth and performance. Collectively, the preliminary analyses in chicken models and bacterial inhibition observed in murine models show that antimicrobial peptides are considerable options for implementation in chickens. This study confirms P1 and P2 significantly reduced the amount of ST in chickens by 2.2 and 1.8 logs respectively (FIG. 69A). Additionally, P1, P2, and P4 reduced the colonization of ST in the liver compared to the untreated positive control (FIG. 69B). These peptides were tested at 50 mg/kg, testing at higher doses may show improved efficacy.

Consistent with the success of the SAAP-148 against biofilm bacteria, P1, P2, and P4 completely eliminated biofilm protected SE and ST (Table 24). These studies collectively demonstrate antimicrobial peptides to control antibiotic resistant Gram-negative pathogens. Since, antimicrobial resistance is important, the ability of Salmonella to develop resistance against lethal and sub-lethal treatment of peptides was test and no resistance was observed. The mode of action of the peptides disrupting the bacterial outer membrane is the reason for the lack of resistance (FIGS. 73 and 74). These results support previous studies that show resistance is less likely to occur when the mode of action is outer membrane targeting. Studies investigating the synergistic effect of outer membrane targeting peptides with antibiotics can help increase efficacy and reduce resistance to those antibiotics.

Peptides (P1, P2, and P4) had no effect on the richness and evenness of chicken cecal microbiota. However, the untreated, Salmonella infected chickens did affect the alpha diversity of the microbiota. It is significant and worth noting that the short-term treatment with the peptides in this study caused no deleterious effect on microbiota differing from antibiotics that have been associated with immune dysfunction and the development of autoimmune disease. Antibiotic treatments have also been linked to increasing susceptibility to intestinal pathogens.

### Conclusion

LGG-derived peptides (P1-NPSRQERR (SEQ ID NO:85), P2-PDENK (SEQ ID NO:86), and P4-MLNERVK (SEQ ID NO:88)) significantly inhibited ST, SE, and other Salmonella serovars in vitro. Furthermore, the antimicrobial properties of the peptides were unaffected by heat and protease treatment, showing it is possible to incorporate them in commercial feed. More importantly, ST did not develop resistance to the peptides in in vitro resistance assays. P1 was able to successfully reduce the colonization of ST by 2.2 logs ad P2 successfully reduced colonization by 1.8 logs in SPF layers 7 days post infection. All peptides (P1, P2, and P4) reduced birds colonized in liver with ST by 30 percent. Glutamic acid and aspartate are critical amino acids in anti-Salmonella activity. Overall, these results show that small peptides are used for controlling Salmonella infection in chickens and thus improving food safety and public health.

### Materials and Methods

Bacterial strains and growth conditions: The bacterial strains, their growth conditions, and sources are detailed in the Table 27. Salmonella enterica subsp. enterica serovar Typhimurium LT2 (ST) was the primary strain used for determining the antibacterial properties of the antimicrobial peptides. Salmonella enterica subsp. enterica serovar Enteritidis (SE) was used as a secondary strain in vitro to confirm the antibacterial activity. The additional, food safety relevant, serovars were used to confirm the cross-protection capabilities of the peptides.

Peptide synthesis: LGG-derived peptides (P1-NPSRQERR (SEQ ID NO:85), P2-PDENK (SEQ ID NO:86), P3-VHTAPK (SEQ ID NO:87), P4-MLNERVK (SEQ ID NO:88), P5-YTRGLPM (SEQ ID NO:118), P6-GKLSNK (SEQ ID NO:119)) were used because previous studies demonstrated LGG's ability to inhibit Salmonella serovars (Closs et al unpublished). All 6 peptides were synthesized by GenScript (NJ, USA) with >95% purity, dissolved in 100% dimethyl sulfoxide (DMSO) and stored at −80° C. until experiments were conducted.

Primary screening for anti-Salmonella activity of peptides: The inhibitory effect of the peptides was accessed as described previously. ST was grown overnight and adjusted to 5×10⁵ CFU/mL; 100 microliters of this measured concentration was placed into the wells of a 96 well plate. Peptides 1-6 were added to the well at 12 mM concentration and plate was incubated in a TECAN Sunrise™ absorbance microplate reader at 37° C. with kinetic absorbance measurements taken every 30 minutes for 12 h. The growth inhibitory activity was calculated with the formula (OD₆₀₀ DMSO treated well-OD₆₀₀ peptide treated well)/OD₆₀₀ DMSO treated well×100%. DMSO treated, Salmonella challenged wells and sterile media with no Salmonella challenge were used as controls. This experiment was done twice to ensure the accuracy and reproducibility of the results.

Determination of minimum inhibitory concentration using dose response analysis: To determine the minimum inhibitory concentration (MIC) of P1, P2, P3, and P4 (selected based on results of primary screening), the methodology of the assay above was used but the peptides were added at 6 mM, 12 mM, 15 mM, and 18 mM. MIC was determined by selecting peptide concentration at which there is no increased growth (no increase in OD), given indication of no ST growth. An independent repeat experiment was conducted. Additionally, to confirm the MIC, and test the efficacy of peptides against another serovar, the experiment was repeated (with two independent experiments) using SE, with peptides added at 15 mM and 18 mM. Following incubation samples were plated to prove bactericidal effect.

Peptide protection against varied Salmonella serovars: To further test the spectrum of the peptides anti-Salmonella capabilities, eight additional Salmonella serovars commonly implicated in foodborne illness (S. Anatum, S. Albany, S. Brenderup, S. Javiana, S. Heidelberg, S. Muenchen, S. Newport, S. Saintpaul) were analyzed. P1, P2, and P4 (selected based on dose response assay results) were added to the assay at their MICs to determine their effect on different serovars. Inhibition was determined using criteria from above.

Heat and protease tolerance of peptides: P1, P2, and P4 were heated for 6 minutes at 86° C. or treated with protease proteinase K, 200 μg/mL at 50° C. for 30 minutes. After which, the treated and/or heated peptides at their respective MICs were evaluated in the inhibition assay with Salmonella. The protocol from above was repeated in the Tecan with the same controls.

Peptides effect against biofilm embedded Salmonella: To analyze the ability of the peptides (P1, P2, and P4) to retain their inhibitory characteristics against biofilm protected Salmonella, Innovotech's MBEC Assay® (Innovotech Inc., AB, Canada) was conducted as previously detailed. Initially, 150 μL of 5×10⁷ CFU/mL adjusted ST or SE suspension in LB media was aliquoted into the wells of MBEC inoculator plates containing polystyrene pegs and incubated for 36 h on a rocker platform at 37° C. to allow the biofilm formation. After biofilm formation, the pegs were washed to remove loosely adherent planktonic bacteria and transferred to a new 96-well plate containing peptides at MICs, with relevant controls, in 200 μL 25% LB media. The diluted LB media was used because it allows for slow bacterial growth to promote biofilm formation due to limited nutrients. The plate was incubated for 18 h at 37° C. with rotation at 150 rpm. Sterile media and DMSO were used as controls. Following incubation, the MICs of peptides in challenge plate were recorded. The pegs were transferred to new 96-well plate containing PBS and sonicated for 30 minutes to disrupt the biofilm. The sonicated suspensions were then ten-fold serially diluted and plated on LB agar plates to enumerate the biofilm protected bacteria and determine the minimum biofilm eradication concentration (MBEC) of peptides. Three independent experiments were conducted to ensure accuracy.

Peptide activity against commensal bacteria: Peptides 1, 2, and 4 were used to assess if they would inhibit commensal microbes. The commensal bacteria used along with their culture requirements are described in Table 25. Peptides at their MICs were added to 100 μL of known concentration of commensal bacteria and incubated under their required conditions. OD₆₀₀ of cultures was measured to assess the effect of peptides on bacterial growth.

Efficacy of peptides in chickens: To assess the efficacy of peptides against ST in chickens, one-day-old specific pathogen free (SPF) layer chickens from a Salmonella free flock at The Ohio State University were obtained and provided with feed and water ad libitum. Chickens (n=10/group) were treated with P1, P2, or P4 at 50 mg/kg body weight twice-daily doses. Peptides were dissolved in water to reach final concentration of 50 mg/kg and orally administered from day 1 to day 7. Early intestinal colonization is important for poultry health and thus is why peptide treatment was begun at day 1. Birds were challenged with 10⁴ CFU of nalidixic acid resistant ST on day 3. At day 10 [7 days post infection (dpi)] chickens were euthanized, and the cecum, liver, and spleen were collected to determine the peptide's ability to reduce ST infection. The cecum was suspended in PBS, homogenized, ten-fold serially diluted, and plated on XLT4 agar plates supplemented with 50 μg/ml nalidixic acid and incubated for 24 h at 37° C. The liver and spleen were individually suspended in PBS and the undiluted homogenized suspension was plated directly and incubated for 24 h at 37° C. One milliliter of the undiluted homogenized liver and spleen was enriched in 9 ml of tetrathionate broth (TTB) for 24 h at 37° C. After incubation they were plated as described above to enumerate ST in each tissue. The body weights of chickens were measured on the day of necropsy (day 10). An untreated positive control group (PC: infected but untreated) and a negative control group (NC: non infected and non-treated) were included in the study. A Mann-Whitney test was used to determine statistical significance (P<0.05) of treatment on ST load in cecum and on body weight.

Cecal microbiome analysis: To evaluate the role P1, P2, and P4 (50 mg/kg) treatment on the cecal microbiome of chickens, 16S rRNA based metagenomic study was conducted as previously described. DNA was extracted from approximately 0.2 g of cecal content using PureLink™ Microbiome DNA Purification Kit (Thermofisher Scientific). After which, RNA was removed with the treatment of RNase A (2 μL of 100 mg/mL solution per sample: Qiagen). NanoDrop 2000c Spectrophotometer (ThermoFisher Scientific) was used to check the quality and measure the quantity of the DNA. The extracted DNA was used in 16S rRNA V4-V5 sequencing done at MCIC (mcic.osu.edu/genomics/illumina-sequencing). Amplicon libraries were prepared using IFU KAPA HiFi HotStart ReadyMixPCR Kit (Roche). PCR products were cleaned using Agencourt AMPure XP beads (BECKMAN COULTER Life Sciences). Nextera XT DNA Library Preparation Kit (Illumina) was used to generate Illumina library and sequencing done with Illumina MiSeq platform generating paired end 300-bp reads. QIIME 2 (Quantitative Insights Into Microbial Ecology) bioinformatics platform (qiime2.org/) was used to conduct metagenomic analysis. Quality control of the raw reads was performed using FastQC 0.11.8 (Babraham Bioinformatics). Trimmomatic-0.33 was used to trim the adaptor and other Illumina-specific sequences (www.usadellab.org/cms/?page=trimmomatic). After which trimmed sequences (fastq.gz) were then imported into QIIME 2 as a manifest file format (PairedEndManifestPhred33V2). DADA2 was then used for the feature table construction and additional filtering of sequences. Taxonomic analysis was performed using Naive Bayes classifiers trained on the Silva 132 99% OTUs (silva-132-99-nb-classifier.qza) database. Phylogenetic diversity was analyzed using align-to-tree-mafft-fasttree pipeline. Additionally, Shannon's diversity index (alpha diversity) and Bray-Curtis distance (beta diversity) were analyzed using core-metrics-phylogenetic pipeline (docs.qiime2.org/2019.7/tutorials/moving-pictures/). Alpha and beta diversity statistical significance were analyzed using Kruskal-Wallis and PERMANOVA tests (P<0.05), respectively. The statistical difference (P<0.05) in the taxonomic composition between the groups was determined using Mann Whitney Test.

Peptide resistance studies: To assess Salmonella's ability to develop resistance to P1 and P2, lethal and sublethal resistance assays were performed as described previously. For lethal resistance assay, approximately 10⁸ CFU of ST was plated on LB agar mixed with 5×MIC of P1 and P2 peptides and incubated for 5 days at 37° C. Sterile plates and DMSO treated Salmonella plates were used as controls. For sublethal resistance assay peptides (P1 and P2) were added at subinhibitory (0.75×MIC) concentrations to 100 μL of ST suspension (˜5×10⁵ CFU/ml) in LB medium in 1.5-ml microcentrifuge tubes. The tubes were incubated at 37° C. with shaking at 125 rpm for 24 h. After the incubation, 20 μL of grown ST culture was mixed with 80 ml of fresh LB medium, and peptides were added again at 0.75×MICs. This procedure was repeated 13 times. Following the 14th passage, the MIC of peptides was determined against ST cultures grown from the 14th passage as described above. The tubes containing sterile LB medium and DMSO-treated ST suspension were used as controls. The lethal and sublethal resistance assays were done in duplicate.

Structural-activity relationship (SAR) study: Structure activity relationship analysis was used to better determine the important amino acids required for Anti-Salmonella activity of P1 and P2. Alanine scanning libraries of P1 and P2 were synthesized from Genscript (Table 28; (www.genscript.com/alanine_scanning.html) and tested for inhibitory activity as described above. Relative importance [(percent growth in analogue-percent growth in original peptide)/(percent growth in DMSO-treated control-percent growth in original peptide)×100] was used to identify crucial amino acids of each amino acid residue of the peptide. Two independent experiments were conducted to ensure accuracy.

To analyze the perceived efficacy of arginine and lysine substitutes reported in previous studies, specific amino acid residues in P1 and P2 were replaced with lysine or arginine to test if the alliterations enhanced anti-Salmonella (ST and SE) activity. The MICs of the arginine or lysine substituted peptides (NPRRQERR (SEQ ID NO:37), NPSRRERR (SEQ ID NO:38), NPRRRERR (SEQ ID NO:39), KDENK (SEQ ID NO:41), PDEKK (SEQ ID NO:42), KDEKK (SEQ ID NO:43), KHTAPK (SEQ ID NO:45)) were determined as described early. Two independent experiments were conducted to ensure accuracy.

Mode of action of peptides analyzed through confocal microscopy: To determine the mode of action of P1 and P2, bacterial cytological profiling was conducted using confocal microscopy as described in. One hundred microliters of ST (5×10 CFU/mL) were treated with 5× MIC of P1 and P2 and then immediately incubated at 37° C. with 180 rpm shaking for 3 hours. Incubated bacteria cultures were then centrifuged and re-suspended in 100 μL sterile water. Following suspension, 1 μg/mL of FM4-64 (Invitrogen™ Molecular Probes™) and 5 μM Syto-9 (Invitrogen™ Molecular Probes™) stains were added and incubated for 45 mins at 4 with 150 rpm. After incubation, the samples were centrifuged, and bacteria was re-suspended in 10 μl sterile water. For microscopic analysis, 3 μL of bacterial suspension was added to glass slides with 1.2% agar and 20% LB media. Untreated ST bacterial culture was used as a control. Leica TCS SP6 confocal scanning microscope (Excitation/emission (nm); FM4-64 (515/640), SYTO-9 (485/498) was used at the Ohio State University's Molecular and Cellular Imaging Center (MCIC; mcic.osu.edu/microscopy).

Transmission electron microscopy: To further visually analyze the inhibitory effects of P1 and P2 on ST, transmission electron microscopy (TEM) was performed as described previously. Briefly, 500 μl of ST (1×10⁹ CFU) were treated with 10×MIC of peptides and incubated as described above (37° C. with 180 rpm shaking for 3 hours). After which, cells were fixed overnight with 3% glutaraldehyde and 2% paraformaldehyde at room temperature. Cells were then washed and postfixed with 1% osmium tetroxide (OsO₄). The washed cells were then dehydrated, with graded ethanol, and embedded with Embed 812 kit (Electron Microscopy Sciences, Pa). Ultrathin (70 nm) sections were prepared on formvar-carbon coated grids and stained with uranyl acetate and lead citrate. Untreated ST was used as a control. TEM was conducted at MCIC using the Hitachi H-7500 microscope.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
List of commensal and probiotic bacteria used in this study.

| Bacterial spp. | Media | Culture conditions | Reference/Source |
|---|---|---|---|
| Enterococcus faecalis | MRS broth | 37° C., anaerobic, | David Francis, SDSU |
| | | 16-18 h | |
| Streptococcus bovis | MRS broth | 37° C., anaerobic, | David Francis, SDSU |
| | | 16-18 h | |
| Levilactobacillus brevis | MRS broth | 37° C., anaerobic, | David Francis, SDSU |
| | | 1-2 days | |
| Lactobacillus acidophilus | MRS broth | 37° C., anaerobic, | David Francis, SDSU |
| | | 1-2 days | |
| Lacticaseibacillus rhamnosus GG | MRS broth | 37° C., anaerobic, | ATCC, Manassas, VA, USA |
| | | 1-2 days | |
| Bifidobacterium longum | MRS broth + | 37° C., anaerobic, | David Francis, SDSU |
| | 0.05% Cysteine | 24 h | |
| Bifidobacterium adolescentis | MRS broth + | 37° C., anaerobic, | David Francis, SDSU |
| | 0.05% Cysteine | 24 h | |
| Bifidobacterium lactis Bb12 | MRS broth + | 37° C., anaerobic, | Christian Hansen Ltd., |
| | 0.05% Cysteine | 24 h | Hørsholm, Denmark |
| Escherichia coli Nissle 1917 | LB broth | 37° C., aerobic, | Dr. Ulrich Sonnenborn, |
| | | 10-12 h, 200 rpm | Ardeypharm GmbH, |
| | | | Herdecke, Germany |
| Escherichia coli G58-1 | LB broth | 37° C., aerobic, | David Francis, SDSU |
| | | 10-12 h, 200 rpm | |
| Bacteroides thetaiotaomicron | MRS broth | 37° C., anaerobic, | David Francis, SDSU |
| | | 4-5 days | |

TABLE 2
Zone of inhibition induced by commensal
and probiotic bacteria against APEC O78.

| | Zone of inhibition (mm ± SD) | |
|---|---|---|
| Bacterial spp. | 12 h | 24 h |
| Enterococcus faecalis | 9.5 ± 0.5 | 0.0 ± 0.0 |
| Streptococcus bovis | 0.0 ± 0.0 | 0.0 ± 0.0 |
| Levilactobacillus brevis | 9.5 ± 0.5 | 0.0 ± 0.0 |
| Lactobacillus acidophilus | 0.0 ± 0.0 | 0.0 ± 0.0 |
| Lacticaseibacillus rhamnosus GG | 14.5 ± 0.5 | 12.5 ± 0.5 |
| Bifidobacterium longum | 0.0 ± 0.0 | 0.0 ± 0.0 |
| Bifidobacterium adolescentis | 12.5 ± 0.5 | 0.0 ± 0.0 |
| Bifidobacterium lactis Bb12 | 13.5 ± 0.5 | 11.5 ± 0.5 |
| Escherichia coli Nissle 1917 | 0.0 ± 0.0 | 0.0 ± 0.0 |
| Escherichia coli G58-1 | 0.0 ± 0.0 | 0.0 ± 0.0 |
| Bacteroides thetaiotaomicron | 9.5 ± 0.5 | 0.0 ± 0.0 |

TABLE 3
Anti-APEC activity of L. rhamnosus GG and
B. lactis Bb12 cell-free supernatants (CFSs).

| | Zone of inhibition (mm ± SD) | | | |
|---|---|---|---|---|
| | | | | <3 kDa |
| | CFS^{†} | 121° C. | Proteinase K | filtrate |
| L. rhamnosus GG | 13.0 ± 0.5 | 12.5 ± 0.5 | 12.0 ± 0.0 | 13.0 ± 0.0 |
| B. lactis Bb12 | 13.0 ± 1.0 | 13.5 ± 0.5 | 11.5 ± 0.5 | 13.5 ± 0.5 |

TABLE 4
pH of co-culture media in presence of L. rhamnosus GG,
B. lactis Bb12, L. acidophilus, and L. brevis.

| | pH (mean ± SD) | | | |
|---|---|---|---|---|
| | 12 h | 24 h | 36 h | 48 h |
| L. rhamnosus GG | 4.47 ± 0.06 | 4.37 ± 0.06 | 4.39 ± 0.01 | 4.35 ± 0.03 |
| B. lactis Bb12 | 4.82 ± 0.06 | 4.12 ± 0.01 | 4.09 ± 0.01 | 4.11 ± 0.01 |
| L. acidophilus | 5.42 ± 0.03 | 4.66 ± 0.02 | 4.54 ± 0.01 | 4.43 ± 0.06 |
| L. brevis | 5.12 ± 0.07 | 4.96 ± 0.04 | 4.83 ± 0.03 | 4.74 ± 0.04 |

TABLE 5
List of peptides identified in L. rhamnosus
GG and B. lactis Bb12 CFSs using LC-MS/MS.

| | Theo. | Accession |
|---|---|---|
| Sequence | MH+ [Da] | number |
| EVKALAEKVLKK | 1355.86 | A0A0R2DJY6 |
| SAVALSAVALSKPGHVNA | 1691.94 | C2JZA7 |
| AVALSAVALSKPGHVNA | 1604.91 | C2JZA7 |
| VALSAVALSKPGHVNA | 1533.87 | C2JZA7 |
| FSAVALSAVALSKPGHVNA* | 1839.01 | C2JZA7 |
| VAGVTLASASTLDKDIKD | 1803.97 | C2JYJ6 |
| LKDVLSSYLSTSSSSSTSK | 1977.00 | A0A180C684 |
| ALSAVALSKPGHVNA | 1434.81 | C2JZA7 |
| AQNGNTNKIEVDNIVYK | 1919.98 | A0A179YFC2 |
| VAGVTLASASTLDKDVKE | 1803.97 | A0A0R2DLD3 |
| VIVVVAAIGGGLNNKGKSSS | 1870.08 | A0A179YAS6 |
| DEVKALAEKVLKK | 1470.89 | A0A0R2DJY6 |
| GNDTPADSAVKARIV | 1513.80 | K8QAJ2 |
| HDVIQNALNAK | 1222.65 | A0A249DEL5 |
| LSSYLSTSSSSSTSK | 1521.73 | A0A180C684 |
| FSQATNAYFIKGA | 1417.71 | A0A2A5L4H0 |
| AADKSQVKVGVLQL | 1455.85 | C2K1D8 |
| AESSDTNLVNAKAA* | 1390.68 | A0A179YN16 |
| ATLAGVGVSGFAATTVHA | 1629.86 | A0A179XCY0 |
| ALDVDGIIAQLKDA | 1441.79 | A0A0H0YQJ8 |
| VQAAQAGDTKPIEV*† | 1426.75 | A0A179YFC2 |
| VNAAQNGNTNKIEVDNIVYK | 2204.13 | A0A179YFC2 |
| VNAAQNGNTNKIEVDNI | 1813.90 | A0A179YFC2 |
| SINRDDYNKAVSDGQDKL | 2037.98 | A0A2A5L4H0 |
| QSQFAQEQSEAAKATQA | 1822.86 | A0A179YJG3 |
| AFDNTDTSLDSTFKSA*† | 1719.77 | A0A180C684 |
| AIAAITDTMKKEGLAE | 1661.88 | K0N9I2 |
| DANKIKEQLEEVGATVTLK | 2086.14 | A0A0H0YQJ8 |
| DTSGKAGTTKISNV | 1378.72 | A0A1Z2F669 |
| EVASKTNDIAGDGTTTA | 1650.78 | A0A0R1WMV7 |
| GLALITAVPQVVRA | 1407.87 | A0A179Y5L8 |
| VTDTSGKAGTTKISNV*† | 1578.83 | A0A1Z2F669 |
| NKVGPKEYIPELNKSL | 1829.02 | A0A179YFC2 |

Table 5 shows the LC-MS/MS analysis of cell-free supernatants (CFSs) identifying 33 peptides of molecular weight less than 3 kDa. These peptides were found in common in both Higher Energy Collision Dissociation (HCD) and Ion-trap-based Collision-induced Dissociation (CID) setting. Table 5 shows the sequences to SEQ ID NOS: 1-33. SEQ ID NO: 1 is EVKALAEKVLKK. SEQ ID NO: 2 is SAVALSAVALSKPGHVNA. SEQ ID NO: 3 is AVALSAVALSKPGHVNA. SEQ ID NO: 4 is VALSAVALSKPGHVNA. SEQ ID NO: 5 is FSAVALSAVALSKPGHVNA*. SEQ ID NO: 6 is VAGVTLASASTLDKDIKD. SEQ ID NO: 7 is LKDVLSSYLSTSSSSSTSK. SEQ ID NO: 8 is ALSAVALSKPGHVNA. SEQ ID NO: 9 is AQNGNTNKIEVDNIVYK. SEQ ID NO: 10 is VAGVTLASASTLDKDVKE. SEQ ID NO: 11 is VIVVVAAIGGGLNNKGKSSS. SEQ ID NO: 12 is DEVKALAEKVLKK. SEQ ID NO: 13 is GNDTPADSAVKARIV. SEQ ID NO: 14 is HDVIQNALNAK. SEQ ID NO: 15 is LSSYLSTSSSSSTSK. SEQ ID NO: 16 is FSQATNAYFIKGA. SEQ ID NO: 17 is AADKSQVKVGVLQL. SEQ ID NO: 18 is AESSDTNLVNAKAA*. SEQ ID NO: 19 is ATLAGVGVSGFAATTVHA. SEQ ID NO: 20 is ALDVDGIIAQLKDA. SEQ ID NO: 21 is VQAAQAGDTKPIEV*t. SEQ ID NO: 22 is VNAAQNGNTNKIEVDNIVYK. SEQ ID NO: 23 is VNAAQNGNTNKIEVDNI. SEQ ID NO: 24 is SINRDDYNKAVSDGQDKL. SEQ ID NO: 25 is QSQFAQEQSEAAKATQA. SEQ ID NO: 26 is AFDNTDTSLDSTFKSA*t. SEQ ID NO: 27 is AIAAITDTMKKEGLAE. SEQ ID NO: 28 is DANKIKEQLEEVGATVTLK. SEQ ID NO: 29 is DTSGKAGTTKISNV. SEQ ID NO: 30 is EVASKTNDIAGDGTTTA. SEQ ID NO: 31 is GLALITAVPQVVRA. SEQ ID NO: 32 is VTDTSGKAGTTKISNV*t. SEQ ID NO: 33 is NKVGPKEYIPELNKSL.

TABLE 6
Relative abundance (%) of bacteria at the
genus level in different treatment groups.

| | Relative abundance (%) | | |
|---|---|---|---|
| | | L. rhamnosus | |
| Genus | NC | GG | PC |
| Akkermansia | 0.00 | 0.09 | 0.00 |
| Escherichia-Shigella | 10.16 | 4.20* | 16.45 |
| [Clostridium] innocuum group | 0.00 | 0.06 | 0.04 |
| Erysipelatoclostridium | 5.13 | 12.94* | 3.60 |
| [Eubacterium]coprostanolingenes group | 0.99 | 1.36 | 1.34 |
| Subdoligranulum | 0.00 | 0.52 | 0.00 |
| Ruminococcus 1 | 0.00 | 0.22 | 0.00 |
| Ruminococcaceae UCG-014 | 0.00 | 0.04 | 0.00 |
| Ruminiclostridium 9 | 3.46 | 5.02 | 7.73 |
| Ruminiclostridium 5 | 0.35 | 1.10 | 0.48 |
| Oscillibacter | 0.49 | 0.17 | 0.48 |
| Negativibacillus | 0.00 | 1.54*^{$} | 0.00 |
| Flavonifractor | 2.68 | 2.24* | 6.73^{≠} |
| DTU089 | 0.00 | 1.02*^{$} | 0.00 |
| Caproiciproducens | 0.77 | 0.28 | 0.05^{≠} |
| Candidatus Soleaferrea | 1.31 | 0.76 | 0.34^{≠} |
| Butyricicoccus | 1.17 | 2.45* | 1.08 |
| Anaerotruncus | 2.03 | 4.93 | 2.33 |
| Clostridtoides | 0.93 | 0.14 | 0.00 |
| Lachnospiraceae (uncultured) | 0.00 | 0.00* | 4.5^{≠} |
| [Ruminococcus] torques group | 24.38 | 18.19 | 23.13 |
| [Ruminococcus] gauvreauil group | 0.00 | 1.57 | 0.09 |
| Sellimonas | 0.77 | 0.27 | 4.04 |
| Lachnoclostridium | 0.00 | 1.11 | 0.00 |
| Blautia | 5.97 | 2.10* | 0.00 |
| Clostridium sensu stricto 1 | 0.00 | 0.64 | 0.90 |
| Lactobacillus | 0.75 | 0.56* | 0.03 |
| Enterococcus | 1.21 | 0.09* | 1.03 |
| Paenibacillus | 0.00 | 0.00 | 2.19 |
| Bacillus | 0.00 | 3.61^{$} | 2.56 |

TABLE 7
L. rhamnosus GG-specific
primers used in this study.

| Primer† | Sequence (5′ to 3′) |
|---|---|
| Lrhamn1 | CAATCTGAATGAACAGTTGTC |
| Lrhamn2 | TATCTTGACCAAACTTGACG |

Table 7 shows the Lacticaseibacillus rhamnosus GG (LGG)-specific primers obtained from Integrated DNA Technologies (IDT). These primers were used for quantitative polymerase chain reaction (qPCR) to detect the presence of LGG in the cecum of LGG-treated chickens. Table 7 shows the sequences to SEQ ID NO: 34-35. SEQ ID NO: 34 is CAATCTGAATGAACAGTTGTC. SEQ ID NO: 35 is TATCTTGACCAAACTTGACG.

TABLE 8
Zone of inhibition of L. rhamnosus GG and B. lactis
Bb12 against other predominant APEC serotypes.

Zone of inhibition (mm)

| | L. rhamnosus GG | B. lactis Bb12 |
|---|---|---|
| O78 | 14 | 13 |
| O1 | 15 | 14 |
| O2 | 15 | 13 |
| O8 | 15 | 12 |
| O15 | 15 | 14 |
| O18 | 15 | 12 |
| O35 | 16 | 14 |
| O109 | 15 | 13 |
| O115 | 18 | 12 |

TABLE 9
The effect of the peptides on the biofilm embedded and intracellular APEC.

| | Efficacy (Log CFU/mL)^{a} | | | | |
|---|---|---|---|---|---|
| | ^{b}Biofilm-embedded | Intracellular APEC | | | |
| Peptide | APEC | 0.25 X | 0.75 X | ^{c}1 X | 1.5 X |
| PN3 | 4.21 ± 0.48 | 2.64 ± 0.23 | 2.59 ± 0.30 | 2.50 ± 0.17 | 0.00 ± 0.00 |
| PN5 | 4.95 ± 0.07 | 2.34 ± 0.11 | 2.30 ± 0.00 | 0.00 ± 0.00 | 0.00 ± 0.00 |
| Untreated | 7.71 ± 0.72 | 2.98 ± 0.87 | 2.98 ± 0.87 | 2.98 ± 0.87 | 2.98 ± 0.87 |

TABLE 10
Zone of inhibition elicited by whole culture and temperature
treated probiotics in agar-well diffusion assay.

| Probiotic | | | | | | |
|---|---|---|---|---|---|---|
| Bacteria | Whole Culture | | 121° C. culture | | 4° C. culture | |
| Candidates | 12 h | 24 h | 12 h | 24 h | 12 h | 24 h |
| L. acidophilus | 14.0 ± 0.5 | 11.5 ± 0.0 | 13 ± 0.5 | 11.5 ± 0.5 | 14 ± 0.5 | 11.5 ± 0.0 |
| L. rhamnosus | 15.5 ± 0.5 | 13 ± 0.5 | 12 ± 0.0 | 10 ± 0.5 | 15.5 ± 0.5 | 14 ± 0.0 |
| GG | | | | | | |
| B. lactis Bb12 | 16.0 ± 0.5 | 14.5 ± 0.25 | 13.5 ± 0.5 | 11 ± 0.25 | 16 ± 0.5 | 14 ± 0.5 |
| E. coli Nissle | 0.0 ± 0.0 | 0.0 ± 0.0 | — | — | — | — |
| 1917* | | | | | | |
| L. brevis | 10.0 ± 0.0 | 0.0 ± 0.0 | 0.0 ± 0.0 | 0.0 ± 0.0 | 9.5 ± 0.5 | 0.0 ± 0.0 |

TABLE 11
pH of probiotic and Salmonella in co-culture media over time.

| | Average pH | | | |
|---|---|---|---|---|
| | 0 h | 6 h | 12 h | 24 h |
| ST-LA | 6.84 ± 0.06 | 6.18 ± 0.09 | 4.92 ± 0.15 | 4.57 ± 0.10 |
| ST-LGG | 6.81 ± 0.10 | 5.91 ± 0.30 | 4.65 ± 0.16 | 4.29 ± 0.13 |
| ST-BB12 | 6.85 ± 0.06 | 5.69 ± 0.31 | 4.50 ± 0.13 | 4.20 ± 0.03 |
| ST-Lbrev | 6.85 ± 0.03 | 5.53 ± 0.13 | 4.98 ± 0.11 | 4.79 ± 0.10 |
| ST | 6.90 ± 0.04 | 6.70 ± 0.27 | 5.48 ± 0.33 | 4.91 ± 0.17 |
| SE-LA | 6.70 ± 0.28 | 5.96 ± 0.11 | 4.94 ± 0.29 | 4.60 ± 0.17 |
| SE-LGG | 6.72 ± 0.26 | 5.23 ± 0.15 | 4.40 ± 0.00 | 4.36 ± 0.10 |
| SE-BB12 | 6.69 ± 0.27 | 5.15 ± 0.26 | 4.36 ± 0.08 | 4.30 ± 0.09 |
| SE-Lbrev | 6.76 ± 0.16 | 5.26 ± 0.13 | 5.06 ± 0.05 | 4.88 ± 0.07 |
| SE | 6.75 ± 0.27 | 6.61 ± 0.41 | 5.91 ± 0.29 | 5.42 ± 0.04 |

TABLE 12
Zone of inhibition elicited by treated cell free supernatants (CFS) against ST in diffusion assay.

| Probiotic | | | | | | | | | | |
|---|---|---|---|---|---|---|---|---|---|---|
| Bacteria | CFS | | 121° C. CFS | | 4° C. CFS | | Proteinase K | | <3 kDa filtrate | |
| Candidates | 12 h | 24 h | 12 h | 24 h | 12 h | 24 h | 12 h | 24 h | 12 h | 24 h |
| LGG | 12.5 ± 0.0 | 9 ± 1.0 | 13 ± 0.0 | 9 ± 0.5 | 12.5 ± 0.5 | 9 ± 1 | 15 ± 0.0 | 12 ± 0.0 | 12.5 ± 0.0 | 9 ± 0.5 |
| BB12 | 12.5 ± 0.0 | 9 ± 0.5 | 12.5 ± 0.0 | 9.5 ± 0.5 | 12 ± 0.0 | 9.5 ± 0.5 | 13 ± 0.0 | 11 ± 0.0 | 12.5 ± 0.0 | 9 ± 0.5 |

TABLE 13
Zone of inhibition elicited by proteolytic treated
CFS against SE in agar well diffusion assay.

| Probiotic Bacteria Candidates | Proteinase K |
|---|---|
| L. rhamnosus GG | 13 ± 0.5 |
| B. lactis Bb12 | 11.5 ± 0.1 |

TABLE 14
Anti-Salmonella activity LGG against Salmonella
serovars in agar well diffusion assay.

| Salmonella serotype | 24 h zone of inhibition (mm ± SD) |
|---|---|
| S. Anatum | 14 ± 0.0 |
| S. Albany | 14 ± 0.0 |
| S. Brenderup | 13 ± 0.50 |
| S. Javiana | 14 ± 0.00 |
| S. Heidelberg | 14 ± 0.50 |
| S. Muenchen | 17 ± 0.50 |
| S. Newport | 14 ± 0.00 |
| S. Saintpaul | 13 ± 0.00 |

TABLE 15
Probiotic derived peptides common
in HCD and CID LC-MS/MS analysis.

| | Synthesized |
|---|---|
| Peptide | denotation |
| EVKALAEKVLKK | |
| SAVALSAVALSKPGHVNA | |
| AVALSAVALSKPGHVNA | |
| VALSAVALSKPGHVNA | |
| FSAVALSAVALSKPGHVNA* | PN-1 |
| VAGVTLASASTLDKDIKD | |
| LKDVLSSYLSTSSSSSTSK | |
| ALSAVALSKPGHVNA | |
| AQNGNTNKIEVDNIVYK | |
| VAGVTLASASTLDKDVKE | |
| VIVVVAAIGGGLNNKGKSSS | |
| DEVKALAEKVLKK | |
| GNDTPADSAVKARIV | |
| HDVIQNALNAK | |
| LSSYLSTSSSSSTSK | |
| FSQATNAYFIKGA | |
| AADKSQVKVGVLQL | |
| AESSDTNLVNAKAA* | PN-2 |
| ATLAGVGVSGFAATTVHA | |
| ALDVDGIIAQLKDA | |
| VQAAQAGDTKPIEV* | PN-3 |
| VNAAQNGNTNKIEVDNIVYK | |
| VNAAQNGNTNKIEVDNI | |
| SINRDDYNKAVSDGQDKL | |
| QSQFAQEQSEAAKATQA | |
| AFDNTDTSLDSTFKSA* | PN-4 |
| AIAAITDTMKKEGLAE | |
| DANKIKEQLEEVGATVTLK | |
| DTSGKAGTTKISNV | |
| EVASKTNDIAGDGTTTA | |
| GLALITAVPQVVRA | |
| VTDTSGKAGTTKISNV* | PN-5 |
| NKVGPKEYIPELNKSL | |

Table 15 shows the five peptides selected for synthesis from the 33 peptides identified by HCD and CID. These five peptides were chosen based on their charge, hydrophobicity, and abundance in LGG and Bifidobacterium lactis (Bb12), which are considered to be strong anti-Salmonella characteristics. Table 15 shows the sequences to SEQ ID NOS: 1-33. SEQ ID NO: 1 is EVKALAEKVLKK. SEQ ID NO: 2 is SAVALSAVALSKPGHVNA. SEQ ID NO: 3 is AVALSAVALSKPGHVNA. SEQ ID NO: 4 is VALSAVALSKPGHVNA. SEQ ID NO: 5 is FSAVALSAVALSKPGHVNA*. SEQ ID NO: 6 is VAGVTLASASTLDKDIKD. SEQ ID NO: 7 is LKDVLSSYLSTSSSSSTSK. SEQ ID NO: 8 is ALSAVALSKPGHVNA. SEQ ID NO: 9 is AQNGNTNKIEVDNIVYK. SEQ ID NO: 10 is VAGVTLASASTLDKDVKE. SEQ ID NO: 11 is VIVVVAAIGGGLNNKGKSSS. SEQ ID NO: 12 is DEVKALAEKVLKK. SEQ ID NO: 13 is GNDTPADSAVKARIV. SEQ ID NO: 14 is HDVIQNALNAK. SEQ ID NO: 15 is LSSYLSTSSSSSTSK. SEQ ID NO: 16 is FSQATNAYFIKGA. SEQ ID NO: 17 is AADKSQVKVGVLQL. SEQ ID NO: 18 is AESSDTNLVNAKAA*. SEQ ID NO: 19 is ATLAGVGVSGFAATTVHA. SEQ ID NO: 20 is ALDVDGIIAQLKDA. SEQ ID NO: 21 is VQAAQAGDTKPIEV*t. SEQ ID NO: 22 is VNAAQNGNTNKIEVDNIVYK. SEQ ID NO: 23 is VNAAQNGNTNKIEVDNI. SEQ ID NO: 24 is SINRDDYNKAVSDGQDKL. SEQ ID NO: 25 is QSQFAQEQSEAAKATQA. SEQ ID NO: 26 is AFDNTDTSLDSTFKSA*t. SEQ ID NO: 27 is AIAAITDTMKKEGLAE. SEQ ID NO: 28 is DANKIKEQLEEVGATVTLK. SEQ ID NO: 29 is DTSGKAGTTKISNV. SEQ ID NO: 30 is EVASKTNDIAGDGTTTA. SEQ ID NO: 31 is GLALITAVPQVVRA. SEQ ID NO: 32 is VTDTSGKAGTTKISNV*t. SEQ ID NO: 33 is NKVGPKEYIPELNKSL.

TABLE 16
Efficacy of peptides against biofilm protected
and intracellular APEC O78 in macrophage cells.

| | Biofilm | | | |
|---|---|---|---|---|
| | protected APEC | Intracellular APEC (log CFU/mL) | | |
| Peptide | (log CFU/mL) | 12 mM | 15 mM | 18 mM |
| P-1 | 0.00 ± 0.00 | 3.57 ± 0.07 | 0.00 ± 0.00 | 0.00 ± 0.00 |
| P-2 | 0.00 ± 0.00 | 3.33 ± 0.03 | 0.00 ± 0.00 | 0.00 ± 0.00 |
| P-3 | 0.00 ± 0.00 | 3.62 ± 0.06 | 2.75 ± 0.31 | 0.00 ± 0.00 |
| Untreated | 7.54 ± 0.07 | 3.75 ± 0.10 | 3.75 ± 0.10 | 3.75 ± 0.10 |

TABLE 17
MICs of the arginine/lysine-
substituted peptide analogues.

| Peptide | MIC (mM) |
|---|---|
| NPSRQERR | 18 |
| NPRRQERR | 12 |
| NPSRRERR | 15 |
| NPRRRERR | 12 |
| PDENK | 12 |
| KDENK | 9 |
| PDEKK | 9 |
| KDEKK | 12 |
| VHTAPK | 18 |
| KHTAPK | >18 |
| VKTAPK | >18 |
| VHTKPK | >18 |
| KKTKPK | >18 |

Table 17 shows the impact of arginine and lysine substitutions on P1, P2, and P3 peptides on APEC activity. Arginine and lysine substitutions improved the anti-APEC activity of P1 and P2 peptides by decreasing the minimal inhibitory concentration (MIC) by 3 to 6 mM. Arginine and lysine substitutions of P3 peptide did not improve inhibitory effects. Table 17 shows the sequences to SEQ ID NOS: 36-48. SEQ ID NO: 36 is NPSRQERR. SEQ ID NO: 37 is NPRRQERR. SEQ ID NO: 38 is NPSRRERR. SEQ ID NO: 39 is NPRRRERR. SEQ ID NO: 40 is PDENK. SEQ ID NO: 41 is KDENK. SEQ ID NO: 42 is PDEKK. SEQ ID NO: 43 is KDEKK. SEQ ID NO: 44 is VHTAPK. SEQ ID NO: 45 is KHTAPK. SEQ ID NO: 46 is VKTAPK. SEQ ID NO: 47 is VHTKPK. SEQ ID NO: 48 is KKTKPK.

TABLE 18
Relative abundance of cecal bacteria at the genus
level in different groups treated with peptides.

| | Relative abundance (%) | | | | |
|---|---|---|---|---|---|
| Organism and dose | NC | P1 | P2 | P3 | PC |
| 50-mg/kg dose | | | | | |
| Escherichia-Shigella | 4.07 | 4.95 | 4.95 | 9.17 | 8.14 |
| Erysipelatoclostridium | 3.79 | 5.53^{a} | 3.84^{a} | 6.22 | 11.82 |
| Ruminiclostridium 9 | 2.25 | 0.00 | 1.25 | 2.14 | 2.80 |
| Ruminiclostridium 5 | 0.00 | 1.69 | 0.23 | 0.00 | 0.00 |
| Oscillibacter | 0.08 | 0.18 | 0.00 | 0.71 | 1.08 |
| Flavonifractor | 0.35 | 0.63 | 0.68 | 1.39 | 0.65 |
| Anaerotruncus | 0.00 | 0.73 | 0.00 | 0.00 | 0.00 |
| Clostridioides | 0.00 | 0.00 | 0.00 | 0.00 | 0.19 |
| Lachnospiraceae (uncultured) | 2.16 | 0.00 | 2.30 | 7.40 | 1.65 |
| Ruminococcus torques group | 8.45 | 7.08 | 9.83 | 13.82 | 6.10 |
| Ruminococcus gnavus group | 0.52 | 0.00 | 0.00 | 0.00 | 0.00 |
| Clostridium sensu stricto 1 | 0.00 | 0.00 | 0.00 | 0.77 | 0.34 |
| Pediococcus | 0.00 | 0.00 | 0.74 | 2.88 | 1.35 |
| Enterococcus | 2.47 | 0.24 | 1.24 | 1.92 | 0.34 |
| Bacteroides | 56.40 | 56.08 | 55.87 | 4.79^{a,b} | 38.09 |
| 100-mg/kg dose | | | | | |
| Escherichia-Shigella | 11.12 | 11.73 | 13.77 | 14.69 | 16.24 |
| Clostridium innocuum group | 2.03 | 0.00^{b} | 0.00^{b} | 0.00^{b} | 0.00^{c} |
| Erysipelatoclostridium | 24.65 | 17.11 | 14.71^{b} | 14.78^{b} | 12.68^{c} |
| Eubacterium coprostanoligenes group | 0.00 | 0.00 | 0.00 | 0.21 | 0.00 |
| UBA1819 | 0.00 | 0.00 | 0.68 | 0.00 | 0.00 |
| Ruminococcacede UCG-005 | 0.57 | 0.56 | 2.19 | 0.00 | 0.00 |
| Ruminiclostridium 9 | 0.00 | 0.00 | 0.78 | 1.52^{a,b} | 0.00 |
| Ruminiclostridium 5 | 0.00 | 0.00 | 0.28 | 0.00 | 0.00 |
| Oscillibacter | 0.00 | 0.00 | 0.13 | 0.76 | 0.00 |
| Flavonifractor | 1.92 | 2.40 | 0.68^{b} | 1.25 | 0.52^{c} |
| Caproiciproducens | 0.43 | 0.00 | 0.00 | 0.34 | 0.00 |
| Butyricicoccus | 2.04 | 0.44^{b} | 1.53 | 0.19^{b} | 0.60 |
| Anaerotruncus | 1.47 | 0.00 | 0.00 | 1.65 | 0.12 |
| Clostridioides | 0.00 | 0.21 | 0.00 | 0.73 | 0.00 |
| Lachnospiraceae (uncultured) | 2.38 | 1.85 | 0.73 | 10.38^{b} | 5.85 |
| Ruminococcus torques group | 9.31 | 7.62 | 10.37 | 8.44 | 10.93 |
| Sellimonas | 2.95 | 0.00^{b} | 0.00^{b} | 1.60^{a} | 0.13^{c} |
| Blautia | 0.00 | 0.00 | 0.00 | 1.07 | 0.00 |
| Clostridiales vadin BB60 group | 0.00 | 0.00 | 0.00 | 0.41^{a} | 0.00 |
| Clostridium sensu stricto 1 | 0.53 | 0.68 | 0.00 | 0.21 | 2.16 |
| Lactococcus | 2.18 | 0.00^{b} | 0.40 | 0.00 | 0.00^{c} |
| Weissella | 0.32 | 1.60 | 2.43^{a} | 0.00 | 0.40 |
| Pediococcus | 0.01 | 0.49 | 1.38^{b} | 0.00 | 0.68 |
| Lactobacillus | 0.00 | 0.00 | 0.00 | 19.62^{a,b} | 0.06 |
| Enterococcus | 5.37 | 4.99 | 2.01 | 2.14 | 3.16 |
| Paenibacillus | 0.27 | 0.00 | 0.00 | 0.00 | 0.00 |
| Bacillus | 1.74 | 2.64 | 0.37 | 0.42 | 2.98 |

TABLE 19
List of APEC serotypes/strains and beneficial microbes used in this study.

| Bacterial spp. | Media | Culture conditions | Source |
|---|---|---|---|
| APEC serotypes | | | |
| APEC O78 | LB broth | 37° C., aerobic, | Dr. Tim Johnson, UMN, MN |
| APEC O1 | | 12 h, 180 rpm | Dr. Tim Johnson, UMN, MN |
| APEC O2 | | | Dr. Tim Johnson, UMN, MN |
| APEC O1-63 | | | Drs. Lisa K Nolan, Catherine |
| | | | M Logue, UGA, GA |
| APEC O2-211 | | | Drs. Lisa K Nolan, Catherine |
| | | | M Logue, UGA, GA |
| APEC O78-53 | | | Drs. Lisa K Nolan, Catherine |
| | | | M Logue, UGA, GA |
| APEC O8 | | | Drs. Lisa K Nolan, Catherine |
| | | | M Logue, UGA, GA |
| APEC O15 | | | Drs. Lisa K Nolan, Catherine |
| | | | M Logue, UGA, GA |
| APEC O18 | | | Drs. Lisa K Nolan, Catherine |
| | | | M Logue, UGA, GA |
| APEC O35 | | | Drs. Lisa K Nolan, Catherine |
| | | | M Logue, UGA, GA |
| APEC O109 | | | Drs. Lisa K Nolan, Catherine |
| | | | M Logue, UGA, GA |
| APEC O115 | | | Drs. Lisa K Nolan, Catherine |
| | | | M Logue, UGA, GA |
| APEC O78-X7122 | | | Dr. Roy Curtiss, III, UF, FL |
| APEC O1-X7235 | | | Dr. Roy Curtiss, III, UF, FL |
| APEC O2-X7302 | | | Dr. Roy Curtiss, III, UF, FL |
| Beneficial microbes | | | |
| Enterococcus faecalis | MRS broth | 37° C., anaerobic, | David Francis, SDSU |
| | | 16-18 h | |
| Streptococcus bovis | MRS broth | 37° C., anaerobic, | David Francis, SDSU |
| | | 16-18 h | |
| Lactobacillus brevis | MRS broth | 37° C., anaerobic, | David Francis, SDSU |
| | | 1-2 days | |
| Lactobacillus acidophilus | MRS broth | 37° C., anaerobic, | David Francis, SDSU |
| | | 1-2 days | |
| Lactobacillus rhamnosus GG | MRS broth | 37° C., anaerobic, | ATCC, Manassas, VA, USA |
| | | 1-2 days | |
| Bifidobacterium longum | MRS broth + | 37° C., anaerobic, | David Francis, SDSU |
| | 0.05% Cysteine | 24 h | |
| Bifidobacterium adolescentis | MRS broth + | 37° C., anaerobic, | David Francis, SDSU |
| | 0.05% Cysteine | 24 h | |
| Bifidobacterium lactis Bb12 | MRS broth + | 37° C., anaerobic, | Christian Hansen Ltd., |
| | 0.05% Cysteine | 24 h | Hørsholm, Denmark |
| Escherichia coli Nissle 1917 | LB broth | 37° C., aerobic, | Dr. Ulrich Sonnenborn, Ardeypharm |
| | | 12 h, 180 rpm | GmbH, Herdecke, Germany |
| Escherichia coli G58-1 | LB broth | 37° C., aerobic, | David Francis, SDSU |
| | | 12 h, 180 rpm | |
| Bacteroides thetaiotaomicron | MRS broth | 37° C., anaerobic, | David Francis, SDSU |
| | | 4-5 days | |

TABLE 20
Primers used in this study.

| Primer | Sequence (5′ to 3′) |
|---|---|
| LptD_F | GCGATCAGAGCGACATCTATAA |
| LptD_R | TGGTTAGCGGAGGCAATAC |
| BamA_F | TCGGTGGTCGTCTCTTCTAT |
| BamA_R | CGTCACGTCTGTACCATAACTC |
| MlaA_F | TGATATGGCGGATGGTCTTTAC |
| MlaA_R | ACTGCGCACGAGTTTCTATC |
| MlaC_F | CTGCCATACGTACAGGTGAAA |
| MlaC_R | ACGGAAAGCGGCAAAGTA |
| LolB_F | AAGTGTACGCCCGTTTCTTC |
| LolB_R | GTTACCCGGTTGAGCATTCA |
| PbgA_F | TCAGCGTGCCGGTTATTT |
| PbgA_R | CGAGGCGATAAAGGCGATAA |
| OmpC_F | CTACACCGGTGGTCTGAAATA |
| OmpC_R | CCTACGCGAGTTGCGTTATAG |
| OmpF_F | ACACGACCAACGAAGAAGTC |
| OmpF_R | CCGTAACTACGGTGTGGTTTAT |
| GAPDH_F | CGGTACCGTTGAAGTGAAAGA |
| GAPDH_R | ACTTCGTCCCATTTCAGGTTAG |

Table 20 shows the primers selected to determined expression of genes essential for bacterial outer membrane integrity. Primers were designed using the PrimerQuest tool and obtained from Integrated DNA Technologies (IDT). Table 20 shows the sequences to SEQ ID NOS: 49-66. SEQ ID NO: 49 is GCGATCAGAGCGACATCTATAA. SEQ ID NO: 50 is TGGTTAGCGGAGGCAATAC. SEQ ID NO: 51 is TCGGTGGTCGTCTCTTCTAT. SEQ ID NO: 52 is CGTCACGTCTGTACCATAACTC. SEQ ID NO: 53 is TGATATGGCGGATGGTCTTTAC. SEQ ID NO: 54 is ACTGCGCACGAGTTTCTATC. SEQ ID NO: 55 is CTGCCATACGTACAGGTGAAA. SEQ ID NO: 56 is ACGGAAAGCGGCAAAGTA. SEQ ID NO: 57 is AAGTGTACGCCCGTTTCTTC. SEQ ID NO: 58 is GTTACCCGGTTGAGCATTCA. SEQ ID NO: 59 is TCAGCGTGCCGGTTATTT. SEQ ID NO: 60 is CGAGGCGATAAAGGCGATAA. SEQ ID NO: 61 is CTACACCGGTGGTCTGAAATA. SEQ ID NO: 62 is CCTACGCGAGTTGCGTTATAG. SEQ ID NO: 63 is ACACGACCAACGAAGAAGTC. SEQ ID NO: 64 is CCGTAACTACGGTGTGGTTTAT. SEQ ID NO: 65 is CGGTACCGTTGAAGTGAAAGA. SEQ ID NO: 66 is ACTTCGTCCCATTTCAGGTTAG.

TABLE 21
MIC₅₀ concentrations of peptides against APEC 078.

| Peptide | MIC₅₀ (mM) |
|---|---|
| P1 | 14.7 |
| P2 | 9.5 |
| P3 | 16.2 |
| P4 | 20.0 |

TABLE 22
Sequence of alanine analogues of peptides.

| Peptide | Sequence |
|---|---|
| P1-1 | APSRQERR |
| P1-2 | NASRQERR |
| P1-3 | NPARQERR |
| P1-4 | NPSAQERR |
| P1-5 | NPSRAERR |
| P1-6 | NPSRQARR |
| P1-7 | NPSRQEAR |
| P1-8 | NPSRQERA |
| P2-1 | ADENK |
| P2-2 | PAENK |
| P2-3 | PDANK |
| P2-4 | PDEAK |
| P2-5 | PDENA |
| P3-1 | AHTAPK |
| P3-2 | VATAPK |
| P3-3 | VHAAPK |
| P3-4 | VHTAAK |
| P3-5 | VHTAPA |

Table 22 shows the alanine analogues synthesized from peptides P1, P2, and P3 using alanine scanning libraries. These analogues were used to test anti-APEC activity. Table 22 shows the sequences to SEQ ID NOS: 67-84. SEQ ID NO: 67 is APSRQERR. SEQ ID NO: 68 is NASRQERR. SEQ ID NO: 69 is NPARQERR. SEQ ID NO: 70 is NPSAQERR. SEQ ID NO: 71 is NPSRAERR. SEQ ID NO: 72 is NPSRQARR. SEQ ID NO: 73 is NPSRQEAR. SEQ ID NO: 74 is NPSRQERA. SEQ ID NO: 75 is ADENK. SEQ ID NO: 76 is PAENK. SEQ ID NO: 77 is PDANK. SEQ ID NO: 78 is PDEAK. SEQ ID NO: 79 is PDENA. SEQ ID NO: 80 is AHTAPK. SEQ ID NO: 81 is VATAPK. SEQ ID NO: 82 is VHAAPK. SEQ ID NO: 83 is VHTAAK. SEQ ID NO: 84 is VHTAPA.

TABLE 23
Spectrum efficacy measured by MIC of
peptides against multiple serovars.

| Salmonella spp. | Peptide 1 | Peptide 2 | Peptide 4 |
|---|---|---|---|
| Salmonella Anatum | 18 mM | 15 mM | 18 mM |
| Salmonella Albany | 18 mM | 15 mM | 18 mM |
| Salmonella Brenderup | 18 mM | 15 mM | 18 mM |
| Salmonella Javiana | 18 mM | 15 mM | 18 mM |
| Salmonella Heidelberg | 18 mM | 15 mM | 18 mM |
| Salmonella Muenchen | 18 mM | 15 mM | 18 mM |
| Salmonella Newport | 18 mM | 15 mM | 18 mM |
| Salmonella Saintpaul | 18 mM | 15 mM | 18 mM |

TABLE 24
Effect of heat and protease treatment on anti-
Salmonella (ST and SE) activities of peptides.

| | MIC (mM) against ST | | | MIC (mM) against SE | | |
|---|---|---|---|---|---|---|
| | No | No | Heat | Protease | Heat | Protease |
| Peptide | treatment | treatment | treatment | treatment | treatment | treatment |
| P-1 | 18 | 18 | 18 | 18 | 18 | 18 |
| P-2 | 15 | 15 | 15 | 15 | 15 | 15 |
| P-4 | 18 | 18 | 18 | 18 | 18 | 18 |

TABLE 25
Inhibitory effect of peptides
against biofilm protected Salmonella.

| | Biofilm embedded ST | Biofilm embedded SE |
|---|---|---|
| Peptide | Log CFU/ml | Log CFU/mL |
| P1-NPSRQERR | 0.00 ± 0.00*** | 0.00 ± 0.00*** |
| P2-PDENK | 0.00 ± 0.00*** | 0.00 ± 0.00*** |
| P4-MLNERVK | 0.00 ± 0.00*** | 0.00 ± 0.00*** |
| Untreated | 6.46 ± 0.09 | 6.77 ± 0.02 |

Table 25 shows the inhibitory effects of peptides against Salmonella treated biofilm. Peptide P1, P2, and P4 eradicated wells containing biofilms protected with either Salmonella typhimurium (ST) or Salmonella Enteritidis (SE). Table 25 shows the sequences to SEQ ID NOS: 85, 86, and 88. SEQ TD NO: 85 is NPSRQERR. SEQ TD NO: 86 is PDEN. SEQ ID NO: 88 is MLNERVK.

TABLE 26
MICs of arginine/lysine
substituted peptide residues.

| | Salmonella | |
|---|---|---|
| Peptide | Challenge | MIC |
| NPSRQERR | ST | 18 mM |
| NPRRQERR | ST | 15 mM |
| NPSRRERR | ST | >18 mM |
| NPRRRERR | ST | 15 mM |
| NPSRQERR | SE | 18 mM |
| NPRRQERR | SE | 18 mM |
| NPSRRERR | SE | >18 mM |
| NPRRQRRR | SE | >18 mM |
| PDENK | ST | 15 mM |
| KDENK | ST | 12 mM |
| PDEKK | ST | 15 mM |
| KDEKK | ST | 15 mM |
| PDENK | SE | 15 mM |
| KDENK | SE | 12 mM |
| PDEKK | SE | 12 mM |
| KDEKK | SE | 15 mM |

Table 26 shows the impact of arginine and lysine substitutions on P1 and P2 peptides on Salmonella activity. Lysine replacement improved the efficacy of peptide P2 enhancing anti-Salmonella activity against ST and SE. Table 26 shows the sequences to SEQ ID NOS: 89-104. SEQ ID NO: 89 is NPSRQERR. SEQ ID NO: 90 is NPRRQERR. SEQ ID NO: 91 is NPSRRERR. SEQ ID NO: 92 is NPRRRERR. SEQ ID NO: 93 is NPSRQERR. SEQ ID NO: 94 is NPRRQERR. SEQ ID NO: 95 is NPSRRERR. SEQ ID NO: 96 is NPRRQRRR. SEQ ID NO: 97 is PDENK. SEQ ID NO: 98 is KDENK. SEQ ID NO: 99 is PDEKK. SEQ ID NO: 100 is KDEKK. SEQ ID NO: 101 is PDENK. SEQ ID NO: 102 is KDENK. SEQ ID NO: 103 is PDEKK. SEQ ID NO: 104 is KDEKK.

TABLE 27
Bacteria, source, and growing conditions of bacteria used in this study.

| Bacteria | Growing Conditions | Source |
|---|---|---|
| S. Typhimurium (nalidixic | LB broth, aerobic 37° C. | Dr. John Gunn, |
| acid resistant) | for 18-24 hours | Ohio State University |
| S. Enteritidis (nalidixic | | Laboratory Collection |
| acid resistant) | | |
| S. Anatum | | Laboratory Collection |
| S. Albany | | Laboratory Collection |
| S. Brenderup | | Laboratory Collection |
| S. Javiana | | Laboratory Collection |
| S. Heidelberg | | Laboratory Collection |
| S. Muenchen | | Laboratory Collection |
| S. Newport | | Laboratory Collection |
| S. Saintpaul | | Laboratory Collection |
| Enterococcus faecalis | MRS broth, 37^{a}° C., anaerobic | David Francis, SDSU |
| | for 16-18 hours | |
| Streptococcus bovis | MRS broth, 37° C., anaerobic | David Francis, SDSU |
| | for 16-18 hours | |
| Lactobacillus brevis | MRS broth, 37° C., anaerobic | David Francis, SDSU |
| | for 1-2 days | |
| Lactobacillus acidophilus | MRS broth, 37° C., anaerobic | David Francis, SDSU |
| | for 24 hours | |
| Lactobacillus rhamnosus GG | MRS broth, 37° C., anaerobic | ATCC, USA |
| | for 24 hours | |
| Bifidobacterlum longum | MRS broth with 0.05% | David Francis, SDSU |
| | cysteine, 37° C., anaerobic | |
| | for 24 hours | |
| Bifidobacterium adolescentis | MRS broth with 0.05% | David Francis, SDSU |
| | cysteine, 37° C., anaerobic | |
| | for 24 hours | |
| Bifidobacterium lactis Bb12 | MRS broth with 0.05% | Christian Hansen, Ltd, |
| | cysteine, 37° C., anaerobic | Hørsholm, Denmark |
| | for 24 hours | |
| Escherichia coli Nissle 1917 | LB broth, aerobic 37° C. for | Dr. Ulrich Sonnenborn, |
| | 12-18 hours, 180 rpm | Ardeypharm GmbH, |
| | | Herdecke, Germany |
| Escherichia coli G58-1 | LB broth, aerobic 37° C. for | David Francis, SDSU |
| | 12-18 hours, 180 rpm | |
| Bacteroides thetaiotaomicron | MRS broth, 37° C., anaerobic | David Francis, SDSU |
| | for 4-5 days | |

TABLE 28
Peptide sequence with amino acids
substituted in alanine scanning analysis.

| Peptide | Sequence | Amino acid substituted |
|---|---|---|
| P1-1 | APSRQERR | Asparagine |
| P1-2 | NASRQERR | Proline |
| P1-3 | NPARQERR | Serine |
| P1-4 | NPSAQERR | Arginine |
| P1-5 | NPSRAERR | Glutamine |
| P1-6 | NPSRQARR | Glutamic acid |
| P1-7 | NPSRQEAR | Arginine |
| P1-8 | NPSRQERA | Arginine |
| P2-1 | ADENK | Proline |
| P2-2 | PAENK | Aspartate |
| P2-3 | PDANK | Glutamic acid |
| P2-4 | PDEAK | Asparagine |
| P2-5 | PDENA | Lysine |

Table 28 shows the alanine analogues synthesized from peptides P1, P2, and P3 using alanine scanning libraries. These analogues were used to test anti-Salmonella activity. Table 26 shows the sequences to SEQ ID NOS: 105-117. SEQ ID NO: 105 is APSRQERR. SEQ ID NO: 106 is NASRQERR. SEQ ID NO: 107 is NPARQERR. SEQ ID NO: 108 is NPSAQERR. SEQ ID NO: 10⁹ is NPSRAERR. SEQ ID NO: 110 is NPSRQARR. SEQ ID NO: 111 is NPSRQEAR. SEQ ID NO: 112 is NPSRQERA. SEQ ID NO: 113 is ADENK. SEQ ID NO: 114 is PAENK. SEQ ID NO: 115 is PDANK. SEQ ID NO: 116 is PDEAK. SEQ ID NO: 117 is PDENA.

TABLE 29
Relative abundance of cecal microbial community
at the genus level by treatment group.

| | Relative Abundance (%) | | | | |
|---|---|---|---|---|---|
| Genus | NC | P1 | P2 | P4 | PC |
| Enterococcus | 0.00 | 3.32 | 0.00 | 0.00 | 0.57 |
| Lactobacillus | 1.02 | 7.00 | 5.57 | 7.01 | 2.92 |
| Pediococcus | 0.00 | 0.00 | 0.00 | 0.75 | 0.00 |
| Clostridium sensu stricto 1 | 0.00 | 0.00 | 0.00 | 0.00 | 0.12 |
| Blautia | 0.00 | 0.00 | 0.00 | 1.60 | 0.00 |
| Epulopiscium | 0.00 | 0.00 | 0.17 | 0.00 | 0.00 |
| Lachnospiraceae NK4A136 group | 0.00 | 0.98 | 0.00 | 0.53 | 0.00 |
| Sellimonas | 2.63 | 2.68 | 1.76 | 0.57 | 2.50 |
| [Ruminococcus] torques group | 22.35 | 9.83 | 23.19 | 19.27 | 20.02 |
| Lachnospiraceae (uncultured) | 0.37 | 8.62 | 4.21 | *11.71 | *4.91 |
| Lachnospiraceae (unidentified) | 27.22 | 34.16 | 37.10 | 44.19 | 35.66 |
| Anaerotruncus | 0.00 | 0.00 | 0.00 | 0.48 | 1.83 |
| Caproiciproducens | 0.00 | 0.00 | 0.63 | 0.00 | 0.00 |
| Flavonifractor | 4.85 | 4.40 | 2.33 | 1.20 | 2.27 |
| Oscillibacter | 4.30 | 2.62 | 0.00 | 1.35 | 1.03 |
| Ruminiclostridium 5 | 1.31 | 0.00 | 0.00 | 0.00 | 0.00 |
| Ruminiclostridium 9 | 0.00† | 0.00† | 0.00† | 0.13† | 1.50 |
| [Eubacterium] coprostanoligenes group | 0.15 | 0.00 | 0.00 | 0.00 | 0.00 |
| Erysipelatoclostridium | 13.30 | 15.34 | 5.56 | 3.51 | 10.03 |
| [Clostridium] innocuum group | 0.00 | 0.00 | 0.00 | 0.00 | 0.08 |
| Escherichia-Shigella | 6.05 | 4.80† | 13.25 | 3.16† | 11.61 |
| Enterobacteriaceae (unidentified) | 16.46 | 6.26 | 6.22 | 4.54 | 4.87 |
| Acinetobacter | 0.00 | 0.00 | 0.00 | 0.00 | 0.08 |

1. A composition comprising: an antimicrobial peptide comprising an amino acid sequence at least 80% identical to SEQ ID NO:21 or SEQ ID NO:32, or a functional variant thereof; and a pharmaceutically acceptable carrier.

2. The composition of claim 1, wherein the antimicrobial peptide comprises an amino acid sequence at least 90% identical to SEQ ID NO:21 or SEQ ID NO:32.

3. The composition of claim 1, wherein the antimicrobial peptide comprises SEQ ID NO: 21.

4. The composition of claim 1, wherein the antimicrobial peptide comprises SEQ ID NO: 32

5. The composition of claim 1, wherein the antimicrobial peptide is 10-20 amino acids in length.

6. A method of treating or preventing a bacterial infection in a subject comprising administering to the subject an effective amount of an antimicrobial peptide comprising an amino acid sequence at least 80% identical to SEQ ID NO:21, SEQ ID NO:32, SEQ ID NO:85, SEQ ID NO:86, or SEQ ID NO:87, or a functional variant thereof.

7. The method of claim 6, wherein the antimicrobial peptide comprises an amino acid sequence at least 90% identical to SEQ ID NO:21, SEQ ID NO:32, SEQ ID NO:85, SEQ ID NO:86, or SEQ ID NO:87.

8. The method of claim 6, wherein the antimicrobial peptide comprises SEQ ID NO: 21.

9. The method of claim 6, wherein the antimicrobial peptide comprises SEQ ID NO: 32.

10. The method of claim 6, wherein the antimicrobial peptide comprises SEQ ID NO: 85.

11. The method of claim 6, wherein the antimicrobial peptide comprises SEQ ID NO: 86.

12. The method of claim 6, wherein the antimicrobial peptide comprises SEQ ID NO: 87.

13. The method of claim 6, wherein the antimicrobial peptide is administered alone or in combination with an additional antimicrobial peptide.

14. The method of claim 6, wherein the antimicrobial peptide is administered in combination with an antibiotic therapy.

15. The method of claim 14, wherein the antibiotic therapy is selected from a tetracycline, a sulfonamide, an aminoglycoside, a quinolone, or a β-lactam.

16. The method of claim 6, wherein the bacterial infection is caused by a bacterial overgrowth.

17. The method of claim 6, wherein the antimicrobial peptide targets a bacterial membrane.

18. The method of claim 17, wherein the bacterial membrane is an outer membrane.

19. The method of claim 17, wherein the bacterial membrane comprises a MlaA-OmpC/F protein system.

20. The method of claim 6, wherein the bacterial infection is caused by an Escherichia coli (E. coli) bacterium.

21. The method of claim 20, wherein the E. coli is an avian pathogenic E. coli (APEC).

22. The method of claim 6, wherein the bacterial infection is caused by a Salmonella bacterium.

23. The method of claim 6, wherein the subject is a chicken.
